(19) United States
(12) Patent Application Publication
(10) Pub. No.: US 2026/0237509 A1
(43) Pub. Date: Aug. 13, 2026

(54) METHODS TO DIAGNOSE, DETECT, OR ASSESS CANCER USING ISOTOPIC ELEMENTAL ANALYSIS

(71) Applicants: LUC Innovations AB, Lund (SE); The Johns Hopkins University, Baltimore, MD (US)

(72) Inventors: Emma Ulrika HAMMARLUND, Lund (SE); Per Kristian MALMBERG, Lund (SE); Kazi Julhash UDDIN, Lund (SE); Kenneth James PIENTA, Baltimore, MD (US)

(73) Assignees: LUC Innovations AB, Lund (SE); The Johns Hopkins University, Baltimore, MD (US)

(21) Appl. No.: 19/153,943

(22) PCT Filed: Feb. 16, 2024
(86) PCT No.: PCT/US2024/016304

§ 371 Date: Aug. 5, 2025

Related U.S. Application Data
(60) Provisional application No. 63/446,785, filed on Feb. 17, 2023.

(51) Int. Cl. G16H 50/20; H01J 49/00

(52) U.S. Cl. CPC G16H 50/20; H01J 49/0036

(57) ABSTRACT

Isotopic fingerprinting was adapted for the human body, and experimental data demonstrated a specific chemical fingerprint of cancer, e.g., prostate cancer, that was “fossilized” in samples such as hair strands or nails of patients. To interpret the isotopic chemical data of the samples, prediction models using machine learning algorithms were developed that allowed for the separation of populations using several dimensionals. Provided are noninvasive cancer diagnosis, detection, or assessment methods that were developed in some aspects by identifying the chemical fingerprint of cancer.

### CROSS-REFERENCE TO RELATED APPLICATIONS

This application claims priority to U.S. Provisional Patent Application No. 63/446,785, filed Feb. 17, 2023, the contents of which is herein incorporated by reference in its entirety for all purposes.

### FIELD

The present disclosure relates to noninvasive cancer diagnosis, detection, or assessment methods that were developed in some aspects, by identifying the chemical fingerprint of cancer. Isotopic fingerprinting was adapted for the human body, and experimental data demonstrated a specific chemical fingerprint of cancer, e.g., prostate cancer, that was “fossilized” in samples such as hair strands or nails of patients. To interpret the isotopic chemical data of the samples, prediction models, such as using machine learning algorithms, were developed that allowed for the separation of populations in a high-dimensional manner.

### BACKGROUND

Cancer is a leading cause of death worldwide, accounting for nearly 10 million deaths in 2020 [1]. Cancer mortality is reduced when cases are detected and treated early. There has been a great deal of interest into the research and development of technologies that promote the earlier detection and diagnosis of cancer. There are many approaches to diagnosing cancer, such as physical examination, laboratory tests, imaging, or by biopsy [2]. Each of these diagnostic techniques has unique pitfalls. The diagnostic may have poor sensitivity or accuracy, require invasive surgery, require expensive instrumentation, or be cost and time prohibitive. A noninvasive and accurate diagnostic would be a powerful tool in cancer screening and monitoring. For example, prostate cancer is typically screened by medical history and physical exam or a prostate-specific antigen (PSA) blood test [3]. Results of the prior tests (e.g., a high PSA) may then require a prostate biopsy for further verification [3]. Of the patients who obtain a biopsy, only about 25% are diagnosed with cancer, which reflects the poor sensitivity and specificity of PSA that results in hundreds of thousands of unnecessary and expensive invasive procedures each year [34].

Accurate noninvasive or minimally invasive diagnostic techniques have significant advantages and outweigh the limitations posed by invasive diagnostic procedures. That said, to date, no noninvasive diagnostic techniques have been introduced that reliably detect or assess cancer and are capable of identifying the type of cancer simultaneously (e.g., pancreatic cancer, colon cancer, etc.). There remains a continuing need for the development of a noninvasive cancer diagnostic technique.

### SUMMARY

Provided herein are methods for detecting, diagnosing, or assessing cancer in a subject. The provided methods are noninvasive and reliably detect or assess cancer in the subject. Thus, in some aspects, provided are noninvasive cancer diagnosis, detection, or assessment methods that were developed in some aspects by identifying the chemical fingerprint of cancer.

Provided herein in some embodiments is a method of detecting whether a subject has a cancer. Provided herein in other embodiments is a method of detecting a cancer in a subject.

In some of any embodiments, the method comprises determining the value for each of at least one isotopic feature for a sample from a subject. In some of any embodiments, the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject. In some of any embodiments, the at least one isotopic feature comprises the overall concentration of an isotopic element measured in the sample of the subject. In some of any embodiments, the at least one isotopic feature comprises a chemical feature determined from the overall concentration of an isotopic element measured in the sample of the subject. In some of any embodiments, the at least one isotopic feature comprises the concentration of an isotope of an isotopic element measured in the sample of the subject. In some of any embodiments, the at least one isotopic feature comprises the concentration of an isotope of an isotopic element measured in the sample of the subject. In some of any embodiments, the at least one isotopic feature comprises a concentration of an element, a concentration of an isotopic element, a concentration of an isotope of the isotopic element, or a normalized value of any thereof. In some of any embodiments, the at least one isotopic feature comprises a concentration of an element measured in the sample of the subject. In some of any embodiments, the at least one isotopic feature comprises a concentration of an isotopic element measured in the sample of the subject. In some of any embodiments, the at least one isotopic feature comprises a concentration of an isotope of the isotopic element measured in the sample of the subject. In some of any embodiments, the at least one isotopic feature comprises a normalized value of a concentration of an element measured in the sample of the subject. In some of any embodiments, the at least one isotopic feature comprises a normalized value of a concentration of an isotopic element measured in the sample of the subject. In some of any embodiments, the at least one isotopic feature comprises a normalized value of a concentration of an isotope of the isotopic element measured in the sample of the subject.

In some of any embodiments, the method comprises inputting the value for each of the at least one isotopic feature from the subject into a prediction model. In some of any embodiments, the prediction model is configured to predict the presence or absence of a cancer in a subject. In some of any embodiments, the prediction model comprises at least one machine learning algorithm trained using values for each of the at least one isotopic feature from a plurality of reference samples. In some of any embodiments, the plurality of reference samples are from a plurality of reference subjects.

In some of any embodiments, the method comprises determining, using the prediction model, whether the subject has the cancer. In some of any embodiments, the method comprises detecting, using the prediction model, the cancer in the subject.

Also provided herein in some embodiments is a method of detecting whether a subject has a cancer, the method comprising: (a) determining the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer.

Also provided herein in some embodiments is a method of detecting whether a subject has a cancer, the method comprising: (a) determining the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer. Also provided herein in some embodiments is a method of detecting a cancer in a subject, the method comprising: (a) determining the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) detecting the cancer in the subject using the prediction model.

Also provided herein in some embodiments is a method of assessing a characteristic of a cancer in a subject.

In some of any embodiments, the method comprises determining the value for each of at least one isotopic feature for a sample from a subject. In some of any embodiments, the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject. In some of any embodiments, the at least one isotopic feature comprises the overall concentration of an isotopic element measured in the sample of the subject. In some of any embodiments, the at least one isotopic feature comprises a chemical feature determined from the overall concentration of an isotopic element measured in the sample of the subject. In some of any embodiments, the at least one isotopic feature comprises the concentration of an isotope of an isotopic element measured in the sample of the subject. In some of any embodiments, the at least one isotopic feature comprises the concentration of an isotope of an isotopic element measured in the sample of the subject. In some of any embodiments, the at least one isotopic feature comprises a concentration of an element, a concentration of an isotopic element, a concentration of an isotope of the isotopic element, or a normalized value of thereof. In some of any embodiments, the at least one isotopic feature comprises a concentration of an element measured in the sample of the subject. In some of any embodiments, the at least one isotopic feature comprises a concentration of an isotopic element measured in the sample of the subject. In some of any embodiments, the at least one isotopic feature comprises a concentration of an isotope of the isotopic element measured in the sample of the subject. In some of any embodiments, the at least one isotopic feature comprises a normalized value of a concentration of an element measured in the sample of the subject. In some of any embodiments, the at least one isotopic feature comprises a normalized value of a concentration of an isotopic element measured in the sample of the subject. In some of any embodiments, the at least one isotopic feature comprises a normalized value of a concentration of an isotope of the isotopic element measured in the sample of the subject.

In some of any embodiments, the method comprises inputting the value for each of the at least one isotopic feature from the subject into a prediction model. In some of any embodiments, the prediction model is configured to predict a characteristic of a cancer in a subject. In some of any embodiments, the prediction model comprises at least one machine learning algorithm trained using values for each of the at least one isotopic feature from a plurality of reference samples. In some of any embodiments, the plurality of reference samples are from a plurality of reference subjects.

In some of any embodiments, the method comprises determining, using the prediction model, the characteristic of the cancer in the subject.

Also provided herein in some embodiments is a method of assessing a characteristic of a cancer in a subject, the method comprising: (a) determining the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

Also provided herein in some embodiments is a method of assessing a characteristic of a cancer in a subject, the method comprising: (a) determining the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

In some of any embodiments, the characteristic of the cancer is the level of tumor burden of the cancer in the subject.

In some of any embodiments, the determining the value for each of the at least one isotopic feature comprises mass spectrometry analysis. In some of any embodiments, the value is a normalized value, and the determining the value for each of the at least one isotopic feature comprises calculating the normalized value from the mass spectrometry analysis.

In some of any embodiments, the mass spectrometry analysis is of a liquid sample of the sample. In some of any embodiments, the sample is a solid sample, and the mass spectrometry analysis is of a liquid sample of the solid sample. In some of any embodiments, the determining the value for each of the at least one isotopic feature comprises preparing a liquid sample of the sample for analysis by mass spectrometry. In some of any embodiments, the determining the value for each of the at least one isotopic feature comprises preparing a liquid sample of the solid sample for analysis by mass spectrometry. In some of any embodiments, the liquid sample is prepared by digestion of the sample. In some of any embodiments, the liquid sample is prepared by digestion of the solid sample.

In some of any embodiments, the mass spectrometry analysis is of a solid sample of the sample. In some of any embodiments, the sample is a solid sample, and the mass spectrometry analysis is of the solid sample. In some of any embodiments, the determining the value for each of the at least one isotopic feature comprises preparing a solid sample of the sample for analysis by mass spectrometry. In some of any embodiments, the determining the value for each of the at least one isotopic feature comprises preparing the solid sample. In some of any embodiments, the solid sample is analyzed via combustion.

In some of any embodiments, the determining the value for each of the at least one isotopic feature comprises mass spectrometry analysis of a liquid sample of the sample or a solid sample of the sample. In some of any embodiments, the sample is a solid sample, and the determining the value for each of the at least one isotopic feature comprises mass spectrometry analysis of a liquid sample of the solid sample or the solid sample.

In some of any embodiments, the determining the value for each of the at least one isotopic feature comprises mass spectrometry analysis of a liquid sample prepared by digestion of the sample or a solid sample of the sample that is analyzed via combustion. In some of any embodiments, the sample is a solid sample, and the determining the value for each of the at least one isotopic feature comprises mass spectrometry analysis of the solid sample or a liquid sample prepared by digestion of the solid sample. In some embodiments, the solid sample is analyzed via combustion.

In some of any embodiments, the determining the value for each of the at least one isotopic feature comprises: (i) preparing a liquid sample of the sample for analysis by mass spectrometry, wherein the liquid sample is prepared by digestion of the sample; (ii) introducing the prepared liquid sample into a mass spectrometer to detect one or more ionization products of isotopic elements present in the prepared liquid sample; and (iii) analyzing the detected one or more ionization products of isotopic elements to generate the value for each of the at least one isotopic feature. In some of any embodiments, the sample is a solid sample, and wherein the determining the value for each of the at least one isotopic feature comprises: (i) preparing a liquid sample of the solid sample for analysis by mass spectrometry, wherein the liquid sample is prepared by digestion of the solid sample; (ii) introducing the prepared liquid sample into a mass spectrometer to detect one or more ionization products of elements and isotopic elements present in the prepared liquid sample; and (iii) analyzing the detected one or more ionization products of elements and isotopic elements to generate the value for each of the at least one isotopic feature.

In some of any embodiments, the determining the value for each of the at least one isotopic feature comprises: (i) preparing a solid sample of the sample for analysis by mass spectrometry; (ii) introducing the prepared solid sample into a mass spectrometer to detect one or more ionization products of isotopic elements present in the prepared solid sample, wherein the detecting comprises combustion of the prepared solid sample; and (iii) analyzing the detected one or more ionization products of isotopic elements to generate the value for each of the at least one isotopic feature. In some of any embodiments, the sample is a solid sample, and wherein the determining the value for each of the at least one isotopic feature comprises: (i) preparing the solid sample for analysis by mass spectrometry; (ii) introducing the prepared solid sample into a mass spectrometer to detect one or more ionization products of elements and isotopic elements present in the prepared solid sample, optionally wherein the detecting comprises combustion of the prepared solid sample; and (iii) analyzing the detected one or more ionization products of elements and isotopic elements to generate the value for each of the at least one isotopic feature.

In some of any embodiments, the determining in step (a), the inputting in step (b), and/or the determining in step (c) is performed by a processor of a computing device. In some of any embodiments, the determining in step (a) is performed by a processor of a computing device. In some of any embodiments, the inputting in step (b) is performed by a processor of a computing device. In some of any embodiments, the determining in step (c) is performed by a processor of a computing device. In some of any embodiments, the determining in step (a), the inputting in step (b), and the determining in step (c) is performed by a processor of a computing device.

Also provided herein in some embodiments is a method of detecting whether a subject has a cancer, the method comprising: (a) receiving, at one or more processors, the value for each of at least one isotopic feature for a sample from a subject, wherein the at least isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample from the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer.

Also provided herein in some embodiments is a method of detecting whether a subject has a cancer, the method comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer

Also provided herein in some embodiments is a method of detecting a cancer in a subject, the method comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) detecting the cancer in the subject using the prediction model

Also provided herein in some embodiments is a method of assessing a characteristic of a cancer in a subject, the method comprising: (a) receiving, at one or more processors, the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting, using at least one processor, the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

Also provided herein in some embodiments is a method of assessing a characteristic of a cancer in a subject, the method comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

In some of any embodiments, the characteristic of the cancer is the level of tumor burden of the cancer in the subject.

In some of any embodiments, the value for each of the at least one isotopic feature is obtained by mass spectrometry analysis. In some of any embodiments, the value is a normalized value, and the determining the value for each of the at least one isotopic feature comprises calculating the normalized value from the mass spectrometry analysis.

In some of any embodiments, the mass spectrometry analysis is of a liquid sample of the sample. In some of any embodiments, the sample is a solid sample, and the mass spectrometry analysis is of a liquid sample of the solid sample. In some of any embodiments, the liquid sample is prepared by digestion of the sample. In some of any embodiments, the liquid sample is prepared by digestion of the solid sample.

In some of any embodiments, the mass spectrometry analysis is of a solid sample of the sample. In some of any embodiments, the sample is a solid sample, and the mass spectrometry analysis is of the solid sample. In some of any embodiments, the solid sample is analyzed via combustion.

In some of any embodiments, the value for each of the at least one isotopic feature is obtained by mass spectrometry analysis of a liquid sample of the sample or a solid sample of the sample. In some of any embodiments, the sample is a solid sample, and the value for each of the at least one isotopic feature is obtained by mass spectrometry analysis of the solid sample or a liquid sample of the solid sample.

In some of any embodiments, the value for each of the at least one isotopic feature is obtained by mass spectrometry analysis of a liquid sample prepared by digestion of the sample or a solid sample of the sample that is analyzed via combustion. In some of any embodiments, the sample is a solid sample, and the value for each of the at least one isotopic feature is obtained by mass spectrometry analysis of the solid sample or a liquid sample prepared by digestion of the solid sample. In some of any embodiments, the solid sample is analyzed via combustion.

In some of any embodiments, prior to step (a), the method comprises determining the value for each of the at least one isotopic feature.

In some of any embodiments, prior to step (a), the method comprises determining the value for each of the at least one isotopic feature, wherein the determining comprises: (i) preparing a liquid sample of the sample for analysis by mass spectrometry, wherein the liquid sample is prepared by digestion of the sample; (ii) introducing the prepared liquid sample into a mass spectrometer to detect one or more ionization products of isotopic elements present in the prepared liquid sample; and (iii) analyzing the detected one or more ionization products of isotopic elements to generate the value for each of the at least one isotopic feature. In some of any embodiments, the sample is a solid sample, and wherein prior to step (a), the method comprises determining the value for each of the at least one isotopic feature, wherein the determining comprises: (i) preparing a liquid sample of the solid sample for analysis by mass spectrometry, wherein the liquid sample is prepared by digestion of the solid sample; (ii) introducing the prepared liquid sample into a mass spectrometer to detect one or more ionization products of elements isotopic elements present in the prepared liquid sample; and (iii) analyzing the detected one or more ionization products of elements and isotopic elements to generate the value for each of the at least one isotopic feature

In some of any embodiments, prior to step (a), the method comprises determining the value for each of the at least one isotopic feature, wherein the determining comprises: (i) preparing a solid sample of the sample for analysis by mass spectrometry; (ii) introducing the prepared solid sample into a mass spectrometer to detect one or more ionization products of isotopic elements present in the prepared solid sample, wherein the detecting comprises combustion of the prepared solid sample; and (iii) analyzing the detected one or more ionization products of isotopic elements to generate the value for each of the at least one isotopic feature. In some of any embodiments, the sample is a solid sample, and wherein prior to step (a), the method comprises determining the value for each of the at least one isotopic feature, wherein the determining comprises: (i) preparing the solid sample for analysis by mass spectrometry; (ii) introducing the prepared solid sample into a mass spectrometer to detect one or more ionization products of elements and isotopic elements present in the prepared solid sample, optionally wherein the detecting comprises combustion of the prepared solid sample; and (iii) analyzing the detected one or more ionization products of elements and isotopic elements to generate the value for each of the at least one isotopic feature

In some of any embodiments, the analyzing the detected one or more ionization products of isotopic elements is with reference to one or more ionization products of elements present in standards. In some of any embodiments, the analyzing the detected one or more ionization products of elements and isotopic elements is with reference to one or more ionization products of elements and isotopic elements present in standards.

In some of any embodiments, the analyzing the detected one or more ionization products of isotopic elements is with reference to one or more ionization products of elements present in standards comprising a plurality of calibration standards and a blank standard. In some of any embodiments, the analyzing the detected one or more ionization products of isotopic elements is with reference to one or more ionization products of elements and isotopic elements present in standards comprising a plurality of calibration standards and a blank standard.

In some of any embodiments, the analyzing the detected one or more ionization products of isotopic elements is with reference to one or more ionization products of elements present in standards comprising an internal standard and/or a reference material. In some of any embodiments, the analyzing the detected one or more ionization products of isotopic elements is with reference to one or more ionization products of elements present in standards comprising an internal standard. In some of any embodiments, the analyzing the detected one or more ionization products of isotopic elements is with reference to one or more ionization products of elements present in standards comprising a reference material. In some of any embodiments, the analyzing the detected one or more ionization products of isotopic elements is with reference to one or more ionization products of elements present in standards comprising an internal standard and a reference material.

In some of any embodiments, the analyzing the detected one or more ionization products of isotopic elements and elements is with reference to one or more ionization products of elements and isotopic elements present in standards comprising an internal standard and/or a reference material. In some of any embodiments, the analyzing the detected one or more ionization products of isotopic elements and elements is with reference to one or more ionization products of elements and isotopic elements present in standards comprising an internal standard. In some of any embodiments, the analyzing the detected one or more ionization products of isotopic elements and elements is with reference to one or more ionization products of elements and isotopic elements present in standards comprising a reference material. In some of any embodiments, the analyzing the detected one or more ionization products of isotopic elements and elements is with reference to one or more ionization products of elements and isotopic elements present in standards comprising an internal standard and a reference material.

In some of any embodiments, the mass spectrometer is further introduced with the standards, wherein the mass spectrometer detects one or more ionization products of elements present in the standards and the prepared liquid or solid sample. In some of any embodiments, the mass spectrometer is further introduced with the standards, wherein the mass spectrometer detects one or more ionization products of elements and isotopic elements present in the standards and the prepared liquid or solid sample.

Also provided herein in some embodiments is a method of detecting whether a subject has a cancer, the method comprising: (a) providing a sample obtained from a subject; (b) preparing a liquid or solid sample for analysis by mass spectrometry from the sample, wherein the liquid sample is prepared by digestion of the sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements present in each of the standards and the prepared liquid or solid sample; (e) analyzing the detected one or more ionization products of isotopic elements to generate the value for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element; (f) receiving, at one or more processors, the value for each of the at least one isotopic feature; (g) inputting, using at least one processor, the value for each of the at least one isotopic feature into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (h) determining, using the prediction model, whether the subject has the cancer.

Also provided herein in some embodiments is a method of detecting whether a subject has a cancer, the method comprising: (a) providing a solid sample obtained from a subject; (b) preparing a liquid sample or the solid sample for analysis by mass spectrometry, wherein the liquid sample is prepared by digestion of the solid sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements and isotopic elements present in each of the standards and the prepared liquid or solid sample; (e) analyzing the detected one or more ionization products of elements and isotopic elements to generate the value for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the value for each of the at least one isotopic feature; (g) inputting, using at least one processor, the value for each of the at least one isotopic feature into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (h) determining, using the prediction model, whether the subject has the cancer.

Also provided herein in some embodiments is a method of detecting a cancer in a subject, the method comprising: (a) providing a solid sample obtained from a subject; (b) preparing a liquid sample or the solid sample for analysis by mass spectrometry, wherein the liquid sample is prepared by digestion of the solid sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements and isotopic elements present in each of the standards and the prepared liquid or solid sample; (e) analyzing the detected one or more ionization products of elements and isotopic elements to generate the value for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the value for each of the at least one isotopic feature; (g) inputting, using at least one processor, the value for each of the at least one isotopic feature into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (h) detecting the cancer in the subject using the prediction model

Also provided herein in some embodiments is a method of assessing a characteristic of a cancer in a subject, the method comprising: (a) providing a sample obtained from a subject; (b) preparing a liquid or solid sample for analysis by mass spectrometry from the sample, wherein the liquid sample is prepared by digestion of the sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements present in each of the standards and the prepared liquid or solid sample; (e) analyzing the detected one or more ionization products of isotopic elements to generate the value for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element; (f) receiving, at one or more processors, the value for each of the at least one isotopic feature; (g) inputting, using at least one processor, the value for each of the at least one isotopic feature into a prediction model configured to predict a characteristic of a cancer in a subject, wherein the prediction model comprises at least one machine learning algorithm trained using values for each of the at least one isotopic feature from a plurality of reference samples from a plurality of reference subjects; and (h) determining, using the prediction model, the characteristic of the cancer in the subject.

Also provided herein in some embodiments is a method of assessing a characteristic of a cancer in a subject, the method comprising: (a) providing a solid sample obtained from a subject; (b) preparing a liquid sample or the solid sample for analysis by mass spectrometry, wherein the liquid sample is prepared by digestion of the solid sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements and isotopic elements present in each of the standards and the prepared liquid or solid sample; (e) analyzing the detected one or more ionization products of elements and isotopic elements to generate the value for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the value for each of the at least one isotopic feature; (g) inputting, using at least one processor, the value for each of the at least one isotopic feature into a prediction model configured to predict a characteristic of a cancer in a subject, wherein the prediction model comprises at least one machine learning algorithm trained using values for each of the at least one isotopic feature from a plurality of reference samples from a plurality of reference subjects; and (h) determining, using the prediction model, the characteristic of the cancer in the subject.

In some of any embodiments, the characteristic of the cancer is the level of tumor burden of the cancer in the subject.

In some of any embodiments, the sample comprises a keratinous tissue, optionally a hard keratinous tissue. In some of any embodiments, the sample comprises a keratinous tissue. In some of any embodiments, the sample comprises a hard keratinous tissue.

In some of any embodiments, the sample is a noninvasive sample, optionally a hair sample or a nail sample. In some of any embodiments, the sample is a noninvasive sample. In some of any embodiments, the sample is a hair sample or a nail sample. In some of any embodiments, the sample is a hair sample. In some of any embodiments, the sample is a nail sample.

In some of any embodiments, the digestion is by acid digestion and/or thermal digestion. In some of any embodiments, the digestion is by acid digestion and thermal digestion.

In some of any embodiments, the acid digestion is with nitric acid (HNO₃). In some of any embodiments, the acid digestion is with nitric acid (HNO₃) and hydrogen peroxide (H₂O₂).

In some of any embodiments, the thermal digestion is by a microwave.

In some of any embodiments, the mass spectrometry is Inductively Coupled Plasma Mass Spectrometry (ICP-MS). In some of any embodiments, the ICP-MS is carried out with kinetic energy discrimination (KED).

In some of any embodiments, the at least one isotopic feature comprises an atomic percent, isotope ratio, fractional abundance, delta, overall concentration, and/or concentration ratio. In some of any embodiments, the at least one isotopic feature comprises an atomic percent. In some of any embodiments, the at least one isotopic feature comprises an isotope ratio. In some of any embodiments, the at least one isotopic feature comprises a fractional abundance. In some of any embodiments, the at least one isotopic feature comprises a delta. In some of any embodiments, the at least one isotopic feature comprises an overall concentration. In some of any embodiments, the at least one isotopic feature comprises a concentration ratio.

In some of any embodiments, the at least one isotopic feature comprises an atomic percent for at least one isotope of an isotopic element, an isotope ratio for at least one isotope of an isotopic element, a fractional abundance for at least one isotope of an isotopic element, and/or a delta for at least one isotope of an isotopic element. In some of any embodiments, the at least one isotopic feature comprises an atomic percent for at least one isotope of an isotopic element. In some of any embodiments, the at least one isotopic feature comprises an isotope ratio for at least one isotope of an isotopic element. In some of any embodiments, the at least one isotopic feature comprises a fractional abundance for at least one isotope of an isotopic element. In some of any embodiments, the at least one isotopic feature comprises a delta for at least one isotope of an isotopic element.

In some of any embodiments, the at least one isotopic feature is a plurality of isotopic features each independently selected from the group consisting of atomic percents, isotope ratios, fractional abundances, deltas, overall concentrations, and concentration ratios.

In some of any embodiments, the plurality of isotopic features comprises an atomic percent for at least one isotope of an isotopic element, an isotope ratio for at least one isotope of an isotopic element, a fractional abundance for at least one isotope of an isotopic element, and/or a delta for at least one isotope of an isotopic element. In some of any embodiments, the plurality of isotopic features comprises an atomic percent for at least one isotope of an isotopic element. In some of any embodiments, the plurality of isotopic features comprises an isotope ratio for at least one isotope of an isotopic element. In some of any embodiments, the plurality of isotopic features comprises a fractional abundance for at least one isotope of an isotopic element. In some of any embodiments, the plurality of isotopic features comprises a delta for at least one isotope of an isotopic element.

In some of any embodiments, the value of each of the at least one isotopic feature is a normalized value of a concentration of an element, a normalized value of a concentration of an isotopic element, or a normalized value of a concentration of an isotope of the isotopic element. In some of any embodiments, the value of each of the at least one isotopic feature is a normalized value of a concentration of an element. In some of any embodiments, the value of each of the at least one isotopic feature is a normalized value of a concentration of an isotopic element. In some of any embodiments, the value of each of the at least one isotopic feature is a normalized value of a concentration of an isotope of the isotopic element.

In some of any embodiments, the normalized value of each of the at least one isotopic feature is an atomic percent (atom %), fractional abundance (F), isotope ratio (R), concentration ratio (CR), or a delta value (delta), or a combination of any of the foregoing. In some of any embodiments, the normalized value of each of the least one isotopic feature is an atomic percent (atom %). In some of any embodiments, the normalized value of each of the least one isotopic feature is a fractional abundance (F). In some of any embodiments, the normalized value of each of the least one isotopic feature is an isotope ratio (R). In some of any embodiments, the normalized value of each of the least one isotopic feature is a concentration ratio (CR). In some of any embodiments, the normalized value of each of the least one isotopic feature is a delta value (delta).

In some of any embodiments, the normalized value of each of the at least one isotopic feature is a delta value (delta). In some of any embodiments, the delta value is a conventional delta value, a study-specific delta value (InH), a non-similar simple-nitrogen based delta value (SimpleNδ), or a non-similar pseudo-nitrogen based delta value (PseudoNδ). In some of any embodiments, the delta value is a conventional delta value. In some of any embodiments, the delta value is a study-specific delta value (InH). In some of any embodiments, the delta value is a non-similar simple-nitrogen based delta value (SimpleNδ). In some of any embodiments, the delta value is a non-similar pseudo-nitrogen based delta value (PseudoNδ).

In some of any embodiments, the isotopic element is Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S.

In some of any embodiments, the isotopic element is Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, or Zn. In some of any embodiments, the isotopic element is Ag. In some of any embodiments, the isotopic element is Al. In some of any embodiments, the isotopic element is B. In some of any embodiments, the isotopic element is Ba. In some of any embodiments, the isotopic element is Br. In some of any embodiments, the isotopic element is Ca. In some of any embodiments, the isotopic element is Co. In some of any embodiments, the isotopic element is Cr. In some of any embodiments, the isotopic element is Cu. In some of any embodiments, the isotopic element is Fe. In some of any embodiments, the isotopic element is Ge. In some of any embodiments, the isotopic element is Hg. In some of any embodiments, the isotopic element is I. In some of any embodiments, the isotopic element is K. In some of any embodiments, the isotopic element is Li. In some of any embodiments, the isotopic element is Mg. In some of any embodiments, the isotopic element is Mn. In some of any embodiments, the isotopic element is Mo. In some of any embodiments, the isotopic element is Na. In some of any embodiments, the isotopic element is Ni. In some of any embodiments, the isotopic element is Pb. In some of any embodiments, the isotopic element is Rb. In some of any embodiments, the isotopic element is Ru. In some of any embodiments, the isotopic element is Se. In some of any embodiments, the isotopic element is Sn. In some of any embodiments, the isotopic element is Sr. In some of any embodiments, the isotopic element is Te. In some of any embodiments, the isotopic element is Ti. In some of any embodiments, the isotopic element is U. In some of any embodiments, the isotopic element is V. In some of any embodiments, the isotopic element is Y. In some of any embodiments, the isotopic element is Zn.

In some of any embodiments, the isotopic element is N, C, S, or O. In some of any embodiments, the isotopic element is N. In some of any embodiments, the isotopic element is C. In some of any embodiments, the isotopic element is S. In some of any embodiments, the isotopic element is O.

In some of any embodiments, the isotopic element is Mo, Cr, Te, C, or Hg. In some of any embodiments, the isotopic element is Mo. In some of any embodiments, the isotopic element is Cr. In some of any embodiments, the isotopic element is Te. In some of any embodiments, the isotopic element is C. In some of any embodiments, the isotopic element is Hg.

In some of any embodiments, the at least one isotopic feature is one isotopic feature. In some of any embodiments, the isotope of the at least one isotopic feature comprises ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr. In some of any embodiments, the isotope of the at least one isotopic feature comprises ⁶⁶Zn. In some of any embodiments, the isotope of the at least one isotopic feature comprises ⁶⁴Zn. In some of any embodiments, the isotope of the at least one isotopic feature comprises ⁶⁸Zn. In some of any embodiments, the isotope of the at least one isotopic feature comprises ⁸⁷Sr. In some of any embodiments, the isotope of the at least one isotopic feature comprises ⁸⁸Sr. In some of any embodiments, the isotope of the at least one isotopic feature comprises ⁸⁶Sr. In some of any embodiments, the isotope of the at least one isotopic feature comprises ⁴⁷Ti. In some of any embodiments, the isotope of the at least one isotopic feature comprises ⁴⁸Ti. In some of any embodiments, the isotope of the at least one isotopic feature comprises ⁴⁶Ti. In some of any embodiments, the isotope of the at least one isotopic feature comprises ⁵⁰Cr. In some of any embodiments, the isotope of the at least one isotopic feature comprises ⁶⁶Zn, ⁶⁴Zn, ⁶⁸Zn, ⁸⁷Sr, ⁸⁸Sr ⁸⁶Sr, ⁴⁷Ti, ⁴⁸Ti, ⁴⁶Ti, and ⁵⁰Cr.

In some of any embodiments, the at least one isotopic feature is a plurality of isotopic features. In some of any embodiments, the plurality of isotopic features is about 2 to about 100 isotopic features. In some of any embodiments, the plurality of isotopic features is 2-100 isotopic features. In some of any embodiments, the plurality of isotopic features comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or more isotopic features. In some of any embodiments, the plurality of isotopic features comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic features.

In some of any embodiments, the at least one isotopic feature is a plurality of isotopic features determined from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or more elements or isotopic elements. In some of any embodiments, the plurality of isotopic features are determined from at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic elements.

In some of any embodiments, the plurality of isotopic features comprises fractional abundances and deltas. In some of any embodiments, the fractional abundance and delta values are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

In some of any embodiments, the plurality of isotopic features comprises fractional abundances, atomic percents, and/or isotope ratios. In some of any embodiments, the fractional abundances, atomic percents, and/or isotope ratios are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, and/or S. In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, and S. In some of any embodiments, the element or the isotopic element of the at least one isotopic feature comprises Li, B, Na, Mg, Al, P, K, Sc, Ca, Ti, V, Cr, Mn, Fe, Co, Ni, Cu, Zn, Ge, As, Se, Br, Rb, Sr, Y, Zr, Nb, Mo, Ru, Ag, Cd, In, Sn, I, Te, Ba, Hg, Pb, U, C, N, O, or S, or a combination of any of the foregoing. In some of any embodiments, the element or the isotopic element of the at least one isotopic feature comprises Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S, or a combination of any of the foregoing. In some of any embodiments, the element or the isotopic element of the at least one isotopic feature comprises Li, B, Mg, Ca, Ti, Cr, Fe, Ni, Cu, Zn, Se, Br, Rb, Sr, Mo, Ag, Sn, Ba, Hg, Pb, U, C, N, S, or a combination of any of the foregoing.

In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, or Zn. In some of any embodiments, the isotopic elements comprise Ag. In some of any embodiments, the isotopic elements comprise Al. In some of any embodiments, the isotopic elements comprise B. In some of any embodiments, the isotopic elements comprise Ba. In some of any embodiments, the isotopic elements comprise Br. In some of any embodiments, the isotopic elements comprise Ca. In some of any embodiments, the isotopic elements comprise Co. In some of any embodiments, the isotopic elements comprise Cr. In some of any embodiments, the isotopic elements comprise Cu. In some of any embodiments, the isotopic elements comprise Fe. In some of any embodiments, the isotopic elements comprise Ge. In some of any embodiments, the isotopic elements comprise Hg. In some of any embodiments, the isotopic elements comprise I. In some of any embodiments, the isotopic elements comprise K. In some of any embodiments, the isotopic elements comprise Li. In some of any embodiments, the isotopic elements comprise Mg. In some of any embodiments, the isotopic elements comprise Mn. In some of any embodiments, the isotopic elements comprise Mo. In some of any embodiments, the isotopic elements comprise Na. In some of any embodiments, the isotopic elements comprise Ni. In some of any embodiments, the isotopic elements comprise Pb. In some of any embodiments, the isotopic elements comprise Rb. In some of any embodiments, the isotopic elements comprise Ru. In some of any embodiments, the isotopic elements comprise Se. In some of any embodiments, the isotopic elements comprise Sn. In some of any embodiments, the isotopic elements comprise Sr. In some of any embodiments, the isotopic elements comprise Te. In some of any embodiments, the isotopic elements comprise Ti. In some of any embodiments, the isotopic elements comprise U. In some of any embodiments, the isotopic elements comprise V. In some of any embodiments, the isotopic elements comprise Y. In some of any embodiments, the isotopic elements comprise Zn. In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, and Zn.

In some of any embodiments, the isotopic elements comprise N, C, S, and/or O. In some of any embodiments, the isotopic elements comprise N. In some of any embodiments, the isotopic elements comprise C. In some of any embodiments, the isotopic elements comprise S. In some of any embodiments, the isotopic elements comprise O. In some of any embodiments, the isotopic elements comprise N, C, S, and O.

In some of any embodiments, the isotopic elements comprise Mo, Cr, Te, C, and/or Hg. In some of any embodiments, the isotopic elements comprise Mo. In some of any embodiments, the isotopic elements comprise Cr. In some of any embodiments, the isotopic elements comprise Te. In some of any embodiments, the isotopic elements comprise C. In some of any embodiments, the isotopic elements comprise Hg. In some of any embodiments, the isotopic elements comprise Mo, Cr, Te, C, and Hg.

In some of any embodiments, at least one isotopic feature comprises a concentration ratio. In some of any embodiments, the concentration ratio is N/C, S/C, O/C, S/N, N/O, or S/O. In some of any embodiments, the concentration ratio is N/C. In some of any embodiments, the concentration ratio is S/C. In some of any embodiments, the concentration ratio is O/C. In some of any embodiments, the concentration ratio is S/N. In some of any embodiments, the concentration ratio is N/O. In some of any embodiments, the concentration ratio is S/O.

In some of any embodiments, the plurality of isotopic features comprises at least one concentration ratio. In some of any embodiments, the at least one concentration ratio comprises N/C, S/C, O/C, S/N, N/O, and/or S/O. In some of any embodiments, the at least one concentration ratio comprises N/C. In some of any embodiments, the at least one concentration ratio comprises S/C. In some of any embodiments, the at least one concentration ratio comprises O/C. In some of any embodiments, the at least one concentration ratio comprises S/N. In some of any embodiments, the at least one concentration ratio comprises N/O. In some of any embodiments, the at least one concentration ratio comprises S/O. In some of any embodiments, the at least one concentration ratio comprises N/C, S/C, O/C, S/N, N/O, and S/O.

In some of any embodiments, the at least one isotopic feature comprises at least 1, 2, 3, 4, 5, 6, 7, 8, 9, or 10 of the fractional abundance (F), atomic percent (atom %), or isotope ratio (R) of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (F34S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO). In some of any embodiments, the at least one isotopic feature comprises the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (F34S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO).

In some of any embodiments, the at least one isotopic feature comprises the fractional abundance, atomic percent, or isotope ratio of ¹³C (F13C) relative to stable isotopes of C. In some of any embodiments, the at least one isotopic feature comprises the fractional abundance, atomic percent, or isotope ratio of ¹⁸O (f18O) relative to stable isotopes of O. In some of any embodiments, the at least one isotopic feature comprises the concentration ratio of sulfur over nitrogen (SdN). In some of any embodiments, the at least one isotopic feature comprises the concentration ratio of nitrogen over carbon (NdC). In some of any embodiments, the at least one isotopic feature comprises the concentration ratio of nitrogen over carbon (NdC). In some of any embodiments, the at least one isotopic feature comprises the fractional abundance, atomic percent, or isotope ratio of ³⁴S (F34S) relative to stable isotopes of S. In some of any embodiments, the at least one isotopic feature comprises the fractional abundance, atomic percent, or isotope ratio of ¹⁵N (F15N) relative to stable isotopes of N. In some of any embodiments, the at least one isotopic feature comprises the concentration ratio of oxygen over carbon (OdC). In some of any embodiments, the at least one isotopic feature comprises the concentration ratio of nitrogen over oxygen (NdO). In some of any embodiments, the at least one isotopic feature comprises the concentration ratio of sulfur over carbon (SdC). In some of any embodiments, the at least one isotopic feature comprises the concentration ratio of sulfur over oxygen (SdO).

In some of any embodiments, the at least one isotopic feature comprises the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (F34S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO).

In some of any embodiments, the at least one isotopic feature comprises at least 1, 2, 3, 4, 5, 6, 7, 8, 9, 10, 11, 12, 13, 14, 15, 16, 17, 18, 19, 20, or more of the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷I, ¹²⁸Te ¹³⁶Ba, ¹³⁷Ba, ¹³C, ²³⁵U, ²³⁸U, ²³Na, ²⁷Al, ³⁴S, ³⁹K, ⁴⁴Ca, ⁴⁸Ti, ⁵⁴Cr, ⁵⁴Fe, ⁵⁷Fe, ⁶²Ni, ⁶³Cu, ⁶⁶Zn, ⁶⁷Zn, ⁶⁸Zn, ⁷⁰Ge, ⁷⁴Ge, ⁷⁴Se, ⁷⁶Se, ⁷⁷Se, ⁷⁹Br, ⁸⁰Se, ⁸²Se, ⁸⁹Y, ⁹⁴Mo, ⁹⁷Mo, and ⁹⁸Mo, each relative to stable isotopes of the isotopic element of the isotope.

In some of any embodiments, the at least one isotopic feature comprises the fractional abundance, atomic percent, or isotope ratio of ¹⁰¹Ru relative to stable isotopes of Ru. In some of any embodiments, the at least one isotopic feature comprises the fractional abundance, atomic percent, or isotope ratio of ¹⁰⁷Ag relative to stable isotopes of Ag. In some of any embodiments, the at least one isotopic feature comprises the fractional abundance, atomic percent, or isotope ratio of ¹⁰⁹Ag relative to stable isotopes of Ag. In some of any embodiments, the at least one isotopic feature comprises the fractional abundance, atomic percent, or isotope ratio of ¹⁰B relative to stable isotopes of B. In some of any embodiments, the at least one isotopic feature comprises the fractional abundance, atomic percent, or isotope ratio of ¹¹⁸Sn relative to stable isotopes of Sn. In some of any embodiments, the at least one isotopic feature comprises the fractional abundance, atomic percent, or isotope ratio of ¹¹⁹Sn relative to stable isotopes of Sn. In some of any embodiments, the at least one isotopic feature comprises the fractional abundance, atomic percent, or isotope ratio of ¹²⁴Sn relative to stable isotopes of Sn. In some of any embodiments, the at least one isotopic feature comprises the fractional abundance, atomic percent, or isotope ratio of ¹²⁴Te relative to stable isotopes of Te. In some of any embodiments, the at least one isotopic feature comprises the fractional abundance, atomic percent, or isotope ratio of ¹²⁷I relative to stable isotopes of I. In some of any embodiments, the at least one isotopic feature comprises the fractional abundance, atomic percent, or isotope ratio of ¹²⁸Te relative to stable isotopes of Te. In some of any embodiments, the at least one isotopic feature comprises the fractional abundance, atomic percent, or isotope ratio of ¹³⁶Ba relative to stable isotopes of Ba. In some of any embodiments, the at least one isotopic feature comprises the fractional abundance, atomic percent, or isotope ratio of ¹³⁷Ba relative to stable isotopes of Ba. In some of any embodiments, the at least one isotopic feature comprises the fractional abundance, atomic percent, or isotope ratio of ¹³C relative to stable isotopes of C. In some of any embodiments, the at least one isotopic feature comprises the fractional abundance, atomic percent, or isotope ratio of ²³⁵U relative to stable isotopes of U. In some of any embodiments, the at least one isotopic feature comprises the fractional abundance, atomic percent, or isotope ratio of ²³⁸U relative to stable isotopes of U. In some of any embodiments, the at least one isotopic feature comprises the fractional abundance, atomic percent, or isotope ratio of ²³Na relative to stable isotopes of Na. In some of any embodiments, the at least one isotopic feature comprises the fractional abundance, atomic percent, or isotope ratio of ²⁷Al relative to stable isotopes of Al. In some of any embodiments, the at least one isotopic feature comprises the fractional abundance, atomic percent, or isotope ratio of ³⁴S relative to stable isotopes of S. In some of any embodiments, the at least one isotopic feature comprises the fractional abundance, atomic percent, or isotope ratio of ³⁹K relative to stable isotopes of K. In some of any embodiments, the at least one isotopic feature comprises the fractional abundance, atomic percent, or isotope ratio of ⁴⁴Ca relative to stable isotopes of Ca. In some of any embodiments, the at least one isotopic feature comprises the fractional abundance, atomic percent, or isotope ratio of ⁴⁸Ti relative to stable isotopes of Ti. In some of any embodiments, the at least one isotopic feature comprises the fractional abundance, atomic percent, or isotope ratio of ⁵⁴Cr relative to stable isotopes of Cr. In some of any embodiments, the at least one isotopic feature comprises the fractional abundance, atomic percent, or isotope ratio of ⁵⁴Fe relative to stable isotopes of Fe. In some of any embodiments, the at least one isotopic feature comprises the fractional abundance, atomic percent, or isotope ratio of ⁵⁷Fe relative to stable isotopes of Fe. In some of any embodiments, the at least one isotopic feature comprises the fractional abundance, atomic percent, or isotope ratio of ⁶²Ni relative to stable isotopes of Ni. In some of any embodiments, the at least one isotopic feature comprises the fractional abundance, atomic percent, or isotope ratio of ⁶³Cu relative to stable isotopes of Cu. In some of any embodiments, the at least one isotopic feature comprises the fractional abundance, atomic percent, or isotope ratio of ⁶⁶Zn relative to stable isotopes of Zn. In some of any embodiments, the at least one isotopic feature comprises the fractional abundance, atomic percent, or isotope ratio of ⁶⁷Zn relative to stable isotopes of Zn. In some of any embodiments, the at least one isotopic feature comprises the fractional abundance, atomic percent, or isotope ratio of ⁶⁸Zn relative to stable isotopes of Zn. In some of any embodiments, the at least one isotopic feature comprises the fractional abundance, atomic percent, or isotope ratio of ⁷⁰Ge relative to stable isotopes of Ge. In some of any embodiments, the at least one isotopic feature comprises the fractional abundance, atomic percent, or isotope ratio of ⁷⁴Ge relative to stable isotopes of Ge. In some of any embodiments, the at least one isotopic feature comprises the fractional abundance, atomic percent, or isotope ratio of ⁷⁴Se relative to stable isotopes of Se. In some of any embodiments, the at least one isotopic feature comprises the fractional abundance, atomic percent, or isotope ratio of ⁷⁶Se relative to stable isotopes of Se. In some of any embodiments, the at least one isotopic feature comprises the fractional abundance, atomic percent, or isotope ratio of ⁷⁷Se relative to stable isotopes of Se. In some of any embodiments, the at least one isotopic feature comprises the fractional abundance, atomic percent, or isotope ratio of ⁷⁹Br relative to stable isotopes of Br. In some of any embodiments, the at least one isotopic feature comprises the fractional abundance, atomic percent, or isotope ratio of ⁸⁰Se relative to stable isotopes of Se. In some of any embodiments, the at least one isotopic feature comprises the fractional abundance, atomic percent, or isotope ratio of ⁸²Se relative to stable isotopes of Se. In some of any embodiments, the at least one isotopic feature comprises the fractional abundance, atomic percent, or isotope ratio of ⁸⁹Y relative to stable isotopes of Y. In some of any embodiments, the at least one isotopic feature comprises the fractional abundance, atomic percent, or isotope ratio of ⁹⁴Mo relative to stable isotopes of Mo. In some of any embodiments, the at least one isotopic feature comprises the fractional abundance, atomic percent, or isotope ratio of ⁹⁷Mo relative to stable isotopes of Mo. In some of any embodiments, the at least one isotopic feature comprises the fractional abundance, atomic percent, or isotope ratio of ⁹⁸Mo relative to stable isotopes of Mo.

In some of any embodiments, the at least one isotopic feature comprises the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷I, ¹²⁸Te, ¹³⁶Ba, ¹³⁷Ba, ¹³C, ²³⁵U, ²³⁸U, ²³Na, ²⁷Al, ³⁴S, ³⁹K, ⁴⁴Ca, ⁴⁸Ti, ⁵⁴Cr, ⁵⁴Fe, ⁵⁷Fe, ⁶²Ni, ⁶³Cu, ⁶⁶Zn, ⁶⁷Zn, ⁶⁸Zn, ⁷⁰Ge, ⁷⁴Ge, ⁷⁴Se, ⁷⁶Se, ⁷⁷Se, ⁷⁹Br, ⁸⁰Se, ⁸²Se, ⁸⁹Y, ⁹⁴Mo, ⁹⁷Mo, and ⁹⁸Mo, each relative to stable isotopes of the isotopic element of the isotope.

In some of any embodiments, the at least one isotopic feature comprises at least 1, 2, 3, 4, 5, 6, 7, 8, 9, 10, or more of the fractional abundance, atomic percent, or isotope ratio of ¹⁰⁷Ag, ¹⁰⁹Ag, ¹¹⁸Sn, ¹²⁷I, ¹³C, ²³⁵U, ²⁷Al, ³⁴S, ⁴⁴Ca, ⁵⁷Fe, ⁶⁷Zn, ⁷⁴Se, ⁹⁴Mo, and ⁹⁷Mo, each relative to stable isotopes of the isotopic element of the isotope.

In some of any embodiments, the at least one isotopic feature comprises the fractional abundance, atomic percent, or isotope ratio of ¹⁰⁷Ag relative to stable isotopes of Ag. In some of any embodiments, the at least one isotopic feature comprises the fractional abundance, atomic percent, or isotope ratio of ¹⁰⁹Ag relative to stable isotopes of Ag. In some of any embodiments, the at least one isotopic feature comprises the fractional abundance, atomic percent, or isotope ratio of ¹¹⁸Sn relative to stable isotopes of Sn. In some of any embodiments, the at least one isotopic feature comprises the fractional abundance, atomic percent, or isotope ratio of ¹²⁷I relative to stable isotopes of I. In some of any embodiments, the at least one isotopic feature comprises the fractional abundance, atomic percent, or isotope ratio of ¹³C relative to stable isotopes of C. In some of any embodiments, the at least one isotopic feature comprises the fractional abundance, atomic percent, or isotope ratio of ²³⁵U relative to stable isotopes of U. In some of any embodiments, the at least one isotopic feature comprises the fractional abundance, atomic percent, or isotope ratio of ²⁷Al relative to stable isotopes of Al. In some of any embodiments, the at least one isotopic feature comprises the fractional abundance, atomic percent, or isotope ratio of ³⁴S relative to stable isotopes of S. In some of any embodiments, the at least one isotopic feature comprises the fractional abundance, atomic percent, or isotope ratio of ⁴⁴Ca relative to stable isotopes of Ca. In some of any embodiments, the at least one isotopic feature comprises the fractional abundance, atomic percent, or isotope ratio of ⁵⁷Fe relative to stable isotopes of Fe. In some of any embodiments, the at least one isotopic feature comprises the fractional abundance, atomic percent, or isotope ratio of ⁶⁷Zn relative to stable isotopes of Zn. In some of any embodiments, the at least one isotopic feature comprises the fractional abundance, atomic percent, or isotope ratio of ⁷⁴Se relative to stable isotopes of Se. In some of any embodiments, the at least one isotopic feature comprises the fractional abundance, atomic percent, or isotope ratio of ⁹⁴Mo relative to stable isotopes of Mo. In some of any embodiments, the at least one isotopic feature comprises the fractional abundance, atomic percent, or isotope ratio of ⁹⁷Mo relative to stable isotopes of Mo.

In some of any embodiments, the at least one isotopic feature comprises the fractional abundance, atomic percent, or isotope ratio of ¹⁰⁷Ag, ¹⁰⁹Ag, ¹¹⁸Sn, ¹²⁷, ¹³C, ²³⁵U, ²⁷Al, ³⁴S, ⁴⁴Ca, ⁵⁷Fe, ⁶⁷Zn, ⁷⁴Se, ⁹⁴Mo, and ⁹⁷Mo, each relative to stable isotopes of the isotopic element of the isotope.

In some of any embodiments, the at least one isotopic feature comprises the fractional abundance, atomic percent, or isotope ratio of ¹²⁷I relative to stable isotopes of I. In some of any embodiments, the at least one isotopic feature comprises the fractional abundance, atomic percent, or isotope ratio of ⁴⁴Ca relative to stable isotopes of Ca. In some of any embodiments, the at least one isotopic feature comprises the fractional abundance, atomic percent, or isotope ratio of ¹²⁷I relative to stable isotopes of I and the fractional abundance, atomic percent, or isotope ratio of ⁴⁴Ca relative to stable isotopes of Ca.

In some of any embodiments, the isotope of the at least one isotopic feature comprises ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing.

In some of any embodiments, the isotopic element of the at least one isotopic feature comprises Zn. In some of any embodiments, the isotope of the at least one isotopic feature comprises ⁶⁴Zn or ⁶⁶Zn. In some of any embodiments, the isotope of the at least one isotopic feature comprises ⁶⁴Zn. the isotope of the at least one isotopic feature comprises ⁶⁶Zn. In some of any embodiments, the isotope of the at least one isotopic feature comprises ⁶⁴Zn and ⁶⁶Zn.

In some of any embodiments, the isotopic element of the at least one isotopic feature comprises S. In some of any embodiments, the isotope of the at least one isotopic feature comprises ³⁴S.

In some of any embodiments, the isotopic element of the at least one isotopic feature comprises N, C, S, or O. In some of any embodiments, the isotopic element of the at least one isotopic feature comprises N. In some of any embodiments, the isotopic element of the at least one isotopic feature comprises C. In some of any embodiments, the isotopic element of the at least one isotopic feature comprises S. In some of any embodiments, the isotopic element of the at least one isotopic feature comprises O. In some of any embodiments, the isotopic element of the at least one isotopic feature comprises N, C, S, and O.

In some of any embodiments, the machine learning algorithm is a supervised machine learning algorithm.

In some of any embodiments, the prediction model is a classification model. In some of any embodiments, the prediction model is a binary classification model. In some of any embodiments, the prediction model is a multiclass classification model.

In some of any embodiments, the machine learning algorithm is Logistic Regression (e.g., Ridge, Lasso, or ElasticNet), Naive Bayes Classifier, GaussianNB, GaussianNBisotonic, GaussianNBsigmoid, Random Forest Classifier, GradientBoosting Classifier, XGBoost Classifer, HistGradientBoosting Classifier, Deep Neural Network Classifier, DecisionTree Classifier, Bagging Classifier, ExtraTrees Classifier, AdaBoost Classifier, CatBoost Classifier, LGBM Classifier, Support Vector Classifier, Perceptron Classifier (e.g., Multi-layer Perceptron Classifier), Deep Learning Classifier for tabular data (e.g., TabNet or TabML), PassiveAggressiveClassifier, or SGD Classifier, or an ensemble algorithm comprising a combination of any of the foregoing.

In some of any embodiments, the machine learning algorithm is a support vector classifier, a decision tree-based ensemble classifier, or a multilayer perceptron classifier. In some of any embodiments, the machine learning algorithm is a support vector classifier. In some of any embodiments, the machine learning algorithm is a decision tree-based ensemble classifier. In some of any embodiments, the machine learning algorithm is a multilayer perceptron classifier.

In some of any embodiments, the prediction model is a regression model.

In some of any embodiments, the machine learning algorithm is an unsupervised machine learning algorithm.

In some of any embodiments, the prediction model is a general model. In some of any embodiments, at least one subject of the plurality of reference subjects lives in a different geographical region, is a different sex, is a different age, is from a different cultural or ethnic group, has a different diet, and/or has a different cancer type, compared to another subject of the plurality of reference subjects. In some of any embodiments, at least one subject of the plurality of reference subjects lives in a different geographical region, is a different sex, is a different age, is from a different cultural or ethnic group, has a different diet, or has a different cancer type, compared to another subject of the plurality of reference subjects. In some of any embodiments, at least one subject of the plurality of reference subjects lives in a different geographical region compared to another subject of the plurality of reference subjects. In some of any embodiments, the plurality of reference subjects are from at least 2, 3, 4, 5, 6, or more, different countries. In some of any embodiments, at least one subject of the plurality of reference subjects is a different sex compared to another subject of the plurality of reference subjects. In some of any embodiments, at least one subject of the plurality of reference subjects is a different age compared to another subject of the plurality of reference subjects. In some of any embodiments, at least one subject of the plurality of reference subjects is from a different cultural or ethnic group compared to another subject of the plurality of reference subjects. In some of any embodiments, at least one subject of the plurality of reference subjects has a different diet compared to another subject of the plurality of reference subjects. In some of any embodiments, at least one subject of the plurality of reference subjects has a different cancer type compared to another subject of the plurality of reference subjects. In some of any embodiments, at least one subject of the plurality of reference subjects lives in a different geographical region, is a different sex, is a different age, is from a different cultural or ethnic group, has a different diet, and has a different cancer type, compared to another subject of the plurality of reference subjects.

In some of any embodiments, the prediction model is a specialized model. In some of any embodiments, each subject of the plurality of reference subjects live in the same geographical region, are the same sex, are the same age, and/or have the same cancer type. In some of any embodiments, each subject of the plurality of reference subjects live in the same geographical region, are the same sex, are the same age, or have the same cancer type. In some of any embodiments, each subject of the plurality of reference subjects live in the same geographical region. In some of any embodiments, each subject of the plurality of reference subjects are the same sex. In some of any embodiments, each subject of the plurality of reference subjects are male. In some of any embodiments, each subject of the plurality of reference subjects are female. In some of any embodiments, each subject of the plurality of reference subjects are the same age. In some of any embodiments, each subject of the plurality of reference subjects have the same cancer type. In some of any embodiments, each subject of the plurality of reference subjects live in the same geographical region, are the same sex, are the same age, and have the same cancer type.

In some of any embodiments, the subject is a mammal. In some of any embodiments, the subject is a human.

In some of any embodiments, the cancer is a blood cancer or is a solid tumor. In some of any embodiments, the cancer is a blood cancer. In some of any embodiments, the blood cancer is a leukemia. a lymphoma. or a multiple myeloma. In some of any embodiments, the blood cancer is a lymphoma. In some of any embodiments, the blood cancer is a multiple myeloma. In some of any embodiments, the blood cancer is a leukemia. In some of any embodiments, the cancer is a solid tumor.

In some of any embodiments, the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

In some of any embodiments, the cancer is prostate cancer, bladder cancer, kidney cancer, head-and-neck cancer, breast cancer, or leukemia. In some of any embodiments, the cancer is prostate cancer. In some of any embodiments, the cancer is bladder cancer. In some of any embodiments, the cancer is kidney cancer. In some of any embodiments, the cancer is head-and-neck cancer. In some of any embodiments, the cancer is breast cancer. In some of any embodiments, the cancer is leukemia.

In some of any embodiments, the method is noninvasive.

In some of any embodiments, the prediction model is built by: (i) determining the values for each of the at least one isotopic feature for the plurality of reference samples; and (ii) using the values for each of the at least one isotopic feature for the plurality of reference samples to train the machine learning algorithm.

In some of any embodiments, some of the plurality of reference subjects are known to have cancer. In some of any embodiments, some of the plurality of reference subjects are assumed to not have cancer. In some of any embodiments, some of the plurality of reference subjects are known to have cancer, and some of the plurality of reference subjects are assumed to not have cancer.

In some of any embodiments, the machine learning algorithm is trained using a plurality of labels. In some of any embodiments, the plurality of labels comprises a label for each of the plurality of reference samples. In some of any embodiments, the machine learning algorithm is trained using a plurality of labels, the plurality of labels comprising a label for each of the plurality of reference samples.

In some of any embodiments, the label indicates if the reference sample is from a reference subject known to have cancer or from a reference subject assumed to not have cancer.

In some of any embodiments, the label indicates the characteristic of the cancer in the reference subject from which the reference sample was obtained.

In some of any embodiments, the label indicates the level of tumor burden of the cancer in the reference subject from which the reference sample was obtained.

In some of any embodiments, the method further comprises, prior to step (a), training the machine learning algorithm using the values for each of the at least one isotopic feature for the plurality of reference samples. In some of any embodiments, the method further comprises, prior to step (a), training the machine learning algorithm using the plurality of labels. In some of any embodiments, the method further comprises, prior to step (a), training the machine learning algorithm using the plurality of labels and the values for each of the at least one isotopic feature for the plurality of reference samples.

In some of any embodiments, the method further comprises preprocessing the values for each of the at least one isotopic feature prior to the training the machine learning algorithm. In some of any embodiments, the preprocessing comprises controlled imputation of missing values. In some of any embodiments, the preprocessing comprises replacing missing values with a zero. In some of any embodiments, the preprocessing comprises data normalization.

In some of any embodiments, cancer is detected in the subject if the prediction model indicates the presence of cancer.

In some of any embodiments, the accuracy of predicting cancer by the method is greater than 50%, greater than 60%, greater than 70%, greater than 80% or greater than 90%, or greater than 95%. In some of any embodiments, the accuracy of predicting cancer by the method is greater than about 70%. In some of any embodiments, the accuracy of predicting cancer is greater than 90%, such as greater than 91%, greater than 92%, greater than 93%, greater than 94% or greater than 95%.

In some of any embodiments, the method further comprises verification of the detected cancer by a method selected from the group consisting of a blood test, a urine test, a biopsy, an endoscopic exam, a lumbar puncture, a pap test, surgery, genetic testing, and imaging.

In some of any embodiments, the method is for diagnosing cancer in the subject. In some of any embodiments, the subject is diagnosed with cancer if the prediction model indicates the presence of cancer. In some of any embodiments, the method is for diagnosing cancer in the subject, wherein the subject is diagnosed with cancer if the prediction model indicates the presence of cancer.

In some of any embodiments, the subject has one or more symptoms that indicate a cancer may be present in the subject.

In some of any embodiments, the method is a preventive screening method for cancer in the subject.

In some of any embodiments, if the subject is diagnosed with cancer, the subject undergoes treatment for the cancer. In some of any embodiments, if the subject is diagnosed with cancer, the method further comprises treating the subject for the cancer.

Also provided herein in some embodiments is a method of treating a cancer in a subject.

In some of any embodiments, the method comprises diagnosing a subject with cancer according to any of the provided methods. In some of any embodiments, the prediction model indicates the presence of cancer.

In some of any embodiments, the method comprises selecting a subject diagnosed with cancer according to any of the provided methods. In some of any embodiments, the prediction model indicates the presence of the cancer.

In some of any embodiments, the method comprises treating the subject for the cancer with a treatment for the cancer.

Also provided herein in some embodiments is a method of treating a cancer in a subject, comprising: (a) diagnosing a subject with cancer according to any of the provided methods, wherein the prediction model indicates the presence of cancer; and (b) treating the subject for the cancer with a treatment for the cancer.

Also provided herein in some embodiments is a method of treating a cancer in a subject, comprising: (a) selecting a subject diagnosed with cancer according to any of the provided methods, wherein the prediction model indicates the presence of the cancer; and (b) treating the subject for the cancer with a treatment for the cancer.

In some of any embodiments, the method is for monitoring cancer treatment in the subject.

In some of any embodiments, the subject has been previously diagnosed with cancer. In some of any embodiments, the subject has been previously diagnosed with cancer and is undergoing treatment, or has been previously diagnosed with cancer and is believed to be in remission. In some of any embodiments, the subject has been previously diagnosed with cancer and is undergoing treatment. In some of any embodiments, the subject has been previously diagnosed with cancer and is believed to be in remission.

In some of any embodiments, if cancer is detected in the subject, cancer treatment for the subject is continued or re-started. In some of any embodiments, if cancer is detected in the subject, cancer treatment for the subject is continued. In some of any embodiments, if cancer is detected in the subject, cancer treatment for the subject is re-started.

In some of any embodiments, if cancer is detected in the subject, the method further comprises continuing or re-starting treatment of the subject for the cancer. In some of any embodiments, if cancer is detected in the subject, the method further comprises continuing treatment of the subject for the cancer. In some of any embodiments, if cancer is detected in the subject, the method further comprises re-starting treatment of the subject for the cancer.

Also provided herein in some embodiments is a method of treating a cancer in a subject, comprising: (a) detecting a cancer in a subject according to any of the provided methods, wherein the prediction model indicates the presence of cancer, and the subject has been previously diagnosed with cancer and is undergoing treatment for the cancer, or has been previously diagnosed with cancer and is believed to be in remission; and (b) treating the subject for the cancer with a treatment for the cancer.

Also provided herein in some embodiments is a method of treating a cancer in a subject, comprising: (a) selecting a subject in which a cancer is detected according to any of the provided methods, wherein the prediction model indicates the presence of the cancer, and the subject has been previously diagnosed with the cancer and is undergoing treatment for the cancer, or has been previously diagnosed with the cancer and is believed to be in remission; and (b) treating the subject for the cancer with a treatment for the cancer.

In some of any embodiments, the treatment comprises chemotherapy, radiotherapy, cryotherapy, photodynamic therapy, laser therapy, immunotherapy, targeted therapy, hormone therapy, surgical procedures to remove the cancer, stem cell transplant, bone marrow transplant, or a combination of any of the foregoing.

In some of any embodiments, the prediction model is built and applied using a computer-program product tangibly embodied in a non-transitory machine-readable storage medium comprising instructions configured to cause one or more data processors to build and apply the prediction model.

Also provided herein in some embodiments is a system for detecting whether a subject has a cancer. Also provided herein in some embodiments is a system for detecting a cancer in a subject.

In some of any embodiments, the system comprises one or more data processors and a non-transitory computer readable storage medium containing instructions. In some of any embodiments, the instructions, when executed on the one or more data processors, cause the one or more data processors to perform actions.

In some of any embodiments, the actions comprising receiving, at one or more processors, the value for each of at least one isotopic feature for a sample from a subject. In some of any embodiments, the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject. In some of any embodiments, the at least one isotopic feature comprises the overall concentration of an isotopic element measured in the sample of the subject. In some of any embodiments, the at least one isotopic feature comprises a chemical feature determined from the overall concentration of an isotopic element measured in the sample of the subject. In some of any embodiments, the at least one isotopic feature comprises the concentration of an isotope of an isotopic element measured in the sample of the subject. In some of any embodiments, the at least one isotopic feature comprises the concentration of an isotope of an isotopic element measured in the sample of the subject. In some of any embodiments, the at least one isotopic feature comprises a concentration of an element, a concentration of an isotopic element, a concentration of an isotope of the isotopic element, or a normalized value of any thereof. In some of any embodiments, the at least one isotopic feature comprises a concentration of an element measured in the sample of the subject. In some of any embodiments, the at least one isotopic feature comprises a concentration of an isotopic element measured in the sample of the subject. In some of any embodiments, the at least one isotopic feature comprises a concentration of an isotope of the isotopic element measured in the sample of the subject. In some of any embodiments, the at least one isotopic feature comprises a normalized value of a concentration of an element measured in the sample of the subject. In some of any embodiments, the at least one isotopic feature comprises a normalized value of a concentration of an isotopic element measured in the sample of the subject. In some of any embodiments, the at least one isotopic feature comprises a normalized value of a concentration of an isotope of the isotopic element measured in the sample of the subject.

In some of any embodiments, the actions comprise inputting, using at least one processor, the value for each of the at least one isotopic feature from the subject into a prediction model. In some of any embodiments, the prediction model is configured to predict the presence or absence of a cancer in a subject. In some of any embodiments, the prediction model comprises at least one machine learning algorithm trained using values for each of the at least one isotopic feature from a plurality of reference samples. In some of any embodiments, the plurality of reference samples are from a plurality of reference subjects.

In some of any embodiments, the actions comprise determining, using the prediction model, whether the subject has the cancer. In some of any embodiments, the actions comprise detecting, using the prediction model, the cancer in the subject.

Also provided herein in some embodiments is a system for detecting whether a subject has a cancer, the system comprising one or more data processors and a non-transitory computer readable storage medium containing instructions which, when executed on the one or more data processors, cause the one or more data processors to perform actions comprising: (a) receiving, at one or more processors, the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer.

Also provided herein in some embodiments is system for detecting whether a subject has a cancer, the system comprising one or more data processors and a non-transitory computer readable storage medium containing instructions which, when executed on the one or more data processors, cause the one or more data processors to perform actions comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer.

Also provided herein in some embodiments is system for detecting a cancer in a subject, the system comprising one or more data processors and a non-transitory computer readable storage medium containing instructions which, when executed on the one or more data processors, cause the one or more data processors to perform actions comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) detecting the cancer in the subject using the prediction model.

Also provided herein in some embodiments is a system for assessing a characteristic of a cancer in a subject.

In some of any embodiments, the system comprises one or more data processors and a non-transitory computer readable storage medium containing instructions. In some of any embodiments, the instructions, when executed on the one or more data processors, cause the one or more data processors to perform actions.

In some of any embodiments, the actions comprising receiving, at one or more processors, the value for each of at least one isotopic feature for a sample from a subject. In some of any embodiments, the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject. In some of any embodiments, the at least one isotopic feature comprises the overall concentration of an isotopic element measured in the sample of the subject. In some of any embodiments, the at least one isotopic feature comprises a chemical feature determined from the overall concentration of an isotopic element measured in the sample of the subject. In some of any embodiments, the at least one isotopic feature comprises the concentration of an isotope of an isotopic element measured in the sample of the subject. In some of any embodiments, the at least one isotopic feature comprises the concentration of an isotope of an isotopic element measured in the sample of the subject. In some of any embodiments, the at least one isotopic feature comprises a concentration of an element, a concentration of an isotopic element, a concentration of an isotope of the isotopic element, or a normalized value of any thereof. In some of any embodiments, the at least one isotopic feature comprises a concentration of an element measured in the sample of the subject. In some of any embodiments, the at least one isotopic feature comprises a concentration of an isotopic element measured in the sample of the subject. In some of any embodiments, the at least one isotopic feature comprises a concentration of an isotope of the isotopic element measured in the sample of the subject. In some of any embodiments, the at least one isotopic feature comprises a normalized value of a concentration of an element measured in the sample of the subject. In some of any embodiments, the at least one isotopic feature comprises a normalized value of a concentration of an isotopic element measured in the sample of the subject. In some of any embodiments, the at least one isotopic feature comprises a normalized value of a concentration of an isotope of the isotopic element measured in the sample of the subject.

In some of any embodiments, the actions comprise inputting, using at least one processor, the value for each of the at least one isotopic feature from the subject into a prediction model. In some of any embodiments, the prediction model is configured to predict a characteristic of a cancer in a subject. In some of any embodiments, the prediction model comprises at least one machine learning algorithm trained using values for each of the at least one isotopic feature from a plurality of reference samples. In some of any embodiments, the plurality of reference samples are from a plurality of reference subjects.

In some of any embodiments, the actions comprise determining, using the prediction model, the characteristic of the cancer in the subject.

Also provided herein in some embodiments is a system for assessing a characteristic of a cancer in a subject, the system comprising one or more data processors and a non-transitory computer readable storage medium containing instructions which, when executed on the one or more data processors, cause the one or more data processors to perform actions comprising: (a) receiving, at one or more processors, the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting, using at least one processor, inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

Also provided herein in some embodiments is a system for assessing a characteristic of a cancer in a subject, the system comprising one or more data processors and a non-transitory computer readable storage medium containing instructions which, when executed on the one or more data processors, cause the one or more data processors to perform actions comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

In some of any embodiments, the characteristic of the cancer is the level of tumor burden of the cancer in the subject.

In some of any embodiments, the value for each of the at least one isotopic feature is obtained by mass spectrometry analysis. In some of any embodiments, the value is a normalized value, and the determining the value for each of the at least one isotopic feature comprises calculating the normalized value from the mass spectrometry analysis.

In some of any embodiments, the mass spectrometry analysis is of a liquid sample of the sample. In some of any embodiments, the sample is a solid sample, and the mass spectrometry analysis is of a liquid sample of the solid sample. In some of any embodiments, the liquid sample is prepared by digestion of the sample. In some of any embodiments, the liquid sample is prepared by digestion of the solid sample.

In some of any embodiments, the mass spectrometry analysis is of a solid sample of the sample. In some of any embodiments, the sample is a solid sample, and the mass spectrometry analysis is of the solid sample. In some of any embodiments, the solid sample is analyzed via combustion. In some of any embodiments, the sample is a solid sample, and the value for each of the at least one isotopic feature is obtained by mass spectrometry analysis of the solid sample or a liquid sample of the solid sample.

In some of any embodiments, the value for each of the at least one isotopic feature is obtained by mass spectrometry analysis of a liquid sample of the sample or a solid sample of the sample. In some of any embodiments, the sample is a solid sample, and the value for each of the at least one isotopic feature is obtained by mass spectrometry analysis of the solid sample or a liquid sample of the solid sample.

In some of any embodiments, the value for each of the at least one isotopic feature is obtained by mass spectrometry analysis of a liquid sample prepared by digestion of the sample or a solid sample of the sample that is analyzed via combustion. In some of any embodiments, the sample is a solid sample, and the value for each of the at least one isotopic feature is obtained by mass spectrometry analysis of the solid sample or a liquid sample prepared by digestion of the solid sample. In some of any embodiments, the solid sample is analyzed via combustion.

In some of any embodiments, the mass spectrometry analysis is performed by: (i) preparing a liquid sample of the sample for analysis by mass spectrometry, wherein the liquid sample is prepared by digestion of the sample; (ii) introducing the prepared liquid sample into a mass spectrometer to detect one or more ionization products of isotopic elements present in the prepared liquid sample; and (iii) analyzing the detected one or more ionization products of isotopic elements to generate the value for each of the at least one isotopic feature. In some of any embodiments, the sample is a solid sample, and the mass spectrometry analysis is performed by: (i) preparing a liquid sample of the sample for analysis by mass spectrometry, wherein the liquid sample is prepared by digestion of the solid sample; (ii) introducing the prepared liquid sample into a mass spectrometer to detect one or more ionization products of isotopic elements present in the prepared liquid sample; and (iii) analyzing the detected one or more ionization products of isotopic elements to generate the value for each of the at least one isotopic feature

In some of any embodiments, the mass spectrometry analysis is performed by: (i) preparing a solid sample of the sample for analysis by mass spectrometry; (ii) introducing the prepared solid sample into a mass spectrometer to detect one or more ionization products of isotopic elements present in the prepared solid sample, wherein the detecting comprises combustion of the prepared solid sample; and (iii) analyzing the detected one or more ionization products of isotopic elements to generate the value for each of the at least one isotopic feature. In some of any embodiments, the sample is a solid sample, and the mass spectrometry analysis is performed by: (i) preparing the solid sample for analysis by mass spectrometry; (ii) introducing the prepared solid sample into a mass spectrometer to detect one or more ionization products of isotopic elements present in the prepared solid sample, optionally wherein the detecting comprises combustion of the prepared solid sample; and (iii) analyzing the detected one or more ionization products of isotopic elements to generate the value for each of the at least one isotopic feature. In some of any embodiments, the sample is a solid sample, and the mass spectrometry analysis is performed by: (i) preparing the solid sample for analysis by mass spectrometry; (ii) introducing the prepared solid sample into a mass spectrometer to detect one or more ionization products of isotopic elements present in the prepared solid sample, wherein the detecting comprises combustion of the prepared solid sample; and (iii) analyzing the detected one or more ionization products of isotopic elements to generate the value for each of the at least one isotopic feature.

In some of any embodiments, the analyzing the detected one or more ionization products of isotopic elements is with reference to one or more ionization products of elements present in standards. In some of any embodiments, the analyzing the detected one or more ionization products of elements and isotopic elements is with reference to one or more ionization products of elements and isotopic elements present in standards.

In some of any embodiments, the analyzing the detected one or more ionization products of isotopic elements is with reference to one or more ionization products of elements present in standards comprising a plurality of calibration standards and a blank standard. In some of any embodiments, the analyzing the detected one or more ionization products of isotopic elements and elements is with reference to one or more ionization products of elements and isotopic elements present in standards comprising a plurality of calibration standards and a blank standard.

In some of any embodiments, the analyzing the detected one or more ionization products of isotopic elements is with reference to one or more ionization products of elements present in standards comprising an internal standard and/or a reference material. In some of any embodiments, the analyzing the detected one or more ionization products of isotopic elements is with reference to one or more ionization products of elements present in standards comprising an internal standard. In some of any embodiments, the analyzing the detected one or more ionization products of isotopic elements is with reference to one or more ionization products of elements present in standards comprising a reference material. In some of any embodiments, the analyzing the detected one or more ionization products of isotopic elements is with reference to one or more ionization products of elements present in standards comprising an internal standard and a reference material.

In some of any embodiments, the analyzing the detected one or more ionization products of isotopic elements and elements is with reference to one or more ionization products of elements and isotopic elements present in standards comprising an internal standard and/or a reference material. In some of any embodiments, the analyzing the detected one or more ionization products of isotopic elements and elements is with reference to one or more ionization products of elements and isotopic elements present in standards comprising an internal standard. In some of any embodiments, the analyzing the detected one or more ionization products of isotopic elements and elements is with reference to one or more ionization products of elements and isotopic elements present in standards comprising a reference material. In some of any embodiments, the analyzing the detected one or more ionization products of isotopic elements and elements is with reference to one or more ionization products of elements and isotopic elements present in standards comprising an internal standard and a reference material.

In some of any embodiments, the mass spectrometer is further introduced with the standards, wherein the mass spectrometer detects one or more ionization products of elements present in the standards and the prepared liquid or solid sample. In some of any embodiments, the mass spectrometer is further introduced with the standards, wherein the mass spectrometer detects one or more ionization products of elements or isotopic elements present in the standards and the prepared liquid or solid sample.

In some of any embodiments, the sample comprises a keratinous tissue, optionally a hard keratinous tissue. In some of any embodiments, the sample comprises a keratinous tissue. In some of any embodiments, the sample comprises a hard keratinous tissue.

In some of any embodiments, the sample is a noninvasive sample, optionally a hair sample or a nail sample. In some of any embodiments, the sample is a noninvasive sample. In some of any embodiments, the sample is a hair sample or a nail sample. In some of any embodiments, the sample is a hair sample. In some of any embodiments, the sample is a nail sample.

In some of any embodiments, the digestion is by acid digestion and/or thermal digestion. In some of any embodiments, the digestion is by acid digestion and thermal digestion.

In some of any embodiments, the acid digestion is with nitric acid (HNO₃). In some of any embodiments, the acid digestion is with nitric acid (HNO₃) and hydrogen peroxide (H₂O₂).

In some of any embodiments, the thermal digestion is by a microwave.

In some of any embodiments, the mass spectrometry is Inductively Coupled Plasma Mass Spectrometry (ICP-MS). In some of any embodiments, the ICP-MS is carried out with kinetic energy discrimination (KED).

In some of any embodiments, the at least one isotopic feature comprises an atomic percent, isotope ratio, fractional abundance, delta, overall concentration, and/or concentration ratio. In some of any embodiments, the at least one isotopic feature comprises an atomic percent. In some of any embodiments, the at least one isotopic feature comprises an isotope ratio. In some of any embodiments, the at least one isotopic feature comprises a fractional abundance. In some of any embodiments, the at least one isotopic feature comprises a delta. In some of any embodiments, the at least one isotopic feature comprises an overall concentration. In some of any embodiments, the at least one isotopic feature comprises a concentration ratio.

In some of any embodiments, the at least one isotopic feature comprises an atomic percent for at least one isotope of an isotopic element, an isotope ratio for at least one isotope of an isotopic element, a fractional abundance for at least one isotope of an isotopic element, and/or a delta for at least one isotope of an isotopic element. In some of any embodiments, the at least one isotopic feature comprises an atomic percent for at least one isotope of an isotopic element. In some of any embodiments, the at least one isotopic feature comprises an isotope ratio for at least one isotope of an isotopic element. In some of any embodiments, the at least one isotopic feature comprises a fractional abundance for at least one isotope of an isotopic element. In some of any embodiments, the at least one isotopic feature comprises a delta for at least one isotope of an isotopic element.

In some of any embodiments, the at least one isotopic feature is a plurality of isotopic features each independently selected from the group consisting of atomic percents, isotope ratios, fractional abundances, deltas, overall concentrations, and concentration ratios.

In some of any embodiments, the plurality of isotopic features comprises an atomic percent for at least one isotope of an isotopic element, an isotope ratio for at least one isotope of an isotopic element, a fractional abundance for at least one isotope of an isotopic element, and/or a delta for at least one isotope of an isotopic element. In some of any embodiments, the plurality of isotopic features comprises an atomic percent for at least one isotope of an isotopic element. In some of any embodiments, the plurality of isotopic features comprises an isotope ratio for at least one isotope of an isotopic element. In some of any embodiments, the plurality of isotopic features comprises a fractional abundance for at least one isotope of an isotopic element. In some of any embodiments, the plurality of isotopic features comprises a delta for at least one isotope of an isotopic element.

In some of any embodiments, the value of each of the at least one isotopic feature is a normalized value of a concentration of an element, a normalized value of a concentration of an isotopic element, or a normalized value of a concentration of an isotope of the isotopic element. In some of any embodiments, the value of each of the at least one isotopic feature is a normalized value of a concentration of an element. In some of any embodiments, the value of each of the at least one isotopic feature is a normalized value of a concentration of an isotopic element. In some of any embodiments, the value of each of the at least one isotopic feature is a normalized value of a concentration of an isotope of the isotopic element.

In some of any embodiments, the normalized value of each of the at least one isotopic feature is an atomic percent (atom %), fractional abundance (F), isotope ratio (R), concentration ratio (CR), or a delta value (delta), or a combination of any of the foregoing. In some of any embodiments, the normalized value of each of the least one isotopic feature is an atomic percent (atom %). In some of any embodiments, the normalized value of each of the least one isotopic feature is a fractional abundance (F). In some of any embodiments, the normalized value of each of the least one isotopic feature is an isotope ratio (R). In some of any embodiments, the normalized value of each of the least one isotopic feature is a concentration ratio (CR). In some of any embodiments, the normalized value of each of the least one isotopic feature is a delta value (delta).

In some of any embodiments, the normalized value of each of the at least one isotopic feature is a delta value (delta). In some of any embodiments, the delta value is a conventional delta value, a study-specific delta value (InH), a non-similar simple-nitrogen based delta value (SimpleNδ), or a non-similar pseudo-nitrogen based delta value (PseudoNδ). In some of any embodiments, the delta value is a conventional delta value. In some of any embodiments, the delta value is a study-specific delta value (InH). In some of any embodiments, the delta value is a non-similar simple-nitrogen based delta value (SimpleNδ). In some of any embodiments, the delta value is a non-similar pseudo-nitrogen based delta value (PseudoNδ).

In some of any embodiments, the isotopic element is Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S.

In some of any embodiments, the isotopic element is Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, or Zn. In some of any embodiments, the isotopic element is Ag. In some of any embodiments, the isotopic element is Al. In some of any embodiments, the isotopic element is B. In some of any embodiments, the isotopic element is Ba. In some of any embodiments, the isotopic element is Br. In some of any embodiments, the isotopic element is Ca. In some of any embodiments, the isotopic element is Co. In some of any embodiments, the isotopic element is Cr. In some of any embodiments, the isotopic element is Cu. In some of any embodiments, the isotopic element is Fe. In some of any embodiments, the isotopic element is Ge. In some of any embodiments, the isotopic element is Hg. In some of any embodiments, the isotopic element is I. In some of any embodiments, the isotopic element is K. In some of any embodiments, the isotopic element is Li. In some of any embodiments, the isotopic element is Mg. In some of any embodiments, the isotopic element is Mn. In some of any embodiments, the isotopic element is Mo. In some of any embodiments, the isotopic element is Na. In some of any embodiments, the isotopic element is Ni. In some of any embodiments, the isotopic element is Pb. In some of any embodiments, the isotopic element is Rb. In some of any embodiments, the isotopic element is Ru. In some of any embodiments, the isotopic element is Se. In some of any embodiments, the isotopic element is Sn. In some of any embodiments, the isotopic element is Sr. In some of any embodiments, the isotopic element is Te. In some of any embodiments, the isotopic element is Ti. In some of any embodiments, the isotopic element is U. In some of any embodiments, the isotopic element is V. In some of any embodiments, the isotopic element is Y. In some of any embodiments, the isotopic element is Zn.

In some of any embodiments, the isotopic element is N, C, S, or O. In some of any embodiments, the isotopic element is N. In some of any embodiments, the isotopic element is C. In some of any embodiments, the isotopic element is S. In some of any embodiments, the isotopic element is O.

In some of any embodiments, the isotopic element is Mo, Cr, Te, C, or Hg. In some of any embodiments, the isotopic element is Mo. In some of any embodiments, the isotopic element is Cr. In some of any embodiments, the isotopic element is Te. In some of any embodiments, the isotopic element is C. In some of any embodiments, the isotopic element is Hg.

In some of any embodiments, the at least one isotopic feature is one isotopic feature. In some of any embodiments, the isotope of the at least one isotopic feature comprises ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr. In some of any embodiments, the isotope of the at least one isotopic feature comprises ⁶⁶Zn. In some of any embodiments, the isotope of the at least one isotopic feature comprises ⁶⁴Zn. In some of any embodiments, the isotope of the at least one isotopic feature comprises ⁶⁸Zn. In some of any embodiments, the isotope of the at least one isotopic feature comprises ⁸⁷Sr. In some of any embodiments, the isotope of the at least one isotopic feature comprises ⁸⁸Sr. In some of any embodiments, the isotope of the at least one isotopic feature comprises ⁸⁶Sr. In some of any embodiments, the isotope of the at least one isotopic feature comprises ⁴⁷Ti. In some of any embodiments, the isotope of the at least one isotopic feature comprises ⁴⁸Ti. In some of any embodiments, the isotope of the at least one isotopic feature comprises ⁴⁶Ti. In some of any embodiments, the isotope of the at least one isotopic feature comprises ⁵⁰Cr. In some of any embodiments, the isotope of the at least one isotopic feature comprises ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and ⁵⁰Cr.

In some of any embodiments, the at least one isotopic feature is a plurality of isotopic features. In some of any embodiments, the plurality of isotopic features is about 2 to about 100 isotopic features. In some of any embodiments, the plurality of isotopic features is 2-100 isotopic features. In some of any embodiments, the plurality of isotopic features comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or more isotopic features. In some of any embodiments, the plurality of isotopic features comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic features.

In some of any embodiments, the at least one isotopic feature is a plurality of isotopic features determined from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or more elements or isotopic elements. In some of any embodiments, the plurality of isotopic features are determined from at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic elements.

In some of any embodiments, the plurality of isotopic features comprises fractional abundances and deltas. In some of any embodiments, the fractional abundance and delta values are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

In some of any embodiments, the plurality of isotopic features comprises fractional abundances, atomic percents, and/or isotope ratios. In some of any embodiments, the fractional abundances, atomic percents, and/or isotope ratios are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, and/or S. In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, and S. In some of any embodiments, the element or the isotopic element of the at least one isotopic feature comprises Li, B, Na, Mg, Al, P, K, Sc, Ca, Ti, V, Cr, Mn, Fe, Co, Ni, Cu, Zn, Ge, As, Se, Br, Rb, Sr, Y, Zr, Nb, Mo, Ru, Ag, Cd, In, Sn, I, Te, Ba, Hg, Pb, U, C, N, O, or S, or a combination of any of the foregoing. In some of any embodiments, the element or the isotopic element of the at least one isotopic feature comprises Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S, or a combination of any of the foregoing. In some of any embodiments, the element or the isotopic element of the at least one isotopic feature comprises Li, B, Mg, Ca, Ti, Cr, Fe, Ni, Cu, Zn, Se, Br, Rb, Sr, Mo, Ag, Sn, Ba, Hg, Pb, U, C, N, S, or a combination of any of the foregoing.

In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, or Zn. In some of any embodiments, the isotopic elements comprise Ag. In some of any embodiments, the isotopic elements comprise Al. In some of any embodiments, the isotopic elements comprise B. In some of any embodiments, the isotopic elements comprise Ba. In some of any embodiments, the isotopic elements comprise Br. In some of any embodiments, the isotopic elements comprise Ca. In some of any embodiments, the isotopic elements comprise Co. In some of any embodiments, the isotopic elements comprise Cr. In some of any embodiments, the isotopic elements comprise Cu. In some of any embodiments, the isotopic elements comprise Fe. In some of any embodiments, the isotopic elements comprise Ge. In some of any embodiments, the isotopic elements comprise Hg. In some of any embodiments, the isotopic elements comprise I. In some of any embodiments, the isotopic elements comprise K. In some of any embodiments, the isotopic elements comprise Li. In some of any embodiments, the isotopic elements comprise Mg. In some of any embodiments, the isotopic elements comprise Mn. In some of any embodiments, the isotopic elements comprise Mo. In some of any embodiments, the isotopic elements comprise Na. In some of any embodiments, the isotopic elements comprise Ni. In some of any embodiments, the isotopic elements comprise Pb. In some of any embodiments, the isotopic elements comprise Rb. In some of any embodiments, the isotopic elements comprise Ru. In some of any embodiments, the isotopic elements comprise Se. In some of any embodiments, the isotopic elements comprise Sn. In some of any embodiments, the isotopic elements comprise Sr. In some of any embodiments, the isotopic elements comprise Te. In some of any embodiments, the isotopic elements comprise Ti. In some of any embodiments, the isotopic elements comprise U. In some of any embodiments, the isotopic elements comprise V. In some of any embodiments, the isotopic elements comprise Y. In some of any embodiments, the isotopic elements comprise Zn. In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, and Zn.

In some of any embodiments, the isotopic elements comprise N, C, S, and/or O. In some of any embodiments, the isotopic elements comprise N. In some of any embodiments, the isotopic elements comprise C. In some of any embodiments, the isotopic elements comprise S. In some of any embodiments, the isotopic elements comprise O. In some of any embodiments, the isotopic elements comprise N, C, S, and O.

In some of any embodiments, the isotopic elements comprise Mo, Cr, Te, C, and/or Hg. In some of any embodiments, the isotopic elements comprise Mo. In some of any embodiments, the isotopic elements comprise Cr. In some of any embodiments, the isotopic elements comprise Te. In some of any embodiments, the isotopic elements comprise C. In some of any embodiments, the isotopic elements comprise Hg. In some of any embodiments, the isotopic elements comprise Mo, Cr, Te, C, and Hg.

In some of any embodiments, at least one isotopic feature comprises a concentration ratio. In some of any embodiments, the concentration ratio is N/C, S/C, O/C, S/N, N/O, or S/O. In some of any embodiments, the concentration ratio is N/C. In some of any embodiments, the concentration ratio is S/C. In some of any embodiments, the concentration ratio is O/C. In some of any embodiments, the concentration ratio is S/N. In some of any embodiments, the concentration ratio is N/O. In some of any embodiments, the concentration ratio is S/O.

In some of any embodiments, the plurality of isotopic features comprises at least one concentration ratio. In some of any embodiments, the at least one concentration ratio comprises N/C, S/C, O/C, S/N, N/O, and/or S/O. In some of any embodiments, the at least one concentration ratio comprises N/C. In some of any embodiments, the at least one concentration ratio comprises S/C. In some of any embodiments, the at least one concentration ratio comprises O/C. In some of any embodiments, the at least one concentration ratio comprises S/N. In some of any embodiments, the at least one concentration ratio comprises N/O. In some of any embodiments, the at least one concentration ratio comprises S/O. In some of any embodiments, the at least one concentration ratio comprises N/C, S/C, O/C, S/N, N/O, and S/O.

In some of any embodiments, the at least one isotopic feature comprises at least 1, 2, 3, 4, 5, 6, 7, 8, 9, or 10 of the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (f³⁴S) relative to stable isotopes of S, the fractional abundance, atomic percent, or isotope ratio of ¹⁵N (f15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO). In some of any embodiments, the at least one isotopic feature comprises the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (f³⁴S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO).

In some of any embodiments, the at least one isotopic feature comprises the fractional abundance, atomic percent, or isotope ratio of ¹³C (F13C) relative to stable isotopes of C. In some of any embodiments, the at least one isotopic feature comprises the fractional abundance, atomic percent, or isotope ratio of ¹⁸O (F18O) relative to stable isotopes of O. In some of any embodiments, the at least one isotopic feature comprises the concentration ratio of sulfur over nitrogen (SdN). In some of any embodiments, the at least one isotopic feature comprises the concentration ratio of nitrogen over carbon (NdC). In some of any embodiments, the at least one isotopic feature comprises the concentration ratio of nitrogen over carbon (NdC). In some of any embodiments, the at least one isotopic feature comprises the fractional abundance, atomic percent, or isotope ratio of ³⁴S (F³⁴S) relative to stable isotopes of S. In some of any embodiments, the at least one isotopic feature comprises the fractional abundance, atomic percent, or isotope ratio of ¹⁵N (F15N) relative to stable isotopes of N. In some of any embodiments, the at least one isotopic feature comprises the concentration ratio of oxygen over carbon (OdC). In some of any embodiments, the at least one isotopic feature comprises the concentration ratio of nitrogen over oxygen (NdO). In some of any embodiments, the at least one isotopic feature comprises the concentration ratio of sulfur over carbon (SdC). In some of any embodiments, the at least one isotopic feature comprises the concentration ratio of sulfur over oxygen (SdO).

In some of any embodiments, the at least one isotopic feature comprises the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (F³⁴S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO).

In some of any embodiments, the at least one isotopic feature comprises at least 1, 2, 3, 4, 5, 6, 7, 8, 9, 10, 11, 12, 13, 14, 15, 16, 17, 18, 19, 20, or more of the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷, ¹²⁸Te ¹³⁶Ba, ¹³⁷Ba, ¹³C, ²³⁵U, ²³⁸U, ²³Na, ²⁷Al, ³⁴S, ³⁹K, ⁴⁴Ca, ⁴⁸Ti, ⁵⁴Cr, ⁵⁴Fe, ⁵⁷Fe, ⁶²Ni, ⁶³Cu, ⁶⁶Zn, ⁶⁷Zn, ⁶⁸Zn, ⁷⁰Ge, ⁷⁴Ge, ⁷⁴Se, ⁷⁶Se, ⁷⁷Se, ⁷⁹Br, ⁸⁰Se, ⁸²Se, ⁸⁹Y, ⁹⁴Mo, ⁹⁷Mo, and ⁹⁸Mo, each relative to stable isotopes of the isotopic element of the isotope.

In some of any embodiments, the at least one isotopic feature comprises the fractional abundance, atomic percent, or isotope ratio of ¹⁰¹Ru relative to stable isotopes of Ru. In some of any embodiments, the at least one isotopic feature comprises the fractional abundance, atomic percent, or isotope ratio of ¹⁰⁷Ag relative to stable isotopes of Ag. In some of any embodiments, the at least one isotopic feature comprises the fractional abundance, atomic percent, or isotope ratio of ¹⁰⁹Ag relative to stable isotopes of Ag. In some of any embodiments, the at least one isotopic feature comprises the fractional abundance, atomic percent, or isotope ratio of ¹⁰B relative to stable isotopes of B. In some of any embodiments, the at least one isotopic feature comprises the fractional abundance, atomic percent, or isotope ratio of ¹¹⁸Sn relative to stable isotopes of Sn. In some of any embodiments, the at least one isotopic feature comprises the fractional abundance, atomic percent, or isotope ratio of ¹¹⁹Sn relative to stable isotopes of Sn. In some of any embodiments, the at least one isotopic feature comprises the fractional abundance, atomic percent, or isotope ratio of ¹²⁴Sn relative to stable isotopes of Sn. In some of any embodiments, the at least one isotopic feature comprises the fractional abundance, atomic percent, or isotope ratio of ¹²⁴Te relative to stable isotopes of Te. In some of any embodiments, the at least one isotopic feature comprises the fractional abundance, atomic percent, or isotope ratio of ¹²⁷I relative to stable isotopes of I. In some of any embodiments, the at least one isotopic feature comprises the fractional abundance, atomic percent, or isotope ratio of ¹²⁸Te relative to stable isotopes of Te. In some of any embodiments, the at least one isotopic feature comprises the fractional abundance, atomic percent, or isotope ratio of ¹³⁶Ba relative to stable isotopes of Ba. In some of any embodiments, the at least one isotopic feature comprises the fractional abundance, atomic percent, or isotope ratio of ¹³⁷Ba relative to stable isotopes of Ba. In some of any embodiments, the at least one isotopic feature comprises the fractional abundance, atomic percent, or isotope ratio of ¹³C relative to stable isotopes of C. In some of any embodiments, the at least one isotopic feature comprises the fractional abundance, atomic percent, or isotope ratio of ²³⁵U relative to stable isotopes of U. In some of any embodiments, the at least one isotopic feature comprises the fractional abundance, atomic percent, or isotope ratio of ²³⁸U relative to stable isotopes of U. In some of any embodiments, the at least one isotopic feature comprises the fractional abundance, atomic percent, or isotope ratio of ²³Na relative to stable isotopes of Na. In some of any embodiments, the at least one isotopic feature comprises the fractional abundance, atomic percent, or isotope ratio of ²⁷Al relative to stable isotopes of Al. In some of any embodiments, the at least one isotopic feature comprises the fractional abundance, atomic percent, or isotope ratio of ³⁴S relative to stable isotopes of S. In some of any embodiments, the at least one isotopic feature comprises the fractional abundance, atomic percent, or isotope ratio of ³⁹K relative to stable isotopes of K. In some of any embodiments, the at least one isotopic feature comprises the fractional abundance, atomic percent, or isotope ratio of ⁴⁴Ca relative to stable isotopes of Ca. In some of any embodiments, the at least one isotopic feature comprises the fractional abundance, atomic percent, or isotope ratio of ⁴⁸Ti relative to stable isotopes of Ti. In some of any embodiments, the at least one isotopic feature comprises the fractional abundance, atomic percent, or isotope ratio of ⁵⁴Cr relative to stable isotopes of Cr. In some of any embodiments, the at least one isotopic feature comprises the fractional abundance, atomic percent, or isotope ratio of ⁵⁴Fe relative to stable isotopes of Fe. In some of any embodiments, the at least one isotopic feature comprises the fractional abundance, atomic percent, or isotope ratio of ⁵⁷Fe relative to stable isotopes of Fe. In some of any embodiments, the at least one isotopic feature comprises the fractional abundance, atomic percent, or isotope ratio of ⁶²Ni relative to stable isotopes of Ni. In some of any embodiments, the at least one isotopic feature comprises the fractional abundance, atomic percent, or isotope ratio of ⁶³Cu relative to stable isotopes of Cu. In some of any embodiments, the at least one isotopic feature comprises the fractional abundance, atomic percent, or isotope ratio of ⁶⁶Zn relative to stable isotopes of Zn. In some of any embodiments, the at least one isotopic feature comprises the fractional abundance, atomic percent, or isotope ratio of ⁶⁷Zn relative to stable isotopes of Zn. In some of any embodiments, the at least one isotopic feature comprises the fractional abundance, atomic percent, or isotope ratio of ⁶⁸Zn relative to stable isotopes of Zn. In some of any embodiments, the at least one isotopic feature comprises the fractional abundance, atomic percent, or isotope ratio of ⁷⁰Ge relative to stable isotopes of Ge. In some of any embodiments, the at least one isotopic feature comprises the fractional abundance, atomic percent, or isotope ratio of ⁷⁴Ge relative to stable isotopes of Ge. In some of any embodiments, the at least one isotopic feature comprises the fractional abundance, atomic percent, or isotope ratio of ⁷⁴Se relative to stable isotopes of Se. In some of any embodiments, the at least one isotopic feature comprises the fractional abundance, atomic percent, or isotope ratio of ⁷⁶Se relative to stable isotopes of Se. In some of any embodiments, the at least one isotopic feature comprises the fractional abundance, atomic percent, or isotope ratio of ⁷⁷Se relative to stable isotopes of Se. In some of any embodiments, the at least one isotopic feature comprises the fractional abundance, atomic percent, or isotope ratio of ⁷⁹Br relative to stable isotopes of Br. In some of any embodiments, the at least one isotopic feature comprises the fractional abundance, atomic percent, or isotope ratio of ⁸⁰Se relative to stable isotopes of Se. In some of any embodiments, the at least one isotopic feature comprises the fractional abundance, atomic percent, or isotope ratio of ⁸²Se relative to stable isotopes of Se. In some of any embodiments, the at least one isotopic feature comprises the fractional abundance, atomic percent, or isotope ratio of ⁸⁹Y relative to stable isotopes of Y. In some of any embodiments, the at least one isotopic feature comprises the fractional abundance, atomic percent, or isotope ratio of ⁹⁴Mo relative to stable isotopes of Mo. In some of any embodiments, the at least one isotopic feature comprises the fractional abundance, atomic percent, or isotope ratio of ⁹⁷Mo relative to stable isotopes of Mo. In some of any embodiments, the at least one isotopic feature comprises the fractional abundance, atomic percent, or isotope ratio of ⁹⁸Mo relative to stable isotopes of Mo.

In some of any embodiments, the at least one isotopic feature comprises the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷I, ¹²⁸Te, ¹³⁶Ba, ¹³⁷Ba, ¹³C, ²³⁵U, ²³⁸U, ²³Na, ²⁷Al, ³⁴S, ³⁹K, ⁴⁴Ca, ⁴⁸Ti, ⁵⁴Cr, ⁵⁴Fe, ⁵⁷Fe, ⁶²Ni, ⁶³Cu, ⁶⁶Zn, ⁶⁷Zn, ⁶⁸Zn, ⁷⁰Ge, ⁷⁴Ge, ⁷⁴Se, ⁷⁶Se, ⁷⁷Se, ⁷⁹Br, ⁸⁰Se, ⁸²Se, ⁸⁹Y, ⁹⁴Mo, ⁹⁷Mo, and ⁹⁸Mo, each relative to stable isotopes of the isotopic element of the isotope.

In some of any embodiments, the at least one isotopic feature comprises at least 1, 2, 3, 4, 5, 6, 7, 8, 9, 10, or more of the fractional abundance, atomic percent, or isotope ratio of ¹⁰⁷Ag, ¹⁰⁹Ag, ¹¹⁸Sn, ¹²⁷I, ¹³C, ²³⁵U, ²⁷Al, ³⁴S, ⁴⁴Ca, ⁵⁷Fe, ⁶⁷Zn, ⁷⁴Se, ⁹⁴Mo, and ⁹⁷Mo, each relative to stable isotopes of the isotopic element of the isotope.

In some of any embodiments, the at least one isotopic feature comprises the fractional abundance, atomic percent, or isotope ratio of ¹⁰⁷Ag relative to stable isotopes of Ag. In some of any embodiments, the at least one isotopic feature comprises the fractional abundance, atomic percent, or isotope ratio of ¹⁰⁹Ag relative to stable isotopes of Ag. In some of any embodiments, the at least one isotopic feature comprises the fractional abundance, atomic percent, or isotope ratio of ¹¹⁸Sn relative to stable isotopes of Sn. In some of any embodiments, the at least one isotopic feature comprises the fractional abundance, atomic percent, or isotope ratio of ¹²⁷I relative to stable isotopes of I. In some of any embodiments, the at least one isotopic feature comprises the fractional abundance, atomic percent, or isotope ratio of ¹³C relative to stable isotopes of C. In some of any embodiments, the at least one isotopic feature comprises the fractional abundance, atomic percent, or isotope ratio of ²³⁵U relative to stable isotopes of U. In some of any embodiments, the at least one isotopic feature comprises the fractional abundance, atomic percent, or isotope ratio of ²⁷Al relative to stable isotopes of Al. In some of any embodiments, the at least one isotopic feature comprises the fractional abundance, atomic percent, or isotope ratio of ³⁴S relative to stable isotopes of S. In some of any embodiments, the at least one isotopic feature comprises the fractional abundance, atomic percent, or isotope ratio of ⁴Ca relative to stable isotopes of Ca. In some of any embodiments, the at least one isotopic feature comprises the fractional abundance, atomic percent, or isotope ratio of ⁵⁷Fe relative to stable isotopes of Fe. In some of any embodiments, the at least one isotopic feature comprises the fractional abundance, atomic percent, or isotope ratio of ⁶⁷Zn relative to stable isotopes of Zn. In some of any embodiments, the at least one isotopic feature comprises the fractional abundance, atomic percent, or isotope ratio of ⁷⁴Se relative to stable isotopes of Se. In some of any embodiments, the at least one isotopic feature comprises the fractional abundance, atomic percent, or isotope ratio of ⁹⁴Mo relative to stable isotopes of Mo. In some of any embodiments, the at least one isotopic feature comprises the fractional abundance, atomic percent, or isotope ratio of ⁹⁷Mo relative to stable isotopes of Mo.

In some of any embodiments, the at least one isotopic feature comprises the fractional abundance, atomic percent, or isotope ratio of ¹⁰⁷Ag, ¹⁰⁹Ag, ¹¹⁸Sn, ¹²⁷, ¹³C, ²³⁵U, ²⁷Al, ³⁴S, ⁴Ca, ⁵⁷Fe, ⁶⁷Zn, ⁷⁴Se, ⁹⁴Mo, and ⁹⁷Mo, each relative to stable isotopes of the isotopic element of the isotope.

In some of any embodiments, the at least one isotopic feature comprises the fractional abundance, atomic percent, or isotope ratio of ¹²⁷I relative to stable isotopes of I. In some of any embodiments, the at least one isotopic feature comprises the fractional abundance, atomic percent, or isotope ratio of ⁴Ca relative to stable isotopes of Ca. In some of any embodiments, the at least one isotopic feature comprises the fractional abundance, atomic percent, or isotope ratio of ¹²⁷I relative to stable isotopes of I and the fractional abundance, atomic percent, or isotope ratio of ⁴Ca relative to stable isotopes of Ca.

In some of any embodiments, the isotope of the at least one isotopic feature comprises ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing.

In some of any embodiments, the isotopic element of the at least one isotopic feature comprises Zn. In some of any embodiments, the isotope of the at least one isotopic feature comprises ⁶⁴Zn or ⁶⁶Zn. In some of any embodiments, the isotope of the at least one isotopic feature comprises ⁶⁴Zn. the isotope of the at least one isotopic feature comprises ⁶⁶Zn. In some of any embodiments, the isotope of the at least one isotopic feature comprises ⁶⁴Zn and ⁶⁶Zn.

In some of any embodiments, the isotopic element of the at least one isotopic feature comprises S. In some of any embodiments, the isotope of the at least one isotopic feature comprises ³⁴S.

In some of any embodiments, the isotopic element of the at least one isotopic feature comprises N, C, S, or O. In some of any embodiments, the isotopic element of the at least one isotopic feature comprises N. In some of any embodiments, the isotopic element of the at least one isotopic feature comprises C. In some of any embodiments, the isotopic element of the at least one isotopic feature comprises S. In some of any embodiments, the isotopic element of the at least one isotopic feature comprises O. In some of any embodiments, the isotopic element of the at least one isotopic feature comprises N, C, S, and O.

In some of any embodiments, the machine learning algorithm is a supervised machine learning algorithm.

In some of any embodiments, the prediction model is a classification model. In some of any embodiments, the prediction model is a binary classification model. In some of any embodiments, the prediction model is a multiclass classification model.

In some of any embodiments, the machine learning algorithm is Logistic Regression (e.g., Ridge, Lasso, or ElasticNet), Naive Bayes Classifier, GaussianNB, GaussianNBisotonic, GaussianNBsigmoid, Random Forest Classifier, GradientBoosting Classifier, XGBoost Classifer, HistGradientBoosting Classifier, Deep Neural Network Classifier, DecisionTree Classifier, Bagging Classifier, ExtraTrees Classifier, AdaBoost Classifier, CatBoost Classifier, LGBM Classifier, Support Vector Classifier, Perceptron Classifier (e.g., Multi-layer Perceptron Classifier), Deep Learning Classifier for tabular data (e.g., TabNet or TabML), PassiveAggressiveClassifier, or SGD Classifier, or an ensemble algorithm comprising a combination of any of the foregoing.

In some of any embodiments, the machine learning algorithm is a support vector classifier, a decision tree-based ensemble classifier, or a multilayer perceptron classifier. In some of any embodiments, the machine learning algorithm is a support vector classifier. In some of any embodiments, the machine learning algorithm is a decision tree-based ensemble classifier. In some of any embodiments, the machine learning algorithm is a multilayer perceptron classifier.

In some of any embodiments, the prediction model is a regression model.

In some of any embodiments, the machine learning algorithm is an unsupervised machine learning algorithm.

In some of any embodiments, the prediction model is a general model. In some of any embodiments, at least one subject of the plurality of reference subjects lives in a different geographical region, is a different sex, is a different age, is from a different cultural or ethnic group, has a different diet, and/or has a different cancer type, compared to another subject of the plurality of reference subjects. In some of any embodiments, at least one subject of the plurality of reference subjects lives in a different geographical region, is a different sex, is a different age, is from a different cultural or ethnic group, has a different diet, or has a different cancer type, compared to another subject of the plurality of reference subjects. In some of any embodiments, at least one subject of the plurality of reference subjects lives in a different geographical region compared to another subject of the plurality of reference subjects. In some of any embodiments, the plurality of reference subjects are from at least 2, 3, 4, 5, 6, or more, different countries. In some of any embodiments, at least one subject of the plurality of reference subjects is a different sex compared to another subject of the plurality of reference subjects. In some of any embodiments, at least one subject of the plurality of reference subjects is a different age compared to another subject of the plurality of reference subjects. In some of any embodiments, at least one subject of the plurality of reference subjects is from a different cultural or ethnic group compared to another subject of the plurality of reference subjects. In some of any embodiments, at least one subject of the plurality of reference subjects has a different diet compared to another subject of the plurality of reference subjects. In some of any embodiments, at least one subject of the plurality of reference subjects has a different cancer type compared to another subject of the plurality of reference subjects. In some of any embodiments, at least one subject of the plurality of reference subjects lives in a different geographical region, is a different sex, is a different age, is from a different cultural or ethnic group, has a different diet, and has a different cancer type, compared to another subject of the plurality of reference subjects.

In some of any embodiments, the prediction model is a specialized model. In some of any embodiments, each subject of the plurality of reference subjects live in the same geographical region, are the same sex, are the same age, and/or have the same cancer type. In some of any embodiments, each subject of the plurality of reference subjects live in the same geographical region, are the same sex, are the same age, or have the same cancer type. In some of any embodiments, each subject of the plurality of reference subjects live in the same geographical region. In some of any embodiments, each subject of the plurality of reference subjects are the same sex. In some of any embodiments, each subject of the plurality of reference subjects are male. In some of any embodiments, each subject of the plurality of reference subjects are female. In some of any embodiments, each subject of the plurality of reference subjects are the same age. In some of any embodiments, each subject of the plurality of reference subjects have the same cancer type. In some of any embodiments, each subject of the plurality of reference subjects live in the same geographical region, are the same sex, are the same age, and have the same cancer type.

In some of any embodiments, the subject is a mammal. In some of any embodiments, the subject is a human.

In some of any embodiments, the cancer is a blood cancer or is a solid tumor. In some of any embodiments, the cancer is a blood cancer. In some of any embodiments, the blood cancer is a leukemia. a lymphoma. or a multiple myeloma. In some of any embodiments, the blood cancer is a lymphoma. In some of any embodiments, the blood cancer is a multiple myeloma. In some of any embodiments, the blood cancer is a leukemia. In some of any embodiments, the cancer is a solid tumor.

In some of any embodiments, the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

In some of any embodiments, the cancer is prostate cancer, bladder cancer, kidney cancer, head-and-neck cancer, breast cancer, or leukemia. In some of any embodiments, the cancer is prostate cancer. In some of any embodiments, the cancer is bladder cancer. In some of any embodiments, the cancer is kidney cancer. In some of any embodiments, the cancer is head-and-neck cancer. In some of any embodiments, the cancer is breast cancer. In some of any embodiments, the cancer is leukemia.

In some of any embodiments, the prediction model is built by: (i) determining the values for each of the at least one isotopic feature for the plurality of reference samples; and (ii) using the values for each of the at least one isotopic feature for the plurality of reference samples to train the machine learning algorithm. In some of any embodiments, the prediction model is trained by: (i) determining the values for each of the at least one isotopic feature for the plurality of reference samples; and (ii) using the values for each of the at least one isotopic feature for the plurality of reference samples to train the machine learning algorithm.

In some of any embodiments, some of the plurality of reference subjects are known to have cancer. In some of any embodiments, some of the plurality of reference subjects are assumed to not have cancer. In some of any embodiments, some of the plurality of reference subjects are known to have cancer, and some of the plurality of reference subjects are assumed to not have cancer.

In some of any embodiments, the machine learning algorithm is trained using a plurality of labels. In some of any embodiments, the plurality of labels comprises a label for each of the plurality of reference samples. In some of any embodiments, the machine learning algorithm is trained using a plurality of labels, the plurality of labels comprising a label for each of the plurality of reference samples.

In some of any embodiments, the label indicates if the reference sample is from a reference subject known to have cancer or from a reference subject assumed to not have cancer.

In some of any embodiments, the label indicates the characteristic of the cancer in the reference subject from which the reference sample was obtained.

In some of any embodiments, the label indicates the level of tumor burden of the cancer in the reference subject from which the reference sample was obtained.

In some of any embodiments, the method further comprises preprocessing the values for each of the at least one isotopic feature prior to the training the machine learning algorithm. In some of any embodiments, the preprocessing comprises controlled imputation of missing values. In some of any embodiments, the preprocessing comprises replacing missing values with a zero. In some of any embodiments, the preprocessing comprises data normalization.

In some of any embodiments, cancer is detected in the subject if the prediction model indicates the presence of cancer.

Also provided herein in some embodiments is a method of building a prediction model for detecting whether a subject has a cancer. Also provided herein in some embodiments is a method of training a prediction model for detecting whether a subject has a cancer. In some of any embodiments, the method comprises determining, for a plurality of reference samples, values for each of at least one isotopic feature. In some of any embodiments, the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples. In some of any embodiments, the at least one isotopic feature comprises the overall concentration of an isotopic element measured in a reference sample of the plurality of reference samples. In some of any embodiments, the at least one isotopic feature comprises a chemical feature determined from the overall concentration of an isotopic element measured in a reference sample of the plurality of reference samples. In some of any embodiments, the at least one isotopic feature comprises the concentration of an isotope of an isotopic element measured in a reference sample of the plurality of reference samples. In some of any embodiments, the at least one isotopic feature comprises the concentration of an isotope of an isotopic element measured in a reference sample of the plurality of reference samples. In some of any embodiments, the at least one isotopic feature comprises a concentration of an element, a concentration of an isotopic element, a concentration of an isotope of the isotopic element, or a normalized value of any thereof, measured in a reference sample of the plurality of reference samples. In some of any embodiments, the at least one isotopic feature comprises a concentration of an element measured in a reference sample of the plurality of reference samples. In some of any embodiments, the at least one isotopic feature comprises a concentration of an isotopic element measured in a reference sample of the plurality of reference samples. In some of any embodiments, the at least one isotopic feature comprises a concentration of an isotope of the isotopic element measured in a reference sample of the plurality of reference samples. In some of any embodiments, the at least one isotopic feature comprises a normalized value of a concentration of an element measured in a reference sample of the plurality of reference samples. In some of any embodiments, the at least one isotopic feature comprises a normalized value of a concentration of an isotopic element measured in a reference sample of the plurality of reference samples. In some of any embodiments, the at least one isotopic feature comprises a normalized value of a concentration of an isotope of the isotopic element measured in a reference sample of the plurality of reference samples.

In some of any embodiments, the plurality of reference samples are from a plurality of reference subjects.

In some of any embodiments, the method comprises building a prediction model. In some of any embodiments, the method comprises training a prediction model. In some of any embodiments, the prediction model is configured to predict the presence or absence of a cancer in a subject. In some of any embodiments, the prediction model comprises at least one machine learning algorithm. In some of any embodiments, the building comprises training the machine learning algorithm using the values for each of the at least one isotopic feature for the plurality of reference samples. In some of any embodiments, the training the machine learning algorithm comprises using the values for each of the at least one isotopic feature for the plurality of reference samples. For instance, the methods involve applying the values for each of the at least one isotopic feature for the plurality of reference samples as input to train the machine learning algorithm.

Also provided herein in some embodiments is a method of building a prediction model for detecting whether a subject has a cancer, the method comprising: (a) determining, for a plurality of reference samples from a plurality of reference subjects, values for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples; and (b) building a prediction model configured to predict the presence or absence of a cancer in a subject, wherein the prediction model comprises at least one machine learning algorithm, and the building comprises training the machine learning algorithm using the values for each of the at least one isotopic feature for the plurality of reference samples.

Also provided herein in some embodiments is a method of training a prediction model for detecting whether a subject has a cancer, the method comprising: (a) determining, for a plurality of reference samples from a plurality of reference subjects, values for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3), measured in a reference sample of the plurality of reference samples; and (b) applying the values for each of the at least one isotopic feature for the plurality of reference samples as input to train a prediction model configured to predict the presence or absence of a cancer in a subject, wherein the prediction model comprises at least one machine learning algorithm.

Also provided herein in some embodiments is a method of building a prediction model for assessing a characteristic of a cancer in a subject. Also provided herein in some embodiments is a method of training a prediction model for assessing a characteristic of a cancer in a subject.

In some of any embodiments, the method comprises determining, for a plurality of reference samples, values for each of at least one isotopic feature. In some of any embodiments, the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples. In some of any embodiments, the at least one isotopic feature comprises the overall concentration of an isotopic element measured in a reference sample of the plurality of reference samples. In some of any embodiments, the at least one isotopic feature comprises a chemical feature determined from the overall concentration of an isotopic element measured in a reference sample of the plurality of reference samples. In some of any embodiments, the at least one isotopic feature comprises the concentration of an isotope of an isotopic element measured in a reference sample of the plurality of reference samples. In some of any embodiments, the at least one isotopic feature comprises the concentration of an isotope of an isotopic element measured in a reference sample of the plurality of reference samples. In some of any embodiments, the at least one isotopic feature comprises a concentration of an element, a concentration of an isotopic element, a concentration of an isotope of the isotopic element, or a normalized value of any thereof, measured in a reference sample of the plurality of reference samples. In some of any embodiments, the at least one isotopic feature comprises a concentration of an element measured in a reference sample of the plurality of reference samples. In some of any embodiments, the at least one isotopic feature comprises a concentration of an isotopic element measured in a reference sample of the plurality of reference samples. In some of any embodiments, the at least one isotopic feature comprises a concentration of an isotope of the isotopic element measured in a reference sample of the plurality of reference samples. In some of any embodiments, the at least one isotopic feature comprises a normalized value of a concentration of an element measured in a reference sample of the plurality of reference samples. In some of any embodiments, the at least one isotopic feature comprises a normalized value of a concentration of an isotopic element measured in a reference sample of the plurality of reference samples. In some of any embodiments, the at least one isotopic feature comprises a normalized value of a concentration of an isotope of the isotopic element measured in a reference sample of the plurality of reference samples.

In some of any embodiments, the plurality of reference samples are from a plurality of reference subjects.

In some of any embodiments, the method comprises building a prediction model. In some of any embodiments, the method comprises training a prediction model. In some of any embodiments, the prediction model is configured to predict a characteristic of a cancer in a subject. In some of any embodiments, the prediction model comprises at least one machine learning algorithm. In some of any embodiments, the building comprises training the machine learning algorithm using the values for each of the at least one isotopic feature for the plurality of reference samples. In some of any embodiments, the training the machine learning algorithm comprise using the values for each of the at least one isotopic feature for the plurality of reference samples.

Also provided herein in some embodiments is a method of building a prediction model for assessing a characteristic of a cancer in a subject, the method comprising: (a) determining, for a plurality of reference samples from a plurality of reference subjects, values for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples; and (b) building a prediction model configured to predict a characteristic of a cancer in a subject, wherein the prediction model comprises at least one machine learning algorithm, and the building comprises training the machine learning algorithm using the values for each of the at least one isotopic feature for the plurality of reference samples.

Also provided herein in some embodiments is a method of training a prediction model for assessing a characteristic of a cancer in a subject, the method comprising: (a) determining, for a plurality of reference samples from a plurality of reference subjects, values for each of at least one isotopic feature, (1) a concentration of an element, (2) a concentration of an isotopic element, (3) a concentration of an isotope of the isotopic element, or (4) a normalized value of any of (1)-(3), measured in a reference sample of the plurality of reference samples; and (b) applying the values for each of the at least one isotopic feature for the plurality of reference samples as input for training a prediction model configured to predict a characteristic of a cancer in a subject, wherein the prediction model comprises at least one machine learning algorithm.

In some of any embodiments, the characteristic of the cancer is the level of tumor burden of the cancer in the subject.

In some of any embodiments, the determining the values for each of the at least one isotopic feature comprises mass spectrometry analysis. In some of any embodiments, the value is a normalized value, and the determining the value for each of the at least one isotopic feature comprises calculating the normalized value from the mass spectrometry analysis.

In some of any embodiments, the determining the values for each of the at least one isotopic feature comprises mass spectrometry analysis of liquid samples of one or more of the reference samples. In some of any embodiments, the reference samples are solid reference samples, and the determining the values for each of the at least one isotopic feature comprises mass spectrometry analysis of liquid samples of one or more of the solid reference samples. In some of any embodiments, the determining the values for each of the at least one isotopic feature comprises preparing liquid samples of one or more of the reference samples for analysis by mass spectrometry. In some of any embodiments, the reference samples are solid reference samples, and the determining the values for each of the at least one isotopic feature comprises preparing liquid samples of one or more of the solid reference samples for analysis by mass spectrometry. In some of any embodiments, the liquid samples are prepared by digestion of one or more of the reference samples. In some of any embodiments, the reference samples are solid reference samples, and the liquid samples are prepared by digestion of one or more of the solid reference samples.

In some of any embodiments, the determining the values for each of the at least one isotopic feature comprises mass spectrometry analysis of solid samples of one or more of the reference samples. In some of any embodiments, the reference samples are solid reference samples, and the determining the values for each of the at least one isotopic feature comprises mass spectrometry analysis of one or more of the solid reference samples. In some of any embodiments, the determining the values for each of the at least one isotopic feature comprises preparing solid samples of one or more of the reference samples for analysis by mass spectrometry. In some of any embodiments, the reference samples are solid reference samples, and the determining the values for each of the at least one isotopic feature comprises preparing one or more of the solid reference samples for analysis by mass spectrometry. In some of any embodiments, the solid samples are analyzed via combustion. In some of any embodiments, the solid reference samples are analyzed via combustion.

In some of any embodiments, the determining the values for each of the at least one isotopic feature comprises mass spectrometry analysis of liquid samples of one or more of the reference samples and/or solid samples of one or more of the reference samples. In some of any embodiments, the reference samples are solid samples, and the determining the values for each of the at least one isotopic feature comprises mass spectrometry analysis one or more of the solid reference samples and/or liquid samples of one or more of the solid reference samples. In some of any embodiments, the determining the values for each of the at least one isotopic feature comprises mass spectrometry analysis of liquid samples of one or more of the reference samples. In some of any embodiments, the determining the values for each of the at least one isotopic feature comprises mass spectrometry analysis of liquid samples of one or more of the reference samples. In some of any embodiments, the determining the values for each of the at least one isotopic feature comprises mass spectrometry analysis of solid samples of one or more of the reference samples. In some of any embodiments, the reference samples are solid samples, and the determining the values for each of the at least one isotopic feature comprises mass spectrometry analysis of one or more of the solid reference samples. In some of any embodiments, the determining the values for each of the at least one isotopic feature comprises mass spectrometry analysis of liquid samples of one or more of the reference samples and solid samples of one or more of the reference samples. In some of any embodiments, the reference samples are solid samples, and the determining the values for each of the at least one isotopic feature comprises mass spectrometry analysis of one or more of the solid reference samples and liquid samples of one or more of the solid reference samples.

In some of any embodiments, the determining the values for each of the at least one isotopic feature comprises mass spectrometry analysis of liquid samples prepared by digestion of one or more of the reference samples and/or solid samples of one or more of the reference samples that are analyzed via combustion. In some of any embodiments, the reference samples are solid samples, and the determining the values for each of the at least one isotopic feature comprises mass spectrometry analysis of one or more of the solid reference samples, optionally wherein the one or more of the solid reference samples are analyzed via combustion, and/or liquid samples prepared by digestion of one or more of the solid reference samples. In some of any embodiments, the reference samples are solid samples, and the determining the values for each of the at least one isotopic feature comprises mass spectrometry analysis of one or more of the solid reference samples, wherein the one or more of the solid reference samples are analyzed via combustion, and/or liquid samples prepared by digestion of one or more of the solid reference samples. In some of any embodiments, the determining the values for each of the at least one isotopic feature comprises mass spectrometry analysis of liquid samples prepared by digestion of one or more of the reference samples. In some of any embodiments, the reference samples are solid samples, and the determining the values for each of the at least one isotopic feature comprises mass spectrometry analysis of liquid samples prepared by digestion of one or more of the solid reference samples. In some of any embodiments, the determining the values for each of the at least one isotopic feature comprises mass spectrometry analysis of solid samples of one or more of the reference samples that are analyzed via combustion. In some of any embodiments, the reference samples are solid samples, and the determining the values for each of the at least one isotopic feature comprises mass spectrometry analysis of one or more of the solid reference samples that are optionally analyzed via combustion. In some of any embodiments, the reference samples are solid samples, and the determining the values for each of the at least one isotopic feature comprises mass spectrometry analysis of one or more of the solid reference samples that are analyzed via combustion. In some of any embodiments, the determining the values for each of the at least one isotopic feature comprises mass spectrometry analysis of liquid samples prepared by digestion of one or more of the reference samples and solid samples of one or more of the reference samples that are analyzed via combustion. In some of any embodiments, the reference samples are solid samples, and the determining the values for each of the at least one isotopic feature comprises mass spectrometry analysis of one or more of the solid reference samples that are optionally analyzed via combustion and liquid samples prepared by digestion of one or more of the solid reference samples. In some of any embodiments, the reference samples are solid samples, and the determining the values for each of the at least one isotopic feature comprises mass spectrometry analysis of one or more of the solid reference samples that are analyzed via combustion and liquid samples prepared by digestion of one or more of the solid reference samples.

In some of any embodiments, the determining the values for each of the at least one isotopic feature comprises: (i) preparing liquid samples of one or more of the reference samples for analysis by mass spectrometry, wherein the liquid samples are prepared by digestion of the one or more reference samples; (ii) introducing the prepared liquid samples into a mass spectrometer to detect one or more ionization products of isotopic elements present in the prepared liquid samples; and (iii) analyzing the detected one or more ionization products of isotopic elements to generate values for one or more of the at least one isotopic feature for the prepared liquid samples. In some of any embodiments, the reference samples are solid samples, and the determining the values for each of the at least one isotopic feature comprises: (i) preparing liquid samples of one or more of the solid reference samples for analysis by mass spectrometry, wherein the liquid samples are prepared by digestion of the one or more solid reference samples; (ii) introducing the prepared liquid samples into a mass spectrometer to detect one or more ionization products of isotopic elements and elements present in the prepared liquid samples; and (iii) analyzing the detected one or more ionization products of isotopic elements and elements to generate values for one or more of the at least one isotopic feature for the prepared liquid samples.

In some of any embodiments, the determining the values for each of the at least one isotopic feature comprises: (i) preparing solid samples of one or more of the reference samples for analysis by mass spectrometry; (ii) introducing the prepared solid samples into a mass spectrometer to detect one or more ionization products of isotopic elements present in the prepared solid samples, wherein the detecting comprises combustion of the prepared solid samples; and (iii) analyzing the detected one or more ionization products of isotopic elements to generate values for one or more of the at least one isotopic feature for the prepared solid samples. In some of any embodiments, the reference samples are solid samples, the determining the values for each of the at least one isotopic feature comprises: (i) preparing one or more of the solid reference samples for analysis by mass spectrometry; (ii) introducing the prepared solid samples into a mass spectrometer to detect one or more ionization products of isotopic elements and elements present in the prepared solid samples, optionally wherein the detecting comprises combustion of the prepared solid samples; and (iii) analyzing the detected one or more ionization products of isotopic elements and elements to generate values for one or more of the at least one isotopic feature for the prepared solid samples. In some of any embodiments, the reference samples are solid samples, the determining the values for each of the at least one isotopic feature comprises: (i) preparing one or more of the solid reference samples for analysis by mass spectrometry; (ii) introducing the prepared solid samples into a mass spectrometer to detect one or more ionization products of isotopic elements and elements present in the prepared solid samples, wherein the detecting comprises combustion of the prepared solid samples; and (iii) analyzing the detected one or more ionization products of isotopic elements and elements to generate values for one or more of the at least one isotopic feature for the prepared solid samples.

In some of any embodiments, the determining and/or building is performed by a processor of a computing device. In some of any embodiments, the determining and/or training is performed by a processor of a computing device. In some of any embodiments, the determining is performed by a processor of a computing device. In some of any embodiments, the building is performed by a processor of a computing device. In some of any embodiments, the training is performed by a processor of a computing device. In some of any embodiments, the determining and building is performed by a processor of a computing device. In some of any embodiments, the determining and training is performed by a processor of a computing device.

Also provided herein in some embodiments is a method of building a prediction model for detecting whether a subject has a cancer, the method comprising: (a) receiving, at one or more processors, values for each of at least one isotopic feature for a plurality of reference samples from a plurality of reference subjects, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples; and (b) building, using at least one processor, a prediction model configured to predict the presence or absence of a cancer in a subject, wherein the prediction model comprises at least one machine learning algorithm, and the building comprises training the machine learning algorithm using the values for each of the at least one isotopic feature for the plurality of reference samples.

Also provided herein in some embodiments is a method of training a prediction model for detecting whether a subject has a cancer, the method comprising: (a) receiving, at one or more processors, values for each of at least one isotopic feature for a plurality of reference samples from a plurality of reference subjects, wherein the value for each of the at least one isotopic feature is: (1) a concentration of an element, (2) a concentration of an isotopic element, (3) a concentration of an isotope of the isotopic element, or (4) a normalized value of any of (1)-(3) measured in a reference sample of the plurality of reference samples; and (b) applying the values for each of the at least one isotopic feature for the plurality of reference samples as input for training, using at least one processor, a prediction model configured to predict the presence or absence of a cancer in a subject, wherein the prediction model comprises at least one machine learning algorithm.

Also provided herein in some embodiments is a method of building a prediction model for assessing a characteristic of a cancer in a subject, the method comprising: (a) receiving, at one or more processors, values for each of at least one isotopic feature for a plurality of reference samples from a plurality of reference subjects, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples; and (b) building, using at least one processor, a prediction model configured to predict a characteristic of a cancer in a subject, wherein the prediction model comprises at least one machine learning algorithm, and the building comprises training the machine learning algorithm using the values for each of the at least one isotopic feature for the plurality of reference samples.

Also provided herein in some embodiments is a method of training a prediction model for assessing a characteristic of a cancer in a subject, the method comprising: (a) receiving, at one or more processors, values for each of at least one isotopic feature for a plurality of reference samples from a plurality of reference subjects, wherein the value for each of the at least one isotopic feature is: (1) a concentration of an element, (2) a concentration of an isotopic element, (3) a concentration of an isotope of the isotopic element, or (4) a normalized value of any of (1)-(3), measured in a reference sample of the plurality of reference samples; and (b) applying the values for each of the at least one isotopic feature for the plurality of reference samples as input for training, using at least one processor, a prediction model configured to predict a characteristic of a cancer in a subject, wherein the prediction model comprises at least one machine learning algorithm.

In some of any embodiments, the characteristic of the cancer is the level of tumor burden of the cancer in the subject.

In some of any embodiments, the values for each of the at least one isotopic feature is obtained by mass spectrometry analysis. In some of any embodiments, the value is a normalized value, and the determining the value for each of the at least one isotopic feature comprises calculating the normalized value from the mass spectrometry analysis.

In some of any embodiments, the values for each of the at least one isotopic feature is obtained by mass spectrometry analysis of liquid samples of one or more of the reference samples. In some of any embodiments, the reference samples are solid reference samples, and the determining the values for each of the at least one isotopic feature comprises mass spectrometry analysis of liquid samples of one or more of the solid reference samples. In some of any embodiments, the liquid samples are prepared by digestion of one or more of the reference samples. In some of any embodiments, the reference samples are solid reference samples, and the liquid samples are prepared by digestion of one or more of the solid reference samples.

In some of any embodiments, the values for each of the at least one isotopic feature is obtained by mass spectrometry analysis of solid samples of one or more of the reference samples. In some of any embodiments, the reference samples are solid reference samples, and the determining the values for each of the at least one isotopic feature comprises mass spectrometry analysis of one or more of the solid reference samples. In some of any embodiments, the reference samples are solid reference samples, and the determining the values for each of the at least one isotopic feature comprises preparing one or more of the solid reference samples for analysis by mass spectrometry. In some of any embodiments, the solid samples are analyzed via combustion. In some of any embodiments, the solid reference samples are analyzed via combustion.

In some of any embodiments, the values for each of the at least one isotopic feature is obtained by mass spectrometry analysis of liquid samples of one or more of the reference samples and/or solid samples of one or more of the reference samples. In some of any embodiments, the reference samples are solid reference samples, and the values for each of the at least one isotopic feature is obtained by mass spectrometry analysis of one or more of the solid reference samples and/or liquid samples of one or more of the solid reference samples. In some of any embodiments, the values for each of the at least one isotopic feature is obtained by mass spectrometry analysis of liquid samples of one or more of the reference samples. In some of any embodiments, the reference samples are solid reference samples, and the values for each of the at least one isotopic feature is obtained by mass spectrometry analysis of liquid samples of one or more of the solid reference samples. In some of any embodiments, the values for each of the at least one isotopic feature is obtained by mass spectrometry analysis of solid samples of one or more of the reference samples. In some of any embodiments, the reference samples are solid reference samples, and the values for each of the at least one isotopic feature is obtained by mass spectrometry analysis of one or more of the solid reference samples. In some of any embodiments, the values for each of the at least one isotopic feature is obtained by mass spectrometry analysis of liquid samples of one or more of the reference samples and or solid samples of one or more of the reference samples. In some of any embodiments, the reference samples are solid reference samples, and the values for each of the at least one isotopic feature is obtained by mass spectrometry analysis of one or more of the solid reference samples and liquid samples of one or more of the solid reference samples.

In some of any embodiments, the values for each of the at least one isotopic feature is obtained by mass spectrometry analysis of liquid samples prepared by digestion of one or more of the reference samples and/or solid samples of one or more of the reference samples that are analyzed via combustion. In some of any embodiments, the reference samples are solid reference samples, and the values for each of the at least one isotopic feature is obtained by mass spectrometry analysis of one or more of the solid reference samples, optionally analyzed via combustion, and/or liquid samples prepared by digestion of one or more of the solid reference samples. In some of any embodiments, the reference samples are solid reference samples, and the values for each of the at least one isotopic feature is obtained by mass spectrometry analysis of one or more of the solid reference samples, analyzed via combustion, and/or liquid samples prepared by digestion of one or more of the solid reference samples. In some of any embodiments, the values for each of the at least one isotopic feature is obtained by mass spectrometry analysis of liquid samples prepared by digestion of one or more of the reference samples. In some of any embodiments, the reference samples are solid reference samples, the values for each of the at least one isotopic feature is obtained by mass spectrometry analysis of liquid samples prepared by digestion of one or more of the reference samples. In some of any embodiments, the values for each of the at least one isotopic feature is obtained by mass spectrometry analysis of solid samples of one or more of the reference samples that are analyzed via combustion. In some of any embodiments, the reference samples are solid reference samples, and the values for each of the at least one isotopic feature is obtained by mass spectrometry analysis of one or more of the solid reference samples, optionally analyzed via combustion. In some of any embodiments, the reference samples are solid reference samples, and the values for each of the at least one isotopic feature is obtained by mass spectrometry analysis of one or more of the solid reference samples analyzed via combustion. In some of any embodiments, the values for each of the at least one isotopic feature is obtained by mass spectrometry analysis of liquid samples prepared by digestion of one or more of the reference samples and solid samples of one or more of the reference samples that are analyzed via combustion. In some of any embodiments, the reference samples are solid reference samples, and the values for each of the at least one isotopic feature is obtained by mass spectrometry analysis of one or more of the solid reference samples, optionally analyzed via combustion, and liquid samples prepared by digestion of one or more of the solid reference samples. In some of any embodiments, the reference samples are solid reference samples, and the values for each of the at least one isotopic feature is obtained by mass spectrometry analysis of one or more of the solid reference samples, analyzed via combustion, and liquid samples prepared by digestion of one or more of the solid reference samples.

In some of any embodiments, prior to step (a), the method comprises determining the values for each of the at least one isotopic feature, wherein the determining comprises: (i) preparing liquid samples of one or more of the reference samples for analysis by mass spectrometry, wherein the liquid samples are prepared by digestion of the one or more reference samples; (ii) introducing the prepared liquid samples into a mass spectrometer to detect one or more ionization products of isotopic elements present in the prepared liquid samples; and (iii) analyzing the detected one or more ionization products of isotopic elements to generate values for one or more of the at least one isotopic feature for the prepared liquid samples. In some of any embodiments, the reference samples are solid reference samples, and prior to step (a), the method comprises determining the values for each of the at least one isotopic feature, wherein the determining comprises: (i) preparing liquid samples of one or more of the solid reference samples for analysis by mass spectrometry, wherein the liquid samples are prepared by digestion of the one or more reference samples; (ii) introducing the prepared liquid samples into a mass spectrometer to detect one or more ionization products of isotopic elements and elements present in the prepared liquid samples; and (iii) analyzing the detected one or more ionization products of isotopic elements and elements to generate values for one or more of the at least one isotopic feature for the prepared liquid samples.

In some of any embodiments, prior to step (a), the method comprises determining the values for each of the at least one isotopic feature, wherein the determining comprises: (i) preparing solid samples of one or more of the reference samples for analysis by mass spectrometry; (ii) introducing the prepared solid samples into a mass spectrometer to detect one or more ionization products of isotopic elements present in the prepared solid samples, wherein the detecting comprises combustion of the prepared solid samples; and (iii) analyzing the detected one or more ionization products of isotopic elements to generate values for one or more of the at least one isotopic feature for the prepared solid samples. In some of any embodiments, the reference samples are solid reference samples, and prior to step (a), the method comprises determining the values for each of the at least one isotopic feature, wherein the determining comprises: (i) preparing one or more of the solid reference samples for analysis by mass spectrometry; (ii) introducing the prepared solid samples into a mass spectrometer to detect one or more ionization products of isotopic elements and elements present in the prepared solid samples, optionally wherein the detecting comprises combustion of the prepared solid samples; and (iii) analyzing the detected one or more ionization products of isotopic elements to generate values for one or more of the at least one isotopic feature for the prepared solid samples. In some of any embodiments, the reference samples are solid reference samples, and prior to step (a), the method comprises determining the values for each of the at least one isotopic feature, wherein the determining comprises: (i) preparing one or more of the solid reference samples for analysis by mass spectrometry; (ii) introducing the prepared solid samples into a mass spectrometer to detect one or more ionization products of isotopic elements and elements present in the prepared solid samples, wherein the detecting comprises combustion of the prepared solid samples; and (iii) analyzing the detected one or more ionization products of isotopic elements to generate values for one or more of the at least one isotopic feature for the prepared solid samples.

In some of any embodiments, the analyzing the detected one or more ionization products of isotopic elements is with reference to one or more ionization products of elements present in standards. In some of any embodiments, the analyzing the detected one or more ionization products of isotopic elements and elements is with reference to one or more ionization products of elements and isotopic elements present in standards.

In some of any embodiments, the analyzing the detected one or more ionization products of isotopic elements is with reference to one or more ionization products of elements present in standards comprising a plurality of calibration standards and a blank standard. In some of any embodiments, the analyzing the detected one or more ionization products of isotopic elements and elements is with reference to one or more ionization products of elements and isotopic elements present in standards comprising a plurality of calibration standards and a blank standard.

In some of any embodiments, the analyzing the detected one or more ionization products of isotopic elements is with reference to one or more ionization products of elements present in standards comprising an internal standard and/or a reference material. In some of any embodiments, the analyzing the detected one or more ionization products of isotopic elements and elements is with reference to one or more ionization products of elements and isotopic elements present in standards comprising an internal standard and/or a reference material. In some of any embodiments, the analyzing the detected one or more ionization products of isotopic elements is with reference to one or more ionization products of elements present in standards comprising an internal standard. In some of any embodiments, the analyzing the detected one or more ionization products of isotopic elements and elements is with reference to one or more ionization products of elements and isotopic elements present in standards comprising an internal standard. In some of any embodiments, the analyzing the detected one or more ionization products of isotopic elements is with reference to one or more ionization products of elements present in standards comprising a reference material. In some of any embodiments, the analyzing the detected one or more ionization products of isotopic elements and elements is with reference to one or more ionization products of elements and isotopic elements present in standards comprising a reference material. In some of any embodiments, the analyzing the detected one or more ionization products of isotopic elements is with reference to one or more ionization products of elements present in standards comprising an internal standard and a reference material. In some of any embodiments, the analyzing the detected one or more ionization products of isotopic elements and elements is with reference to one or more ionization products of elements and isotopic elements present in standards comprising an internal standard and a reference material.

In some of any embodiments, the mass spectrometer is further introduced with the standards, wherein the mass spectrometer detects one or more ionization products of elements present in the standards and the prepared liquid or solid samples. In some of any embodiments, the mass spectrometer is further introduced with the standards, wherein the mass spectrometer detects one or more ionization products of elements and isotopic elements present in the standards and the prepared liquid or solid samples.

Also provided herein in some embodiments is a method of building a prediction model for detecting whether a subject has a cancer, the method comprising: (a) providing a plurality of reference samples obtained from a plurality of reference subjects; (b) preparing liquid and/or solid samples for analysis by mass spectrometry from the plurality of reference samples, wherein the liquid samples are prepared by digestion of one or more of the reference samples; (c) providing standards comprising a plurality of calibration standards, a blank standard, an internal standard, and/or a reference material; (d) introducing the standards and the prepared liquid or solid samples individually into a mass spectrometer to detect one or more ionization products of elements present in each of the standards and the prepared liquid or solid samples; (e) analyzing the detected one or more ionization products of isotopic elements to generate values for each of at least one isotopic feature for the plurality of reference samples, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element; (f) receiving, at one or more processors, the values for each of the at least one isotopic feature; and (g) building, using at least one processor, a prediction model configured to predict the presence or absence of a cancer in a subject, wherein the prediction model comprises at least one machine learning algorithm, and the building comprises training the machine learning algorithm using the values for each of the at least one isotopic feature for the plurality of reference samples.

Also provided herein in some embodiments is a method of training a prediction model for detecting whether a subject has a cancer, the method comprising: (a) providing a plurality of solid reference samples obtained from a plurality of reference subjects; (b) preparing liquid samples and/or solid samples for analysis by mass spectrometry from the plurality of solid reference samples, wherein the liquid samples are prepared by digestion of one or more of the solid reference samples; (c) providing standards comprising a plurality of calibration standards, a blank standard, an internal standard, and/or a reference material; (d) introducing the standards and the prepared liquid or solid samples individually into a mass spectrometer to detect one or more ionization products of elements and isotopic elements present in each of the standards and the prepared liquid or solid samples; (e) analyzing the detected one or more ionization products of isotopic elements and elements to generate values for each of at least one isotopic feature for the plurality of reference samples, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the values for each of the at least one isotopic feature; and (g) applying the values for each of the at least one isotopic feature for the plurality of reference samples as input for training, using at least one processor, a prediction model configured to predict the presence or absence of a cancer in a subject, wherein the prediction model comprises at least one machine learning algorithm.

Also provided herein in some embodiments is a method of building a prediction model for assessing a characteristic of a cancer in a subject, the method comprising: (a) providing a plurality of reference samples obtained from a plurality of reference subjects; (b) preparing liquid and/or solid samples for analysis by mass spectrometry from the plurality of reference samples, wherein the liquid samples are prepared by digestion of one or more of the reference samples; (c) providing standards comprising a plurality of calibration standards, a blank standard, an internal standard, and/or a reference material; (d) introducing the standards and the prepared liquid or solid samples individually into a mass spectrometer to detect one or more ionization products of elements present in each of the standards and the prepared liquid or solid samples; (e) analyzing the detected one or more ionization products of isotopic elements to generate values for each of at least one isotopic feature for the plurality of reference samples, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element; (f) receiving, at one or more processors, the values for each of the at least one isotopic feature; and (g) building, using at least one processor, a prediction model configured to predict a characteristic of a cancer in a subject, wherein the prediction model comprises at least one machine learning algorithm, and the building comprises training the machine learning algorithm using the values for each of the at least one isotopic feature for the plurality of reference samples.

Also provided herein in some embodiments is a method of training a prediction model for assessing a characteristic of a cancer in a subject, the method comprising: (a) providing a plurality of solid reference samples obtained from a plurality of reference subjects; (b) preparing liquid samples and/or solid samples for analysis by mass spectrometry from the plurality of solid reference samples, wherein the liquid samples are prepared by digestion of one or more of the solid reference samples; (c) providing standards comprising a plurality of calibration standards, a blank standard, an internal standard, and/or a reference material; (d) introducing the standards and the prepared liquid or solid samples individually into a mass spectrometer to detect one or more ionization products of elements and isotopic elements present in each of the standards and the prepared liquid or solid samples; (e) analyzing the detected one or more ionization products of elements and isotopic elements to generate values for each of at least one isotopic feature for the plurality of reference samples, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the values for each of the at least one isotopic feature; and (g) applying the values for each of the at least one isotopic feature for the plurality of reference samples as input for training, using at least one processor, a prediction model configured to predict a characteristic of a cancer in a subject, wherein the prediction model comprises at least one machine learning algorithm.

In some of any embodiments, the characteristic of the cancer is the level of tumor burden of the cancer in the subject.

In some of any embodiments, the plurality of reference samples each comprise a keratinous tissue, optionally a hard keratinous tissue. In some of any embodiments, the plurality of reference samples each comprise a keratinous tissue. In some of any embodiments, the plurality of reference samples each comprise a hard keratinous tissue.

In some of any embodiments, the plurality of reference samples are noninvasive samples, optionally hair samples and/or nail samples. In some of any embodiments, the plurality of reference samples are noninvasive samples. In some of any embodiments, the plurality of reference samples are hair samples and/or nail samples. In some of any embodiments, the plurality of reference samples are hair samples. In some of any embodiments, the plurality of reference samples are nail samples. In some of any embodiments, the plurality of reference samples are hair samples and nail samples.

In some of any embodiments, the digestion is by acid digestion and/or thermal digestion. In some of any embodiments, the digestion is by acid digestion and thermal digestion.

In some of any embodiments, the acid digestion is with nitric acid (HNO₃). In some of any embodiments, the acid digestion is with nitric acid (HNO₃) and hydrogen peroxide (H₂O₂).

In some of any embodiments, the thermal digestion is by a microwave.

In some of any embodiments, the mass spectrometry is Inductively Coupled Plasma Mass Spectrometry (ICP-MS). In some of any embodiments, the ICP-MS is carried out with kinetic energy discrimination (KED).

In some of any embodiments, the at least one isotopic feature comprises an atomic percent, isotope ratio, fractional abundance, delta, overall concentration, and/or concentration ratio. In some of any embodiments, the at least one isotopic feature comprises an atomic percent. In some of any embodiments, the at least one isotopic feature comprises an isotope ratio. In some of any embodiments, the at least one isotopic feature comprises a fractional abundance. In some of any embodiments, the at least one isotopic feature comprises a delta. In some of any embodiments, the at least one isotopic feature comprises an overall concentration. In some of any embodiments, the at least one isotopic feature comprises a concentration ratio.

In some of any embodiments, the at least one isotopic feature comprises an atomic percent for at least one isotope of an isotopic element, an isotope ratio for at least one isotope of an isotopic element, a fractional abundance for at least one isotope of an isotopic element, and/or a delta for at least one isotope of an isotopic element. In some of any embodiments, the at least one isotopic feature comprises an atomic percent for at least one isotope of an isotopic element. In some of any embodiments, the at least one isotopic feature comprises an isotope ratio for at least one isotope of an isotopic element. In some of any embodiments, the at least one isotopic feature comprises a fractional abundance for at least one isotope of an isotopic element. In some of any embodiments, the at least one isotopic feature comprises a delta for at least one isotope of an isotopic element.

In some of any embodiments, the at least one isotopic feature is a plurality of isotopic features each independently selected from the group consisting of atomic percents, isotope ratios, fractional abundances, deltas, overall concentrations, and concentration ratios.

In some of any embodiments, the plurality of isotopic features comprises an atomic percent for at least one isotope of an isotopic element, an isotope ratio for at least one isotope of an isotopic element, a fractional abundance for at least one isotope of an isotopic element, and/or a delta for at least one isotope of an isotopic element. In some of any embodiments, the plurality of isotopic features comprises an atomic percent for at least one isotope of an isotopic element. In some of any embodiments, the plurality of isotopic features comprises an isotope ratio for at least one isotope of an isotopic element. In some of any embodiments, the plurality of isotopic features comprises a fractional abundance for at least one isotope of an isotopic element. In some of any embodiments, the plurality of isotopic features comprises a delta for at least one isotope of an isotopic element.

In some of any embodiments, the value of each of the at least one isotopic feature is a normalized value of a concentration of an element, a normalized value of a concentration of an isotopic element, or a normalized value of a concentration of an isotope of the isotopic element. In some of any embodiments, the value of each of the at least one isotopic feature is a normalized value of a concentration of an element. In some of any embodiments, the value of each of the at least one isotopic feature is a normalized value of a concentration of an isotopic element. In some of any embodiments, the value of each of the at least one isotopic feature is a normalized value of a concentration of an isotope of the isotopic element.

In some of any embodiments, the normalized value of each of the at least one isotopic feature is an atomic percent (atom %), fractional abundance (F), isotope ratio (R), concentration ratio (CR), or a delta value (delta), or a combination of any of the foregoing. In some of any embodiments, the normalized value of each of the least one isotopic feature is an atomic percent (atom %). In some of any embodiments, the normalized value of each of the least one isotopic feature is a fractional abundance (F). In some of any embodiments, the normalized value of each of the least one isotopic feature is an isotope ratio (R). In some of any embodiments, the normalized value of each of the least one isotopic feature is a concentration ratio (CR). In some of any embodiments, the normalized value of each of the least one isotopic feature is a delta value (delta).

In some of any embodiments, the normalized value of each of the at least one isotopic feature is a delta value (delta). In some of any embodiments, the delta value is a conventional delta value, a study-specific delta value (InH), a non-similar simple-nitrogen based delta value (SimpleNδ), or a non-similar pseudo-nitrogen based delta value (PseudoNδ). In some of any embodiments, the delta value is a conventional delta value. In some of any embodiments, the delta value is a study-specific delta value (InH). In some of any embodiments, the delta value is a non-similar simple-nitrogen based delta value (SimpleNδ). In some of any embodiments, the delta value is a non-similar pseudo-nitrogen based delta value (PseudoNδ).

In some of any embodiments, the isotopic element is Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S.

In some of any embodiments, the isotopic element is Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, or Zn. In some of any embodiments, the isotopic element is Ag. In some of any embodiments, the isotopic element is Al. In some of any embodiments, the isotopic element is B. In some of any embodiments, the isotopic element is Ba. In some of any embodiments, the isotopic element is Br. In some of any embodiments, the isotopic element is Ca. In some of any embodiments, the isotopic element is Co. In some of any embodiments, the isotopic element is Cr. In some of any embodiments, the isotopic element is Cu. In some of any embodiments, the isotopic element is Fe. In some of any embodiments, the isotopic element is Ge. In some of any embodiments, the isotopic element is Hg. In some of any embodiments, the isotopic element is I. In some of any embodiments, the isotopic element is K. In some of any embodiments, the isotopic element is Li. In some of any embodiments, the isotopic element is Mg. In some of any embodiments, the isotopic element is Mn. In some of any embodiments, the isotopic element is Mo. In some of any embodiments, the isotopic element is Na. In some of any embodiments, the isotopic element is Ni. In some of any embodiments, the isotopic element is Pb. In some of any embodiments, the isotopic element is Rb. In some of any embodiments, the isotopic element is Ru. In some of any embodiments, the isotopic element is Se. In some of any embodiments, the isotopic element is Sn. In some of any embodiments, the isotopic element is Sr. In some of any embodiments, the isotopic element is Te. In some of any embodiments, the isotopic element is Ti. In some of any embodiments, the isotopic element is U. In some of any embodiments, the isotopic element is V. In some of any embodiments, the isotopic element is Y. In some of any embodiments, the isotopic element is Zn.

In some of any embodiments, the isotopic element is N, C, S, or O. In some of any embodiments, the isotopic element is N. In some of any embodiments, the isotopic element is C. In some of any embodiments, the isotopic element is S. In some of any embodiments, the isotopic element is O.

In some of any embodiments, the isotopic element is Mo, Cr, Te, C, or Hg. In some of any embodiments, the isotopic element is Mo. In some of any embodiments, the isotopic element is Cr. In some of any embodiments, the isotopic element is Te. In some of any embodiments, the isotopic element is C. In some of any embodiments, the isotopic element is Hg.

In some of any embodiments, the at least one isotopic feature is one isotopic feature. In some of any embodiments, the isotope of the at least one isotopic feature comprises ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr. In some of any embodiments, the isotope of the at least one isotopic feature comprises ⁶⁶Zn. In some of any embodiments, the isotope of the at least one isotopic feature comprises ⁶⁴Zn. In some of any embodiments, the isotope of the at least one isotopic feature comprises ⁶⁸Zn. In some of any embodiments, the isotope of the at least one isotopic feature comprises ⁸⁷Sr. In some of any embodiments, the isotope of the at least one isotopic feature comprises ⁸⁸Sr. In some of any embodiments, the isotope of the at least one isotopic feature comprises ⁸⁶Sr. In some of any embodiments, the isotope of the at least one isotopic feature comprises ⁴⁷Ti. In some of any embodiments, the isotope of the at least one isotopic feature comprises ⁴⁸Ti. In some of any embodiments, the isotope of the at least one isotopic feature comprises ⁴⁶Ti. In some of any embodiments, the isotope of the at least one isotopic feature comprises ⁵⁰Cr. In some of any embodiments, the isotope of the at least one isotopic feature comprises ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and ⁵⁰Cr.

In some of any embodiments, the at least one isotopic feature is a plurality of isotopic features. In some of any embodiments, the plurality of isotopic features is about 2 to about 100 isotopic features. In some of any embodiments, the plurality of isotopic features is 2-100 isotopic features. In some of any embodiments, the plurality of isotopic features comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or more isotopic features. In some of any embodiments, the plurality of isotopic features comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic features.

In some of any embodiments, the at least one isotopic feature is a plurality of isotopic features determined from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or more elements or isotopic elements. In some of any embodiments, the plurality of isotopic features are determined from at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic elements.

In some of any embodiments, the plurality of isotopic features comprises fractional abundances and deltas. In some of any embodiments, the fractional abundance and delta values are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

In some of any embodiments, the plurality of isotopic features comprises fractional abundances, atomic percents, and/or isotope ratios. In some of any embodiments, the fractional abundances, atomic percents, and/or isotope ratios are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, and/or S. In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, and S. In some of any embodiments, the element or the isotopic element of the at least one isotopic feature comprises Li, B, Na, Mg, Al, P, K, Sc, Ca, Ti, V, Cr, Mn, Fe, Co, Ni, Cu, Zn, Ge, As, Se, Br, Rb, Sr, Y, Zr, Nb, Mo, Ru, Ag, Cd, In, Sn, I, Te, Ba, Hg, Pb, U, C, N, O, or S, or a combination of any of the foregoing. In some of any embodiments, the element or the isotopic element of the at least one isotopic feature comprises Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S, or a combination of any of the foregoing. In some of any embodiments, the element or the isotopic element of the at least one isotopic feature comprises Li, B, Mg, Ca, Ti, Cr, Fe, Ni, Cu, Zn, Se, Br, Rb, Sr, Mo, Ag, Sn, Ba, Hg, Pb, U, C, N, S, or a combination of any of the foregoing.

In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, or Zn. In some of any embodiments, the isotopic elements comprise Ag. In some of any embodiments, the isotopic elements comprise Al. In some of any embodiments, the isotopic elements comprise B. In some of any embodiments, the isotopic elements comprise Ba. In some of any embodiments, the isotopic elements comprise Br. In some of any embodiments, the isotopic elements comprise Ca. In some of any embodiments, the isotopic elements comprise Co. In some of any embodiments, the isotopic elements comprise Cr. In some of any embodiments, the isotopic elements comprise Cu. In some of any embodiments, the isotopic elements comprise Fe. In some of any embodiments, the isotopic elements comprise Ge. In some of any embodiments, the isotopic elements comprise Hg. In some of any embodiments, the isotopic elements comprise I. In some of any embodiments, the isotopic elements comprise K. In some of any embodiments, the isotopic elements comprise Li. In some of any embodiments, the isotopic elements comprise Mg. In some of any embodiments, the isotopic elements comprise Mn. In some of any embodiments, the isotopic elements comprise Mo. In some of any embodiments, the isotopic elements comprise Na. In some of any embodiments, the isotopic elements comprise Ni. In some of any embodiments, the isotopic elements comprise Pb. In some of any embodiments, the isotopic elements comprise Rb. In some of any embodiments, the isotopic elements comprise Ru. In some of any embodiments, the isotopic elements comprise Se. In some of any embodiments, the isotopic elements comprise Sn. In some of any embodiments, the isotopic elements comprise Sr. In some of any embodiments, the isotopic elements comprise Te. In some of any embodiments, the isotopic elements comprise Ti. In some of any embodiments, the isotopic elements comprise U. In some of any embodiments, the isotopic elements comprise V. In some of any embodiments, the isotopic elements comprise Y. In some of any embodiments, the isotopic elements comprise Zn. In some of any embodiments, the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, and Zn.

In some of any embodiments, the isotopic elements comprise N, C, S, and/or O. In some of any embodiments, the isotopic elements comprise N. In some of any embodiments, the isotopic elements comprise C. In some of any embodiments, the isotopic elements comprise S. In some of any embodiments, the isotopic elements comprise O. In some of any embodiments, the isotopic elements comprise N, C, S, and O.

In some of any embodiments, the isotopic elements comprise Mo, Cr, Te, C, and/or Hg. In some of any embodiments, the isotopic elements comprise Mo. In some of any embodiments, the isotopic elements comprise Cr. In some of any embodiments, the isotopic elements comprise Te. In some of any embodiments, the isotopic elements comprise C. In some of any embodiments, the isotopic elements comprise Hg. In some of any embodiments, the isotopic elements comprise Mo, Cr, Te, C, and Hg.

In some of any embodiments, at least one isotopic feature comprises a concentration ratio. In some of any embodiments, the concentration ratio is N/C, S/C, O/C, S/N, N/O, or S/O. In some of any embodiments, the concentration ratio is N/C. In some of any embodiments, the concentration ratio is S/C. In some of any embodiments, the concentration ratio is O/C. In some of any embodiments, the concentration ratio is S/N. In some of any embodiments, the concentration ratio is N/O. In some of any embodiments, the concentration ratio is S/O.

In some of any embodiments, the plurality of isotopic features comprises at least one concentration ratio. In some of any embodiments, the at least one concentration ratio comprises N/C, S/C, O/C, S/N, N/O, and/or S/O. In some of any embodiments, the at least one concentration ratio comprises N/C. In some of any embodiments, the at least one concentration ratio comprises S/C. In some of any embodiments, the at least one concentration ratio comprises O/C. In some of any embodiments, the at least one concentration ratio comprises S/N. In some of any embodiments, the at least one concentration ratio comprises N/O. In some of any embodiments, the at least one concentration ratio comprises S/O. In some of any embodiments, the at least one concentration ratio comprises N/C, S/C, O/C, S/N, N/O, and S/O.

In some of any embodiments, the at least one isotopic feature comprises at least 1, 2, 3, 4, 5, 6, 7, 8, 9, or 10 of the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (F³⁴S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO).

In some of any embodiments, the at least one isotopic feature comprises the fractional abundance, atomic percent, or isotope ratio of ¹³C (F13C) relative to stable isotopes of C. In some of any embodiments, the at least one isotopic feature comprises the fractional abundance, atomic percent, or isotope ratio of ¹⁸O (F18O) relative to stable isotopes of O. In some of any embodiments, the at least one isotopic feature comprises the concentration ratio of sulfur over nitrogen (SdN). In some of any embodiments, the at least one isotopic feature comprises the concentration ratio of nitrogen over carbon (NdC). In some of any embodiments, the at least one isotopic feature comprises the concentration ratio of nitrogen over carbon (NdC). In some of any embodiments, the at least one isotopic feature comprises the fractional abundance, atomic percent, or isotope ratio of ³⁴S (f³⁴S) relative to stable isotopes of S. In some of any embodiments, the at least one isotopic feature comprises the fractional abundance, atomic percent, or isotope ratio of ¹⁵N (F15N) relative to stable isotopes of N. In some of any embodiments, the at least one isotopic feature comprises the concentration ratio of oxygen over carbon (OdC). In some of any embodiments, the at least one isotopic feature comprises the concentration ratio of nitrogen over oxygen (NdO). In some of any embodiments, the at least one isotopic feature comprises the concentration ratio of sulfur over carbon (SdC). In some of any embodiments, the at least one isotopic feature comprises the concentration ratio of sulfur over oxygen (SdO).

In some of any embodiments, the at least one isotopic feature comprises the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (f³⁴S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO).

In some of any embodiments, the at least one isotopic feature comprises at least 1, 2, 3, 4, 5, 6, 7, 8, 9, 10, 11, 12, 13, 14, 15, 16, 17, 18, 19, 20, or more of the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷I, ¹²⁸Te ¹³⁶Ba, ¹³⁷Ba, ¹³C, ²³⁵U, ²³⁸U, ²³Na, ²⁷Al, ³⁴S, ³⁹K, ⁴⁴Ca, ⁴⁸Ti, ⁵⁴Cr, ⁵⁴Fe, ⁵⁷Fe, ⁶²Ni, ⁶³Cu, ⁶⁶Zn, ⁶⁷Zn, ⁶⁸Zn, ⁷⁰Ge, ⁷⁴Ge, ⁷⁴Se, ⁷⁶Se, ⁷⁷Se, ⁷⁹Br, ⁸⁰Se, ⁸²Se, ⁸⁹Y, ⁹⁴Mo, ⁹⁷Mo, and ⁹⁸Mo, each relative to stable isotopes of the isotopic element of the isotope.

In some of any embodiments, the at least one isotopic feature comprises the fractional abundance, atomic percent, or isotope ratio of ¹⁰¹Ru relative to stable isotopes of Ru. In some of any embodiments, the at least one isotopic feature comprises the fractional abundance, atomic percent, or isotope ratio of ¹⁰⁷Ag relative to stable isotopes of Ag. In some of any embodiments, the at least one isotopic feature comprises the fractional abundance, atomic percent, or isotope ratio of 109Ag relative to stable isotopes of Ag. In some of any embodiments, the at least one isotopic feature comprises the fractional abundance, atomic percent, or isotope ratio of ¹⁰B relative to stable isotopes of B. In some of any embodiments, the at least one isotopic feature comprises the fractional abundance, atomic percent, or isotope ratio of ¹¹⁸Sn relative to stable isotopes of Sn. In some of any embodiments, the at least one isotopic feature comprises the fractional abundance, atomic percent, or isotope ratio of ¹¹⁹Sn relative to stable isotopes of Sn. In some of any embodiments, the at least one isotopic feature comprises the fractional abundance, atomic percent, or isotope ratio of ¹²⁴Sn relative to stable isotopes of Sn. In some of any embodiments, the at least one isotopic feature comprises the fractional abundance, atomic percent, or isotope ratio of ¹²⁴Te relative to stable isotopes of Te. In some of any embodiments, the at least one isotopic feature comprises the fractional abundance, atomic percent, or isotope ratio of ¹²⁷I relative to stable isotopes of I. In some of any embodiments, the at least one isotopic feature comprises the fractional abundance, atomic percent, or isotope ratio of ¹²⁸Te relative to stable isotopes of Te. In some of any embodiments, the at least one isotopic feature comprises the fractional abundance, atomic percent, or isotope ratio of ¹³⁶Ba relative to stable isotopes of Ba. In some of any embodiments, the at least one isotopic feature comprises the fractional abundance, atomic percent, or isotope ratio of ¹³⁷Ba relative to stable isotopes of Ba. In some of any embodiments, the at least one isotopic feature comprises the fractional abundance, atomic percent, or isotope ratio of ¹³C relative to stable isotopes of C. In some of any embodiments, the at least one isotopic feature comprises the fractional abundance, atomic percent, or isotope ratio of ²³⁵U relative to stable isotopes of U. In some of any embodiments, the at least one isotopic feature comprises the fractional abundance, atomic percent, or isotope ratio of ²³⁸U relative to stable isotopes of U. In some of any embodiments, the at least one isotopic feature comprises the fractional abundance, atomic percent, or isotope ratio of ²³Na relative to stable isotopes of Na. In some of any embodiments, the at least one isotopic feature comprises the fractional abundance, atomic percent, or isotope ratio of ²⁷Al relative to stable isotopes of Al. In some of any embodiments, the at least one isotopic feature comprises the fractional abundance, atomic percent, or isotope ratio of ³⁴S relative to stable isotopes of S. In some of any embodiments, the at least one isotopic feature comprises the fractional abundance, atomic percent, or isotope ratio of ³⁹K relative to stable isotopes of K. In some of any embodiments, the at least one isotopic feature comprises the fractional abundance, atomic percent, or isotope ratio of ⁴⁴Ca relative to stable isotopes of Ca. In some of any embodiments, the at least one isotopic feature comprises the fractional abundance, atomic percent, or isotope ratio of ⁴⁸Ti relative to stable isotopes of Ti. In some of any embodiments, the at least one isotopic feature comprises the fractional abundance, atomic percent, or isotope ratio of ⁵⁴Cr relative to stable isotopes of Cr. In some of any embodiments, the at least one isotopic feature comprises the fractional abundance, atomic percent, or isotope ratio of ⁵⁴Fe relative to stable isotopes of Fe. In some of any embodiments, the at least one isotopic feature comprises the fractional abundance, atomic percent, or isotope ratio of ⁵⁷Fe relative to stable isotopes of Fe. In some of any embodiments, the at least one isotopic feature comprises the fractional abundance, atomic percent, or isotope ratio of ⁶²Ni relative to stable isotopes of Ni. In some of any embodiments, the at least one isotopic feature comprises the fractional abundance, atomic percent, or isotope ratio of ⁶³Cu relative to stable isotopes of Cu. In some of any embodiments, the at least one isotopic feature comprises the fractional abundance, atomic percent, or isotope ratio of ⁶⁶Zn relative to stable isotopes of Zn. In some of any embodiments, the at least one isotopic feature comprises the fractional abundance, atomic percent, or isotope ratio of ⁶⁷Zn relative to stable isotopes of Zn. In some of any embodiments, the at least one isotopic feature comprises the fractional abundance, atomic percent, or isotope ratio of ⁶⁸Zn relative to stable isotopes of Zn. In some of any embodiments, the at least one isotopic feature comprises the fractional abundance, atomic percent, or isotope ratio of ⁷⁰Ge relative to stable isotopes of Ge. In some of any embodiments, the at least one isotopic feature comprises the fractional abundance, atomic percent, or isotope ratio of ⁷⁴Ge relative to stable isotopes of Ge. In some of any embodiments, the at least one isotopic feature comprises the fractional abundance, atomic percent, or isotope ratio of ⁷⁴Se relative to stable isotopes of Se. In some of any embodiments, the at least one isotopic feature comprises the fractional abundance, atomic percent, or isotope ratio of ⁷⁶Se relative to stable isotopes of Se. In some of any embodiments, the at least one isotopic feature comprises the fractional abundance, atomic percent, or isotope ratio of ⁷⁷Se relative to stable isotopes of Se. In some of any embodiments, the at least one isotopic feature comprises the fractional abundance, atomic percent, or isotope ratio of ⁷⁹Br relative to stable isotopes of Br. In some of any embodiments, the at least one isotopic feature comprises the fractional abundance, atomic percent, or isotope ratio of ⁸⁰Se relative to stable isotopes of Se. In some of any embodiments, the at least one isotopic feature comprises the fractional abundance, atomic percent, or isotope ratio of ⁸²Se relative to stable isotopes of Se. In some of any embodiments, the at least one isotopic feature comprises the fractional abundance, atomic percent, or isotope ratio of ⁸⁹Y relative to stable isotopes of Y. In some of any embodiments, the at least one isotopic feature comprises the fractional abundance, atomic percent, or isotope ratio of ⁹⁴Mo relative to stable isotopes of Mo. In some of any embodiments, the at least one isotopic feature comprises the fractional abundance, atomic percent, or isotope ratio of ⁹⁷Mo relative to stable isotopes of Mo. In some of any embodiments, the at least one isotopic feature comprises the fractional abundance, atomic percent, or isotope ratio of ⁹⁸Mo relative to stable isotopes of Mo.

In some of any embodiments, the at least one isotopic feature comprises the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷I, ¹²⁸Te, ¹³⁶Ba, ¹³⁷Ba, ¹³C, ²³⁵U, ²³⁸U, ²³Na, ²⁷Al, ³⁴S, ³⁹K, ⁴⁴Ca, ⁴⁸Ti, ⁵⁴Cr, ⁵⁴Fe, ⁵⁷Fe, ⁶²Ni, ⁶³Cu, ⁶⁶Zn, ⁶⁷Zn, ⁶⁸Zn, ⁷⁰Ge, ⁷⁴Ge, ⁷⁴Se, ⁷⁶Se, ⁷⁷Se, ⁷⁹Br, ⁸⁰Se, ⁸²Se ⁸⁹Y, ⁹⁴Mo, ⁹⁷Mo, and ⁹⁸Mo, each relative to stable isotopes of the isotopic element of the isotope.

In some of any embodiments, the at least one isotopic feature comprises at least 1, 2, 3, 4, 5, 6, 7, 8, 9, 10, or more of the fractional abundance, atomic percent, or isotope ratio of ¹⁰⁷Ag, ¹⁰⁹Ag, ¹¹⁸Sn, ¹²⁷I, ¹³C, ²³⁵U, ²⁷Al, ³⁴S, ⁴⁴Ca, ¹⁷Fe, ⁶⁷Zn, ⁷⁴Se, ⁹⁴Mo, and ⁹⁷Mo, each relative to stable isotopes of the isotopic element of the isotope.

In some of any embodiments, the at least one isotopic feature comprises the fractional abundance, atomic percent, or isotope ratio of ¹⁰⁷Ag relative to stable isotopes of Ag. In some of any embodiments, the at least one isotopic feature comprises the fractional abundance, atomic percent, or isotope ratio of ¹⁰⁹Ag relative to stable isotopes of Ag. In some of any embodiments, the at least one isotopic feature comprises the fractional abundance, atomic percent, or isotope ratio of ¹¹⁸Sn relative to stable isotopes of Sn. In some of any embodiments, the at least one isotopic feature comprises the fractional abundance, atomic percent, or isotope ratio of ¹²⁷I relative to stable isotopes of L In some of any embodiments, the at least one isotopic feature comprises the fractional abundance, atomic percent, or isotope ratio of ¹³C relative to stable isotopes of C. In some of any embodiments, the at least one isotopic feature comprises the fractional abundance, atomic percent, or isotope ratio of ²³⁵U relative to stable isotopes of U. In some of any embodiments, the at least one isotopic feature comprises the fractional abundance, atomic percent, or isotope ratio of ²⁷Al relative to stable isotopes of Al. In some of any embodiments, the at least one isotopic feature comprises the fractional abundance, atomic percent, or isotope ratio of ³⁴S relative to stable isotopes of S. In some of any embodiments, the at least one isotopic feature comprises the fractional abundance, atomic percent, or isotope ratio of ⁴⁴Ca relative to stable isotopes of Ca. In some of any embodiments, the at least one isotopic feature comprises the fractional abundance, atomic percent, or isotope ratio of ⁵⁷Fe relative to stable isotopes of Fe. In some of any embodiments, the at least one isotopic feature comprises the fractional abundance, atomic percent, or isotope ratio of ⁶⁷Zn relative to stable isotopes of Zn. In some of any embodiments, the at least one isotopic feature comprises the fractional abundance, atomic percent, or isotope ratio of ⁷⁴Se relative to stable isotopes of Se. In some of any embodiments, the at least one isotopic feature comprises the fractional abundance, atomic percent, or isotope ratio of ⁹⁴Mo relative to stable isotopes of Mo. In some of any embodiments, the at least one isotopic feature comprises the fractional abundance, atomic percent, or isotope ratio of ⁹⁷Mo relative to stable isotopes of Mo.

In some of any embodiments, the at least one isotopic feature comprises the fractional abundance, atomic percent, or isotope ratio of ¹⁰⁷Ag, ¹⁰⁹Ag, ¹¹⁸Sn, ¹²⁷, ¹³C, ²³⁵U, ²⁷Al, ³⁴S, ⁴⁴Ca, ⁵⁷Fe, ⁶⁷Zn, ⁷⁴Se, ⁹⁴Mo, and ⁹⁷Mo, each relative to stable isotopes of the isotopic element of the isotope.

In some of any embodiments, the at least one isotopic feature comprises the fractional abundance, atomic percent, or isotope ratio of ¹²⁷I relative to stable isotopes of I. In some of any embodiments, the at least one isotopic feature comprises the fractional abundance, atomic percent, or isotope ratio of ⁴Ca relative to stable isotopes of Ca. In some of any embodiments, the at least one isotopic feature comprises the fractional abundance, atomic percent, or isotope ratio of ¹²⁷I relative to stable isotopes of I and the fractional abundance, atomic percent, or isotope ratio of ⁴Ca relative to stable isotopes of Ca.

In some of any embodiments, the isotope of the at least one isotopic feature comprises ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁵Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing.

In some of any embodiments, the isotopic element of the at least one isotopic feature comprises Zn. In some of any embodiments, the isotope of the at least one isotopic feature comprises ⁶⁴Zn or ⁶⁶Zn. In some of any embodiments, the isotope of the at least one isotopic feature comprises ⁶⁴Zn. the isotope of the at least one isotopic feature comprises ⁶⁶Zn. In some of any embodiments, the isotope of the at least one isotopic feature comprises ⁶⁴Zn and ⁶⁶Zn.

In some of any embodiments, the isotopic element of the at least one isotopic feature comprises S. In some of any embodiments, the isotope of the at least one isotopic feature comprises ³⁴S.

In some of any embodiments, the isotopic element of the at least one isotopic feature comprises N, C, S, or O. In some of any embodiments, the isotopic element of the at least one isotopic feature comprises N. In some of any embodiments, the isotopic element of the at least one isotopic feature comprises C. In some of any embodiments, the isotopic element of the at least one isotopic feature comprises S. In some of any embodiments, the isotopic element of the at least one isotopic feature comprises O. In some of any embodiments, the isotopic element of the at least one isotopic feature comprises N, C, S, and O.

In some of any embodiments, the machine learning algorithm is a supervised machine learning algorithm.

In some of any embodiments, the prediction model is a classification model. In some of any embodiments, the prediction model is a binary classification model. In some of any embodiments, the prediction model is a multiclass classification model.

In some of any embodiments, the machine learning algorithm is Logistic Regression (e.g., Ridge, Lasso, or ElasticNet), Naive Bayes Classifier, GaussianNB, GaussianNBisotonic, GaussianNBsigmoid, Random Forest Classifier, GradientBoosting Classifier, XGBoost Classifer, HistGradientBoosting Classifier, Deep Neural Network Classifier, DecisionTree Classifier, Bagging Classifier, ExtraTrees Classifier, AdaBoost Classifier, CatBoost Classifier, LGBM Classifier, Support Vector Classifier, Perceptron Classifier (e.g., Multi-layer Perceptron Classifier), Deep Learning Classifier for tabular data (e.g., TabNet or TabML), PassiveAggressiveClassifier, or SGD Classifier, or an ensemble algorithm comprising a combination of any of the foregoing.

In some of any embodiments, the machine learning algorithm is a support vector classifier, a decision tree-based ensemble classifier, or a multilayer perceptron classifier. In some of any embodiments, the machine learning algorithm is a support vector classifier. In some of any embodiments, the machine learning algorithm is a decision tree-based ensemble classifier. In some of any embodiments, the machine learning algorithm is a multilayer perceptron classifier.

In some of any embodiments, the machine learning algorithm is an unsupervised machine learning algorithm.

In some of any embodiments, the prediction model is a general model. In some of any embodiments, at least one subject of the plurality of reference subjects lives in a different geographical region, is a different sex, is a different age, is from a different cultural or ethnic group, has a different diet, and/or has a different cancer type, compared to another subject of the plurality of reference subjects. In some of any embodiments, at least one subject of the plurality of reference subjects lives in a different geographical region, is a different sex, is a different age, is from a different cultural or ethnic group, has a different diet, or has a different cancer type, compared to another subject of the plurality of reference subjects. In some of any embodiments, at least one subject of the plurality of reference subjects lives in a different geographical region compared to another subject of the plurality of reference subjects. In some of any embodiments, the plurality of reference subjects are from at least 2, 3, 4, 5, 6, or more, different countries. In some of any embodiments, at least one subject of the plurality of reference subjects is a different sex compared to another subject of the plurality of reference subjects. In some of any embodiments, at least one subject of the plurality of reference subjects is a different age compared to another subject of the plurality of reference subjects. In some of any embodiments, at least one subject of the plurality of reference subjects is from a different cultural or ethnic group compared to another subject of the plurality of reference subjects. In some of any embodiments, at least one subject of the plurality of reference subjects has a different diet compared to another subject of the plurality of reference subjects. In some of any embodiments, at least one subject of the plurality of reference subjects has a different cancer type compared to another subject of the plurality of reference subjects. In some of any embodiments, at least one subject of the plurality of reference subjects lives in a different geographical region, is a different sex, is a different age, is from a different cultural or ethnic group, has a different diet, and has a different cancer type, compared to another subject of the plurality of reference subjects.

In some of any embodiments, the prediction model is a specialized model. In some of any embodiments, each subject of the plurality of reference subjects live in the same geographical region, are the same sex, are the same age, and/or have the same cancer type. In some of any embodiments, each subject of the plurality of reference subjects live in the same geographical region, are the same sex, are the same age, or have the same cancer type. In some of any embodiments, each subject of the plurality of reference subjects live in the same geographical region. In some of any embodiments, each subject of the plurality of reference subjects are the same sex. In some of any embodiments, each subject of the plurality of reference subjects are male. In some of any embodiments, each subject of the plurality of reference subjects are female. In some of any embodiments, each subject of the plurality of reference subjects are the same age. In some of any embodiments, each subject of the plurality of reference subjects have the same cancer type. In some of any embodiments, each subject of the plurality of reference subjects live in the same geographical region, are the same sex, are the same age, and have the same cancer type.

In some of any embodiments, the plurality of reference subjects are mammals. In some of any embodiments, the plurality of reference subjects are humans.

In some of any embodiments, the cancer is a blood cancer or is a solid tumor. In some of any embodiments, the cancer is a blood cancer. In some of any embodiments, the blood cancer is a leukemia. a lymphoma. or a multiple myeloma. In some of any embodiments, the blood cancer is a lymphoma. In some of any embodiments, the blood cancer is a multiple myeloma. In some of any embodiments, the blood cancer is a leukemia. In some of any embodiments, the cancer is a solid tumor.

In some of any embodiments, the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, tadrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

In some of any embodiments, the cancer is prostate cancer, bladder cancer, kidney cancer, head-and-neck cancer, breast cancer, or leukemia. In some of any embodiments, the cancer is prostate cancer. In some of any embodiments, the cancer is bladder cancer. In some of any embodiments, the cancer is kidney cancer. In some of any embodiments, the cancer is head-and-neck cancer. In some of any embodiments, the cancer is breast cancer. In some of any embodiments, the cancer is leukemia.

In some of any embodiments, some of the plurality of reference subjects are known to have cancer. In some of any embodiments, some of the plurality of reference subjects are assumed to not have cancer. In some of any embodiments, some of the plurality of reference subjects are known to have cancer, and some of the plurality of reference subjects are assumed to not have cancer.

In some of any embodiments, the machine learning algorithm is trained using a plurality of labels. In some of any embodiments, the plurality of labels comprises a label for each of the plurality of reference samples. In some of any embodiments, the machine learning algorithm is trained using a plurality of labels, the plurality of labels comprising a label for each of the plurality of reference samples.

In some of any embodiments, the label indicates if the reference sample is from a reference subject known to have cancer or from a reference subject assumed to not have cancer.

In some of any embodiments, the label indicates the characteristic of the cancer in the reference subject from which the reference sample was obtained.

In some of any embodiments, the label indicates the level of tumor burden of the cancer in the reference subject from which the reference sample was obtained.

In some of any embodiments, the method further comprises preprocessing the values for each of the at least one isotopic feature prior to the training the machine learning algorithm. In some of any embodiments, the preprocessing comprises controlled imputation of missing values. In some of any embodiments, the preprocessing comprises replacing missing values with a zero. In some of any embodiments, the preprocessing comprises data normalization.

In some of any embodiments, cancer is detected in the subject if the prediction model indicates the presence of cancer.

In some of any embodiments, the prediction model is built using a computer-program product tangibly embodied in a non-transitory machine-readable storage medium comprising instructions configured to cause one or more data processors to build the prediction model. In some of any embodiments, the prediction model is trained using a computer-program product tangibly embodied in a non-transitory machine-readable storage medium comprising instructions configured to cause one or more data processors to train the prediction model.

Also provided herein in some embodiments is a method for determining a value of at least one isotopic feature of an isotope in a solid sample from a subject.

In some of any embodiments, the method comprises determining the value for each of at least one isotopic feature of an isotope in a solid sample from a subject. In some of any embodiments, the at least one isotopic feature comprises the concentration of the isotope measured in the solid sample from the subject. In some of any embodiments, the at least one isotopic feature comprises a normalized value of the concentration of the isotope measured in the solid sample from the subject.

Also provided herein in some embodiments is a method for determining a value of at least one isotopic feature of an isotope in a solid sample from a subject, the method comprising: (a) detecting, by mass spectrometry, one or more ionization products of isotopic elements present in a solid sample from a subject, optionally wherein the solid sample is analyzed via combustion, or in a liquid sample prepared by digestion of the solid sample, wherein the one or more ionization products are at least one isotope selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or a combination of any of the foregoing; and (b) determining a value of at least one isotopic feature, wherein the at least one isotopic feature is a concentration of the at least one isotope or a normalized value thereof.

In some of any embodiments, the value of each of the at least one isotopic feature is a normalized value of the concentration of the at least one isotope.

In some of any embodiments, the method further comprises normalizing the value of each of the at least one isotopic feature.

In some of any embodiments, the normalized value of each of the at least one isotopic feature is an atomic percent (atom %), fractional abundance (F), isotope ratio (R), or a delta value (delta), or a combination of any of the foregoing. In some of any embodiments, the normalized value of each of the at least one isotopic feature is an atomic percent (atom %). In some of any embodiments, the normalized value of each of the at least one isotopic feature is a fractional abundance (F). In some of any embodiments, the normalized value of each of the at least one isotopic feature is an isotope ratio (R). In some of any embodiments, the normalized value of each of the at least one isotopic feature a delta value (delta).

Also provided herein is a method for determining a value of at least one isotopic feature of an isotope in a solid sample from a subject, the method comprising: (a) detecting, by mass spectrometry, one or more ionization products of isotopic elements present in a solid sample from a subject, optionally wherein the solid sample is analyzed via combustion, or in a liquid sample prepared by digestion of the solid sample, wherein the one or more ionization products are at least one isotope selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or a combination of any of the foregoing; (b) determining a value of at least one isotopic feature, wherein the at least one isotopic feature is a concentration of the at least one isotope; (c) normalizing the value the concentration of the at least one isotope, wherein the normalized value of each of the at least one isotopic feature is an atomic percent (atom %), fractional abundance (F), isotope ratio (R), or a delta value (delta), or a combination of any of the foregoing.

Also provided herein is a method for determining a value of at least one isotopic feature of an isotope in a solid sample from a subject, the method comprising: (a) detecting, by mass spectrometry, one or more ionization products of isotopic elements present in a solid sample from a subject, wherein the solid sample is analyzed via combustion, or in a liquid sample prepared by digestion of the solid sample, wherein the one or more ionization products are at least one isotope selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or a combination of any of the foregoing; (b) determining a value of at least one isotopic feature, wherein the at least one isotopic feature is a concentration of the at least one isotope; (c) normalizing the value the concentration of the at least one isotope, wherein the normalized value of each of the at least one isotopic feature is an atomic percent (atom %), fractional abundance (F), isotope ratio (R), or a delta value (delta), or a combination of any of the foregoing.

In some of any embodiments, the normalized value of each of the at least one isotopic feature is an atomic percent (atom %), fractional abundance (F), isotope ratio (R), or a delta value (delta), or a combination of any of the foregoing. In some of any embodiments, the normalized value of each of the at least one isotopic feature is an atomic percent (atom %). In some of any embodiments, the normalized value of each of the at least one isotopic feature is a fractional abundance (F). In some of any embodiments, the normalized value of each of the at least one isotopic feature is an isotope ratio (R). In some of any embodiments, the normalized value of each of the at least one isotopic feature a delta value (delta).

In some of any embodiments, the normalized value of each of the at least one isotopic feature is a delta value (delta). In some of any embodiments, the delta value is a conventional delta value, a study-specific delta value (InH), a non-similar simple-nitrogen based delta value (SimpleNδ), or a non-similar pseudo-nitrogen based delta value (PseudoNδ).

In some of any embodiments, the determining the value of each of the at least one isotopic feature is with reference to one or more isotopic feature present in standards comprising a plurality of calibration standards and a blank standard. In some of any embodiments, the determining the value of each of the at least one isotopic feature is with reference to one or more isotopic feature present in standards comprising an internal standard and/or a reference material. In some of any embodiments, the determining the value of each of the at least one isotopic feature is with reference to one or more isotopic feature present in standards comprising an internal standard. In some of any embodiments, the determining the value of each of the at least one isotopic feature is with reference to one or more isotopic feature present in standards comprising a reference material. In some of any embodiments, the determining the value of each of the at least one isotopic feature is with reference to one or more isotopic feature present in standards comprising an internal standard and a reference material.

In some of any embodiments, the method further comprises introducing the standards into the mass spectrometer, wherein the mass spectrometer detects one or more ionization products of isotopic elements present in the standards and the prepared liquid or solid sample. In some of any embodiments, the method further comprises introducing the standards into the mass spectrometer, wherein the mass spectrometer detects one or more ionization products of isotopic elements present in the standards and the prepared liquid sample. In some of any embodiments, the method further comprises introducing the standards into the mass spectrometer, wherein the mass spectrometer detects one or more ionization products of isotopic elements present in the standards and the solid sample.

In some of any embodiments, the sample comprises a keratinous tissue, optionally a hard keratinous tissue. In some of any embodiments, the sample comprises a hard keratinous tissue. In some of any embodiments, the sample is a noninvasive sample, optionally a hair sample or a nail sample. In some of any embodiments, the sample is a hair sample. In some of any embodiments, the sample is a nail sample.

In some of any embodiments, the digestion is by acid digestion and/or thermal digestion. In some of any embodiments, the digestion is by acid digestion. In some of any embodiments, the digestion is by thermal digestion. In some of any embodiments, the digestion is by acid digestion and thermal digestion. In some of any embodiments, the acid digestion is with nitric acid (HNO₃). In some of any embodiments, the acid digestion is with nitric acid (HNO₃) and hydrogen peroxide (H₂O₂). In some of any embodiments, the thermal digestion is by a microwave.

In some of any embodiments, the mass spectrometry is Inductively Coupled Plasma Mass Spectrometry (ICP-MS). In some of any embodiments, the ICP-MS is carried out with kinetic energy discrimination (KED).

In some of any embodiments, the at least one isotope is one isotope. In some of any embodiments, the at least one isotope is ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr. In some of any embodiments, the at least one isotope is ⁶⁶Zn. In some of any embodiments, the at least one isotope is ⁶⁴Zn. In some of any embodiments, the at least one isotope is ⁶⁸Zn. In some of any embodiments, the at least one isotope is 87Sr. In some of any embodiments, the at least one isotope is ⁸⁸Sr. In some of any embodiments, the at least one isotope is ⁸⁶Sr. In some of any embodiments, the at least one isotope is ⁴⁷Ti. In some of any embodiments, the at least one isotope is ⁴⁸Ti. In some of any embodiments, the at least one isotope is 46Ti. In some of any embodiments, the at least one isotope is ⁵⁰Cr. In some of any embodiments, the at least one isotope is ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and ⁵⁰Cr.

In some of any embodiments, the isotope is ⁶⁴Zn, ⁶⁶Zn, or ³⁴S. In some of any embodiments, the isotope is ⁶⁴Zn. In some of any embodiments, the isotope is ⁶⁶Zn. In some of any embodiments, the isotope is ³⁴S.

In some of any embodiments, the at least one isotope is a plurality of isotopes. In some of any embodiments, the plurality of isotopes is about 2 to about 59 isotopes. In some of any embodiments, the plurality of isotopes is 2-59 isotopes. In some of any embodiments, the at least one isotope is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 isotopes.

In some of any embodiments, the subject is suspected of having cancer.

In some of any embodiments, the method further comprises selecting a subject that is suspected of having a cancer.

In some of any embodiments, the subject has one or more symptoms that indicate a cancer may be present in the subject.

In some of any embodiments, the cancer is a blood cancer or is a solid tumor. In some of any embodiments, the blood cancer is a leukemia, a lymphoma or a multiple myeloma. In some of any embodiments, the blood cancer is a leukemia. In some of any embodiments, the blood cancer is lymphoma. In some of any embodiments, the blood cancer is a multiple myeloma.

In some of any embodiments, the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

In some of any embodiments, the cancer is prostate cancer, bladder cancer, kidney cancer, head-and-neck cancer, breast cancer or leukemia. In some of any embodiments, the cancer is prostate cancer. In some of any embodiments, the cancer is bladder cancer. In some of any embodiments, the cancer is kidney cancer. In some of any embodiments, the cancer is head-and-neck cancer. In some of any embodiments, the cancer is breast cancer. In some of any embodiments, the cancer is leukemia.

In some of any embodiments, the subject is a mammal. In some of any embodiments, the subject is a human.

In some of any embodiments, the method is noninvasive.

Also provided herein is a kit for determining a value of at least one isotopic feature in a sample from a subject.

In some of any embodiments, the value for each of at least one isotopic feature of an isotope in a subject. In some of any embodiments, the at least one isotopic feature comprises the concentration of the isotope measured in the sample from the subject. In some of any embodiments, the at least one isotopic feature comprises a normalized value of the concentration of the isotope measured in the sample from the subject.

Also provided herein is a kit for determining a value of at least one isotopic feature in a sample from a subject, wherein the at least one isotopic feature is a concentration of at least one isotope or a normalized value thereof, wherein the at least one isotope is selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, ²³⁸U, or any combination of the foregoing, and wherein the kit comprises: (i) a reference material comprising a verified standard for the at least one isotope; (ii) packaging material; and (iii) instructions for using the kit, wherein the instructions are for determining a value of the at least one isotopic feature.

In some of any embodiments, the value of each of the at least one isotopic feature is a normalized value of the concentration of the at least one isotope. In some of any embodiments, the normalized value of each of the at least one isotopic feature is an atomic percent (atom %), fractional abundance (F), isotope ratio (R), or a delta value (delta), or a combination of any of the foregoing. In some of any embodiments, the normalized value of each of the at least one isotopic feature is an atomic percent (atom %). In some of any embodiments, the normalized value of each of the at least one isotopic feature is a fractional abundance (F). In some of any embodiments, the normalized value of each of the at least one isotopic feature is an isotope ratio (R). In some of any embodiments, the normalized value of each of the at least one isotopic feature is a delta value (delta).

In some of any embodiments, the normalized value of each of the at least one isotopic feature is a delta value (delta). In some of any embodiments, the delta value is a conventional delta value, a study-specific delta value (InH), a non-similar simple-nitrogen based delta value (SimpleNδ), or a non-similar pseudo-nitrogen based delta value (PseudoNδ). In some of any embodiments, the delta value is a conventional delta value. In some of any embodiments, the delta value is a study-specific delta value (InH). In some of any embodiments, the delta value is a non-similar simple-nitrogen based delta value (SimpleNδ). In some of any embodiments, the delta value is a non-similar pseudo-nitrogen based delta value (PseudoNδ).

In some of any embodiments, the value of each of the at least one isotopic feature is with reference to one or more isotopic feature present in the reference material.

In some of any embodiments, the kit further comprises a calibration standard, a blank standard, and/or an internal standard. In some of any embodiments, the kit further comprises a calibration standard. In some of any embodiments, the kit further comprises a blank standard. In some of any embodiments, the kit further comprises an internal standard. In some of any embodiments, the kit further comprises a calibration standard, a blank standard, and an internal standard.

In some of any embodiments, the sample comprises a keratinous tissue, optionally a hard keratinous tissue. In some of any embodiments, the sample comprises a hard keratinous tissue. In some of any embodiments, the sample is a noninvasive sample, optionally a hair sample or a nail sample. In some of any embodiments, the sample is a hair sample. In some of any embodiments, the sample is a nail sample.

In some of any embodiments, the sample is a digested sample. In some of any embodiments, the kit comprises instructions for digesting the sample. In some of any embodiments, the digesting is by acid digestion and/or thermal digestion. In some of any embodiments, the digesting is by acid digestion. In some of any embodiments, the digesting is by thermal digestion. In some of any embodiments, the digesting is by acid digestion and thermal digestion. In some of any embodiments, the acid digesting is with nitric acid (HNO₃). In some of any embodiments, the acid digesting is with nitric acid (HNO₃) and hydrogen peroxide (H₂O₂). In some of any embodiments, the thermal digestion is by a microwave.

In some of any embodiments, the at least one isotope is one isotope. In some of any embodiments, the at least one isotope is ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr. In some of any embodiments, the at least one isotope is ⁶⁶Zn. In some of any embodiments, the at least one isotope is ⁶⁴Zn. In some of any embodiments, the at least one isotope is ⁶⁸Zn. In some of any embodiments, the at least one isotope is ⁸⁷Sr. In some of any embodiments, the at least one isotope is ⁸⁸Sr. In some of any embodiments, the at least one isotope is ⁸⁶Sr. In some of any embodiments, the at least one isotope is ⁴⁷Ti. In some of any embodiments, the at least one isotope is ⁴⁸Ti. In some of any embodiments, the at least one isotope is ⁴⁶Ti. In some of any embodiments, the at least one isotope is ⁵⁰Cr. In some of any embodiments, the at least one isotope is ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and ⁵⁰Cr.

In some of any embodiments, the isotope is ⁶⁴Zn, ⁶⁶Zn, or ³⁴S. In some of any embodiments, the isotope is ⁶⁴Zn. In some of any embodiments, the isotope is ⁶⁶Zn. In some of any embodiments, the isotope is ³⁴S.

In some of any embodiments, the at least one isotope is a plurality of isotopes. In some of any embodiments, the plurality of isotopes is about 2 to about 59 isotopes. In some of any embodiments, the plurality of isotopes is 2-59 isotopes. In some of any embodiments, the at least one isotope is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 isotopes.

In some of any embodiments, the subject is suspected of having cancer.

In some of any embodiments, the method further comprises selecting a subject that is suspected of having a cancer.

In some of any embodiments, the subject has one or more symptoms that indicate a cancer may be present in the subject.

In some of any embodiments, the cancer is a blood cancer or is a solid tumor. In some of any embodiments, the blood cancer is a leukemia, a lymphoma or a multiple myeloma. In some of any embodiments, the blood cancer is a leukemia. In some of any embodiments, the blood cancer is lymphoma. In some of any embodiments, the blood cancer is a multiple myeloma.

In some of any embodiments, the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

In some of any embodiments, the cancer is prostate cancer, bladder cancer, kidney cancer, head-and-neck cancer, breast cancer or leukemia. In some of any embodiments, the cancer is prostate cancer. In some of any embodiments, the cancer is bladder cancer. In some of any embodiments, the cancer is kidney cancer. In some of any embodiments, the cancer is head-and-neck cancer. In some of any embodiments, the cancer is breast cancer. In some of any embodiments, the cancer is leukemia.

In some of any embodiments, the subject is a mammal. In some of any embodiments, the subject is a human.

Also provided herein is a method for determining the value of the at least one isotopic feature from the sample from the subject, comprising using the kit of any embodiments according to the instructions.

Also provided herein is an isotope profile identified by mass spectrometry, wherein the isotope profile is from a solid sample from a subject, wherein the isotope profile comprises a value of at least one isotopic feature of at least one isotope selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing, and wherein the at least one isotopic feature is a concentration of the at least one isotope. Also provided herein is an isotope profile identified by mass spectrometry, wherein the isotope profile is from a solid sample from a subject, wherein the isotope profile comprises a value of at least one isotopic feature of at least one isotope selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing, and wherein the at least one isotopic feature is a concentration of the at least one isotope or a normalized value of the concentration of the at least one isotope.

In some of any embodiments, the value of the at least one isotopic feature is a normalized value of the concentration of the at least one isotope. In some of any embodiments, the normalized value of each of the at least one isotopic feature is an atomic percent (atom %), fractional abundance (F), isotope ratio (R), or a delta value (delta), or a combination of any of the foregoing. In some of any embodiments, the normalized value of each of the at least one isotopic feature is an atomic percent (atom %). In some of any embodiments, the normalized value of each of the at least one isotopic feature is a fractional abundance (F). In some of any embodiments, the normalized value of each of the at least one isotopic feature is an isotope ratio (R). In some of any embodiments, the normalized value of each of the at least one isotopic feature is a delta value (delta).

In some of any embodiments, the normalized value of each of the at least one isotopic feature is a delta value (delta). In some of any embodiments, the delta value is a conventional delta value, a study-specific delta value (InH), a non-similar simple-nitrogen based delta value (SimpleNδ), or a non-similar pseudo-nitrogen based delta value (PseudoNδ). In some of any embodiments, the delta value is a conventional delta value. In some of any embodiments, the delta value is a study-specific delta value (InH). In some of any embodiments, the delta value is a non-similar simple-nitrogen based delta value (SimpleNδ). In some of any embodiments, the delta value is a non-similar pseudo-nitrogen based delta value (PseudoNδ).

In some of any embodiments, the sample comprises a keratinous tissue, optionally a hard keratinous tissue. In some of any embodiments, the sample comprises a hard keratinous tissue. In some of any embodiments, the sample is a noninvasive sample, optionally a hair sample or a nail sample. In some of any embodiments, the sample is a hair sample. In some of any embodiments, the sample is a nail sample.

In some of any embodiments, the solid sample is digested into a prepared liquid sample for mass spectrometry. In some of any embodiments, the digestion is by acid digestion and/or thermal digestion. In some of any embodiments, the digestion is by acid digestion. In some of any embodiments, the digestion is by thermal digestion. In some of any embodiments, the digestion is by acid digestion and thermal digestion. In some of any embodiments, the acid digestion is with nitric acid (HNO₃). In some of any embodiments, the acid digestion is with nitric acid (HNO₃) and hydrogen peroxide (H₂O₂). In some of any embodiments, the thermal digestion is by a microwave.

In some of any embodiments, the mass spectrometry is Inductively Coupled Plasma Mass Spectrometry (ICP-MS). In some of any embodiments, the ICP-MS is carried out with kinetic energy discrimination (KED).

In some of any embodiments, the at least one isotope is one isotope. In some of any embodiments, the at least one isotope is ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr. In some of any embodiments, the at least one isotope is ⁶⁶Zn. In some of any embodiments, the at least one isotope is ⁶⁴Zn. In some of any embodiments, the at least one isotope is ⁶⁸Zn. In some of any embodiments, the at least one isotope is ⁸⁷Sr. In some of any embodiments, the at least one isotope is ¹⁸Sr. In some of any embodiments, the at least one isotope is ⁸⁶Sr. In some of any embodiments, the at least one isotope is ⁴⁷Ti. In some of any embodiments, the at least one isotope is ⁴⁸Ti. In some of any embodiments, the at least one isotope is ⁴⁶Ti. In some of any embodiments, the at least one isotope is ⁵⁰Cr. In some of any embodiments, the at least one isotope is ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and ⁵⁰Cr.

In some of any embodiments, the isotope is ⁶⁴Zn, ⁶⁶Zn, or ³⁴S. In some of any embodiments, the isotope is ⁶⁴Zn. In some of any embodiments, the isotope is ⁶⁶Zn. In some of any embodiments, the isotope is ³⁴S.

In some of any embodiments, the at least one isotope is a plurality of isotopes. In some of any embodiments, the plurality of isotopes is about 2 to about 59 isotopes. In some of any embodiments, the plurality of isotopes is 2-59 isotopes. In some of any embodiments, the at least one isotope is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 isotopes.

In some of any embodiments, the subject is suspected of having cancer.

In some of any embodiments, the method further comprises selecting a subject that is suspected of having a cancer.

In some of any embodiments, the subject has one or more symptoms that indicate a cancer may be present in the subject.

In some of any embodiments, the cancer is a blood cancer or is a solid tumor. In some of any embodiments, the blood cancer is a leukemia, a lymphoma or a multiple myeloma. In some of any embodiments, the blood cancer is a leukemia. In some of any embodiments, the blood cancer is lymphoma. In some of any embodiments, the blood cancer is a multiple myeloma.

In some of any embodiments, the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

In some of any embodiments, the cancer is prostate cancer, bladder cancer, kidney cancer, head-and-neck cancer, breast cancer or leukemia. In some of any embodiments, the cancer is prostate cancer. In some of any embodiments, the cancer is bladder cancer. In some of any embodiments, the cancer is kidney cancer. In some of any embodiments, the cancer is head-and-neck cancer. In some of any embodiments, the cancer is breast cancer. In some of any embodiments, the cancer is leukemia.

In some of any embodiments, the subject is a mammal. In some of any embodiments, the subject is a human.

Also provided herein is a method for determining whether a subject has a cancer, the method comprising the step of comparing one or more isotopic features of an isotope profile of any embodiments to one or more corresponding isotopic features of (i) individuals characterized as having cancer, (ii) individuals characterized as being cancer free, or (iii) both individuals characterized as having cancer and individuals characterized as being cancer free.

In some of any embodiments, the method comprises the step of comparing one or more isotopic features of an isotope profile of any embodiments to one or more corresponding isotopic features of (i) individuals characterized as having cancer, (ii) individuals characterized as being cancer free, or (iii) both individuals characterized as having cancer and individuals characterized as being cancer free. In some of any embodiments, the method comprises the step of comparing one or more isotopic features of an isotope profile of any embodiments to one or more corresponding isotopic features of individuals characterized as having cancer. In some of any embodiments, the method comprises the step of comparing one or more isotopic features of an isotope profile of any embodiments to one or more corresponding isotopic features of individuals characterized as being cancer free. In some of any embodiments, the method comprises the step of comparing one or more isotopic features of an isotope profile of any embodiments to one or more corresponding isotopic features of both individuals characterized as having cancer and individuals characterized as being cancer free.

In some of any embodiments, the step of comparing is carried out by inputting the value for one or more isotopic features from the isotope profile into a prediction model configured to predict the presence or absence of a cancer in the subject, wherein the prediction model comprises at least one machine learning algorithm trained using values for one or more corresponding isotopic features from a plurality of reference samples from a plurality of reference subjects.

In some of any embodiments, the prediction model determines whether the subject has cancer.

In some of any embodiments, the plurality of reference subjects comprises (i) individuals characterized as having cancer, (ii) individuals characterized as being cancer free, or (iii) both individuals characterized as having cancer and individuals characterized as being cancer free. In some of any embodiments, the plurality of reference subjects comprises individuals characterized as having cancer. In some of any embodiments, the plurality of reference subjects comprises individuals characterized as being cancer free. In some of any embodiments, the plurality of reference subjects comprises both individuals characterized as having cancer and individuals characterized as being cancer free.

In some of any embodiments, the isotope profile is received as a data file that is communicated electronically from a physician, laboratory, or other service provider. In some of any embodiments, the isotope profile is received as a data file that is communicated electronically from a physician. In some of any embodiments, the isotope profile is received as a data file that is communicated electronically from a laboratory. In some of any embodiments, the isotope profile is received as a data file that is communicated electronically from another service provider.

In some of any embodiments, the determination of whether the subject has cancer is communicated electronically to a physician, laboratory, or other service provider, optionally wherein the electronic communication comprises (i) a comparison of isotopic features of the subject's isotope profile with corresponding isotopic features from a plurality of reference samples from a plurality of reference subjects, (ii) a determination of the probability that the subject has cancer, or (iii) both (i) and (ii). In some of any embodiments, the determination of whether the subject has cancer is communicated electronically to a physician, laboratory, or other service provider, wherein the electronic communication comprises a comparison of isotopic features of the subject's isotope profile with corresponding isotopic features from a plurality of reference samples from a plurality of reference subjects. In some of any embodiments, the determination of whether the subject has cancer is communicated electronically to a physician, laboratory, or other service provider, optionally wherein the electronic communication comprises a determination of the probability that the subject has cancer. In some of any embodiments, the determination of whether the subject has cancer is communicated electronically to a physician, laboratory, or other service provider, wherein the electronic communication comprises a comparison of isotopic features of the subject's isotope profile with corresponding isotopic features from a plurality of reference samples from a plurality of reference subjects, and a determination of the probability that the subject has cancer.

In some of any embodiments, the isotope profile comprises at least one normalized value for the concentration of at least one isotope of the isotope profile. In some of any embodiments, the normalized value is an atomic percent (atom %), fractional abundance (F), isotope ratio (R), or a delta value (delta), or a combination of any of the foregoing. In some of any embodiments, the normalized value is an atomic percent (atom %). In some of any embodiments, the normalized is a fractional abundance (F). In some of any embodiments, the normalized value is an isotope ratio (R). In some of any embodiments, the normalized value is a delta value (delta).

In some of any embodiments, the normalized value is a delta value (delta). In some of any embodiments, the delta value is a conventional delta value, a study-specific delta value (InH), a non-similar simple-nitrogen based delta value (SimpleNδ), or a non-similar pseudo-nitrogen based delta value (PseudoNδ). In some of any embodiments, the delta value is a conventional delta value. In some of any embodiments, the delta value is a study-specific delta value (InH). In some of any embodiments, the delta value is a non-similar simple-nitrogen based delta value (SimpleNδ). In some of any embodiments, the delta value is a non-similar pseudo-nitrogen based delta value (PseudoNδ).

Also provided herein is a method for determining whether a subject has a cancer, the method comprising: (a) determining a normalized value for the concentration of the at least one isotope of the isotope profile of any embodiments, and (b) determining, based on the normalized value, whether the subject has a cancer.

In some of any embodiments, the normalized value is an atomic percent (atom %), fractional abundance (F), isotope ratio (R), or a delta value (delta), or a combination of any of the foregoing. In some of any embodiments, the normalized value is an atomic percent (atom %). In some of any embodiments, the normalized is a fractional abundance (F). In some of any embodiments, the normalized value is an isotope ratio (R). In some of any embodiments, the normalized value is a delta value (delta).

In some of any embodiments, the normalized value is a delta value (delta). In some of any embodiments, the delta value is a conventional delta value, a study-specific delta value (InH), a non-similar simple-nitrogen based delta value (SimpleNδ), or a non-similar pseudo-nitrogen based delta value (PseudoNδ). In some of any embodiments, the delta value is a conventional delta value. In some of any embodiments, the delta value is a study-specific delta value (InH). In some of any embodiments, the delta value is a non-similar simple-nitrogen based delta value (SimpleNδ). In some of any embodiments, the delta value is a non-similar pseudo-nitrogen based delta value (PseudoNδ).

Also provided herein is a method of treating a cancer in a subject, comprising: (a) selecting a subject detected as having a cancer according to the method of any embodiments; and (b) treating the subject for the cancer with a treatment for the cancer.

In some of any embodiments, the treatment comprises chemotherapy, radiotherapy, cryotherapy, photodynamic therapy, laser therapy, immunotherapy, targeted therapy, hormone therapy, surgical procedures to remove the cancer, stem cell transplant, bone marrow transplant, or a combination of any of the foregoing. In some of any embodiments, the treatment comprises chemotherapy. In some of any embodiments, the treatment comprises radiotherapy. In some of any embodiments, the treatment comprises cryotherapy. In some of any embodiments, the treatment comprises photodynamic therapy. In some of any embodiments, the treatment comprises laser therapy. In some of any embodiments, the treatment comprises immunotherapy. In some of any embodiments, the treatment comprises targeted therapy. In some of any embodiments, the treatment comprises hormone therapy. In some of any embodiments, the treatment comprises surgical procedures to remove the cancer. In some of any embodiments, the treatment comprises stem cell transplant. In some of any embodiments, the treatment comprises bone marrow transplant.

### BRIEF DESCRIPTION OF THE FIGURES

FIG. 1A. A schematic of a chemical fingerprint of climate change that is recorded in shells since water with the lighter isotope of oxygen is preferentially evaporated and transported from the ocean to a storage in continental glaciers. In the case of global warming, water with ¹⁶O melts from land and shifts the oceanic mass balance of ¹⁸O/¹⁶O, which is recorded in shells over time (t). The change is expressed as deviations in permil (% c) from an agreed upon reference baseline.

FIG. 1B. A schematic of an isotypic fingerprint of tumor transformation in tissue samples, such as hair, that are distant from a tumor during cancer progression. A change in the rate of cell turnover or proliferation is recorded in hair since, for example, molybdenum (Mo) could be used at a rate so that cancer cells incorporate proportionally more of the heavier isotope (⁹⁵Mo) compared to than during tissue homeostasis. Hair would therefore record a shift towards proportionally more of the lighter isotope (⁹⁴Mo) and, thus, lower ratios of ⁹⁵Mo/⁹⁴Mo (and changes of other mass balances also) when cancer is growing. In this example, the change is expressed as deviations in permil (‰) from a reference baseline.

FIG. 1C. An overview of an exemplary workflow. In this workflow, hair and nail specimens from human cancer patients and healthy donors are subjected to combustion (solids) or digestion (solutes) for elemental analysis using bulk mass spectrometry (e.g., ICP-MS). Data generated by bulk mass spectrometry, e.g., ICP-MS, are processed and used to develop prediction models, e.g., using machine learning algorithm(s). The prediction model can be a classification model, such as a binary or multiclass classification model. The validated prediction model will be later available to determine or predict cancer, e.g., cancer probability, when fed with adequate training data with similar chemical information from humans with and without cancer. Alternatively, the prediction model can have a continuous outcome, e.g., be a regression model, for instance where the risk of cancer growth or level of tumor burden is indicated. In one embodiment, the operation includes collection of samples (e.g., hair strands or nail clippings) that are combusted or digested and analyzed for their overall concentrations of a number of isotopic elements (and concentrations of their individual isotopes), calculations of isotopic compositions of elements, and the use of machine learning using a number of isotopic features. Exemplary isotopic elements and isotopic features are discussed below.

FIG. 2. Frequently used machine learning algorithms to develop classification models, such as decision tree-based (e.g., decision tree-based ensemble) or deep learning algorithms.

FIG. 3A. Schematic representation of a decision tree-based algorithm, the random forest learning algorithm. Steps in the random forest algorithm can include bootstrapping and aggregation. During bootstrapping, the training dataset can be resampled into several small datasets, which can then be used to build the decision trees. The size of the bootstrapped dataset can remain the same, but bootstrapped decision trees can be different from each other. Based on test data, decision trees can make predictions, and, in the aggregation step, predictions can be combined for a final prediction. For a classification problem, the final prediction (e.g., 1 or 0, for binary classification) can be made by majority voting (e.g., the majority of either 0 or 1 “wins” and equals the prediction). For a regression problem, the final prediction can be an average value, e.g., the mean or median value of the predictions. Figure was adapted from [29].

FIG. 3B. Schematic representation of a deep learning algorithm. In a deep neural network (DNN) model, each node of the input data layer can be fully connected to the hidden layer nodes. The first hidden layer can take input data, multiply it by a number calculated by the algorithm known as weight, and add a bias before applying a nonlinear activation function. The second hidden layer can take the first hidden layer as input and so on until it reaches the output layer. Figure was adapted from [29].

FIG. 3C. Schematic representation of an autoencoder learning algorithm. The autoencoder can determine latent variables by reducing the dimensions during encoding. Then, it can decode the data into a similar form using the latent variables. Figure was adapted from [29].

FIG. 4. A brief workflow to explain how a binary classification model was developed.

FIG. 5. Raw data may be processed and divided into training and test samples. Training samples may also include samples for validation. Training samples can be used to build multiple decision trees using, for example, the XGBoost algorithm. The collection of trees can then be used to determine or predict cancer, e.g., the probability of cancer, using test samples.

FIG. 6. A brief workflow to explain how a prediction can be made using a binary classification model.

FIG. 7. Performance of five classifiers when trained on isotopic features of major elements (fractional abundances, atomic percents, isotope ratios, and delta values of N, C, S, and O and concentration ratios N/C, S/C, O/C, S/N, N/O, and S/O) in hair from patients with prostate cancer.

FIG. 8. The receiver operating characteristic (ROC) curves (true positives plotted against false positives) achieved using eight different machine-learning algorithms on major and minor element data. ROC curves were produced using data from repeated constructions of the predictive model where the training and test samples were sampled from the original data 500 times in 500 different ways. In the 500 repeated constructions, training data (80%) was used to construct a model, while the other samples (20%) were used to test the model. Therefore, each ROC curve represents 500 repeated constructions of its respective predictive model when samples were randomly selected for training and testing. The shaded area indicates the mean (+/−) standard deviation.

FIG. 9. Accuracy and root-mean-square error (RMSE) values plotted against iterations (or numbers of trees) were achieved for both training and test data (major and minor elements) with eight different machine-learning algorithms.

FIG. 10. Confusion matrices were acquired using ten different machine-learning algorithms on major and minor element data.

FIG. 11. Global SHapley Additive exPlanations (SHAP) values for prostate cancer and control samples, analyzed for major elements only (atomic percents and fractional abundances of the stable isotopes of N, C, S, and O and overall concentration ratios N/C, S/C, O/C, S/N, N/O, and S/O). The figure shows feature leverage, calculated according to their contribution in the prediction for the test sample. Labels denote atomic percent of an element with ‘_atom’ and concentration ratios between two elements with a ‘d’.

FIG. 12. Global SHAP values for prostate, kidney/renal, and bladder cancer samples were analyzed for major and minor elements (atomic percent). In this case, the atomic percent values account for the percent of the concentration of one isotope (e.g., ⁴⁴Ca) over all isotopes measured for the element (e.g., Ca). The figure shows feature leverage, calculated according to their contribution in the prediction for the test sample.

FIGS. 13A-13E. By increasing the dimensions of features, control and cancer samples were separated, here using an example of identification potential that is increasing when going from one (FIG. 13A), through 2 (FIG. 13B), 3 (FIG. 13C), 4 (FIG. 13D), and 5 (FIG. 13E) dimensions.

FIG. 14A. To define a decision boundary is, for humans, simple if there is a clear separation between two groups of data. Figure was adapted from [29].

FIG. 14B. To define a decision boundary is, for humans, difficult when data is complex. Figure was adapted from [29].

FIG. 14C. To define a decision boundary of complex data is facilitated by computational power. For example, the decision boundary of the data points of FIG. 14B can be identified using a Kernel function. Figure was adapted from [29].

FIG. 15A. A sample set of actual delta values (δ¹³C; obtained from a comparison of the fractional abundance of the two most common carbon isotopes ¹²C and ¹³C in the sample and in a reference material) are presented in a one-dimensional dot plot.

FIG. 15B. A sample set of actual delta values (δ³⁴S; obtained from a comparison of the fractional abundance of the two most common sulfur isotopes ³²S and ³⁴S in the sample and in a reference material) are presented in a one-dimensional dot plot.

FIG. 16A. Hair samples from US males with and without prostate cancer were analyzed for isotopes of major and minor elements and fractional abundances (F) were calculated. In the case of F⁶⁴Zn and F⁶⁶Zn, the figure shows a Z score for how values from samples with cancer deviate from the mean value of control samples. One way ANOVA with multiple comparisons was used to calculate p value.

FIG. 16B. Performance (accuracy) of one machine-learning (XGBoost) algorithm when trained on an increasing number (up to 25) of isotopic features (fractional abundances) of major and minor elements in hair from US males with and without prostate cancer.

FIG. 17. After evaluating ten different machine-learning classifiers, the XGBoost classifier was used to train on isotopic features (fractional abundances) of major and minor elements in hair from US male patients with prostate cancer. The figure shows performance of subsequent testing of the algorithm with 79 cancer samples and 125 control samples, using 20 or 59 features.

FIGS. 18A-18B. Cross-validation of one model (acquired with XGBoost classifier) by randomly selecting different sets of training and testing samples for each iteration. Accuracy values plotted against iterations were achieved for test samples using 20 (FIG. 18A) and 59 (FIG. 18B) isotopic features (fractional abundances) of major and minor elements in hair from US males with and without prostate cancer.

FIG. 19. The Receiver Operating Curve (ROC) plots the true positive rate (sensitivity) against false positive rate (1-specificity) achieved using the XGBoost-generated algorithm trained on 20 (left) and 59 (right) isotopic features (fractional abundances) of major and minor elements in hair from US males with and without prostate cancer. The shaded area indicates the mean (+/−) standard deviation achieved when cross-validating with 500 iterations of the model. The Area Under the Curve (AUC) indicates the model's performance above random (>0.5).

FIGS. 20A-20B. Accuracy values plotted against iterations were achieved when using XGBoost Classifier for test samples with data consisting of 20 (FIG. 20A) and 50 (FIG. 20B) isotopic features (fractional abundances) of major and minor elements in hair from US males and females with and without prostate, skin, bladder, kidney, colon, Hodgkin's' lymphoma, rectal, testicular, thyroid, tonsil, hematological and/or breast cancer.

FIG. 21. The Receiver Operating Curve (ROC) plots the true positive rate (sensitivity) against false positive rate (1-specificity) achieved using the XGBoost-generated algorithm trained on isotopic features (fractional abundances) of major and minor elements in hair from US males and females with and without prostate, skin, bladder, kidney, colon, Hodgkin's' lymphoma, rectal, testicular, thyroid, tonsil and/or hematological cancer. The shaded area indicates the mean (+/−) standard deviation achieved when cross-validating with 500 iterations of the model. The Area Under the Curve (AUC) indicates the model's performance above random (>0.5).

FIG. 22. XGBoost-generated algorithm trained on isotopic features (fractional abundances) of major and minor elements in hair from US males and females with and without prostate, skin, bladder, kidney, colon, Hodgkin's' lymphoma, rectal, testicular, thyroid, tonsil and/or hematological cancer.

FIGS. 23A-23B. Accuracy values plotted against iterations were achieved when using XGBoost Classifier for test samples with data consisting of 20 (FIG. 23A) and 59 (FIG. 23B) isotopic features (fractional abundances) of major and minor elements in hair from US and Sweden males and females with and without prostate, skin, bladder, kidney, colon, Hodgkin's' lymphoma, rectal, testicular, thyroid, tonsil, hematological and/or breast cancer.

FIG. 24. The Receiver Operating Curve (ROC) plots the true positive rate (sensitivity) against false positive rate (1-specificity) achieved using the XGBoost-generated algorithm trained on isotopic features (fractional abundances) of major and minor elements in hair from US and Sweden males and females with and without prostate, skin, bladder, kidney, colon, Hodgkin's' lymphoma, rectal, testicular, thyroid, tonsil, hematological and/or breast cancer. The shaded area indicates the mean (+/−) standard deviation achieved when cross-validating with 500 iterations of the model. The Area Under the Curve (AUC) indicates the model's performance above random (>0.5).

FIG. 25. XGBoost-generated algorithm trained on isotopic features (fractional abundances) of major and minor elements in hair from US and Sweden males and females with and without prostate, skin, bladder, kidney, colon, Hodgkin's' lymphoma, rectal, testicular, thyroid, tonsil, hematological and/or breast cancer.

FIG. 26. Accuracy values plotted against iterations were achieved when using XGBoost Classifier for both test samples with data consisting of isotopic features (δⁿX_{lnH} values) of major and minor elements in hair from US and Sweden males and females with and without prostate, skin, bladder, kidney, colon, Hodgkin's' lymphoma, rectal, testicular, thyroid, tonsil, hematological and/or breast cancer.

### DETAILED DESCRIPTION OF THE DISCLOSURE

Provided herein are methods for detecting, diagnosing, or assessing cancer in a subject. In some embodiments, the methods are for detecting a cancer in a subject. In some embodiments, the methods are for diagnosing a cancer in a subject. In some embodiments, the methods are for assessing a cancer in a subject. In some embodiments, the methods are for treating a cancer in a subject, based on diagnosing a subject with a cancer according to any of the provided methods.

In some embodiments, the cancer is detected, diagnosed, or assessed based on a sample from the subject. In some embodiments, the sample is a sample collected noninvasively from the subject (noninvasive sample). In some embodiments, the sample contains tissue that is not from the site of the cancer to be detected, diagnosed, or assessed. In some embodiments, the sample contains tissue that is distant from the site of the cancer to be detected, diagnosed, or assessed. In some embodiments, the sample is a hair sample. In some embodiments, the sample is a nail sample.

In some embodiments, the cancer is detected, diagnosed, or assessed based on at least one isotopic feature of an isotopic element for the sample. In some embodiments, the at least isotopic feature includes the overall concentration of an isotopic element (e.g., C). In some embodiments, the at least one isotopic feature includes a chemical feature determined from the overall concentration of the isotopic element. In some embodiments, the at least one isotopic feature includes the concentration of an isotope (e.g., ¹³C) of an isotopic element (e.g., C). In some embodiments, the at least one isotopic feature includes a chemical feature determined from the concentration of an isotope of the isotopic element. In some embodiments, a chemical feature may be a normalized value of a concentration of an element, such as isotopic element, or an isotope. In some embodiments, a value for each of the at least one isotopic feature is obtained for the sample. In some embodiments, a value for each of the at least one isotopic feature is determined for the sample.

In some embodiments, the cancer is detected, diagnosed, or assessed using a prediction model. In some embodiments, the value for each of the at least one isotopic feature is provided as input to the prediction model. In some embodiments, the prediction model is configured to predict the presence or absence of the cancer in the subject. In some embodiments, the prediction model is configured to diagnose the cancer in the subject.

In some embodiments, the prediction model is configured to predict a characteristic of the subject or the cancer in the subject. In some embodiments, the prediction model is configured to predict the type of cancer in the subject. In some embodiments, the prediction model is configured to predict if the cancer is local or has metastasized. In some embodiments, the prediction model is configured to predict the level of tumor burden of the cancer in the subject. In some embodiments, the prediction model is configured to predict the risk of cancer development in the subject. In some embodiments, the prediction model is configured to predict the risk of tumor growth of the cancer in the subject.

In some embodiments, the prediction model involves at least one machine learning algorithm. In some embodiments, the machine learning algorithm is trained using values for each of the at least one isotopic feature. In some embodiments, the values for each of the at least one isotopic feature are from a plurality of samples. In some embodiments, the plurality of samples are from a plurality of reference subjects. In some embodiments, the plurality of reference subjects include subjects other than the subject for which the cancer is to be detected, diagnosed, or assessed. In some embodiments, the plurality of reference subjects does not include the subject for which the cancer is to be detected, diagnosed, or assessed.

Stable isotope chemistry is widely used by, e.g., geoscientists, to track changes in the rates and pathways of chemical turnover that occurred on Earth in deep time. While the changes previously occurred within living systems, the chemical remains of this turnover can be preserved as isotopic imprints (or ratios or fingerprints) in present-day geological samples (e.g., rocks, fossilized shells, or minerals). The fingerprint represents a measurement of the proportions of the various isotopes of an element. Due to the difference in the number of neutrons in the nuclei of an atom, stable isotopes of an element display differences in atomic mass and therefore also slight differences in bond strength (and distance) to other atoms in a molecular structure. Generally, the energy required to dislocate an atom with weak bonds (e.g., light isotope) to other atoms in a molecule is lower than the energy required to dislocate an atom with stronger bonds (e.g., heavy isotope) to other atoms in a molecule. Therefore, the resulting isotopic ratio of an element can mirror conditions under which a stable isotopic element was used or transported between its different sources (supply) and sinks (storage); such as, for example, how intensely this element was used. To follow and measure the usage and transport of elements in this way can be a powerful tool for at least two reasons: it can bypass the limitations of measuring changes in only the overall elemental concentrations (e.g., when the source is small and the system large), and an isotopic signal can be detected both at the source of change (primary) and in the surrounding environment (reflecting). For example, by acquiring and analyzing a rock sample (e.g., previous marine sediment) of a determined age from the shallow settings (e.g., the reflection), it is possible to determine what was once happening in the deep ocean (e.g., the primary signal) [4].

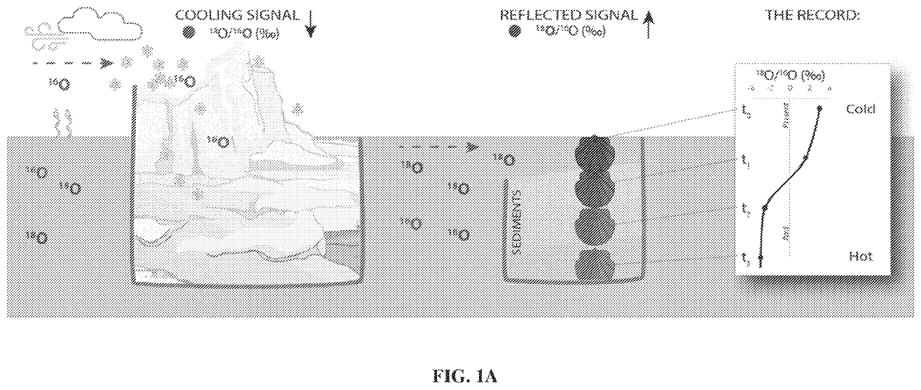

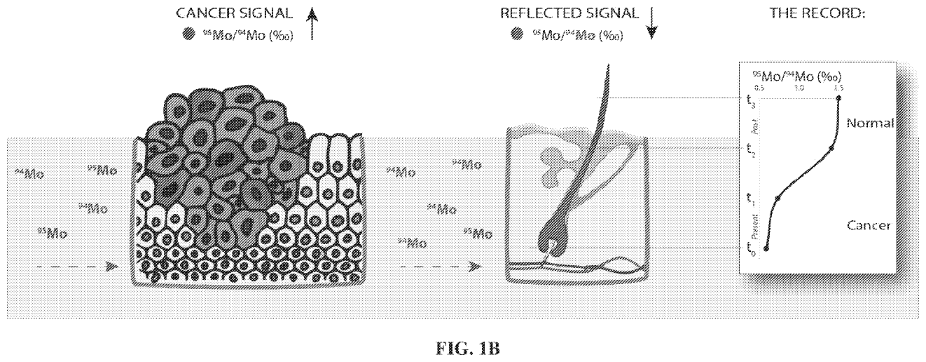

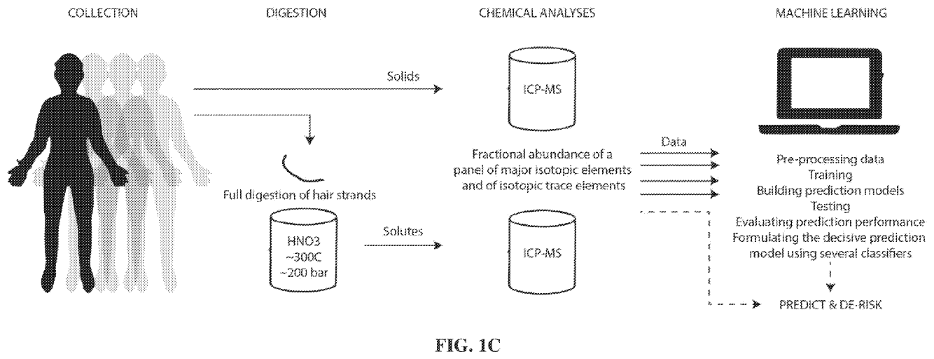

The naturally occurring ratio of an element's heavy (more neutrons) and lighter (fewer neutrons) isotopes is altered as a result of transformations in tissue, cultural environment, geographical region, age, and/or sex. For example, water with the lighter isotope of oxygen (¹⁶O) is preferentially evaporated, transported, and stored in glaciers. During global cooling, glaciers grow and capture proportionally more of the lighter isotope (¹⁶O) such that, in parallel, more of the heavy oxygen isotope (¹⁸O) is left in the ocean for animals to use when building shells (made of CaCO₃) (FIG. 1A). This mechanism allows for a ‘fingerprint’ of global cooling over time that can be detected in both the ice (low ¹⁸O/¹⁶O ratio) and in shells (high ¹⁸O/¹⁶O ratio). Over geological timescales, however, the shells are best preserved and provide a temporal record of climate change [38]. Thus, isotope methodology allows the fingerprinting of transformations also in tissues, even when sampling spatially or temporally far away from the source of alteration and without prior knowledge of the details of its underlying mechanisms. The technology described herein uses hair as a temporal record of elemental turnover.

To date, no attempt has yet characterized the chemical reflection or isotopic fingerprint of tumor transformation in tissues that are distant from a tumor during cancer disease progression, for example tissues that can be non-invasively sampled, such as strands of hair, fingernails, or toenails. In some aspects, studies have shown that a tumor existing in the human body metabolizes molecules (like sugars) and elements at a different rate than non-cancerous (non-transformed) tissue, and studies have demonstrated isotopic signatures in the metabolites and blood of cancer patients [5-7]. Higher cancer cell proliferation may offer a chemical mechanism by which elements are used in an altered way (e.g., rates, amount, and re-cycling) than in tissues of the healthy person. This mechanism may lead to the proportions of an element's heavier and lighter isotopes being altered as a result of tissue transformation (primary signal). The different rate of metabolism of cancer cells may alter the overall concentration of these isotopic elements (and concentrations of their individual isotopes) both in the growing tumor and in the surrounding milieu, e.g., tissue, muscles, or serum, as well as in co-factors to key enzymes. These differences in the surrounding milieu (reflective signal) can be recorded in, e.g., blood or serum. However, the extent to which the reflective signal can also accumulate and be recorded over longer time-scales in materials at a distance from the tumor, e.g., hair, fingernails, and toenails, has not been tested. Isotopic fingerprinting using tissues such as hair, fingernails, or toenails can provide certain advantages. For example, the chemical record in these tissues can be less susceptible to daily chemical variations than would, e.g., blood and serum. These tissues can also be sampled non-invasively.

To date, no attempt (in either the geosciences, medical sciences, or forensic sciences) has yet used machine learning that incorporates features from multiple isotopic features simultaneously (a multi-element approach) to characterize conditions in a setting. To date, no attempt has yet used machine learning (supervised or unsupervised) for a multi-element approach to characterize tumor growth within the human body. To date, no attempt has yet used machine learning (supervised or unsupervised) for a multi-element approach to characterize the presence of tumor growth via a chemical fingerprint preserved in tissues that are distant from the tumor. To date, no attempt has yet utilized the broad range of isotopic elements and features (including those representing natural variability within the human population) preserved in tissues to build (e.g., train) viable predictive models with a low risk to overfit (compared to how ultraclean samples that perfectly represent the sought conditions will contribute to models that are susceptible to minor variations and contamination in test samples so that the models thus overfit to the ultraclean samples and do not generalize to other samples that may be collected).

In the present disclosure, the method of stable isotope geochemistry (here also called isotopic fingerprinting) was adapted for use with animal (e.g., mammalian) subjects, e.g., humans, and a specific isotopic fingerprint that was fossilized or reflected in hair strands and nails of the subjects was obtained, which provided information on the existence of a primary tumor, if any. To interpret the data, machine learning algorithms were used to identify the presence of cancer, and what kind of cancer, in the subjects in a high-dimensional manner.

Broadly, the present disclosure relates to methods of noninvasively detecting, diagnosing, or assessing cancer in a subject by doing a chemical analysis of the stable isotopic elements of a sample, e.g., hair or nails, from the subject and inputting the results into a predictive model built using samples from a plurality of subjects, e.g., from a representative number of subjects. The methods described herein have the potential to detect cancer in undiagnosed people (cancer detection screen) as well as monitor for the presence of cancer after therapy (cancer monitoring screen). The methods described herein can also be used to assess cancer, e.g., characterize tumor burden, cancer risk, cancer growth risk, or tumor metastasis, in a subject. In some embodiments, the methods described herein combine the chemical analysis of at least one sample, obtained non-invasively, using mass spectrometry with a machine learning algorithm to identify patients having cancer. In some embodiments, persons identified as having cancer by noninvasive detection undergo biopsy to identify the cancer. In some embodiments, the subject diagnosed with cancer or where cancer was detected further undergoes treatment of the cancer, for instance chemotherapy, radiotherapy, cryotherapy, photodynamic therapy, laser therapy, immunotherapy, targeted therapy, hormone therapy, surgical procedures to remove the cancer, stem cell transplant, bone marrow transplant, or any combination thereof.

Advantageously, the methods and systems described herein using isotopic fingerprinting can be utilized to noninvasively diagnose, detect, or assess cancer in a subject. The methods and systems are an enormous advancement in the cancer diagnosis, detection, and assessment arts, representing a relatively simple way to identify the presence of even minute amounts of cancer in the subject, often well before any other means of diagnosis and detection, which are often expensive and/or invasive, can even find the cancer. Once a prediction model is established, such as for a specific type of cancer, and an isotopic chemical analysis of a sample is obtained, e.g., using ICP-MS, the prediction model can be used to, e.g., conclude that the subject does, or does not, have cancer, or to assess the extent or level of tumor burden. It should be appreciated that each type of cancer can have its own prediction model or models, and as such, one sample (e.g., hair or nail) in some embodiments can be run through at least 1, 2, 3, 4, 5, 6, 7, 8, 9, 10, or more different prediction models, wherein each prediction model is specific to a type of cancer. In some embodiments, the prediction model for a type of cancer is location dependent (e.g., North America versus Europe versus Asia or other setting with specific habits of food and drink intake). In some embodiments, the prediction model for a type of cancer is biological gender dependent.

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

The section headings used herein are for organizational purposes only and are not to be construed as limiting the subject matter described.

### I. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

The term “about” or “approximately” as used in connection with number indicates that the a given value for the number can vary by up to 10%. For example, “about 100” means an amount of from 90-110. In some instances, “about” and “approximately” are used to provide flexibility to a numerical range endpoint by providing that a given value may be “slightly above” or “slightly below” the endpoint without affecting the desired result, for example, +/−5%. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of” the embodiments or elements presented herein, whether explicitly set forth or not.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Sample,” “specimen,” “sample from a subject,” “biological sample,” and “patient sample” are used interchangeably herein, in some embodiments to refer to a sample of hair from anywhere on the mammal's body; fingernails; toenails; claws; or hooves. In some embodiments, the sample is a non-invasive sample. In some embodiments, a noninvasive sample is any sample that involves only a minor intervention to obtain from a subject. For example, a noninvasive sample can be one that does not involve use of a needle, surgery, general anesthesia, or sedation.

“Subject” and “patient” as used herein interchangeably refer to any vertebrate, for instance a mammal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, a non-human primate (for example, a monkey, such as a cynomolgus or rhesus monkey, chimpanzee, etc.), or a human). In some embodiments, the subject is a human. In some embodiments, the subject has been previously diagnosed with cancer. In some embodiments, the subject is a human who is suspected of having cancer. In some embodiments, the subject is a human who is not suspected of having cancer. In some embodiments, the subject is a human in cancer remission, and it needs to be determined if they are still in cancer remission. In some embodiments, the subject is undergoing treatment for cancer. In some embodiments, the subject is a human who is suspected of having metastatic cancer. In some embodiments, the subject is a human who is suspected of having localized cancer.

As used herein, a “system” refers to a plurality of real and/or abstract elements operating together for a common purpose. In some embodiments, a “system” is an integrated assemblage of hardware and/or software elements. In some embodiments, each component of the system interacts with one or more other elements and/or is related to one or more other elements. In some embodiments, a system refers to a combination of components and software for controlling and directing methods.

“Treat,” “treating,” or “treatment” are each used interchangeably herein to describe reversing, alleviating, or inhibiting the progress of a disease and/or injury, or one or more symptoms of such disease, to which such term applies. For example, treatment can include the administration of chemotherapy, the administration of radiotherapy, cryotherapy, photodynamic therapy, laser therapy, immunotherapy, targeted therapy, hormone therapy, surgical procedures to remove the cancer, stem cell transplant, bone marrow transplant, or any combination thereof. Depending on the condition of the subject, the terms can also refer to preventing a disease, for instance preventing the onset of a disease, or preventing the symptoms associated with a disease. A treatment may be either performed in an acute or chronic way. The terms can also refer to reducing the severity of a disease or symptoms associated with such disease prior to affliction with the disease. In some embodiments, the prevention or amelioration of the severity of a disease prior to affliction refers to administration of a pharmaceutical composition to a subject that is not at the time of administration afflicted with the disease. In some embodiments, the prevention or amelioration of the severity of a disease refers to administration of a pharmaceutical composition to a subject that is at the time of administration afflicted with the disease. In some embodiments, the prevention or amelioration of the severity of a disease refers to the surgical removal of a body part based on familial history and/or genetic testing. “Preventing” can also refer to preventing the recurrence of a disease or of one or more symptoms associated with such disease. “Treatment” and “therapeutically” refer to the act of treating, as “treating” is defined above.

As defined herein, a “stable isotope” corresponds to an isotope that has a half-life greater than the age of the Solar System, ~10⁹ years (i.e., not subject to radioactive decay). Hence, this includes the primordial nuclides that can be considered radioactive but are stable on a geological timescale.

As defined herein, “isotopic feature” is used to describe any value that can be calculated based on the amount (concentration) of an isotopic element (e.g., C) or an isotope (e.g., ¹³C) of an isotopic element (e.g., C) and of ratios between isotopes within or between elements. As used herein, “isotopic element” refers to an element having one stable isotope or more. Exemplary isotopic features are described in [35], as well as hereinbelow.

As defined herein, “atom percent”, “atom percent values”, and “A %” are used interchangeably. Atom percent is calculated by dividing the amount (concentration) of the elemental isotope of interest (e.g., ¹³C) by the total amount (concentration) of one or more stable isotopes of interest, times 100. The one or more stable isotopes of interest include at least one stable isotope that is not the elemental isotope of interest. The one or more stable isotopes of interest can be or include stable isotopes of the same element as the elemental isotope of interest. For example, atom percent can be calculated by dividing the amount (concentration) of the elemental isotope of interest (e.g., ¹³C) by the total amount (concentration) of one or more stable isotopes of interest of that element (e.g., ¹²C+¹³C), times 100. In some embodiments, the one or more stable isotopes of interest can also be or include stable isotopes of other elements of interest. For example, atom percent can be calculated by dividing the amount (concentration) of the elemental isotope of interest (e.g., ¹³C) by the amount (concentration) of one or more stable isotopes of other elements of interest (e.g., ⁵¹Ti), times 100. The one or more stable isotopes of interest can also include stable isotopes of multiple elements of interest. The multiple elements of interest can include or not include the element of the elemental isotope of interest.

As defined herein, “fractional abundance”, “isotopic fractional abundance”, “F”, and “f” are used interchangeably. A fractional abundance is calculated by dividing the amount (concentration) of the elemental isotope of interest (e.g., ¹³C) by the total amount (concentration) of one or more stable isotopes of interest. The one or more stable isotopes of interest include stable isotopes of the same element as the elemental isotope of interest. For example, a fractional abundance can be calculated by dividing the amount (concentration) of the elemental isotope of interest (e.g., ¹³C) by the total amount (concentration) of one or more stable isotopes of interest of that element (e.g., ¹²C+¹³C) or by 1 minus the total amount (concentration) of the isotope of interest (e.g., ¹³C).

As defined herein, “isotope ratio”, “isotope ratios”, and “R” are used interchangeably. An isotope ratio is calculated by dividing the amount (concentration) of the elemental isotope of interest (e.g., ¹⁸O) by the total amount (concentration) of other stable isotopes of that element (e.g., ¹⁶O+¹⁷O, which can be denoted as 1-¹⁸O). As defined herein, an “enriched” (high value) or “depleted” (low value) sample are used to describe a sample that has a higher or lower isotope ratio, respectively, of an isotope, e.g., ¹⁸O/(1-¹⁸O), than the value of the same isotope ratio in a reference material (whether certified, lab-specific, or matrix-relevant). In some embodiments, the concentration of the elemental isotope of interest can also be divided with the stable isotopes of other elements of interest. For example, an isotope ratio can be calculated by dividing the amount (concentration) of the elemental isotope of interest (e.g., ¹³C) by the amount (concentration) of one or more stable isotopes of other elements of interest (e.g., ⁵¹Ti). An isotope ratio can be calculated by dividing the fractional abundance (from concentrations) of the elemental isotopes of interest (e.g., ¹³C/¹²C) by the fractional abundance (from concentrations) of one or more stable isotopes of other elements of interest (e.g., ¹⁵N/¹⁴N). The one or more stable isotopes of interest that make up the denominator can also include stable isotopes of multiple elements of interest. The one or more stable isotopes of interest in the denominator can also include stable isotopes of multiple elements of interest. The multiple elements of interest can include or not include the element of the elemental isotope of interest.

As defined herein, “delta values”, “delta symbols”, “delta”, “d”, and “δ” are used interchangeably. “Delta values” or “delta symbols” are known in the geochemical arts as a way to express the relative difference of isotope ratios between a sample and an agreed-upon reference material. For carbon, a common delta value is calculated using the formula:

$${δ}^{13} {C}_{Standard} = [ \frac{{( {}^{13} F / ( 1 - {}^{13} F ) )}_{sample}}{{{(}^{13} F / ( 1 - {}^{13} F ) )}_{Standard}} - 1 ] .$$

The delta value can be expressed as is or as permil (‰), that implies a multiplication of the value by 1000. For example, a δ¹³C value can be expressed as 0.0021 or 2.1% e. Representative formulas for other elements are easily determined by the person skilled in the art. As defined herein, a “high” or “low” delta value (e.g., δ¹³C) are used to describe a sample that is enriched (e.g., in the proportion of ¹³C rendering a high value) or depleted (e.g., the proportion of in ¹³C rendering a low value), respectively, to a context-dependent reference value or standard. The fractional abundance of an isotope can also be compared to the fractional abundance of an isotope or another element, where the latter serves as a reference value. For some delta values, the standards used are those used conventionally, like air for nitrogen or the Vienna Pee Dee Belemnite (V-PDB) for oxygen delta values. Reference materials can also be used within a study or laboratory to offer the development of novel delta values. Delta values can be converted to atomic percent values, when using the agreed upon R for the reference material or the conversion [37].

As defined herein, “overall concentration” can be used to refer to the total concentration of an isotopic element, including all stable isotopes of the isotopic element that are measured in a sample. “Overall concentration” can also be used to refer to the concentration of a non-isotopic element that does not have stable isotopes.

As defined herein, “concentration ratio” is used to describe a ratio between the overall concentration of one isotopic element (e.g., N) normalized to the overall concentration of another element (e.g., C), giving a ratio (e.g., N/C). A concentration ratio can also be used to describe the ratio between the overall concentration of a non-isotopic element and the overall concentration of a non-isotopic element or an isotopic element.

As defined herein, “noninvasive” is intended to describe the methods of diagnosing, detecting, or assessing cancer as described herein, wherein the cancer is diagnosed, detected, or assessed using a sample from a subject such as the subject's hair (from anywhere on the subject's body), fingernails, skin flakes, toenails, claws, or hooves. The methods of diagnosing, detecting, or assessing cancer as described herein in some embodiments do not require any fluid (e.g., blood, plasma, serum, lymph, etc.) or tissue that would require invasive sampling from the subject, but can also be performed with such fluids or tissues. It is understood that following the diagnosis, detection, or assessment of cancer using the methods described herein, additional testing, such as invasive testing, may be ordered.

As defined herein, “mass spectrometry” can include at least one of inductivity coupled plasma mass spectrometry (ICP-MS), multiple collector ICP-MS (MC-ICP-MS), Sector Field ICP-MS (SF-ICP-MS), gas or noble gas isotope ratio mass spectrometry (IRMS), elemental analysis (EA-IRMS), gas chromatography IRMS (GC-IRMS or GCC-IRMS), liquid chromatography IMRS (LC-IRMS), thermal ionization mass spectrometry (TIMS), pyrolysis gas chromatography mass spectrometry (Pyr-GC-MS), secondary ion mass spectrometry (SIMS), or any mass balance device that determines the balance of masses in the material.

As defined herein, “internal standard” can include one or more solutions or materials used to monitor the performance and reliability of the method for mass spectrometry, for example a solution including Indium (In).

As defined herein, “calibration standard” can include one or more solutions or materials used to constrain the concentrations of elements in the sample. For example, standard solutions ranging from 0.01-100 μg/mL of elements of interest (e.g., Zn, Cu, Mn) can be prepared by serial dilution of 10 μg/mL single multielement stock solution from Agilent, USA.

As defined herein, “reference material” can include one or more solutions or materials used to assess the reliability of the measurements, for example the mix of ammonium sulfate and oxalic acid that is certified by the International Atomic Energy Agency (IAEA) with known delta values for carbon (C) and (N) called IAEA-C7. The reference material can be certified or “certified reference material” or “CRM” (e.g., via IAEA) or used over time within a specific laboratory.

As defined herein, “matrix-relevant reference material” can include one or more solutions or materials used to assess (and compensate for) matrix that are specific to the matrix of the samples. For example, in analyses of hair by ICP-MS, a matrix-relevant reference material from hair is included (digested to a solution or as a solid) such as the certified reference material (CRM) prepared from human hair called IAEA-085.

### II. PROCESS OF BUILDING A PREDICTION MODEL AND USE THEREOF

In a first aspect, methods of building and testing a prediction model are described herein. Once built, a prediction model can be used to diagnose, detect, or assess cancer in a subject.

The initiation of cancer is a long process that starts at a hidden site. The present disclosure is based on the finding that the isotopic fingerprint of a tumor can be reflected elsewhere in the body distant from the tumor, such that non-invasive chemical analysis of samples such as hair strands or nails can reveal the initiation of cancer early. By bulk analyses of the chemistry of samples of hair strands or nail clippings from cancer patients and healthy controls, it is possible to develop a prediction model that can be used to determine or predict cancer, e.g., determine the probability of the presence of cancer, in a patient. Assessment of cancer in the subject, such as the characterization of tumor burden, cancer risk, cancer growth risk, or tumor metastasis, can also be performed with the provided prediction models.

In some embodiments, the building of a prediction model and use thereof entail the analyses of the chemistry preserved in noninvasively sampled material (e.g., hair strands or nail clippings) from a representative number of subjects that is used to build the prediction model and, once the prediction model is built, the identification of whether the chemistry preserved in noninvasively sampled material (e.g., hair strands or nail clippings) from an individual subject indicates the growth of a tumor within the subject (FIG. 1C). In some embodiments, a prediction model for predicting the presence of cancer in a subject is built based on chemical features of isotopic elements (isotopic features) that are measured in such samples obtained from a representative number of subjects. In some embodiments, the samples include hair or nail samples. The analyses of the samples can include the acquirement of concentration data for the major and minor elements and their isotopes. With this data, various isotopic features, such as at least one of atomic percents, isotope ratios, fractional abundances, delta values, overall concentrations, and concentration ratios, can be calculated and used as input into the model. In some embodiments, the various isotopic features are calculated using certified reference materials, matrix-relevant reference materials, matrix-relevant and certified reference materials, different isotopic elements within the sample, or a combination of these.

In some embodiments, the prediction model can be used to determine, based on the value for each of the at least one isotopic feature for the subject, whether the subject has cancer. In some embodiments, the prediction model can be used to diagnose cancer in the subject. In some embodiments, the prediction model results in an output indicating the presence or absence of cancer. In some embodiments, the prediction model results in an output that is or indicates the probability of the presence of cancer. In some embodiments, a probability greater than 0.5 indicates the presence of cancer. In some embodiments, the prediction model results in an output indicating the type of cancer. In some embodiments, the prediction model results in an output indicating a characteristic of the subject or cancer, e.g., tumor burden, cancer risk, cancer growth risk, or tumor metastasis.

The prediction models provided herein may be “general models” or “specialized models.” General models represent a prediction model applicable to a large number of subjects, e.g., all subjects of a particular sex and/or all subjects in particular region (e.g., country). Specialized models are predictions models trained for a particular subject, region, and/or sex. Specialized models are typically derived from architype non-specialized models.

In some embodiments, the prediction model is a general model. In some embodiments, the general model is trained using isotopic features from a plurality of reference subjects. In embodiments, the general model is built using input data from a plurality of cancer subjects and a plurality of healthy (also referred to as normal or control subjects). In some embodiments, the plurality of reference subjects (e.g., cancer subjects or healthy subjects) used for training the general model generally include greater than 20 subjects, such as greater than 50, 60, 70, 80, 90, 100, 200, 300, 400, 500 or more subjects. In some embodiments, the plurality of reference subjects used to train the general model span multiple populations (e.g., ethnicities, ages, sexes), cultures, continents or countries (e.g., geographical regions), cancers, and/or socio-economical settings. In some embodiments, at least one subject of the plurality of reference subjects lives in a different geographical region, is a different sex, is a different age, is from a different cultural or ethnic group, has a different diet and/or has a different cancer type, compared to another subject of the plurality of reference subjects.

In some embodiments, the plurality of reference subjects used to train a general model comprises subjects from different geographical regions and/or cultures. For instance, in many cases subjects from different geographical regions may exhibit differences in diet such as due to exposure to different food and water sources or because such a region is enriched in a particular culture or ethnic group that may exhibit a different diet. Without wishing to be bound by theory, differences in diet or water availability may impact the chemical data. In some embodiments, a geographical region is a region that can be defined by any of a number of single or multiple features, generally with similar characteristics including but not limited to climate, weather, groundwater, terrain, vegetation, wildlife, or topographical characteristics. In some embodiments, a geographical region is a continent, a country or is any physical region within a country that is characterized by a similar characteristic. In some embodiments, at least one subject, such as any of at least one, two, three, four, five, 10, 20, 30, 40, 50, 60, or more, of the plurality of reference subjects lives in a different geographical region compared to another subject of the plurality of reference subjects. In some embodiments, the plurality of reference subjects span at least about two, three, four, five, 10, 15, 20, or more, geographical regions. In some embodiments, the plurality of reference subjects span at least about two, three, four, five, 10, 15, 20, or more, countries.

In some embodiments, the plurality of reference subjects used to train a general model comprises both males and females. In some embodiments, at least one subject, such as any of at least one, two, three, four, five, 10, 20, 30, 40, 50, 60, or more, of the plurality of reference subjects is a different sex compared to another subject of the plurality of reference subjects.

In some embodiments, the plurality of reference subjects used to train a general model comprises subjects of different ages. In some embodiments, at least one subject, such as any of at least one, two, three, four, five, 10, 20, 30, 40, 50, 60, or more, of the plurality of reference subjects is a different age from another subject of the plurality of reference subjects.

In some embodiments, the plurality of reference subjects used to train a general model comprises subjects having different cancer types. In some embodiments, at least one subject, such as any of at least one, two, three, four, five, 10, 20, 30, 40, 50, 60, or more, of the plurality of reference subjects has a different cancer compared to another subject of the plurality of reference subjects.

In some embodiments, the prediction model is a specialized model. In some embodiments, the specialized model is trained using isotopic features from a plurality of reference subjects. In embodiments, the specialized model is built using input data from a plurality of cancer subjects and a plurality of healthy (also referred to as normal or control subjects). In some embodiments, the plurality of reference subjects (e.g., cancer subjects or healthy subjects) used for training the specialized model generally include greater than 20 subjects, such as greater than 50, 60, 70, 80, 90, 100, 200, 300, 400, 500 or more subjects. In some embodiments, the plurality of reference subjects used to train the specialized model are from the same population (e.g., ethnicity, age, sex), culture, continent or country (e.g., geographical region), cancer, and/or socio-economical setting.

In some embodiments, the plurality of reference subjects used to train a specialized model are subjects from the same geographical region. In some embodiments, each subject of the plurality of reference subjects lives in the same geographical region. In some embodiments, each subject of the plurality of reference subjects lives in the same country. In some embodiments, each subject of the plurality of reference subjects lives in the same state. In some embodiments, the plurality of reference subjects used to train a specialized model are from the same geographical region and are the same sex. In some embodiments, the plurality of reference subjects used to train a specialized model are from the same country and are the same sex. In some embodiments, the plurality of reference subjects used to train a specialized model are from the same country and are male.

In some embodiments, the plurality of reference subjects used to train a specialized model are the same sex. In some embodiments, each subject of the plurality of reference subjects is male. In some embodiments, each subject of the plurality of reference subjects is female.

In some embodiments, the plurality of reference subjects used to train a specialized model comprises subjects of the same age.

In some embodiments, the plurality of reference subjects used to train a specialized are from the same geographical region, are the same age, are the same sex, and have the same cancer type.

### A. Samples and Feature Determination

In some embodiments, the disclosed methods and systems may be used with any of a variety of samples that are collected from a subject (e.g., a patient). In some embodiments, the sample is a noninvasive sample. In some embodiments, the noninvasive sample is any sample that involves only a minor intervention to obtain from a subject. For example, the noninvasive sample is one that does not involve use of a needle, surgery, general anesthesia, or sedation.

In some embodiments, the sample is a solid sample. In some embodiments, the sample contains keratinous tissue. In some embodiments, the sample contains hard keratinous tissue.

In some embodiments, the sample is from hair, nails, or skin. In some embodiments, the sample is a hair sample. In some embodiments, the sample is a nail sample. In some embodiments, the nails can include fingernails or toenails. In some embodiments, the sample is a skin sample. In some embodiments, the skin can include skin flakes. In some embodiments, the sample can be collected directly by the subject and need not be obtained by a medical provider.

In some embodiments, the sample is a hair sample. In some embodiments, the hair sample is from the head of the subject. In some embodiments, the sample is from the beard of the subject. In some embodiments, the sample is from the pubic hair of the subject. In some embodiments, the sample is from the underarm hair of the subject. In some embodiments, the sample is from the body hair of the subject. In some embodiments, the sample is from the chest hair of the subject. In some embodiments, the sample is from the back hair of the subject. In some embodiments, the sample is from the abdominal hair of the subject. In some embodiments, the hair sample is about a teaspoon of hair. In some embodiments, the hair sample is between 0.25 and 2 inches in length. In some embodiments, the hair sample is between 0.5 and 2 inches in length. In particular embodiments, the hair sample is no longer than about 1.5 inches in length and generally at least 0.5 inches in length. In particular embodiments, the hair sample is no longer than about 1.5 inches in length and generally at least 0.25 inches in length. In some embodiments, the hair sample is between about 0.75 inches and about 1.5 inches. In some embodiments, the hair sample is between about 1.0 inches and about 1.5 inches in length. In some embodiments, the hair sample that is collected has not been subjected to any treatment of the collected hair, such as a medicated shampoo (e.g., containing selenium or zinc), bleaching or dying of the hair, or treatment of the hair with a hair spray, gel, hair cream, oil, or other such treatments. Alternatively, the hair sample can be one that has been subjected to treatment of the hair, such as using thermal or chemical means, e.g., for dyeing or perming the hair.

In some embodiments, the sample is collected from dry hair (e.g., dried for at least 4 hours after washing) and within 24 hours of washing the hair. In some embodiments, the sample is a hair sample from the head of the subject, generally including the newer growth of hair such as near the scalp.

In some embodiments, the sample is a nail sample. In some embodiments, the nail sample includes a nail clipping of 1-4 mm in length, such as 2-3 mm in length from at least one nail. In some embodiments, the nail is a fingernail. In some embodiments, the nail is a toenail. In some embodiments, the nail sample includes 2-10 clippings from different nails. In some embodiments, the nail sample includes 6-8 clippings from different fingernails, such as clippings from each of the 10 fingernails. In some embodiments, the nail sample is about 5 mg to about 100 mg in weight, such as about 5 mg, about 10 mg, about 20 mg, about 30 mg, about 40 mg, about 50 mg, about 60 mg, about 70 mg, about 80 mg, about 90 mg or about 100 mg, or any value between any of the foregoing. In some embodiments, the nail sample that is collected has not been subjected to any treatment of the collected nail, such as with a nail polish.

In other embodiments, the sample is a sample that involves more than minor intervention to obtain the sample from the subject. In some embodiments, obtaining the sample involves use of a needle, surgery, general anesthesia, or sedation. In some embodiments, obtaining the sample involves use of a needle. In some embodiments, obtaining the sample involves drawing blood from the subject. In some embodiments, the sample is blood or a blood component. In some embodiments, the sample is whole blood. In some embodiments, the sample is plasma. In some embodiments, the sample is serum.

In some embodiments, the sample is collected into a container suitable for storage prior to isotope analysis. In some embodiments, the container is a glass container. In some embodiments, the container is a polyethylene container, such as a high-density polyethylene (HDPE) container. In some embodiments, the container is a polypropylene container. In some embodiments, the container is a Polytetrafluoroethylene (PTFE) container. In some embodiments, the container is a perfluoroalkozy (PFA) container. In some embodiments, the container is a vessel, such as a tube, paper envelope, or paper or plastic bag.

In some embodiments, the samples are collected in North America, Europe, Asia, or other continents. In any of the provided methods and systems involving a plurality of samples, the plurality of samples in some embodiments include samples from multiple continents. In other embodiments, the plurality of samples include samples from only one continent.

In some embodiments, prior to isotopic analysis the samples are prepared. The methods of preparation can include methods that dissolve the isotopic elements from its matrix and minimize chemical contamination from other sources, such as the container or reagents. In some embodiments, 0.1 mg to 50 mg of sample are processed for analysis, such as by transferring such amount into a new container for analysis. In some embodiments, at or about 0.2 to 20 mg, 0.2 to 10 mg, 0.2 to 5 mg, 0.2 to 1 mg, or 0.1 to 0.5 mg are processed. In some embodiments, at or about 0.2 mg, about 0.5 mg, about 1 mg, about 5 mg, about 10 mg, about 20 mg, about 30 mg, about 40 mg, or about 50 mg of the sample are processed. In some embodiments, at or about 50 mg of the sample are processed. In some embodiments, the container is a glass container. In some embodiments, the container is a polyethylene container, such as an HDPE container. In some embodiments, the container is a polypropylene container. In some embodiments, the container is a Polytetrafluoroethylene (PTFE) container or a modified PTFE (TFM) container. The modified PTFE of a TFM container is copolymerized PTFE that introduces an oxygen molecule and results in a higher density material, which in some aspects results in smaller deformations under high temperature and high pressure and smaller permeability. In some embodiments, the container is a PFA container. In particular embodiments, the container is a PFA digestion vessel. In some embodiments, the container is a tube with a cap. In some embodiments, the samples are stored at about 2° C. to 6° C., such as about 4° C. until analysis.

In some embodiments, the samples are washed prior to the homogenization or digestion process. In some embodiments, the samples are washed with one or more different solvents, such as a non-ionic detergent (e.g., Triton X-100), acetone, and/or deionized water. In some embodiments, the washing procedure involves stirring of the samples with different solvents in the following sequence: 0.5% Triton-X 100, deionized water, and acetone, using a mechanical shaker, followed by repeated rinsing with deionized water. Prior to analysis, the samples can be dried, such as by heating to about 60° C., for example using a drying oven.

In some embodiments, the samples are combusted before analysis. In some embodiments, the samples are combusted before analysis with MS using a so-called elemental analyzer (EA). In some embodiments, the solid sample (e.g., hair or nail) are then packed into cups of tin or silver. In some embodiments, a catalyst like vanadium pentoxide (VO₅) is added to the sample in the cup to improve immediate combustion of more refractory materials samples.

In some embodiments, the samples (e.g., hair or nail) are processed by homogenization for analysis. In some embodiments, the homogenization methods including grinding or digestion of the samples. In some embodiments, the sample digestion produces a liquid sample from the sample. Digestion of the sample may be helpful for the detection of elements or isotopic elements that are less abundant in the sample. For example, trace elements (minors) or isotopic elements can benefit by digestion of the sample for preparation of a liquid sample prior to analysis by mass spectrometry. In some embodiments, the sample digestion is an acid digestion and/or thermal digestion. In some embodiments, the sample digestion is an acid digestion and thermal digestion. Any of a variety of methods can be used to digest the samples that dissolves the analytes and decomposes solids while avoiding loss or contamination of the sample [33, 36]. In some embodiments, sample digestion is by subjecting the sample to nitric acid (HNO₃) treatment. In some embodiments, the HNO₃ is a 65% w/v solution. Concentrated HNO₃ (e.g., 14 M and 65%) is a strong oxidizing agent that can liberate trace elements from many materials, either with or without thermal heating. In provided aspects, nitric acid can prevent or reduce elements and chemicals from adhering to the container walls, and thus can ensure that the total amount of elements/chemicals will be available for analysis, and hence measured.

In some embodiments, the volume of HNO₃ for digestion is any volume to sufficiently digest the sample, such as about 0.2 mL to 2 mL. In some embodiments, HNO₃ is added to the sample and allowed to digest for 6 to 72 hours, such as at or about 1 day or 2 days. In some embodiments, the digestion is for about 2 days or 48 hours. In some embodiments, nitric acid (HNO₃) and hydrogen peroxide (H₂O₂) are used in the digestion process. The choice to use HNO₃ only or a combination of HNO₃ and H₂O₂ is within the level of a skilled artisan. In some embodiments, 0.2 mL to 0.5 mL of HNO₃ (e.g., 65% w/v solution) and 0.2 mL to 0.5 mL of H₂O₂ (e.g., 30% w/v solution) is added to the sample for digestion. The HNO₃ and H₂O₂ can be added simultaneously or sequentially. In one example, about 0.5 mL HNO₃ and 0.5 mL H₂O₂ are added to the sample for digestion. In another example, about 0.5 mL HNO₃ and 0.5 mL H₂O₂ are added to the sample for digestion. In another method, the sample is first digested with HNO₃, such as overnight, and then H₂O₂ is added, optionally after first drying the sample.

In some embodiments, the sample digestion is by acid digestion and thermal digestion. In some embodiments, acid digestion (e.g., with HNO₃ or HNO₃ and H₂O₂ or even HNO₃, H₂O₂, and HF) is carried out with thermal heating of the sample. The sample can be heated by electric heating or a microwave. In some embodiments, a heating plate or other heating device is used to heat the sample to about 80° C. to 150° C., such as at or about 90° C. or about 100° C. The sample can be heated for 1 hour to 4 hours, such as about 2 hours. In some embodiments, the sample is digested in a chamber with both high temperature (up to 300 degrees C.) and pressure (up to 240 bar). The sample can be digested at these conditions in under 1 hour. In some embodiments, about 0.2 mL to 0.5 mL of HNO₃ is added and the sample is heated at about 90° C. for 2 hours, then is allowed to cool and then about 0.2 mL to 0.5 mL of H₂O₂ is added and the sample is heated at about 90° C. for about 1 hour. In some embodiments, microwave assisted acid digestion can be used in which microwave heating is carried out with a suitable laboratory microwave unit. In some embodiments, temperatures in the range of 200-300° C. (far in excess of the boiling points of the acids) can be generated, which can accelerate the digestion process. In some embodiments, the sample and acid(s) are placed in a suitable vessel container, such as a fluorocarbon polymer (e.g., PFA or TFM) or quartz microwave vessel or vessel liner. In some embodiments, the microwave system is able to provide 600W to 1200 W of power. The power setting can be adjusted to a power of between 40% and 100% of the power, such as 40%, 50%, 60%, 70%, 80%, 90%, 95% of the power. In some embodiments, the power is adjusted to between 50% and 60%. In some embodiments, the power is adjusted to provide 300 W to about 360W of power, such as at or about 325 W of power. The container (e.g., vessel) is sealed and heated in the microwave unit for a specified period of time, such as 10 minutes to 1 hour, for example about 20 minutes to about 40 minutes, such as at or about 30 minutes. In some embodiments, the sample is predigested using acid digestion on a heat block prior to microwave digestion. In some embodiments, about 0.5 ml of HNO₃ and 0.5 ml of H₂O₂ is added to the sample and heated at about 325 W for 30 min. In some embodiments, after cooling to room temperature, 9 ml of ultrapure water is added. Variations of the digestion methods, including acid volume, temperature and timing of digestion, can be empirically determined by a skilled artisan.

In embodiments in which samples are processed by heating, the contents of the container are cooled. In some embodiments, the contents of the container are filtered, centrifuged, or allowed to settle. Undigested residue can be centrifuged and further digested with HF. In some embodiments, the contents of the container are diluted to volume and analyzed. In some embodiments, dilution is with an HNO₃, deionized water, or ultraclean deionized water.

In some embodiments, samples do not contain more than 1,000 ppm total dissolved content so as not to clog the sample injection orifices and to guarantee accurate measurements. If they do, or are suspected to, sample dilutions can be made accordingly. When elements in samples exceed calibrated concentration values, dilution can also be performed.

In some embodiments, the value of an isotopic feature is determined for the sample. In some embodiments, the isotopic feature is the overall concentration of at least one isotopic element. In some embodiments, the isotopic feature is determined from the overall concentration of at least one isotopic element. In some embodiments, the overall concentration of at least one isotopic element is measured in the sample. In some embodiments, the overall concentration is measured by mass spectrometry.

In some embodiments, the isotopic feature is the concentration of an isotope of an isotopic element. In some embodiments, the isotopic feature is a chemical feature determined from the concentration of an isotope of an isotopic element. In some embodiments, the concentration of an isotope of an isotopic element is measured in the sample. In some embodiments, the concentration of the isotope is measured by mass spectrometry.

In some embodiments, the mass spectrometry is Inductively Coupled Plasma Mass Spectrometry (ICP-MS). In some embodiments, the reliability of the MS is monitored using internal standards.

For determination of isotope concentrations, samples can be introduced into an inductively coupled plasma mass spectrometer (ICP-MS) to detect one or more ionization products of elements and isotopic elements present in each sample. In some embodiments, the methods are carried out with a calibration standard that is individually introduced into the mass spectrometer to detect one or more ionization products of elements and isotopic elements present in the calibration standard. In provided embodiments, an absolute concentration of the one or more detected elements in each sample relative to the one or more detected ionization products of elements (e.g., isotopic elements comprising one or more isotopes of the element) in the calibration standard can be determined. In some embodiments, each of the calibration standards comprise elements and isotopic elements that correspond to the isotope that is of natural abundance for that element or isotopic element. In some embodiments, each of the calibration standards is provided in at least two different concentrations, and in some embodiments at least three different concentrations or at least four different concentrations. In one example, the calibration standards are prepared as 0, 4, 10, and 50 ppb (w/w) solutions by serial dilution of a stock standard. In another example, calibration standards are prepared as 0, 2, 10 and 50 ppb (w/w) solutions by serial dilution of a stock standard. In some embodiments, the standards have a concentration range that covers the expected concentrations in the samples analyzed. In some aspects, the plurality of calibration standards are utilized, e.g., because the ICP-MS measures counts per second (cps) of elements on its detector, not actual concentrations of elements, which are not linearly related to cps. In some embodiments, the plurality of calibration standards is entailed in one multi-elemental standard stock solution. Molecules and elements can be ionized by the high temperature of an argon plasma flame in the ICP-MS. The resulting ions can be separated by their mass-to-charge ratio and counted on the device mass detector. The numbers of ions hitting the detector at a specific mass/charge ratio can be recorded as counts per second. Calibration standards of determined concentrations of elements can be read into (and stored into memory of) the ICP-MS, and the result correlated to cps. Hence, specific values of cps can be assigned to the calibration standard's respective concentration. In some aspects, a blank sample is introduced into the mass spectrometer. The blank sample can include deionized water and HNO₃, as well as the internal standard (e.g., 97.5% v/v deionized water, 1.5% v/v HNO₃, and about 1% v/v internal standard). A calibration regression formula can be produced from the calibration standards and blank sample, which can be calculated by the processor associated with the ICP-MS and stored in memory. Using the calibration regression formula for each element, further measured counts per seconds (from samples) can be converted into concentrations of that element in a sample. Using the known abundance of each stable isotope of an element (e.g., natural abundance), the measured concentration for the element can be divided into known concentrations of each stable isotope of that element.

In some embodiments, an internal standard is added to the samples for the monitoring of MS' performance and reliability, such as due to a discriminatory loss of analyte during injection, ionization, or detection. In some embodiments, the internal standards are typically elements that are assumed not to occur in the samples like, e.g., Scandium (Sc) or Indium (In), or are assumed to be present in untraceable amounts, and therefore are not being calibrated for; this can typically be an element with only one isotope, or one very significant isotope. Furthermore, the internal standard can be in close proximity (in terms of their atomic number) to the measured, and calibrated, elements. In some embodiments, the internal standard is a combination of two or three elements present in the internal standard at known concentrations. In certain embodiments, the three elements are each present in the internal standard at a concentration within a range of about 0.05 to about 100 mg/L, such as from about 0.1 to about 50 mg/L. Exemplary internal standards include IAEA-085, ERM-DB001, IA-R068 (soy protein, δ¹³C=−25.22‰, δ¹⁵N=+0.99‰), IA-R038 (L-alanine, δ¹³C=−24.99‰, δ¹⁵N=−0.65‰), IA-R069 (tuna protein, δ¹³C=−18.88‰, δ¹⁵N=+11.60‰), and a mixture of IA-R046 and IAEA-C7 (ammonium sulfate and oxalic acid, δ¹³C=−14.48‰, δ¹⁵N=+22.04‰).

In some embodiments, calibration standards, blank samples, reference materials, and matrix-relevant reference materials are added for purposes of internal correction, such as due to loss of analyte during sample preparation, injection, ionization or matrix-specific retainment of elements. In some embodiments, the calibration standards contain elements that are similar to the measured, and calibrated, elements. In some embodiments, the calibration standard is a combination of two or three elements present in the samples. In certain embodiments, the elements are each present in the calibration standard or reference material at a concentration within a range of about 0.05 to about 100 mg/L, such as from about 0.1 to about 50 mg/L. In certain embodiments, the elemental concentrations that are each present in the sampled calibration standard or reference material are in close proximity (in terms of weight or concentration) to what will be measured in the sample. In certain embodiments, the elemental concentrations that are overall present in the sampled calibration standards or reference materials range (in terms of weight or concentration) to what will be present and measured in the samples. Exemplary calibration standards or reference material include IA-R068 (soy protein, δ¹³C=−25.22‰, δ¹⁵N=+0.99‰), IA-R038 (L-alanine, δ¹³C=−24.99‰, δ¹⁵N=−0.65‰), IA-R069 (tuna protein, δ¹³C=−18.88‰, δ¹⁵N=+11.60‰), and a mixture of IA-R046 and IAEA-C7 (ammonium sulfate and oxalic acid, δ¹³C=−14.48‰, δ¹⁵N=+22.04‰). Exemplary reference materials that are matrix-relevant are IAEA-085 (hair) and ERM-DB001 (hair).

In some embodiments, once the overall concentration of a given isotopic element or the concentration of an isotope of an isotopic element in a sample is determined, values of at least one isotopic feature related to the overall concentration or the concentration of the isotope can be determined. In some embodiments, the value of at least one isotopic feature is the overall concentration of an element. In some embodiments, the value of at least one isotopic feature is the overall concentration of an isotopic element. In some embodiments, the value of at least one isotopic feature is a chemical feature determined from the overall concentration of the isotopic element. In some embodiments, the chemical feature is a concentration ratio of the isotopic element to another element. In some embodiments, the other element is an isotopic element. In some embodiments, the other element is a non-isotopic element. Any of a number of other normalization strategies can be applied to determine a chemical feature determined from an overall concentration of an isotopic element.

In some embodiments, the at least one isotopic element can be any element having stable isotopes. In some embodiments, the provided methods and systems also involve the use of features of elements not having stable isotopes.

In some embodiments, at least one isotopic element is the concentration of an isotope of an isotopic element. In some embodiments, at least one isotopic feature is a chemical feature determined from the concentration of an isotope of an isotopic element. Any of a number of other normalization strategies can be applied to determine a chemical feature determined from a concentration of an isotope. In some embodiments, the chemical feature is an atomic percent, isotope ratio, fractional abundance, or delta for the isotope. In some embodiments, at least one isotopic feature is selected from (1) a concentration of an element, (2) a concentration of an isotopic element, (3) a concentration of an isotope of the isotopic element, or (4) a normalized value of any of (1)-(3). In some embodiments, at least one isotopic feature is a normalized value of any of (1)-(3). In some embodiments, at least one isotopic feature is a normalized value of a concentration of an element. In some embodiments, at least one isotopic feature is a normalized value of a concentration of an isotopic element. In some embodiments, at least one isotopic feature is a normalized value of a concentration of an isotope of the isotopic element.

The normalized values of the concentration of an element, concentration of an isotopic element, and concentration of an isotope of the isotopic element, and their relationships to each other, may be expressed by normalization in a number of different formats. In some embodiments, the normalized value of the at least one isotopic feature is selected from an atomic percent (atom %), isotope ratio (R), fractional abundance (F), delta value (delta), overall concentration, and concentration ratio (CR). In some embodiments, the normalized value of the at least one isotopic feature includes an atomic percent, isotope ratio, fractional abundance, delta value, overall concentration, and/or concentration ratio. In some embodiments, the normalized value of the at least one isotopic feature is a fractional abundance. In some embodiments, the normalized value of the at least one isotopic feature is a delta value. In some embodiments, the normalized value of the at least one isotopic feature is a plurality of features including any combination of atomic percents, isotope ratios, fractional abundances, delta values, overall concentrations, and concentration ratios. In some embodiments, the normalized value of the at least one isotopic feature is a plurality of features including fractional abundances and delta values. In some embodiments, the normalized value of the at least one isotopic feature is a plurality of features including fractional abundances, delta values, and concentration ratios. In some embodiments, the normalized value of the at least one isotopic feature is a plurality of features including atomic percents, isotope ratios, fractional abundances, deltas, overall concentrations, and concentration ratios. In some embodiments, the normalized values of the at least one isotopic feature are used to build the prediction model. In some embodiments, the normalized values of the at least one isotopic feature are used to train at least one machine learning algorithm that is comprised by the prediction model. In the methods, the calibration standard is chosen for measurement of isotopic elements.

In some embodiments, the normalized value of the at least one isotopic feature includes an atom percent of an isotope of an isotopic element. In some embodiments, the atom percent of the isotope is relative to stable isotopes of the isotopic element of the isotope. In some embodiments, the atom percent of the isotope is relative to stable isotopes of other isotopic elements. In some embodiments, the atom percent of the isotope is relative to stable isotopes of the isotopic element of the isotope and stable isotopes of other isotopic elements.

In some embodiments, the normalized value of the at least one isotopic feature includes an isotope ratio of an isotope of an isotopic element. In some embodiments, the isotope ratio of the isotope is relative to stable isotopes of the isotopic element of the isotope. In some embodiments, the isotope ratio of the isotope is relative to stable isotopes of other isotopic elements. In some embodiments, the isotope ratio of the isotope is relative to stable isotopes of the isotopic element of the isotope and stable isotopes of other isotopic elements.

In some embodiments, the normalized value of the at least one isotopic feature includes a fractional abundance of an isotope of an isotopic element.

In some embodiments, the normalized value of the at least one isotopic feature includes a delta value of an isotope of an isotopic element. In some embodiments, the delta value is a conventional delta value, a study-specific delta value (InH), a non-similar simple-nitrogen based delta value (SimpleNδ), or a non-similar pseudo-nitrogen based delta value (PseudoNδ). By normalizing the value of the at least one isotopic feature to a delta value, the results for the isotopic composition of major elements (e.g., C, N, S, O) that are reported in delta values may be amalgamated without the need to convert the delta values of major elements to a fractional abundance or atomic percent. In some embodiments, the delta value is converted to a fractional abundance or an atomic percent. Working examples herein exemplify methods utilizing delta values and demonstrate their conversions to other normalized values, such as fractional abundance or atomic percent.

In some embodiments, the delta value is a conventional delta value. Conventional delta values result in normalization of a fractional abundance of an isotope of an isotopic element in the sample, to the fractional abundance of the same isotope of the same isotopic element in a certified reference material. Certified reference materials are characterized by specific total concentrations of isotopic elements. For example, in some embodiments the certified reference material is IAEA-085 (certified for total concentrations of Hg, Fe, and Zn) or ERM-DB001 (certified for total concentrations of As, Cd, Cu, Hg, Pb, Se and Zn). The conventional delta value with element X is calculated using the formula, where ‘R’ is the absolute isotopic ratio for the certified standards in the certified reference material for the isotopes of interest:

$${δ}^{n} {X}_{Standard} = \frac{{}^{n} {R}_{Samp1e}}{{}^{n} {R}_{Standard}} - 1.$$

In some embodiments, the delta value is a study-specific delta value (InH). Study-specific delta values result in normalization of a fractional abundance of an isotope of an isotopic element in the sample, to the fractional abundance of the same isotope of the same isotopic element in a study-specific reference material. In some embodiments, the study-specific reference material is developed from homogenized hair obtained from control subjects. In some embodiments, the study-specific reference material is InH. The study-specific reference material may be compared to a certified reference material to ensure durability. In some embodiments, the study-specific delta value is calculated using the formula:

$${δ}^{n} {X}_{InH} = [ \frac{{{(}^{n} F / ( 1 - {}^{n} F ) )}_{Sample}}{{{(}^{n} F / ( 1 - {}^{n} F ) )}_{InH}} - 1 ] .$$

In some embodiments, the delta value is determined from the isotopic balance of isotope of N in the sample. In some embodiments, the delta value is a non-similar simple-nitrogen based delta value (i.e., SimpleNδ). For a non-similar simple-nitrogen based delta value, the ratio in the denominator in the sample is ¹⁵N, which is normalized to ¹⁴N in the sample. The SimpleNδ delta value is calculated using the formula:

$$SimpleN {δ}^{n} X = [ \frac{( {F}^{n} X / ( 1 - {F}^{n} X ) )}{( {F}^{15} N / ( 1 - {F}^{15} N ) )} - 1 ] / 1000$$

In some embodiments, the delta value is a non-similar pseudo-nitrogen based delta value (i.e., PseudoNδ). For a non-similar pseudo-nitrogen based delta value, the ratio in the denominator is in the sample ¹⁵N, which is normalized to (¹⁴N+¹⁵N) in the sample. The PseudoNδ delta value is calculated using the formula:

$$PseudoN {δ}^{n} X = [ ( \frac{{F}^{n} X}{{F}^{15} N} - 1 ) / 1000 ]$$

Thus, a fractional abundance (ⁿX) in the sample may be normalized to N in the sample in two ways (e.g., SimpleNδⁿX and PseudoNδⁿX).

In some embodiments, the at least one isotopic feature includes an overall concentration of an isotopic element. Overall concentrations of non-isotopic elements can also be used in the provided methods and systems.

In some embodiments, the at least one isotopic feature includes a concentration ratio of an isotopic element. Concentration ratios involving non-isotopic elements, including concentration ratios involving only non-isotopic elements, can also be used in the provided methods and systems.

Any number of isotopic elements and isotopic features thereof (as well as optional features from non-isotopic elements) can be used to build the prediction model. As shown by Working Examples herein, an advantage of the machine learning methods is that a priori knowledge of any one particular isotopic signature or profile of a cancer is not necessary. Results have surprisingly found high levels of accuracy in predicting presence of a cancer with an isotopic feature of a single isotope or a few isotopes, and further that the prediction power, such as by improved accuracy, can increase with any of a number of different combinations of isotopes.

In some embodiments, the at least one isotopic feature is one isotopic feature. In some embodiments, the isotope of the at least one isotopic feature may include ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr. In some embodiments, the isotope of the at least one isotopic feature is or includes ⁶⁴Zn. In some embodiments, the isotope of the at least one isotopic feature is or includes ⁶⁶Zn. In some embodiments, the isotope of the at least one isotopic feature is or includes ⁶⁸Zn. In some embodiments, the isotope of the at least one isotopic feature is or includes ⁸⁶Sr. In some embodiments, the isotope of the at least one isotopic feature is or includes ⁸⁷Sr. In some embodiments, the isotope of the at least one isotopic feature is or includes ⁸⁸Sr. In some embodiments, the isotope of the at least one isotopic feature is or includes ⁴⁶Ti. In some embodiments, the isotope of the at least one isotopic feature is or includes ⁴⁷Ti. In some embodiments, the isotope of the at least one isotopic feature is or includes ⁴⁸Ti. In some embodiments, the isotope of the at least one isotopic feature is or includes ⁵⁰Cr.

Although, like the geosciences, it is possible to develop isotopic analysis methods that are based on one or only a few elements, the predictive capacity for the methods described herein can in some instances be increased if isotopic features of a plurality of isotopic elements are included. In some embodiments, the at least one isotopic element is a plurality of isotopic elements that includes between about 2 and about 100 isotopic elements. In some embodiments, the at least one isotopic element is a plurality of isotopic elements that includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more isotopic elements. In some embodiments, the plurality of isotopic elements includes at least 2 isotopic elements. In some embodiments, the plurality of isotopic elements includes at least 3 isotopic elements. In some embodiments, the plurality of isotopic elements includes at least 4 isotopic elements. In some embodiments, the plurality of isotopic elements includes at least 5 isotopic elements. In some embodiments, the plurality of isotopic elements includes at least 6 isotopic elements. In some embodiments, the plurality of isotopic elements includes at least 7 isotopic elements. In some embodiments, the plurality of isotopic elements includes at least 8 isotopic elements. In some embodiments, the plurality of isotopic elements includes at least 9 isotopic elements. In some embodiments, the plurality of isotopic elements includes at least 10 isotopic elements. In some embodiments, the plurality of isotopic elements includes at least 15 isotopic elements. In some embodiments, the plurality of isotopic elements includes at least 20 isotopic elements. In some embodiments, the plurality of isotopic elements includes at least 25 isotopic elements. In some embodiments, the plurality of isotopic elements includes at least 30 isotopic elements. In some embodiments, the plurality of isotopic elements includes at least 35 isotopic elements. In some embodiments, the plurality of isotopic elements includes at least 40 isotopic elements. In some embodiments, the plurality of isotopic elements includes at least 45 isotopic elements. In some embodiments, the plurality of isotopic elements includes at least 50 isotopic elements.

In some embodiments, the at least one isotopic element includes a major element, such as 1, 2, 3, or 4 elements selected from C, N, O, and S. In some embodiments, the at least one isotopic element includes a major element selected from C, N, O, and S. In some embodiments, the at least one isotopic element includes C. Exemplary stable isotopes of carbon include ¹²C and ¹³C. In some embodiments, the at least one isotopic element includes N. Exemplary stable isotopes of N include ¹⁴N and ¹⁵N. In some embodiments, the at least one isotopic element includes O. Exemplary stable isotopes of O include ¹⁶O, ¹⁷O and ¹⁸O. In some embodiments, the at least one isotopic element includes S. Exemplary stable isotopes of S include ³²S, ³³S, ³⁴S, and ³⁶S. In some embodiments, the at least one isotopic element includes the major elements C, N, O, and S.

In some embodiments, predictive capacity can be increased if isotopic features of minor isotopic elements are included. In some embodiments, the at least one isotopic element includes one or more minor isotopic elements. In some embodiments, the minor isotopic elements are selected from Li, B, Na, Mg, Al, P, K, Sc, Ca, Ti, V, Cr, Mn, Fe, Co, Ni, Cu, Zn, Ge, As, Se, Br, Rb, Sr, Y, Zr, Nb, Mo, Ru, Ag, Cd, In, Sn, I, Te, Ba, Hg, Pb, and U. In some embodiments, the minor isotopic elements are selected from Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, and Zn. Exemplary stable isotopes of Zn include Zn, ⁶⁴Zn, ⁶⁶Zn, ⁶⁷Zn, ⁶⁸Zn, and ⁷⁰Zn. Exemplary stable isotopes of Cu include ⁶³Cu and ⁶⁵Cu. In some embodiments, the at least one isotopic element includes between about 2 and about 100 isotopic elements. In some embodiments, the at least one isotopic element includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more minor isotopic elements. In some embodiments, the at least one isotopic element includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or more minor isotopic elements. In some embodiments, the plurality of isotopic elements includes at least 2 minor isotopic elements. In some embodiments, the plurality of isotopic elements includes at least 3 minor isotopic elements. In some embodiments, the plurality of isotopic elements includes at least 4 minor isotopic elements. In some embodiments, the plurality of isotopic elements includes at least 5 minor isotopic elements. In some embodiments, the plurality of isotopic elements includes at least 6 minor isotopic elements. In some embodiments, the plurality of isotopic elements includes at least 7 minor isotopic elements. In some embodiments, the plurality of isotopic elements includes at least 8 minor isotopic elements. In some embodiments, the plurality of isotopic elements includes at least 9 minor isotopic elements. In some embodiments, the plurality of isotopic elements includes at least 10 minor isotopic elements. In some embodiments, the plurality of isotopic elements includes at least 15 minor isotopic elements. In some embodiments, the plurality of isotopic elements includes at least 20 minor isotopic elements. In some embodiments, the plurality of isotopic elements includes at least 25 minor isotopic elements. In some embodiments, the plurality of isotopic elements includes at least 30 minor isotopic elements. In some embodiments, the plurality of isotopic elements includes at least 35 minor isotopic elements. In some embodiments, the plurality of isotopic elements includes at least 40 minor isotopic elements. In some embodiments, the plurality of isotopic elements includes at least 45 minor isotopic elements. In some embodiments, the plurality of isotopic elements includes at least 50 minor isotopic elements.

In some embodiments, predictive capacity is increased if isotopic features of major and minor elements are included. In some embodiments, the at least one isotopic element includes major and minor elements.

In some aspects, an isotopic fingerprint for detecting, diagnosing, or assessing cancer in a subject is more robust than the natural variations that are induced by differences in, e.g., food preferences, water source, gender, or age. For example, eating habits can affect carbon and nitrogen isotopes (δ¹³C and δ¹⁵N), the menstrual cycle can affect iron (Fe) isotopes, and geography can affect δ²H and δ¹⁸O [8-10]. Complexity can further be increased due to the sampling bias [11, 12]. In some embodiments, the isotopic features include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more minor isotopic chemical elements, in addition to the major elements C, N, S, and O, wherein the minor isotopic elements are selected from Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, and Zn. In some embodiments, the minor isotopic elements are selected from Li, B, Mg, Ca, Ti, Cr, Fe, Ni, Cu, Zn, Se, Br, Rb, Sr, Mo, Ag, Sn, Ba, Hg, Pb, and U. In some embodiments, the isotopic features include any isotopic elements Li, B, Mg, Ca, Ti, Cr, Fe, Ni, Cu, Zn, Se, Br, Rb, Sr, Mo, Ag, Sn, Ba, Hg, Pb, U, C, N, S, or a combination of any of the foregoing.

In some embodiments, the at least one isotopic feature is a plurality of features comprising between about 2 and about 100 isotopic features. In some embodiments, the at least one isotopic feature is a plurality of features comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more isotopic features. In some embodiments, the plurality of isotopic features includes at least 2 isotopic features. In some embodiments, the plurality of isotopic features includes at least 3 isotopic features. In some embodiments, the plurality of isotopic features includes at least 4 isotopic features. In some embodiments, the plurality of isotopic features includes at least 5 isotopic features. In some embodiments, the plurality of isotopic features includes at least 6 isotopic features. In some embodiments, the plurality of isotopic features includes at least 7 isotopic features. In some embodiments, the plurality of isotopic features includes at least 8 isotopic features. In some embodiments, the plurality of isotopic features includes at least 9 isotopic features. In some embodiments, the plurality of isotopic features includes at least 10 isotopic features. In some embodiments, the plurality of isotopic features includes at least 15 isotopic features. In some embodiments, the plurality of isotopic features includes at least 20 isotopic features. In some embodiments, the plurality of isotopic features includes at least 25 isotopic features. In some embodiments, the plurality of isotopic features includes at least 30 isotopic features. In some embodiments, the plurality of isotopic features includes at least 35 isotopic features. In some embodiments, the plurality of isotopic features includes at least 40 isotopic features. In some embodiments, the plurality of isotopic features includes at least 45 isotopic features. In some embodiments, the plurality of isotopic features includes at least 50 isotopic features.

In some embodiments, the plurality of isotopic features are or are determined from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more isotopic elements or isotopes thereof. In some embodiments, the plurality of isotopic features are determined from at least 2 isotopic elements. In some embodiments, the plurality of isotopic features are determined from at least 3 isotopic elements. In some embodiments, the plurality of isotopic features are determined from at least 4 isotopic elements. In some embodiments, the plurality of isotopic features are determined from at least 5 isotopic elements. In some embodiments, the plurality of isotopic features are determined from at least 6 isotopic elements. In some embodiments, the plurality of isotopic features are determined from at least 7 isotopic elements. In some embodiments, the plurality of isotopic features are determined from at least 8 isotopic elements. In some embodiments, the plurality of isotopic features are determined from at least 9 isotopic elements. In some embodiments, the plurality of isotopic features are determined from at least 10 isotopic elements. In some embodiments, the plurality of isotopic features are determined from at least 15 isotopic elements. In some embodiments, the plurality of isotopic features are determined from at least 20 isotopic elements. In some embodiments, the plurality of isotopic features are determined from at least 25 isotopic elements. In some embodiments, the plurality of isotopic features are determined from at least 30 isotopic elements. In some embodiments, the plurality of isotopic features are determined from at least 35 isotopic elements. In some embodiments, the plurality of isotopic features are determined from at least 40 isotopic elements. In some embodiments, the plurality of isotopic features are determined from at least 45 isotopic elements. In some embodiments, the plurality of isotopic features are determined from at least 50 isotopic elements.

In some embodiments, the plurality of isotopic features includes any combination of atomic percents, isotope ratios, fractional abundances, deltas, overall concentrations, and concentration ratios. In some embodiments, the plurality of isotopic features include fractional abundances and deltas (e.g., conventional delta for major isotopes). In some embodiments, the plurality of isotopic features include fractional abundances, deltas, and concentration ratios. In some embodiments, each atomic percent of an isotope of an isotopic element is relative to stable isotopes of the isotopic element of the isotope. In some embodiments, each isotope ratio of an isotope of an isotopic element is relative to stable isotopes of the isotopic element of the isotope.

In some embodiments, the isotopic features, e.g., atomic percents, isotope ratios, fractional abundances, deltas, overall concentrations, and/or concentration ratios, are determined from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more isotopic elements.

In some embodiments, the at least one isotopic feature includes a concentration ratio. In some embodiments, the concentration ratio is at least 1, 2, 3, 4, 5, or 6 of N/C, S/C, O/C, S/N, N/O, and S/O. In some embodiments, the at least one isotopic feature includes N/C. In some embodiments, the at least one isotopic feature includes S/C. In some embodiments, the at least one isotopic feature includes O/C. In some embodiments, the at least one isotopic feature includes S/N. In some embodiments, the at least one isotopic feature includes N/O. In some embodiments, the at least one isotopic feature includes S/O. In some embodiments, the at least one isotopic feature includes N/C, S/C, O/C, S/N, N/O, and S/O.

In some embodiments, the at least one isotopic feature includes a concentration ratio involving Fe. In some embodiments, the concentration ratio is Fe/C or Fe/S. In some embodiments, the at least one isotopic feature includes Fe/C and Fe/S.

In some embodiments, the at least one isotopic feature includes an overall concentration. In some embodiments, the at least one isotopic feature includes an overall concentration of Fe.

In some embodiments, the at least one isotopic feature includes at least 1, 2, 3, 4, 5, 6, 7, 8, 9, 10, 15, 20, 25, 30, 35, 40, 45, 50, 55, 60, 65, 70, 75, 80, 85, or 90 of the fractional abundance, atomic percent, or isotope ratio of ¹⁰B, ¹¹B, ⁴⁶Ti, ⁴⁷Ti, ⁴⁸Ti, ⁴⁹Ti, ⁵⁰Ti, ⁵⁰Cr, ⁵²Cr, ⁵³Cr, ⁵⁴Cr, ⁵⁴Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁰Ge, ⁷²Ge, ⁷⁴Ge, ⁷⁴Se, ⁷⁶Se, ⁷⁷Se, ⁷⁸Se, ⁸⁰Se, ⁸²Se, ⁷⁹Br, ⁸¹Br, ⁸⁵Rb, ⁸⁷Rb, ⁸⁶Sr, ⁸⁷Sr, ⁸⁸Sr, ⁹²Mo, ⁹⁴Mo, ⁹⁵Mo, ⁹⁶Mo, ⁹⁷Mo, ⁹⁸Mo, ¹⁰⁰Mo, ¹⁰⁷Ag, ¹⁰⁹Ag, ¹¹⁶Sn, ¹¹⁷Sn, ¹¹Sn, ¹¹⁹Sn, ¹²⁰Sn, ¹²⁴Sn, ¹²⁴Te, ¹²⁵Te, ¹²⁶Te, ¹²⁸Te, ¹³Te, ¹³⁵Ba, ¹³⁶Ba, ¹³⁷Ba, ¹⁹⁸Hg, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, ²³⁸U, ⁶Li, ⁷Li, ²⁴Mg, ²⁵Mg, ²⁶Mg, ²³Na, ²⁷Al, ³⁹K, ¹⁰¹Ru, ⁴⁴Ca, ⁵¹V, ⁵⁵Mn, ⁵⁹Co, ⁸⁹Y, ¹²⁷I, ¹⁵N, ¹³C, ³⁴S, or ¹⁶O. In some embodiments, the at least one isotopic feature includes at least 1, 2, 3, 4, 5, 6, 7, 8, 9, 10, 15, 20, 25, 30, 35, 40, 45, 50, 55, 60, 65, 70, 75, 80, 85, or 90 of the fractional abundance, atomic percent, or isotope ratio of ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁶Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U. In some embodiments, the atomic percent or isotope ratio of each isotope is relative to stable isotopes of the isotopic element of the isotope (e.g., relative to stable isotopes of C for the atomic percent or isotopic ratio of ¹³C).

In some embodiments, the at least one isotopic feature includes at least 1, 2, 3, 4, 5, 6, 7, 8, 9, or 10 of the fractional abundance, atomic percent, or isotope ratio of ¹³C (F13C), the fractional abundance, atomic percent, or isotope ratio of ¹⁸O (F18O), the concentration ratio of sulfur over nitrogen (SdN), the concentration ratio of nitrogen over carbon (NdC), the fractional abundance, atomic percent, or isotope ratio of ³⁴S (F³⁴S), the fractional abundance, atomic percent, or isotope ratio of ¹⁵N (F15N), the concentration ratio of oxygen over carbon (OdC), the concentration ratio of nitrogen over oxygen (NdO), the concentration ratio of sulfur over carbon (SdC), and the concentration ratio of sulfur over oxygen (SdO). In some embodiments, the at least one isotopic feature includes F13C, F18O, SdN, NdC, F³⁴S, F15N, OdC, NdO, SdC, and SdO. In some embodiments, the atomic percent or isotope ratio of each isotope is relative to stable isotopes of the isotopic element of the isotope (e.g., relative to stable isotopes of C for the atomic percent or isotopic ratio of ¹³C).

In some embodiments, the at least one isotopic feature includes at least 1, 2, 3, 4, 5, 6, 7, 8, 9, 10, 11, 12, 13, 14, 15, 16, 17, 18, 19, or 20 or more of the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷I, ¹²⁸Te, ¹³⁶Ba, ¹³⁷Ba, ¹³C (also denoted as ¹³C_atom), ²³⁵U, ²³⁸U, ²³Na, ²⁷Al, ³⁴S (also denoted as ³⁴S_atom), ³⁹K, ⁴⁴Ca, ⁴⁸Ti, ⁵⁴Cr, ⁵⁴Fe, ⁵⁷Fe, ⁶²Ni, ⁶³Cu, ⁶⁶Zn, ⁶⁷Zn, ⁶⁸Zn, ⁷⁰Ge, ⁷⁴Ge, ⁷⁴Se, ⁷⁶Se, ⁷⁷Se, ⁷⁹Br, ⁸⁰Se, ⁸²Se, ⁸⁹Y, ⁹⁴Mo, ⁹⁷Mo, and ⁹⁸Mo. In some embodiments, the atomic percent or isotope ratio of the isotope is relative to stable isotopes of the isotopic element of the isotope (e.g., relative to stable isotopes of C for the atomic percent or isotopic ratio of ¹³C).

In some embodiments, the at least one isotopic feature includes at least 1, 2, 3, 4, 5, 6, 7, 8, 9, 10, or more of the fractional abundance, atomic percent, or isotope ratio of ¹⁰⁷Ag, ¹⁰⁹Ag, ¹¹⁸Sn, ¹²⁷I, ¹³C (also denoted as ¹³C_atom), ²³⁵U, ²⁷Al, ³⁴S (also denoted as ³⁴S_atom), ⁴⁴Ca, ⁵⁷Fe, ⁶⁷Zn, ⁷⁴Se, ⁹⁴Mo, and ⁹⁷Mo. In some embodiments, the atomic percent or isotope ratio of each isotope is relative to stable isotopes of the isotopic element of the isotope (e.g., relative to stable isotopes of C for the atomic percent or isotopic ratio of ¹³C).

In some embodiments, the at least one isotopic feature includes the fractional abundance, atomic percent, or isotope ratio of ¹⁰⁷Ag. In some embodiments, the at least one isotopic feature includes the fractional abundance, atomic percent, or isotope ratio of ¹⁰⁹Ag. In some embodiments, the at least one isotopic feature includes the fractional abundance, atomic percent, or isotope ratio of ¹¹⁸Sn. In some embodiments, the at least one isotopic feature includes the fractional abundance, atomic percent, or isotope ratio of ¹²⁷I. In some embodiments, the at least one isotopic feature includes the fractional abundance, atomic percent, or isotope ratio of ¹³C (also denoted as ¹³C_atom). In some embodiments, the at least one isotopic feature includes the fractional abundance, atomic percent, or isotope ratio of ²³¹U. In some embodiments, the at least one isotopic feature includes the fractional abundance, atomic percent, or isotope ratio of ²⁷Al. In some embodiments, the at least one isotopic feature includes the fractional abundance, atomic percent, or isotope ratio of ³⁴S (also denoted as ³⁴S_atom). In some embodiments, the at least one isotopic feature includes the fractional abundance, atomic percent, or isotope ratio of ⁴⁴Ca. In some embodiments, the at least one isotopic feature includes the fractional abundance, atomic percent, or isotope ratio of ⁵⁷Fe. In some embodiments, the at least one isotopic feature includes the fractional abundance, atomic percent, or isotope ratio of ⁶⁴Zn. In some embodiments, the at least one isotopic feature includes the fractional abundance, atomic percent, or isotope ratio of ⁶⁶Zn. In some embodiments, the at least one isotopic feature includes the fractional abundance, atomic percent, or isotope ratio of ⁶⁷Zn. In some embodiments, the at least one isotopic feature includes the fractional abundance, atomic percent, or isotope ratio of ³⁴S. In some embodiments, the at least one isotopic feature includes the fractional abundance, atomic percent, or isotope ratio of ⁷⁴Se. In some embodiments, the at least one isotopic feature includes the fractional abundance, atomic percent, or isotope ratio of ⁹⁴Mo. In some embodiments, the at least one isotopic feature includes the fractional abundance, atomic percent, or isotope ratio of ⁹⁷Mo. In some embodiments, the at least one isotopic feature includes the fractional abundance, atomic percent, or isotope ratio of ¹²⁷I and the fractional abundance, atomic percent, or isotope ratio of ⁴⁴Ca. In some embodiments, the atomic percent or isotope ratio of each isotope is relative to stable isotopes of the isotopic element of the isotope (e.g., relative to stable isotopes of C for the atomic percent or isotopic ratio of ¹³C).

In some embodiments, the sample is obtained, and processed to obtain isotopic features, from a subject or representative numbers of subjects for purposes of building or training the prediction model. In some embodiments, a representative number of subjects is at least 20 subjects, or at least 50 subjects, or at least 100 subjects, or at least 1,000 subjects, or at least 5,000 subjects, or at least 10,000 subjects, or at least 50,000, or at least 100,000 subjects. In some embodiments, the subjects span multiple representative populations, cultures, continents, and/or socio-economical settings. In some embodiments, the subjects are from a single population, culture, continent, and/or socio-economical setting.

In some embodiments, the prediction model, e.g., machine learning model, is trained using isotopic features from a plurality of reference subjects. In embodiments, the prediction model is built using input data from a plurality of cancer subjects and a plurality of healthy (also referred to as normal or control subjects). In some embodiments, the plurality of subjects (e.g., cancer subjects or healthy subjects) used for training the prediction model generally include greater than 20 subjects, such as greater than 50, 60, 70, 80, 90, 100, 200, 300, 400, 500 or more subjects. In some embodiments, the plurality of subjects spans multiple populations, cultures, continents, and/or socio-economical settings. In some embodiments, the plurality of subjects is from a single population, culture, continent, and/or socio-economical setting.

For building the prediction model, a cancer subject is a subject that has been diagnosed with cancer using a conventional invasive method for cancer detection, such as by using biopsy. In some embodiments, a healthy or normal subject is a control subject that does not suffer from or has not been diagnosed or suspected of having a cancer, such as assessed by methods known in the art. In aspects of the method, the prediction model is established for a specific type of cancer, such that all cancer subjects of the plurality used for training the model have been diagnosed with the specific cancer, whereas all healthy or normal subjects have not been diagnosed or known or suspected of having any cancer. In some aspects, the prediction model is established for detecting, diagnosing, or assessing a plurality of specific types of cancer. Once built or trained, a sample can be obtained, and processed to obtain isotopic features, from any subject for which it desired to detect or diagnose the presence or absence of cancer or to assess the cancer in the subject, such as described in Section III.

In some embodiments, the prediction model can be established for cancers such as a hematological cancer, a lymphoma, a myeloma, a leukemia, a neurological cancer, skin cancer, sarcoma, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, or a vascular cancer.

In some embodiments of the methods and uses described herein, the cancer is lung cancer, thyroid cancer, ovarian cancer, colorectal cancer, prostate cancer, cancer of the pancreas, cancer of the esophagus, liver cancer, breast cancer, skin cancer, or mesothelioma. In some embodiments, the cancer is mesothelioma, such as malignant mesothelioma. In some embodiments, cancer includes, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; tonsil cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the solid tumor is defined as a nascent entity of “multicellularity” growing within the body of the animal or “host,” where its chemical characteristics depend on, but are not limited to, growth rate and the functions of the tissue.

In particular embodiments, the prediction model is established for prostate, kidney, renal, head and neck, skin, bladder, colon, Hodgkin's lymphoma, rectal, testicular, thyroid, tonsil, and/or hematological cancers. In some embodiments, the cancer is prostate cancer. In some embodiments, the cancer is a kidney cancer. In some embodiments, the cancer is a renal cancer. In some embodiments, the cancer is a head and neck cancer.

### B. Prediction Model Development Process

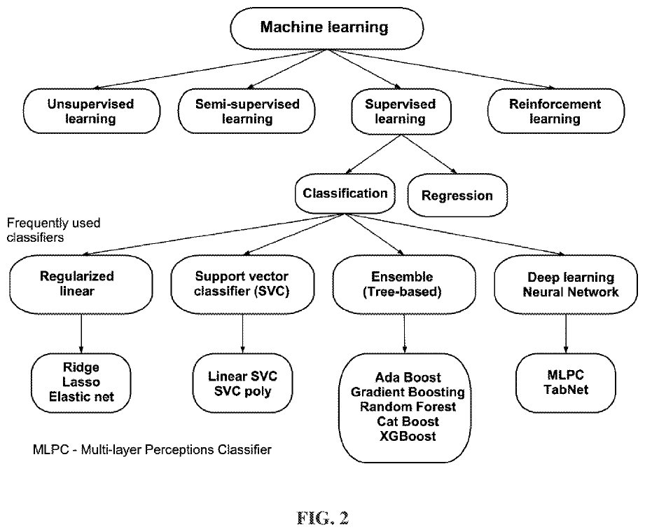

The provided methods are in some aspects based on a prediction model built (e.g., trained) using a machine learning algorithm. Briefly, machine learning algorithms are a subset of artificial intelligence algorithms that can be used for supervised and unsupervised learning (FIG. 2). In some embodiments, the machine learning algorithm of the prediction model is a supervised machine learning algorithm. In some embodiments, the machine learning algorithm of the prediction model is an unsupervised machine learning algorithm.

Regularized linear algorithms (e.g., ridge, least absolute shrinkage and selection operator (lasso), and elastic net) are among the machine learning algorithms that can be used for regression problems, and they can also be applied for classification problems. Non-linear algorithms such as decision tree-based algorithms can also be used. Exemplary decision tree related algorithms include Random Forest Classifier, GradientBoosting Classifier, XGBoost Classifer, HistGradientBoosting Classifier, Bagging Classifier, ExtraTrees Classifier, AdaBoost Classifier, CatBoost Classifier, and Light GBM Classifier, as well as corresponding algorithms for regression models. Decision tree-related algorithms can use ensemble methods, which can in some instances perform better than a base Decision Tree Classifier or Regressor. Kernel-based algorithms, such as support vector classifiers (SVC) or regressors, can use various functions such as linear, sigmoid, polynomial, etc., functions, and can be useful for classification or regression. A branch of machine learning algorithm, deep learning, has also been used for classification or regression and can also be useful. Multi-layer perceptrons (MLP), e.g., Multi-layer Perceptron classifiers or regressors, are a type of feed-forward neural network that can be used. Recently, several deep learning algorithms that can be used to handle tabular data have been developed, such as TabNet and TabML.

In some embodiments, the prediction model is a classification model. In some embodiments, the classification model is configured to predict the presence or absence of a cancer, which can be a type of cancer that the prediction model is configured to predict, e.g., predicts the presence or absence of prostate cancer. In some embodiments, the classification model is configured to diagnose a cancer. In some embodiments, the classification model is configured to predict a characteristic of the subject or the cancer in the subject. In some embodiments, the classification model is configured to predict a characteristic of the cancer in the subject. In some embodiments, the classification model is configured to predict the type of cancer in the subject. In some embodiments, the classification model is configured to predict if the cancer is local or has metastasized.

In some embodiments, the prediction model is a binary classification model (e.g., predicts the presence or absence of cancer, which can be a type of cancer that the prediction model is configured to predict, e.g., predicts the presence or absence of prostate cancer). In some embodiments, the prediction model is a multiclass classification model (e.g., predicts the presence or absence of cancer, for instance including the type of cancer among a plurality of cancers that the prediction model is configured to predict, e.g., predicts the presence of prostate cancer, the presence of head and neck cancer, or the absence of prostate cancer and head and neck cancer). In some embodiments, the machine learning algorithm is a classification algorithm, which can be a binary or multiclass classification algorithm.

In some embodiments, the prediction model is a regression model. In some embodiments, the regression model is configured to predict a characteristic of a subject or a cancer in the subject. In some embodiments, the regression model is configured to predict a characteristic of a cancer in the subject. In some embodiments, the regression model is configured to predict the level of tumor burden of the cancer in the subject. In some embodiments, the regression model is configured to predict the risk of cancer development in the subject. In some embodiments, the regression model is configured to predict the risk of tumor growth of the cancer in the subject. In some embodiments, the machine learning algorithm is a regression algorithm.

In some embodiments, the machine learning algorithm is a regularized linear classifier. In some embodiments, the machine learning algorithm is a ridge classifier. In some embodiments, the machine learning algorithm is a lasso classifier. In some embodiments, the machine learning algorithm is an elastic net classifier.

In some embodiments, the machine learning algorithm is a regularized linear regressor. In some embodiments, the machine learning algorithm is a ridge regressor. In some embodiments, the machine learning algorithm is a lasso regressor. In some embodiments, the machine learning algorithm is an elastic net regressor.

In some embodiments, the machine learning algorithm is a support vector classifier (SVC). In some embodiments, the machine learning algorithm is a linear SVC. In some embodiments, the machine learning algorithm is a kernel function SVC. In some embodiments, the kernel function is a polynomial kernel (e.g., SVC poly). In some embodiments, the kernel function is a Gaussian kernel. In some embodiments, the kernel function is a Gaussian Kernel Radial Basis Function kernel. In some embodiments, the kernel function is a sigmoid kernel.

In some embodiments, the machine learning algorithm is a support vector regressor. In some embodiments, the machine learning algorithm is a linear support vector regressor. In some embodiments, the machine learning algorithm is a kernel function support vector regressor. In some embodiments, the kernel function is a polynomial kernel. In some embodiments, the kernel function is a Gaussian kernel. In some embodiments, the kernel function is a Gaussian Kernel Radial Basis Function kernel. In some embodiments, the kernel function is a sigmoid kernel.

In some embodiments, the machine learning algorithm is a decision tree-based ensemble classifier. In some embodiments, the machine learning algorithm is an XGBoost classifier. In some embodiments, the machine learning algorithm is a Cat Boost classifier. In some embodiments, the machine learning algorithm is a Random Forest classifier.

In some embodiments, the machine learning algorithm is a decision tree-based ensemble regressor. In some embodiments, the machine learning algorithm is an XGBoost regressor. In some embodiments, the machine learning algorithm is a Cat Boost regressor. In some embodiments, the machine learning algorithm is a Random Forest regressor.

In some embodiments, the machine learning algorithm is a deep learning algorithm. In some embodiments, the machine learning algorithm is an MLPC. In some embodiments, the machine learning algorithm is a TabNet classifier.

In some embodiments, the machine learning algorithm is an MLP regressor. In some embodiments, the machine learning algorithm is a TabNet regressor.

In some embodiments, multiple machine learning algorithms can be assessed, and model parameters optimized, using the selected features to assess the best prediction model. In some embodiments, the prediction model involves multiple machine learning algorithms, for instance any combination of the machine learning algorithms described herein. In some embodiments, the machine learning algorithm is a decision tree-based ensemble classifier. In some embodiments, the decision tree-based ensemble classifier is a random forest classifier. In some embodiments, the machine learning algorithm is a decision tree-based ensemble regressor. In some embodiments, the decision tree-based ensemble regressor is a random forest regressor.

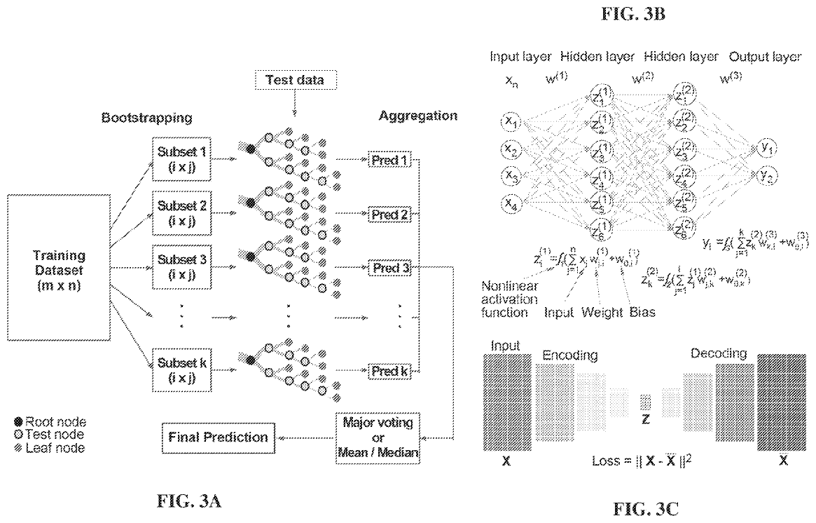

Random forest is a popular machine learning algorithm built on decision trees. Generally, a decision tree keeps growing data in the root node, and according to a test rule (representing the branch), until it reaches a decision (representing a leaf node or output). The internal nodes can represent different features [13]. Each internal node can break the data into a small subset until it meets a particular condition. It can be a white-box-type algorithm, as each step can be understood, interpreted, and visualized. The random forest algorithm can randomly split (bootstrapping) training data into several subsets and use each subset to build a decision tree (FIG. 3A). The use of multiple random decision trees for prediction can increase the prediction accuracy [14]. The random forest algorithm has several usages in data science. This algorithm can be used for feature selection, by which informative features can be identified and used to build machine learning models [15].

In some embodiments, the machine learning algorithm is a deep learning algorithm. Deep learning algorithm models can automatically discover appropriate representations for regression or classification problems upon being trained with suitable data. During training, the algorithm can take the raw input and process it through hidden layers using nonlinear activation functions. The algorithm can minimize certain loss functions by defining values for the weights and biases (FIG. 3B). In some embodiments, gradient descent is used to find the minima. Gradients for all modules can be determined by using the chain rule for derivatives, a procedure that is known as backpropagation (starting from the output and moving toward the input) [16].

Deep learning algorithms can also be used for feature engineering. The unsupervised deep learning method referred to as Autoencoder can be used to reduce the dimension of features. An autoencoder can learn hidden (latent) variables from the observed data through the mapping of higher-dimensional data onto a lower-dimensional latent space. An autoencoder can comprise encoding layers and decoding layers. The encoding layers can project higher-dimensional input data onto a lower-dimensional space and decoding layers can reconstruct the lower-dimensional latent space back to the higher-dimensional data similar to input (FIG. 3C). In some embodiments, an autoencoder can use nonlinear activation functions for data compression and can discover nonlinear explanatory features. In some aspects, it can be used to reduce features from higher-dimensional biological data to uncover a biologically relevant latent space [17, 18]. The autoencoder has previously been incorporated into the model-X knockoffs for feature selection. Recently, Candes et al. [19]proposed a new framework of model-X knockoffs for achieving the false discovery rate (FDR) control in feature selection, bypassing the use of conventional p values. The silent idea of the model-X knockoffs is to construct the so-called “model-X knockoff variables,” which can perfectly mimic the dependence structure of original variables but are conditionally independent of the response. The autoencoder [20] implementation of model-X knockoffs is publicly available and can be used for feature selection.

In some embodiments, the methods described herein use Shapley Additive exPlanations (SHAP), which is a game theoretic approach to analyze and explain the decisions of a machine learning model. However, it will be appreciated that other techniques are contemplated to understand and interpret predictions made by machine learning models. For example, alternatively or additionally, gradient based approaches such as integrated gradients, back propagation approaches such as DeepLIFT, model agnostic techniques such as Local Interpretable Model-agnostic Explanations (LIME), neural network and attention weight approaches such as Attention-Based Neural Network Models, or Deep Taylor Decomposition approaches such as Layer-wise Relevance Propagation (LRP), may be used to understand and interpret predictions made by machine learning models. The core idea behind SHAP-based explanations of machine learning models is to use fair allocation results from cooperative game theory to allocate credit for a model's output among its input features. In other words, the SHAP explanation method can break down a prediction to show the impact of each feature (e.g., isotopic fractional abundance of a specific isotope). In order to do this, the SHAP explanation method computes Shapley values from cooperative game theory. Features contribute to the model's output or prediction with different magnitude and sign, which can be accounted for by the Shapley values. Accordingly, Shapley values can represent estimates of each feature's importance (magnitude of the contribution or influence) as well as the direction (sign). Features with positive Shapley values increase the prediction value of the detection of cancer, whereas features with negative Shapley values decrease the prediction value of the detection of cancer. The averages of absolute Shapley values may then be used to rank and sort the importance of each feature.

In some embodiments, the provided method is a method of building a prediction model for detecting whether a subject has a cancer, the method comprising: (a) determining, for each of a plurality of reference samples from a plurality of reference subjects, the value for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the reference sample; and (b) building a prediction model configured to predict the presence or absence of a cancer in a subject, wherein the prediction model comprises at least one machine learning algorithm, and the building comprises training the machine learning algorithm using the values for each of the at least one isotopic feature for the plurality of reference samples.

In some embodiments, the provided method is a method of training a prediction model for detecting whether a subject has a cancer, the method comprising: (a) determining, for a plurality of reference samples from a plurality of reference subjects, values for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3), measured in a reference sample of the plurality of reference samples; and (b) applying the values for each of the at least one isotopic feature for the plurality of reference samples as input to train a prediction model configured to predict the presence or absence of a cancer in a subject, wherein the prediction model comprises at least one machine learning algorithm

In some embodiments, the provided method is a method of building a prediction model for assessing a characteristic of a cancer in a subject, the method comprising: (a) determining, for each of a plurality of reference samples from a plurality of reference subjects, the value for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the reference sample; and (b) building a prediction model configured to predict a characteristic of a cancer in a subject, wherein the prediction model comprises at least one machine learning algorithm, and the building comprises training the machine learning algorithm using the values for each of the at least one isotopic feature for the plurality of reference samples.

In some embodiments, the provided method is a method of training a prediction model for assessing a characteristic of a cancer in a subject, the method comprising: (a) determining, for a plurality of reference samples from a plurality of reference subjects, values for each of at least one isotopic feature, (1) a concentration of an element, (2) a concentration of an isotopic element, (3) a concentration of an isotope of the isotopic element, or (4) a normalized value of any of (1)-(3), measured in a reference sample of the plurality of reference samples; and (b) applying the values for each of the at least one isotopic feature for the plurality of reference samples as input for training a prediction model configured to predict a characteristic of a cancer in a subject, wherein the prediction model comprises at least one machine learning algorithm

In some embodiments, the provided method is a method of building a prediction model for detecting whether a subject has a cancer, the method comprising: (a) receiving, at one or more processors, the value for each of at least one isotopic feature for each of a plurality of reference samples from a plurality of reference subjects, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the reference sample; and (b) building, using at least one processor, a prediction model configured to predict the presence or absence of a cancer in a subject, wherein the prediction model comprises at least one machine learning algorithm, and the building comprises training the machine learning algorithm using the values for each of the at least one isotopic feature for the plurality of reference samples.

In some embodiments, the provided method is a method of training a prediction model for detecting whether a subject has a cancer, the method comprising: (a) receiving, at one or more processors, values for each of at least one isotopic feature for a plurality of reference samples from a plurality of reference subjects, wherein the value for each of the at least one isotopic feature is: (1) a concentration of an element, (2) a concentration of an isotopic element, (3) a concentration of an isotope of the isotopic element, or (4) a normalized value of any of (1)-(3) measured in a reference sample of the plurality of reference samples; and (b) applying the values for each of the at least one isotopic feature for the plurality of reference samples as input for training, using at least one processor, a prediction model configured to predict the presence or absence of a cancer in a subject, wherein the prediction model comprises at least one machine learning algorithm.

In some embodiments, the provided method is a method of building a prediction model for assessing a characteristic of a cancer in a subject, the method comprising: (a) receiving, at one or more processors, the value for each of at least one isotopic feature for each of a plurality of reference samples from a plurality of reference subjects, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the reference sample; and (b) building, using at least one processor, a prediction model configured to predict a characteristic of a cancer in a subject, wherein the prediction model comprises at least one machine learning algorithm, and the building comprises training the machine learning algorithm using the values for each of the at least one isotopic feature for the plurality of reference samples.

In some embodiments, the provided method is method of training a prediction model for assessing a characteristic of a cancer in a subject, the method comprising: (a) receiving, at one or more processors, values for each of at least one isotopic feature for a plurality of reference samples from a plurality of reference subjects, wherein the value for each of the at least one isotopic feature is: (1) a concentration of an element, (2) a concentration of an isotopic element, (3) a concentration of an isotope of the isotopic element, or (4) a normalized value of any of (1)-(3),t measured in a reference sample of the plurality of reference samples; and (b) applying the values for each of the at least one isotopic feature for the plurality of reference samples as input for training, using at least one processor, a prediction model configured to predict a characteristic of a cancer in a subject, wherein the prediction model comprises at least one machine learning algorithm.

In some embodiments, the provided method is a method of building a prediction model for detecting whether a subject has a cancer, the method comprising: (a) providing a plurality of reference samples obtained from a plurality of reference subjects; (b) preparing liquid and/or solid samples for analysis by mass spectrometry from the plurality of reference samples, wherein the liquid samples are prepared by digestion of one or more of the reference samples; (c) providing standards comprising a plurality of calibration standards, a blank standard, an internal standard, and/or a reference material; (d) introducing the standards and the prepared liquid or solid samples individually into a mass spectrometer to detect one or more ionization products of elements present in each of the standards and the prepared liquid or solid samples; (e) analyzing the detected one or more ionization products of isotopic elements to generate the value for each of at least one isotopic feature for each of the plurality of reference samples, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element; (f) receiving, at one or more processors, the values for each of the at least one isotopic feature; and (g) building, using at least one processor, a prediction model configured to predict the presence or absence of a cancer in a subject, wherein the prediction model comprises at least one machine learning algorithm, and the building comprises training the machine learning algorithm using the values for each of the at least one isotopic feature for the plurality of reference samples.

In some embodiments, the provided method is method of training a prediction model for detecting whether a subject has a cancer, the method comprising: (a) providing a plurality of reference samples obtained from a plurality of reference subjects; (b) preparing liquid and/or solid samples for analysis by mass spectrometry from the plurality of reference samples, wherein the liquid samples are prepared by digestion of one or more of the reference samples; (c) providing standards comprising a plurality of calibration standards, a blank standard, an internal standard, and/or a reference material; (d) introducing the standards and the prepared liquid or solid samples individually into a mass spectrometer to detect one or more ionization products of elements present in each of the standards and the prepared liquid or solid samples; (e) analyzing the detected one or more ionization products of isotopic elements to generate values for each of at least one isotopic feature for the plurality of reference samples, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the values for each of the at least one isotopic feature; and (g) applying the values for each of the at least one isotopic feature for the plurality of reference samples as input for training, using at least one processor, a prediction model configured to predict the presence or absence of a cancer in a subject, wherein the prediction model comprises at least one machine learning algorithm.

In some embodiments, the provided method is a method of building a prediction model for assessing a characteristic of a cancer in a subject, the method comprising: (a) providing a plurality of reference samples obtained from a plurality of reference subjects; (b) preparing liquid and/or solid samples for analysis by mass spectrometry from the plurality of reference samples, wherein the liquid samples are prepared by digestion of one or more of the reference samples; (c) providing standards comprising a plurality of calibration standards, a blank standard, an internal standard, and/or a reference material; (d) introducing the standards and the prepared liquid or solid samples individually into a mass spectrometer to detect one or more ionization products of elements present in each of the standards and the prepared liquid or solid samples; (e) analyzing the detected one or more ionization products of isotopic elements to generate the value for each of at least one isotopic feature for each of the plurality of reference samples, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element; (f) receiving, at one or more processors, the values for each of the at least one isotopic feature; and (g) building, using at least one processor, a prediction model configured to predict a characteristic of a cancer in a subject, wherein the prediction model comprises at least one machine learning algorithm, and the building comprises training the machine learning algorithm using the values for each of the at least one isotopic feature for the plurality of reference samples.

In some embodiments, the provided method is a method of training a prediction model for assessing a characteristic of a cancer in a subject, the method comprising: (a) providing a plurality of reference samples obtained from a plurality of reference subjects; (b) preparing liquid and/or solid samples for analysis by mass spectrometry from the plurality of reference samples, wherein the liquid samples are prepared by digestion of one or more of the reference samples; (c) providing standards comprising a plurality of calibration standards, a blank standard, an internal standard, and/or a reference material; (d) introducing the standards and the prepared liquid or solid samples individually into a mass spectrometer to detect one or more ionization products of elements present in each of the standards and the prepared liquid or solid samples; (e) analyzing the detected one or more ionization products of isotopic elements to generate values for each of at least one isotopic feature for the plurality of reference samples, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the values for each of the at least one isotopic feature; and (g) applying the values for each of the at least one isotopic feature for the plurality of reference samples as input for training, using at least one processor, a prediction model configured to predict a characteristic of a cancer in a subject, wherein the prediction model comprises at least one machine learning algorithm.

In some embodiments, the prediction model is built and applied using a computer-program product tangibly embodied in a non-transitory machine-readable storage medium, including instructions configured to cause one or more data processors to build and apply the prediction model.

In some embodiments, the prediction model is trained and applied using a computer-program product tangibly embodied in a non-transitory machine-readable storage medium, including instructions configured to cause one or more data processors to train and apply the prediction model.

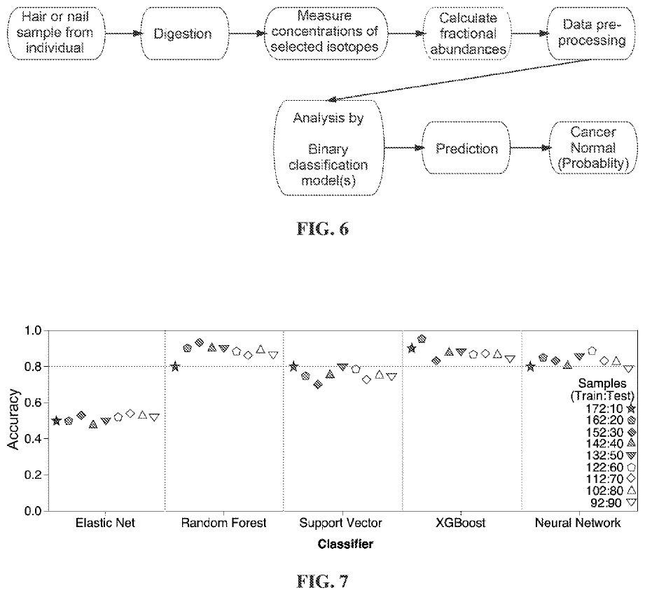

A brief explanation of an exemplary prediction model development process (FIG. 4), when using isotopic features from a cancer patient and healthy donors in a multielement machine learning algorithm approach, is as follows. The explanation encompasses how one or several machine learning algorithms can be used, when data pre-processing can be used, and how the prediction model can be built (e.g., trained), continuously improved, and used to identify whether a new sample stems from a person with or without cancer (FIG. 6).

### 1. Machine Learning Algorithms

In some embodiments, the method utilizes single or multiple machine learning algorithms to build a prediction model, and for formulating the final prediction. In some embodiments, eXtreme Gradient Boosting (XGBoost) classification algorithm is used [21]. In some embodiments, at least one of several other algorithms, such as logistic regression with regularization [22], random forest [23], CatBoost [24], support vector machines [27], deep learning for tabular data—TabNet [28], or a relevant machine learning algorithm, is used. In some embodiments, for the final prediction model, several of these can be combined to optimize predictive power.

### 2. Data for Training, Testing, and Pre-Processing

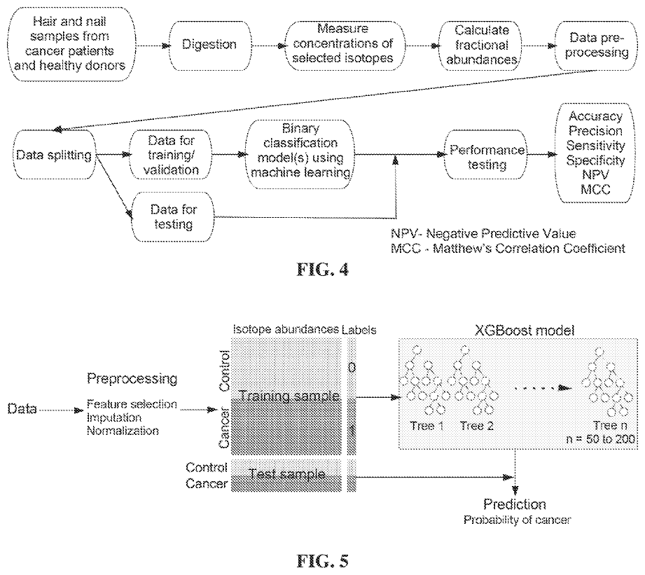

In some embodiments, samples from a representative number of subjects are collected, wherein a majority are used for training (TrainingS) the prediction model, and a smaller proportion will be used only for testing (TestingS). In some embodiments, out of the TrainingS, some are kept for validation (e.g., for deep learning algorithms or for automatic optimization of the number of trees), see FIG. 5.

In some embodiments, the method includes data preprocessing. This pre-processing can include controlled imputation of missing values, replacing missing values with zero, and/or normalization of the data (e.g., a normalized value of a concentration of an element, a normalized value of a concentration of an isotopic element, or a normalized a concentration of an isotope of the isotopic element). Normalized values may include calculating an atomic percent (atom %), fractional abundance (F), isotope ratio (R), concentration ratio (CR), or a delta value (delta), or a combination of any of the foregoing. For example, normalization of the data may calculate a conventional delta value, a study-specific delta value (InH), a non-similar simple-nitrogen based delta value (SimpleNδ), or a non-similar pseudo-nitrogen based delta value (PseudoNδ). In some embodiments, data preprocessing regardless of the algorithm will permit the use of a reduced number of isotopic features, relative to algorithms without data-preprocessing, which can eventually reduce analysis costs.

### 3. Prediction Model Training

To build (e.g., train) a prediction model using a machine learning algorithm, samples from cancer patients can be labeled as “1” and samples from healthy donors can be labeled as “0.” Other labeling schemes for distinguishing between samples from cancer patients and healthy donors can also be identified and used by the skilled artisan. In some embodiments, the isotopic features can include overall concentrations of isotopes of X=5-36 or more different chemical elements, which can result in a minimum of at least 50 isotopic features, preserved in hair samples from these patients and healthy donors (i.e., controls). Isotopic features can include atomic percents; fractional abundances; deltas; both fractional abundances and deltas; overall concentrations, e.g., of at least one of N, C, S, and O; concentration ratios, e.g., of at least one of N/C, S/C, O/C, S/N, N/O, and S/O; and any combination thereof (FIG. 7). In some embodiments, the machine learning algorithm uses at least Y isotopic features, wherein Y=10, 20, 30, 40, 50, 60, 70, 80, or more. In some embodiments, at least 10 isotopic features are used in building (e.g., training) the prediction model. In some embodiments, at least 20 features are used in building (e.g., training) the prediction model. In some embodiments, at least 30 features are used in building (e.g., training) the prediction model. In some embodiments, at least 40 features are used in building (e.g., training) the prediction model. In some embodiments, at least 50 features are used in building (e.g., training) the prediction model. In some embodiments, at least 60 features are used in building (e.g., training) the prediction model. In some embodiments, at least 70 features are used in building (e.g., training) the prediction model.

In some embodiments, once the prediction model is built (e.g., trained) using the TrainingS samples, the predictive performance can be tested using the samples set aside for testing (TestingS). In essence, the isotopic features of a sample of unknown label (TestingS) is probed via the trained machine learning algorithm.

In some embodiments, the model's predictive performance is assessed using one or multiple parameters selected from Accuracy (the ratio of correctly predicted instances to the total number of samples, which is the true negative and true positive divided with all outcomes), Area under the Curve (“AUC”) (quantifies the ability of the prediction model to distinguish between classes as the normalized aggregate of the area under the receiving operating curve (“ROC”)), Precision or Positive Predictive Value (“PPV”) (true positive divided with true positives plus false positives), Sensitivity (e.g., True Positive Rate (“TPR”) or Detection Rate (“DR”)) (true positive divided with true positives plus false negatives), Specificity (true negatives divided with true negative plus false positives), F1 score (which represents the harmonic mean of accuracy and recall; ranging from 0 to 1, with higher F-measure values indicating better classification ability), Matthew's correlation coefficient (“MCC”) (generated directly from the confusion matrix showing the correlation between observed and anticipated classifications, wherein a coefficient of +1 implies a perfect forecast, a coefficient of −1 shows total discrepancy between predicted and actual values, and a coefficient of zero indicates that the prediction is no better than random prediction), Cohen's Kappa (quantifies the level of agreement between two annotators on a classification problem, beyond what would be expected by chance), Jaccard index (the size of the intersection divided by the size of the union of two sets of labels; the Jaccard index compares the set of predicted labels for a sample with the corresponding set of true labels), Negative Predictive Value (“NPV”) (ratio of true negative instances correctly identified by the model to the total number of actual negative instances), Average precision score (summarizes the precision-recall curve by calculating the weighted mean of the precisions achieved at each threshold, with the weights reflecting the increase in recall from the previous threshold), False discovery rate (“FDR”) (calculated as the ratio of false positive predictions made by the model to the total number of positive predictions, including both false positives and true positives), or any combination of the foregoing.

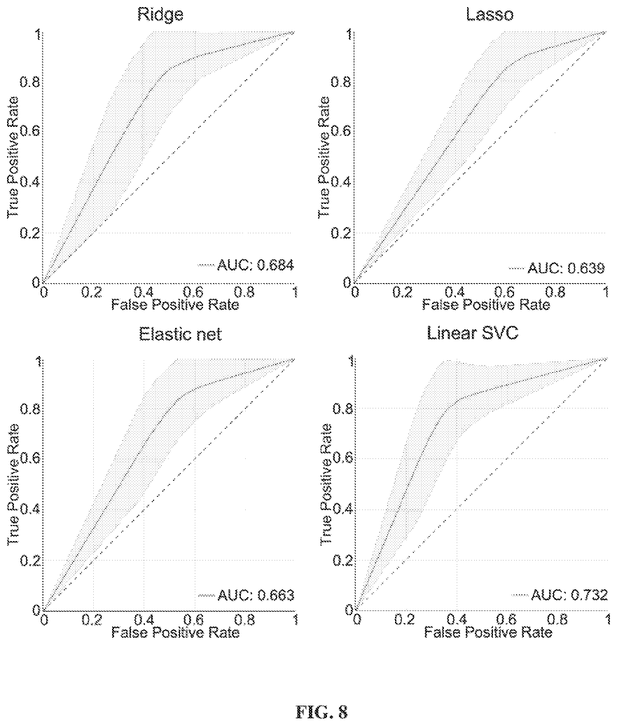

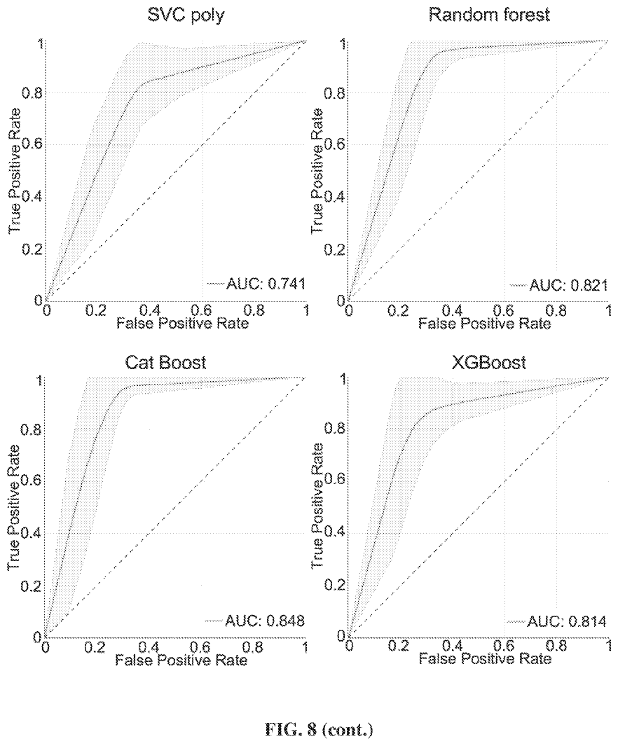

In some embodiments, the standard deviation of the predictive model is evaluated by randomly varying which samples are used for training and testing. In this way, a model can be constructed hundreds of times to determine mean performance plus or minus the standard deviation in performance (above and below the mean), see FIG. 8. Determining if model performance is acceptable can depend on a variety of factors that are within the level of skill of the skilled artisan to evaluate. In some instances, e.g., depending on how the model is to be used in conjunction with other steps for detecting, diagnosing, or assessing cancer, it may be preferable to prioritize reducing the number of false negatives. In some instances, it may be preferable to prioritize reducing the number of false positives. In some instances, balancing the number of false negatives and false positives may be preferable. In some instances, high sensitivity may be preferable. In some instances, high specificity may be preferable. In some embodiments, balancing sensitivity and specificity may be preferable.

In some embodiments, the performance of the prediction model is evaluated using Accuracy. In some embodiments, the accuracy of predicting cancer by the prediction model is greater than about 50%, such as greater than any of about 60%, about 65%, about 70%, about 80%, about 85%, about 90%, and about 95%. In some embodiments, the accuracy of predicting cancer by the prediction model is greater than about 70%, such as greater than any of about 80%, about 85%, about 90%, and about 95%. In some embodiments, the accuracy of predicting cancer by the prediction model is greater than about 90%, such as greater than any of about 91%, about 92%, about 93%, about 94%, about 95%, about 96%, about 97%, about 98%, and about 99%.

### 4. Continuous Development of the Prediction Model

In some embodiments, each time new samples are added to the model (e.g., from a new region, cancer type, controls), the model can be retrained with the updated samples (e.g., old samples+new). In some embodiments, each time new samples are added, all previously used algorithms can be evaluated and compared for performance changes. For the cancer types that the prediction model demonstrates acceptable performance, the model can be used immediately (e.g., in combination with medical consultancy). Thus, although the final prediction model can be continuously developed, it can also be continually used. In addition, the final prediction model can continuously evolve with the field of machine learning.

The database of samples with a known coupling to cancer or control can continuously grow and contribute to increased performance of the predictive model. For example, a prediction acquired via the chemistry of hair could lead to a biopsy and definite cancer diagnosis. In this case, the sample can contribute to the known samples in the database that can be used to train the next level of models. Furthermore, the study can continuously collect samples that can be characterized to represent differences in cancer subtypes, continental regions, medical histories, or sociological differences. These samples can be used to train the models to also identify chemical markers of these differences. The models can be continuously developed with this growing bulk of information. Therefore, the capacity of the decisive model can grow over time to pick up smaller differences in the lifestyle of the person that contributes the sample.

### 5. Use of the Built Prediction Model for New Samples

New samples from people with an unknown diagnosis can be analyzed to obtain chemical data (see, e.g., FIG. 1C). In some embodiments, the isotopic features of the new sample are obtained by determining, e.g., the values of isotopic fractional abundances and deltas of at least one isotopic element measured in the new sample. The chemical data of these samples is investigated through the trained prediction model built, e.g., using the TrainingS samples. Using the same algorithm used to build the prediction model, the isotopic features of the new sample from a person with an unknown diagnosis can be put through the prediction model. The overall output value for the prediction model can be used to calculate the probability that the chemistry preserved in hair represents a body where a tumor is growing. This information can assist the medical team in their evaluation of further tests or treatments.

### III. METHODS AND SYSTEMS OF DETECTING, DIAGNOSING, OR ASSESSING CANCER IN A SUBJECT USING A PREDICTION MODEL

In some embodiments, the prediction models can be used to detect whether a subject has cancer, such as in connection with diagnosing or monitoring cancer in a subject. In some embodiments, the prediction models can be used to assess a characteristic of the cancer or the subject, e.g., the level of tumor burden of the cancer in the subject. In some embodiments, isotopic features from a sample to be tested are determined, such as using methods similar to those described in Section II.A. In some embodiments, a test sample (e.g., hair or nail sample) from a subject to be tested for cancer is obtained or collected, processed and subjected to digestion (e.g., acid digestion with optional thermal digestion) or combustion, and the contents of the sample are analyzed by ICP-MS analysis for isotopic elements to determine isotope concentrations and, from the concentrations, at least one isotopic feature for the test sample. In some embodiments, the cancer is detected, diagnosed, or assessed using a machine learning algorithm that has been trained using values of the at least one isotopic feature, as determined from samples collected from a plurality of subjects with a particular cancer and also healthy or normal subjects. In some embodiments, the machine learning algorithm results in a binary output indicating the presence or absence of cancer. In some embodiments, the machine learning algorithm results in an output that is or indicates the probability of the presence of cancer. In some embodiments, the machine learning algorithm results in an output that is or indicates the extent of tumor burden. The confidence or probability threshold may be set by the user as desired, given the tolerance for inaccurate classification. In some embodiments, a probability greater than 0.5 indicates the presence of cancer. In some embodiments, test data including at least one isotopic feature associated with a sample from a subject to be tested in inputted into the trained prediction model, which then classifies the subject according to the model to determine whether the subject has cancer. The test data can include the isotopic features discussed herein.

In a second aspect, a method of noninvasively detecting the presence of cancer in a subject by doing a chemical analysis of the stable isotopic elements of a sample, e.g., hair or nails, is described. The methods described herein have the potential to detect cancer in undiagnosed people (cancer detection screen) as well as monitor for the presence of cancer after therapy (cancer monitoring screen). In some embodiments, the subject where cancer was detected is diagnosed with cancer and may further undergo treatment of said cancer. In some embodiments, the cancer treatment includes chemotherapy, radiotherapy, cryotherapy, photodynamic therapy, laser therapy, immunotherapy, targeted therapy, hormone therapy, surgical procedures to remove the cancer, stem cell transplant, bone marrow transplant, or any combination thereof. In some embodiments, the subject has previously received a treatment for the cancer and the provided methods are used as a cancer monitoring screen to assess the efficacy of the treatment.

In some embodiments, the provided method is a method of detecting whether a subject has a cancer, the method comprising: (a) determining the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer. In some embodiments, the provided method is a method of detecting whether a subject has a cancer, the method comprising: (a) determining the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer.

In some embodiments, the provided method is a method of detecting a cancer in a subject, the method comprising: (a) determining the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) detecting the cancer in the subject using the prediction model. In some embodiments, the provided method is a method of assessing a characteristic of a cancer in a subject, the method comprising: (a) determining the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

In some embodiments, the provided method of assessing a characteristic of a cancer in a subject, the method comprising: (a) determining the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

In some embodiments, the provided method is a method of detecting whether a subject has a cancer, the method comprising: (a) receiving, at one or more processors, the value for each of at least one isotopic feature for a sample from a subject, wherein the at least isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample from the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer.

In some embodiments, the provided method is a method of detecting whether a subject has a cancer, the method comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer.

In some embodiments, the provided method is a method of detecting a cancer in a subject, the method comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) detecting the cancer in the subject using the prediction model.

In some embodiments, the provided method is a method of assessing a characteristic of a cancer in a subject, the method comprising: (a) receiving, at one or more processors, the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting, using at least one processor, the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

In some embodiments, the provided method is a method of assessing a characteristic of a cancer in a subject, the method comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

In some embodiments, the provided method is a method of detecting whether a subject has a cancer, the method comprising: (a) providing a sample obtained from a subject; (b) preparing a liquid or solid sample for analysis by mass spectrometry from the sample, wherein the liquid sample is prepared by digestion of the sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements present in each of the standards and the prepared liquid or solid sample; (e) analyzing the detected one or more ionization products of isotopic elements to generate the value for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element; (f) receiving, at one or more processors, the value for each of the at least one isotopic feature; (g) inputting, using at least one processor, the value for each of the at least one isotopic feature into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (h) determining, using the prediction model, whether the subject has the cancer.

In some embodiments, the provided method is a method of detecting whether a subject has a cancer, the method comprising: (a) providing a solid sample obtained from a subject; (b) preparing a liquid sample or the solid sample for analysis by mass spectrometry, wherein the liquid sample is prepared by digestion of the solid sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements and isotopic elements present in each of the standards and the prepared liquid or solid sample; (e) analyzing the detected one or more ionization products of elements and isotopic elements to generate the value for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the value for each of the at least one isotopic feature; (g) inputting, using at least one processor, the value for each of the at least one isotopic feature into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (h) determining, using the prediction model, whether the subject has the cancer.

In some embodiments, the provided method is a method of detecting a cancer in a subject, the method comprising: (a) providing a solid sample obtained from a subject; (b) preparing a liquid sample or the solid sample for analysis by mass spectrometry, wherein the liquid sample is prepared by digestion of the solid sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements and isotopic elements present in each of the standards and the prepared liquid or solid sample; (e) analyzing the detected one or more ionization products of elements and isotopic elements to generate the value for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the value for each of the at least one isotopic feature; (g) inputting, using at least one processor, the value for each of the at least one isotopic feature into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (h) detecting the cancer in the subject using the prediction model.

In some embodiments, the provided method is a method of assessing a characteristic of a cancer in a subject, the method comprising: (a) providing a sample obtained from a subject; (b) preparing a liquid or solid sample for analysis by mass spectrometry from the sample, wherein the liquid sample is prepared by digestion of the sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements present in each of the standards and the prepared liquid or solid sample; (e) analyzing the detected one or more ionization products of isotopic elements to generate the value for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element; (f) receiving, at one or more processors, the value for each of the at least one isotopic feature; (g) inputting, using at least one processor, the value for each of the at least one isotopic feature into a prediction model configured to predict a characteristic of a cancer in a subject, wherein the prediction model comprises at least one machine learning algorithm trained using values for each of the at least one isotopic feature from a plurality of reference samples from a plurality of reference subjects; and (h) determining, using the prediction model, the characteristic of the cancer in the subject.

In some embodiments, the provided method is a method of assessing a characteristic of a cancer in a subject, the method comprising: (a) providing a solid sample obtained from a subject; (b) preparing a liquid sample or the solid sample for analysis by mass spectrometry, wherein the liquid sample is prepared by digestion of the solid sample; (c) providing standards comprising a plurality of calibration standards, a blank standard, an internal standard, and/or a reference material; (d) introducing the standards and the prepared liquid or solid sample individually into a mass spectrometer to detect one or more ionization products of elements and isotopic elements present in each of the standards and the prepared liquid or solid sample; (e) analyzing the detected one or more ionization products of elements and isotopic elements to generate the value for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3); (f) receiving, at one or more processors, the value for each of the at least one isotopic feature; (g) inputting, using at least one processor, the value for each of the at least one isotopic feature into a prediction model configured to predict a characteristic of a cancer in a subject, wherein the prediction model comprises at least one machine learning algorithm trained using values for each of the at least one isotopic feature from a plurality of reference samples from a plurality of reference subjects; and (h) determining, using the prediction model, the characteristic of the cancer in the subject.

In still other embodiments, a system for detecting whether a subject has a cancer is described, the system comprising one or more data processors and a non-transitory computer readable storage medium containing instructions which, when executed on the one or more data processors, cause the one or more data processors to perform actions comprising: (a) receiving, at one or more processors, the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer.

In still other embodiments, a system for detecting whether a subject has a cancer is described, the system comprising one or more data processors and a non-transitory computer readable storage medium containing instructions which, when executed on the one or more data processors, cause the one or more data processors to perform actions comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and (c) determining, using the prediction model, whether the subject has the cancer.

In some embodiments, a system for assessing a characteristic of a cancer in a subject is described, the system comprising one or more data processors and a non-transitory computer readable storage medium containing instructions which, when executed on the one or more data processors, cause the one or more data processors to perform actions comprising: (a) receiving, at one or more processors, the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject; (b) inputting, using at least one processor, inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

In some embodiments, a system for assessing a characteristic of a cancer in a subject is described, the system comprising one or more data processors and a non-transitory computer readable storage medium containing instructions which, when executed on the one or more data processors, cause the one or more data processors to perform actions comprising: (a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject; (b) inputting, using at least one processor, inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and (c) determining, using the prediction model, the characteristic of the cancer in the subject.

In some embodiments, a prediction model is applied using a computer-program product tangibly embodied in a non-transitory machine-readable storage medium, including instructions configured to cause one or more data processors to apply the prediction model as described herein.

In some embodiments, the prediction model was previously built using the methods described herein. In some embodiments, the prediction model was built using an alternative method.

Aspects of the provided methods also include treatment methods that include treating a subject for a cancer in which the cancer has been detected or the subject has been diagnosed as having cancer, by the methods as described. In some embodiments, the treatment method includes determining whether a cancer is detected in a subject by any of the provided methods of detecting cancer, and then administering to a subject in which cancer is detected an effective treatment for treating the cancer. In some embodiments, the treatment method includes diagnosing a subject as having a cancer by any of the provided methods of diagnosing cancer, and then administering to a subject diagnosed with cancer an effective treatment for treating the cancer.

Aspects of the provided methods also include treatment methods that include treating a subject for a cancer in which the cancer has been characterized as having a particular characteristic, by the methods as described. In some embodiments, the particular characteristic is the cancer having metastasized. In some embodiments, the particular characteristic is the cancer having remained localized. In some embodiments, the particular characteristic is a threshold level of tumor burden. In some embodiments, the treatment method includes determining the particular characteristic by any of the provided methods of detecting cancer, and then administering to a subject in which cancer is characterized as having the particular characteristic an effective treatment for treating the cancer.

In some embodiments, the treatment methods include obtaining the test data including at least one isotopic feature. In some embodiments, a test sample (e.g., hair or nail sample) is obtained from the subject, and the test sample is processed to prepare the sample for isotopic analysis by ICP-MS. In some embodiments, the test sample is processed using procedures as described in Section II.A. In some embodiments, the subject is a subject suspected of having cancer, such as due to one or more symptoms that may indicate a cancer is possible; or is a subject in which it is desired to screen for a cancer, such as due to a particular age of the subject or other risk factor, in accord with preventive treatment methods.

In some embodiments, the cancer is a blood cancer or is a solid tumor. In some embodiments, the blood cancer is a leukemia, a lymphoma or a multiple myeloma. In some embodiments, the cancer is a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, or a vascular cancer.

In some embodiments, the cancer is lung cancer, thyroid cancer, ovarian cancer, colorectal cancer, prostate cancer, cancer of the pancreas, cancer of the esophagus, liver cancer, breast cancer, skin cancer, or mesothelioma. In some embodiments, the cancer is mesothelioma, such as malignant mesothelioma. In some embodiments, the cancer is selected from leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; tonsil cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is prostate cancer. In some embodiments, the cancer is a kidney cancer. In some embodiments, the cancer is a renal cancer. In some embodiments, the cancer is a head and neck cancer. In some embodiments, the cancer is a breast cancer. In some embodiments, the cancer is a bladder cancer. In some embodiments, the cancer is a leukemia.

In some embodiments, the presence of cancer can be verified using any one of the following techniques, some of which are invasive: a blood test, a urine test, a biopsy, an endoscopic exam (e.g., a cystoscopy, a colonoscopy, an endoscopic retrograde cholangiopancreatography (ERCP), an esophagogastroduodenoscopy, sigmoidoscopy, bronchoscopy), a lumbar puncture, a pap test, surgery, genetic testing, and imaging (e.g., a computerized tomography (CT) scan, a bone scan, a magnetic resonance imaging (MRI) scan, a positron emission tomography (PET) scan, an ultrasound, a mammogram, a lymphangiogram (LAG), nuclear medicine imaging, and an X-ray).

In some embodiments of the methods and uses described herein, a cancer is treated by inhibiting or reducing or decreasing or arresting further growth or spread of the cancer or tumor. In some embodiments of the methods and uses described herein, a cancer is treated by inhibiting or reducing the size (e.g., volume or mass) of the cancer or tumor by at least 5%, at least 10%, at least 25%, at least 50%, at least 75%, at least 90% or at least 99% relative to the size of the cancer or tumor prior to treatment. In some embodiments of the methods and uses described herein, a cancer is treated by reducing the quantity of the cancers or tumors in the patient by at least 5%, at least 10%, at least 25%, at least 50%, at least 75%, at least 90% or at least 99% relative to the quantity of the cancers or tumors prior to treatment.

In some embodiments, treatments of cancers are known to a skilled artisan, including clinical care providers. The particular choice of treatment can be empirically determined and depends on various factors, such as the type of cancer, the severity of cancer, and the age or health of the subject. In some embodiments, effective cancer treatments include chemotherapy, radiotherapy, cryotherapy, photodynamic therapy, laser therapy, immunotherapy, targeted therapy, hormone therapy, surgical procedures to remove the cancer, stem cell transplant, bone marrow transplant, or any combination thereof.

For example, localized breast cancer can be treated by lumpectomy, mastectomy, or EBRT. Systemic treatment in the neoadjuvant, adjuvant, or metastatic settings can include antihormonal agents, e.g., tamoxifen, aromatase inhibitors, Herceptin, chemotherapy, e.g., cyclophosphamide, methotrexate, fluorouracil, docetaxel, cisplatin, a targeted agent, e.g., Olaparib, and/or immunotherapy, e.g., pembrolizumab.

For example, localized prostate cancer can be treated by prostatectomy, focal therapy, e.g., cryotherapy, brachytherapy, or EBRT. Systemic treatment in the neoadjuvant, adjuvant, or metastatic settings can include castration agents, e.g., leuprolide, other hormonal agents, e.g., abiraterone, enzalutamide, chemotherapy, e.g., docetaxel, cabazitaxel, a targeted agent, e.g., Olaparib.

For example, localized lung cancer can be treated by surgical resection or definitive radiation therapy, e.g., SABR. Systemic treatment in the neoadjuvant, adjuvant, or metastatic settings can include chemotherapy, e.g., cisplatin, gemcitabine, docetaxel, vinorelbine, etoposide, targeted agents, e.g., Osimertinib, and/or immunotherapy, e.g., Atezolizumab.

For example, localized colon cancer can be treated by surgical resection. Systemic treatment in the adjuvant or metastatic setting can include chemotherapy, e.g., a 5-fluorouracil based regimen, a capecitabine based regimen, a targeted agent, e.g., cetuximab, panitumumab, bevacizumab, lapatinib, and/or an immunotherapy, e.g., nivolumab, ipilimumab, pembrolizumab.

In some embodiments, treatments for cancer include localized therapy, such as local surgery or local radiotherapy. In some embodiments, local radiation therapy may include, for example, external beam radiation (EBRT), stereotactic body radiation (SBRT), charged particle therapy (such as proton beam therapy (PBT)), selective internal radiation therapy (SIRT), or ablation therapy (such as radiofrequency ablation (RFA) or microwave ablation (MW A)). Localized therapy may also include, for example, other treatments such as percutaneous ethanol injection therapy (PEIT), transarterial radioembolization (TARE), transarterial chemoembolization (TACE), highly-focused ultrasound (HIFU), irreversible electroporation (IRE), or more invasive surgical procedures (such as tissue resection or transplantation).

In some embodiments, effective treatments include a multi-targeted tyrosine kinase inhibitor (TKI). Exemplary multi-targeted TKIs include axitinib, brivanib, cabozantinib, cediranib, donofenib, dovitinib, lenvatinib, linifanib, nintedanib, regorafenib, sorafenib, and sunitinib.

In some embodiments, treatments include an immunotherapy. Exemplary immunotherapies include immune checkpoint inhibitors, such as inhibitors against cytotoxic T-lymphocyte antigen-4 (CTLA4), programmed death-1 (PD-1), or programmed death-1 ligand (PD-Li). In some embodiments, the immunotherapy includes an antibody or fragment targeting an immune checkpoint, such as, for example, an anti-CTLA4 antibody (such as tremelimumab or ipilimumab), an anti-PD-1 antibody (such as nivolumab, pembrolizumab, camrelizumab, or tislelizumab), or an anti-PD-L1 antibody (such as avelumab, atezolizumab, or durvalumab).

In some embodiments, treatment include a chemotherapy. Chemotherapy may include one or more of a fluoropyrimidine (e.g., gemcitabine, capecitabine, doxifluridine, fluorouracil, irinotecan, or tegafur (optionally in combination with uracil)), a platinum agent (e.g., cisplatin or oxaliplatin), or a taxane (such as docetaxel or paclitaxel).

In some embodiments, measuring or determining a value of at least one isotopic feature in a sample from a subject as described herein provides for an isotope profile of the subject. In some embodiments, an isotope profile of a subject is provided. The isotope profile can be from a solid sample from a subject (e.g., a keratinous tissue, optionally a hard keratinous tissue such as a hair or nail sample). In some embodiments, the solid sample is digested (e.g., by acid digestion and/or thermal digestion) into a prepared liquid sample for mass spectrometry as described herein. In some embodiments, the isotope profile can be used as input to a prediction model, including any of the prediction models described herein such as a machine learning algorithm, using methods as described herein.

In some embodiments, the isotope profile can comprise a value of at least one isotopic feature of at least one isotope selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se ⁸²Se ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing, and wherein the at least one isotopic feature is a concentration of the at least one isotope.

In some embodiments, an isotope profile comprises a value of at least one isotopic feature of at least one isotope selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, Ni, ⁶⁰Ni, ⁶¹Ni, ⁶²Ni, ⁶³Cu, Cu, ⁶⁴Zn, ⁶⁶Zn, 67Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing, and wherein the at least one isotopic feature is a concentration of the at least one isotope or a normalized value of the concentration of the at least one isotope.

In some embodiments, the value of the at least one isotopic feature of an isotope profile is a normalized value of the concentration of the at least one isotope. In some embodiments, the value of a plurality of isotopic features of an isotope profile are normalized value of the concentrations of the isotopes of the isotope profile. In some embodiments, the value of all isotopic features of an isotope profile are normalized value of the concentrations of the isotopes of the isotope profile. In some embodiments, the normalized value is an atomic percent (atom %), fractional abundance (F), isotope ratio (R), or a delta value (delta), or a combination of any of the foregoing. In some embodiments, the normalized value is a fractional abundance (F). In some embodiments, the normalized value is a delta value. In some embodiments, delta value is a conventional delta value, a study-specific delta value (InH), a non-similar simple-nitrogen based delta value (SimpleNδ), or a non-similar pseudo-nitrogen based delta value (PseudoNδ).

In some embodiments, an isotope profile comprises, consists, or consists essentially of one isotope, e.g., ⁶⁶Zn, ⁶⁴Zn, ⁶⁸Zn, ⁸⁷Sr, ⁸⁸Sr, ⁵⁶Sr, ⁴⁷Ti, ⁴⁸Ti, ⁴⁶Ti, and/or ⁵⁰Cr, e.g., ⁶⁴Zn, ⁶⁶Zn, or ³⁴S.

In some embodiments, an isotope profile comprises, consists, or consists essentially of a plurality of isotopes. In some embodiments, the plurality is from 2 to 59 isotopes, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 isotopes. In some embodiments, the plurality of isotopes of an isotope profile comprises, consists, or consists essentially of a concentration of two or all three of ⁶⁴Zn, ⁶⁶Zn, and/or ³⁴S.

An isotope profile can be used in a method of detecting cancer. For example, in some embodiments, the one or more isotopic features of an isotope profile as described herein (e.g., derived from the keratinous tissue of a subject), may be compared to one or more corresponding isotopic features (i.e., reference isotopic features) of reference subjects, e.g., (i) individuals characterized as having cancer, (ii) individuals characterized as being cancer free, or (iii) both individuals characterized as having cancer and individuals characterized as being cancer free. The comparison may be carried out in any manner that permits a determination of whether the isotope profile reflects that a subject from whom the isotope profile is obtained does or does not have cancer, or that there is a likelihood that the subject does or does not have cancer.

In some embodiments, for example, the comparison of one or more isotopic features of an isotope profile as described herein may simply comprise a direct comparison of the isotopic features to known values or known ranges of values of one or more corresponding reference isotopic features of reference subjects, e.g., (i) individuals characterized as having cancer, (ii) individuals characterized as being cancer free, or (iii) both individuals characterized as having cancer and individuals characterized as being cancer free. In such embodiments, for example, an isotopic feature in the isotope profile such as the concentration of ⁶⁴Zn (including a normalized value of the concentration as described above) the isotope profile may be compared to a reference concentration (including a normalized value of the concentration) of ⁶⁴Zn, or a range of reference of concentrations (or range of normalized reference concentrations) of ⁶⁴Zn, in such reference subjects, e.g., (i) individuals characterized as having cancer, (ii) individuals characterized as being cancer free, or (iii) both individuals characterized as having cancer and individuals characterized as being cancer free. Similarly, for example, the concentration of ⁶⁶Zn and/or ³⁴S (including normalized values of such concentration(s)) may be compared to the concentration (including normalized value of such concentration(s)) or range of concentrations(s) (including a range of normalized values of such concentration(s)) of ⁶⁶Zn and/or ³⁴S) in reference subjects, e.g., (i) individuals characterized as having cancer, (ii) individuals characterized as being cancer free, or (iii) both individuals characterized as having cancer and individuals characterized as being cancer free. And so on for each isotopic feature in the isotope profile for which such a reference value or range of reference values is known. In such embodiments involving a direct comparison of one or more isotopic features of an isotope profile to known values or ranges of known values (including normalized values), for example, if one isotopic feature of the isotope profile is the same as a reference value or within a reference range of reference values for the corresponding isotopic feature of persons characterized as having cancer, then the subject may be indicated as having, or determined to have cancer or possibly have cancer. Likewise, if a plurality of isotopic features of the isotope profile are the same as the reference values or within a reference range of reference values for the corresponding isotopic features of persons characterized as having cancer, then the subject may be indicated as having, or determined to have cancer or as possibly have cancer.

In other embodiments, the comparison is an analysis that is carried out by inputting the value for one or more isotopic features (including normalized values) from the isotope profile into a prediction model (as described in detail herein) configured to predict the presence or absence of a cancer in the subject. In such embodiments, the prediction model can comprise at least one machine learning algorithm as described herein trained using values for one or more corresponding isotopic features from a plurality of reference samples from a plurality of reference subjects. In such embodiments, the prediction model performs the analysis and makes a determination of whether the subject from whom the isotope profile is obtained does or does not have cancer, or that there is a likelihood that the subject does or does not have cancer.

In embodiments, the isotope profile is obtained (as described herein) at a physician's office and the comparison is performed at the physician's office in a manner as described above, e.g., by comparison with one or more reference values or via a prediction model, and the determination of whether the subject from whom the isotope profile is obtained does or does not have cancer, or that there is a likelihood that the subject does or does not have cancer, is communicated to the subject. In some embodiments, the isotope profile is communicated from the physician's office, e.g., in written form or electronically (e.g., as a data file), to a different party that performs a comparison as described above and then communicates the results to the physician's office, which in turn communicates the determination to the subject.

In some embodiments, the isotope profile is not obtained at a physician's office but rather by a laboratory that carries out the process of obtaining the isotope profile as described herein using a keratinous sample received from a physician's office or directly from a subject (including receipt of the sample from a physician's office or directly from a subject by mail or courier). In such embodiments, the comparison may be performed by the laboratory in a manner as described above, e.g., by comparison with one or more reference values or via a prediction model, and the determination of whether the subject from whom the isotope profile is obtained does or does not have cancer, or that there is a likelihood that the subject does or does not have cancer, is communicated directly to the subject and/or to the subject's physician. In some embodiments, the isotope profile is communicated from the laboratory, e.g., in written form or electronically (e.g., as a data file), to a different party that performs a comparison as described above, e.g., by comparison with one or more reference values or via a prediction model, and then communicates the results (i) to the laboratory, which in turn relays the determination to the subject, and/or the subject's physician, or (ii) directly to the subject and/or the subject's physician, and optionally to the laboratory. In such embodiments, the third party that performs the comparison receives the isotope profile of a subject (e.g., in written form or via electric communication, e.g., as a data file), performs the comparison (e.g., by comparison with one or more reference values or via a prediction model), and then communicates the determination, in written form and/or via electronic communication, to one or more of the isotope profile provider (e.g., a physician's office or laboratory) and/or the subject. The determination may include, for example, (i) a comparison of isotopic features of the subject's isotope profile with corresponding isotopic features from a plurality of reference samples from a plurality of reference subjects, whether obtained by a direct comparison of values or ranges of values, or by a representation of the results of the comparison/analysis from the prediction model, and (ii) a determination of the probability that the subject has cancer, or (iii) both (i) and (ii).

### IV. COMPUTING UNITS

In some embodiments, the systems and methods described herein may be partially or fully implemented by a computer that executes one or more particular functions embodied in computer programs. Such computer systems can include one or more programs configured to execute one or more processors for the computer system to perform such methods. One or more steps of the computer-implemented methods may be performed automatically. The computer system may include one or more computing nodes. For example, a system may include two or more computing nodes (e.g., servers, computers, routers, or other types of electronic devices that include a network interface), which may be connected and configured to communicate and execute the methods over said network on one or more computing nodes of the network.

In some embodiments, the methods described herein for detecting, diagnosing, or assessing cancer may be a computer-implemented method using a specifically designed machine or system that includes a prediction model with a trained machine-learning algorithm, which may be stored on a non-transitory computer readable memory of the computer or system. The computer generally includes one or more processors that can access the memory. The one or more processors can receive test data, e.g., values of isotopic features from a subject, which may also be stored on the memory. The one or more processors can access the prediction model, and can input the test data e.g., values of isotopic features from a subject, into the model. The one or more processors and the trained machine-learning model can then predict the presence of absence of cancer or a characteristic of the subject or cancer.

In some embodiments, the methods and systems described herein utilize a computing unit comprising an input interface designed for receiving input data and an output interface designed to output results. The computing unit can be designed to host at least one prediction model, e.g., to process the input data using the at least one prediction model in order to get results. In some embodiments, the computing unit hosts more than one prediction model. In some embodiments, the computing unit comprises a memory to save and provide the at least one prediction model. Each prediction model can be trained to determine a cancer indicating, e.g., the probability that a subject has cancer. For example, some prediction models can be trained for different patient-groups, e.g., based on global location, and can be selected based on patient-relating information in the input data.

A report may be generated that identifies whether the subject has cancer or characteristics of the subject or cancer. In some embodiments, the report identifies whether the subject has cancer based on a binary classification of “yes” or “no.” In some embodiments, the report identifies whether the subject has cancer based on a probability of the cancer (e.g., a probability of greater than 0.5 indicates the subject likely has cancer). The report may be, for example, an electronic medical record or a printed report, which can be transmitted to the subject or a healthcare provider (doctor, clinic, etc.) for the subject. The report may be used to make healthcare decisions, such as the method by which the cancer in the subject is treated.

The report may be displayed on an electronic display or customized interface. For example, in some embodiments, the computer-implemented method may automatically generate the report, and may automatically display the generated report on an electronic display or customized interface.

Computing units and/or computing devices according to one or more example embodiments may be implemented using hardware, software, and/or a combination thereof. For example, hardware devices may be implemented using processing circuitry including, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result.

In some embodiments, software includes a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter. For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Any of the disclosed systems or methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above-mentioned embodiments and/or to perform the method of any of the above-mentioned embodiments.

Computing units and/or computing devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the methods described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Bluray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the systems or methods described herein, or they may be known devices that are altered and/or modified to manage the systems and/or methods described herein.

A hardware device, such as a computer processing device, may run an operating system (OS) and one or more software applications that run on the OS. The computer processing device also may access, store, manipulate, process, and create data in response to execution of the software. For simplicity, one or more embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions. The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Examples of the non-transitory computer-readable medium include rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include memory cards; and media with a built-in ROM, including ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The prediction models may be present in the computing unit itself or in a memory used by the computing unit. For example, in some embodiments, information about the architecture and parameters of the prediction models are present in a memory and a chosen prediction model is downloaded from the memory into (a random access memory of) the computing unit.

In some embodiments, a selection unit is designed for automatically selecting one of these prediction models. The selection can be based on a predefined selection scheme and on data input into the system to be processed by the prediction model(s). The selection scheme may be a table stored in a memory of the computing system or a decision tree hardwired in the algorithm of the selection unit. Depending on the inputted data (e.g., chemical data, data comprising information about the patient such as gender, continental regions, medical histories, or sociological differences), a prediction model of a plurality of models can be selected by the selection unit based on the selection scheme. In some embodiments, the selection unit can evaluate which prediction model (architecture and training) would be optimal for the subject being tested, e.g., providing the best results for that subject.

Accordingly, in some other embodiments, a computer-program product tangibly embodied in a non-transitory machine-readable storage medium is described, wherein the computer-program product is used to diagnose, detect, or assess cancer in a subject, wherein the computer-program product includes instructions configured to cause one or more data processors to perform one or more steps of any of the described methods.

In some embodiments, the prediction model was built using the methods described herein. In some embodiments, the prediction model was built using alternative methods, as understood by the person skilled in the art.

Specific structural and functional details disclosed herein are merely representative for purposes of describing example embodiments. The present invention may, however, be embodied in many alternate forms and should not be construed as limited to only the embodiments set forth herein.

### V. EXEMPLARY EMBODIMENTS

Among the provided embodiments are:

1. A method of detecting whether a subject has a cancer, the method comprising:
(a) determining the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject;
(b) inputting the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(c) determining, using the prediction model, whether the subject has the cancer.

2. A method of assessing a characteristic of a cancer in a subject, the method comprising:
(a) determining the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject;
(b) inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and
(c) determining, using the prediction model, the characteristic of the cancer in the subject.

3. The method of embodiment 2, wherein the characteristic of the cancer is the level of tumor burden of the cancer in the subject.

4. The method of any one of embodiments 1-3, wherein the determining the value for each of the at least one isotopic feature comprises mass spectrometry analysis of a liquid sample prepared by digestion of the sample or a solid sample of the sample that is analyzed via combustion.

5. The method of any one of embodiments 1-4, wherein the determining the value for each of the at least one isotopic feature comprises:
(i) preparing a liquid sample of the sample for analysis by mass spectrometry, wherein the liquid sample is prepared by digestion of the sample;
(ii) introducing the prepared liquid sample into a mass spectrometer to detect one or more ionization products of isotopic elements present in the prepared liquid sample; and
(iii) analyzing the detected one or more ionization products of isotopic elements to generate the value for each of the at least one isotopic feature.

6. The method of any one of embodiments 1-4, wherein the determining the value for each of the at least one isotopic feature comprises:
(i) preparing a solid sample of the sample for analysis by mass spectrometry;
(ii) introducing the prepared solid sample into a mass spectrometer to detect one or more ionization products of isotopic elements present in the prepared solid sample, wherein the detecting comprises combustion of the prepared solid sample; and
(iii) analyzing the detected one or more ionization products of isotopic elements to generate the value for each of the at least one isotopic feature.

7. The method of any one of embodiments 1-6, wherein the determining in step
(a), the inputting in step (b), and/or the determining in step (c) is performed by a processor of a computing device.

8. A method of detecting whether a subject has a cancer, the method comprising:
(a) receiving, at one or more processors, the value for each of at least one isotopic feature for a sample from a subject, wherein the at least isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample from the subject;
(b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(c) determining, using the prediction model, whether the subject has the cancer.

9. A method of assessing a characteristic of a cancer in a subject, the method comprising:
(a) receiving, at one or more processors, the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject;
(b) inputting, using at least one processor, the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and
(c) determining, using the prediction model, the characteristic of the cancer in the subject.

10. The method of embodiment 9, wherein the characteristic of the cancer is the level of tumor burden of the cancer in the subject.

11. The method of any one of embodiments 8-10, wherein the value for each of the at least one isotopic feature is obtained by mass spectrometry analysis of a liquid sample prepared by digestion of the sample or a solid sample of the sample that is analyzed via combustion.

12. The method of any one of embodiments 8-11, wherein prior to step (a), the method comprises determining the value for each of the at least one isotopic feature, wherein the determining comprises:
(i) preparing a liquid sample of the sample for analysis by mass spectrometry, wherein the liquid sample is prepared by digestion of the sample;
(ii) introducing the prepared liquid sample into a mass spectrometer to detect one or more ionization products of isotopic elements present in the prepared liquid sample; and
(iii) analyzing the detected one or more ionization products of isotopic elements to generate the value for each of the at least one isotopic feature.

13. The method of any one of embodiments 8-11, wherein prior to step (a), the method comprises determining the value for each of the at least one isotopic feature, wherein the determining comprises:
(i) preparing a solid sample of the sample for analysis by mass spectrometry;
(ii) introducing the prepared solid sample into a mass spectrometer to detect one or more ionization products of isotopic elements present in the prepared solid sample, wherein the detecting comprises combustion of the prepared solid sample; and
(iii) analyzing the detected one or more ionization products of isotopic elements to generate the value for each of the at least one isotopic feature.

14. The method of any one of embodiments 5-7, 12, and 13, wherein the analyzing the detected one or more ionization products of isotopic elements is with reference to one or more ionization products of elements present in standards comprising a plurality of calibration standards and a blank standard.

15. The method of any one of embodiments 5-7 and 12-14, wherein the analyzing the detected one or more ionization products of isotopic elements is with reference to one or more ionization products of elements present in standards comprising an internal standard and/or a reference material.

16. The method of embodiment 14 or embodiment 15, wherein the mass spectrometer is further introduced with the standards, wherein the mass spectrometer detects one or more ionization products of elements present in the standards and the prepared liquid or solid sample.

17. A method of detecting whether a subject has a cancer, the method comprising:
(a) providing a sample obtained from a subject;
(b) preparing a liquid or solid sample for analysis by mass spectrometry from the sample, wherein the liquid sample is prepared by digestion of the sample;
(c) providing standards comprising a plurality of calibration standards, a blank standard, an internal standard, and/or a reference material;
(d) introducing the standards and the prepared liquid or solid sample individually into a mass spectrometer to detect one or more ionization products of elements present in each of the standards and the prepared liquid or solid sample;
(e) analyzing the detected one or more ionization products of isotopic elements to generate the value for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element;
(f) receiving, at one or more processors, the value for each of the at least one isotopic feature;
(g) inputting, using at least one processor, the value for each of the at least one isotopic feature into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(h) determining, using the prediction model, whether the subject has the cancer.

18. A method of assessing a characteristic of a cancer in a subject, the method comprising:
(a) providing a sample obtained from a subject;
(b) preparing a liquid or solid sample for analysis by mass spectrometry from the sample, wherein the liquid sample is prepared by digestion of the sample;
(c) providing standards comprising a plurality of calibration standards, a blank standard, an internal standard, and/or a reference material;
(d) introducing the standards and the prepared liquid or solid sample individually into a mass spectrometer to detect one or more ionization products of elements present in each of the standards and the prepared liquid or solid sample;
(e) analyzing the detected one or more ionization products of isotopic elements to generate the value for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element;
(f) receiving, at one or more processors, the value for each of the at least one isotopic feature;
(g) inputting, using at least one processor, the value for each of the at least one isotopic feature into a prediction model configured to predict a characteristic of a cancer in a subject, wherein the prediction model comprises at least one machine learning algorithm trained using values for each of the at least one isotopic feature from a plurality of reference samples from a plurality of reference subjects; and
(h) determining, using the prediction model, the characteristic of the cancer in the subject.

19. The method of embodiment 18, wherein the characteristic of the cancer is the level of tumor burden of the cancer in the subject.

20. The method of any one of embodiments 1-19, wherein the sample comprises a keratinous tissue, optionally a hard keratinous tissue.

21. The method of any one of embodiments 1-20, wherein the sample is a noninvasive sample, optionally a hair sample or a nail sample.

22. The method of any one of embodiments 4, 5, 7, 11, 12, and 14-21, wherein the digestion is by acid digestion and/or thermal digestion.

23. The method of any one of embodiments 4, 5, 7, 11, 12, and 14-22, wherein the digestion is by acid digestion and thermal digestion.

24. The method of embodiment 22 or embodiment 23, wherein the acid digestion is with nitric acid (HNO₃).

25. The method of any one of embodiments 22-24, wherein the acid digestion is with nitric acid (HNO₃) and hydrogen peroxide (H₂O₂).

26. The method of any one of embodiments 22-25, wherein the thermal digestion is by a microwave.

27. The method of any one of embodiments 4-7 and 11-26, wherein the mass spectrometry is Inductively Coupled Plasma Mass Spectrometry (ICP-MS).

28. The method of embodiment 27, wherein the ICP-MS is carried out with kinetic energy discrimination (KED).

29. The method of any one of embodiments 1-28, wherein the at least one isotopic feature comprises an atomic percent, isotope ratio, fractional abundance, delta, overall concentration, and/or concentration ratio.

30. The method of any one of embodiments 1-29, wherein the at least one isotopic feature comprises an atomic percent for at least one isotope of an isotopic element, an isotope ratio for at least one isotope of an isotopic element, a fractional abundance for at least one isotope of an isotopic element, and/or a delta for at least one isotope of an isotopic element.

31. The method of any one of embodiments 1-30, wherein the at least one isotopic feature is a plurality of isotopic features each independently selected from the group consisting of atomic percents, isotope ratios, fractional abundances, deltas, overall concentrations, and concentration ratios.

32. The method of embodiment 31, wherein the plurality of isotopic features comprises an atomic percent for at least one isotope of an isotopic element, an isotope ratio for at least one isotope of an isotopic element, a fractional abundance for at least one isotope of an isotopic element, and/or a delta for at least one isotope of an isotopic element.

33. The method of any one of embodiments 1-32, wherein the isotopic element is Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S.

34. The method of any one of embodiments 1-32, wherein the isotopic element is N, C, S, or O.

35. The method of any one of embodiments 1-32, wherein the isotopic element is Mo, Cr, Te, C, or Hg.

36. The method of any one of embodiments 31-35, wherein the plurality of isotopic features comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic features.

37. The method of any one of embodiments 31-36, wherein the plurality of isotopic features are determined from at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic elements.

38. The method of any one of embodiments 31-37, wherein the plurality of isotopic features comprises fractional abundances and deltas.

39. The method of embodiment 38, wherein the fractional abundance and delta values are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

40. The method of any one of embodiments 31-39, wherein the plurality of isotopic features comprises fractional abundances, atomic percents, and/or isotope ratios.

41. The method of embodiment 40, wherein the fractional abundances, atomic percents, and/or isotope ratios are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

42. The method of any one of embodiments 37-41, wherein the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, and/or S.

43. The method of any one of embodiments 37-42, wherein the isotopic elements comprise N, C, S, and/or O.

44. The method of any one of embodiments 37-43, wherein the isotopic elements comprise Mo, Cr, Te, C, and/or Hg.

45. The method of any one of embodiments 1-44, wherein at least one isotopic feature comprises a concentration ratio.

46. The method of embodiment 45, wherein the concentration ratio is N/C, S/C, O/C, S/N, N/O, or S/O.

47. The method of any one of embodiments 31-46, wherein the plurality of isotopic features comprises at least one concentration ratio.

48. The method of embodiment 47, wherein the at least one concentration ratio comprises N/C, S/C, O/C, S/N, N/O, and/or S/O.

49. The method of any one of embodiments 1-48, wherein the at least one isotopic feature comprises at least 1, 2, 3, 4, 5, 6, 7, 8, 9, or 10 of the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (f³⁴S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO).

50. The method of any one of embodiments 1-49, wherein the at least one isotopic feature comprises at least 1, 2, 3, 4, 5, 6, 7, 8, 9, 10, 11, 12, 13, 14, 15, 16, 17, 18, 19, 20, or more of the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷I, ¹²⁸Te, ¹³⁶Ba, ¹³⁷Ba, ¹³C, ²³⁵U, ²³⁸U, ²³Na, ²⁷Al, ³⁴S, ³⁹K, ⁴⁴Ca, ⁴⁸Ti, ⁵⁴Cr, ⁵⁴Fe, ⁵⁷Fe, ⁶²Ni, ⁶³Cu, ⁶⁶Zn, ⁶⁷Zn, ⁶⁸Zn, ⁷⁰Ge ⁷⁴Ge, ⁷⁴Se, ⁷⁶Se, ⁷⁷Se, ⁷⁹Br, ⁸⁰Se, ⁸²Se, ⁸⁹Y, ⁹⁴Mo, ⁹⁷Mo, and ⁹⁸Mo, each relative to stable isotopes of the isotopic element of the isotope.

51. The method of any one of embodiments 1-50, wherein the at least one isotopic feature comprises at least 1, 2, 3, 4, 5, 6, 7, 8, 9, 10, or more of the fractional abundance, atomic percent, or isotope ratio of ¹⁰⁷Ag, ¹⁰⁹Ag, ¹¹⁸Sn, ¹²⁷I, ¹³C, ²³⁵U, ²⁷Al, ³⁴S, ⁴⁴Ca, ⁵⁷Fe, ⁶⁷Zn, ⁷⁴Se, ⁹⁴Mo, and ⁹⁷Mo, each relative to stable isotopes of the isotopic element of the isotope.

52. The method of any one of embodiments 1-51, wherein the at least one isotopic feature comprises the fractional abundance, atomic percent, or isotope ratio of ¹²I relative to stable isotopes of I and the fractional abundance, atomic percent, or isotope ratio of ⁴⁴Ca relative to stable isotopes of Ca.

53. The method of any one of embodiments 1-52, wherein the machine learning algorithm is a supervised machine learning algorithm.

54. The method of any one of embodiments 1, 2, 4-9, 11-18, and 20-53, wherein the prediction model is a classification model.

55. The method of any one of embodiments 1, 2, 4-9, 11-18, and 20-54, wherein the prediction model is a binary classification model.

56. The method of any one of embodiments 1, 2, 4-9, 11-18, and 20-54, wherein the prediction model is a multiclass classification model.

57. The method of any one of embodiments 1, 2, 4-9, 11-18, and 20-56, wherein the machine learning algorithm is Logistic Regression (e.g., Ridge, Lasso, or ElasticNet), Naive Bayes Classifier, GaussianNB, GaussianNBisotonic, GaussianNBsigmoid, Random Forest Classifier, GradientBoosting Classifier, XGBoost Classifer, HistGradientBoosting Classifier, Deep Neural Network Classifier, DecisionTree Classifier, Bagging Classifier, ExtraTrees Classifier, AdaBoost Classifier, CatBoost Classifier, LGBM Classifier, Support Vector Classifier, Perceptron Classifier (e.g., Multi-layer Perceptron Classifier), Deep Learning Classifier for tabular data (e.g., TabNet or TabML), PassiveAggressiveClassifier, or SGD Classifier, or an ensemble algorithm comprising a combination of any of the foregoing.

58. The method of any one of embodiments 1, 2, 4-9, 11-18, and 20-57, wherein the machine learning algorithm is a support vector classifier, a decision tree-based ensemble classifier, or a multilayer perceptron classifier.

59. The method of any one of embodiments 2-7, 9-16, and 18-53, wherein the prediction model is a regression model.

60. The method of any one of embodiments 1-52, wherein the machine learning algorithm is an unsupervised machine learning algorithm.

61. The method of any one of embodiments 1-60, wherein the subject is a mammal.

62. The method of any one of embodiments 1-61, wherein the subject is a human.

63. The method of any one of embodiments 1-62, wherein the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

64. The method of any one of embodiments 1-63, wherein the cancer is prostate cancer, bladder cancer, or kidney cancer.

65. The method of any one of embodiments 1-64, wherein the method is noninvasive.

66. The method of any one of embodiments 1-65, wherein the prediction model is built by:
(i) determining the values for each of the at least one isotopic feature for the plurality of reference samples; and
(ii) using the values for each of the at least one isotopic feature for the plurality of reference samples to train the machine learning algorithm.

67. The method of any one of embodiments 1-66, wherein some of the plurality of reference subjects are known to have cancer, and some of the plurality of reference subjects are assumed to not have cancer.

68. The method of any one of embodiments 1-59 and 61-67, wherein the machine learning algorithm is trained using a plurality of labels, the plurality of labels comprising a label for each of the plurality of reference samples.

69. The method of embodiment 68, wherein the label indicates if the reference sample is from a reference subject known to have cancer or from a reference subject assumed to not have cancer.

70. The method of embodiment 68, wherein the label indicates the characteristic of the cancer in the reference subject from which the reference sample was obtained.

71. The method of embodiment 68 or embodiment 70, wherein the label indicates the level of tumor burden of the cancer in the reference subject from which the reference sample was obtained.

72. The method of any one of embodiments 1-71, wherein the method further comprises, prior to step (a), training the machine learning algorithm using the values for each of the at least one isotopic feature for the plurality of reference samples.

73. The method of any one of embodiments 1-59 and 61-72, wherein the method further comprises, prior to step (a), training the machine learning algorithm using the plurality of labels and the values for each of the at least one isotopic feature for the plurality of reference samples.

74. The method of embodiment 72 or embodiment 73, wherein the method further comprises preprocessing the values for each of the at least one isotopic feature prior to the training the machine learning algorithm.

75. The method of embodiment 74, wherein the preprocessing comprises controlled imputation of missing values or replacing missing values with a zero.

76. The method of embodiment 74 or embodiment 75, wherein the preprocessing comprises data normalization.

77. The method of any one of embodiments 1, 4-8, 11-17, 20-69, and 72-76, wherein cancer is detected in the subject if the prediction model indicates the presence of cancer.

78. The method of embodiment 77, wherein the method further comprises verification of the detected cancer by a method selected from the group consisting of a blood test, a urine test, a biopsy, an endoscopic exam, a lumbar puncture, a pap test, surgery, genetic testing, and imaging.

79. The method of any one of embodiments 1, 4-8, 11-17, 20-69, and 72-78, wherein the method is for diagnosing cancer in the subject, wherein the subject is diagnosed with cancer if the prediction model indicates the presence of cancer.

80. The method of any one of embodiments 1-79, wherein the subject has one or more symptoms that indicate a cancer may be present in the subject.

81. The method of any one of embodiments 1, 4-8, 11-17, 20-69, and 72-79, wherein the method is a preventive screening method for cancer in the subject.

82. The method of any one of embodiments 1, 4-8, 11-17, 20-69, and 72-81, wherein if the subject is diagnosed with cancer, the subject undergoes treatment for the cancer.

83. The method of any one of embodiments 1, 4-8, 11-17, 20-69, and 72-82, wherein if the subject is diagnosed with cancer, the method further comprises treating the subject for the cancer.

84. A method of treating a cancer in a subject, comprising:
(a) diagnosing a subject with cancer according to the method of any one of embodiments 1, 4-8, 11-17, 20-69, and 72-83, wherein the prediction model indicates the presence of cancer; and
(b) treating the subject for the cancer with a treatment for the cancer.

85. The method of any one of embodiments 1-78 and 80, wherein the method is for monitoring cancer treatment in the subject.

86. The method of any one of embodiments 1-78, 80, and 85, wherein the subject has been previously diagnosed with cancer and is undergoing treatment, or has been previously diagnosed with cancer and is believed to be in remission.

87. The method of embodiment 85 or embodiment 86, wherein if cancer is detected in the subject, cancer treatment for the subject is continued or re-started.

88. The method of any one of embodiments 85-87, wherein if cancer is detected in the subject, the method further comprises continuing or re-starting treatment of the subject for the cancer.

89. A method of treating a cancer in a subject, comprising:
(a) detecting a cancer in a subject according to the method of any one of embodiments 1, 4-8, 11-17, 20-69, and 72-78, 80, and 85-88, wherein the prediction model indicates the presence of cancer, and the subject has been previously diagnosed with cancer and is undergoing treatment for the cancer, or has been previously diagnosed with cancer and is believed to be in remission; and
(b) treating the subject for the cancer with a treatment for the cancer.

90. The method of any one of embodiments 82-89, wherein the treatment comprises chemotherapy, radiotherapy, cryotherapy, photodynamic therapy, laser therapy, immunotherapy, targeted therapy, hormone therapy, surgical procedures to remove the cancer, stem cell transplant, bone marrow transplant, or a combination of any of the foregoing.

91. The method of any one of embodiments 1-90, wherein the prediction model is built and applied using a computer-program product tangibly embodied in a non-transitory machine-readable storage medium comprising instructions configured to cause one or more data processors to build and apply the prediction model.

92. A system for detecting whether a subject has a cancer, the system comprising one or more data processors and a non-transitory computer readable storage medium containing instructions which, when executed on the one or more data processors, cause the one or more data processors to perform actions comprising:
(a) receiving, at one or more processors, the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject;
(b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(c) determining, using the prediction model, whether the subject has the cancer.

93. A system for assessing a characteristic of a cancer in a subject, the system comprising one or more data processors and a non-transitory computer readable storage medium containing instructions which, when executed on the one or more data processors, cause the one or more data processors to perform actions comprising:
(a) receiving, at one or more processors, the value for each of at least one isotopic feature for a sample from a subject, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in the sample of the subject;
(b) inputting, using at least one processor, inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and
(c) determining, using the prediction model, the characteristic of the cancer in the subject.

94. The system of embodiment 93, wherein the characteristic of the cancer is the level of tumor burden of the cancer in the subject.

95. The system of any one of embodiments 92-94, wherein the value for each of the at least one isotopic feature is obtained by mass spectrometry analysis of a liquid sample prepared by digestion of the sample or a solid sample of the sample that is analyzed via combustion.

96. The system of embodiment 95, wherein the mass spectrometry analysis is performed by:
(i) preparing a liquid sample of the sample for analysis by mass spectrometry, wherein the liquid sample is prepared by digestion of the sample;
(ii) introducing the prepared liquid sample into a mass spectrometer to detect one or more ionization products of isotopic elements present in the prepared liquid sample; and
(iii) analyzing the detected one or more ionization products of isotopic elements to generate the value for each of the at least one isotopic feature.

97. The system of embodiment 95, wherein the mass spectrometry analysis is performed by:
(i) preparing a solid sample of the sample for analysis by mass spectrometry;
(ii) introducing the prepared solid sample into a mass spectrometer to detect one or more ionization products of isotopic elements present in the prepared solid sample, wherein the detecting comprises combustion of the prepared solid sample; and
(iii) analyzing the detected one or more ionization products of isotopic elements to generate the value for each of the at least one isotopic feature.

98. The system of embodiment 96 or embodiment 97, wherein the analyzing the detected one or more ionization products of isotopic elements is with reference to one or more ionization products of elements present in standards comprising a plurality of calibration standards and a blank standard.

99. The system of any one of embodiments 96-98, wherein the analyzing the detected one or more ionization products of isotopic elements is with reference to one or more ionization products of elements present in standards comprising an internal standard and/or a reference material.

100. The system of embodiment 98 or embodiment 99, wherein the mass spectrometer is further introduced with the standards, wherein the mass spectrometer detects one or more ionization products of elements present in the standards and the prepared liquid or solid sample.

101. The system of any one of embodiments 92-100, wherein the sample comprises keratinous tissue, optionally hard keratinous tissue.

102. The system of any one of embodiments 92-101, wherein the sample is a noninvasive sample, optionally a hair sample or a nail sample.

103. The system of any one of embodiments 95-102, wherein the digestion is by acid digestion and/or thermal digestion.

104. The system of any one of embodiments 95-103, wherein the digestion is by acid digestion and thermal digestion.

105. The system of embodiment 103 or embodiment 104, wherein the acid digestion is with nitric acid (HNO₃).

106. The system of any one of embodiments 103-105, wherein the acid digestion is with nitric acid (HNO₃) and hydrogen peroxide (H₂O₂).

107. The system of any one of embodiments 103-106, wherein the thermal digestion is by a microwave.

108. The system of any one of embodiments 95-107, wherein the mass spectrometry is Inductively Coupled Plasma Mass Spectrometry (ICP-MS).

109. The system of embodiment 108, wherein the ICP-MS is carried out with kinetic energy discrimination (KED).

110. The system of any one of embodiments 92-109, wherein the at least one isotopic feature comprises an atomic percent, isotope ratio, fractional abundance, delta, overall concentration, and/or concentration ratio.

111. The system of any one of embodiments 92-110, wherein the at least one isotopic feature comprises an atomic percent for at least one isotope of an isotopic element, an isotope ratio for at least one isotope of an isotopic element, a fractional abundance for at least one isotope of an isotopic element, and/or a delta for at least one isotope of an isotopic element.

112. The system of any one of embodiments 92-111, wherein the at least one isotopic feature is a plurality of isotopic features each independently selected from the group consisting of atomic percents, isotope ratios, fractional abundances, deltas, overall concentrations, and concentration ratios.

113. The system of embodiment 112, wherein the plurality of isotopic features comprises an atomic percent for at least one isotope of an isotopic element, an isotope ratio for at least one isotope of an isotopic element, a fractional abundance for at least one isotope of an isotopic element, and/or a delta for at least one isotope of an isotopic element.

114. The system of any one of embodiments 92-113, wherein the isotopic element is Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S.

115. The system of any one of embodiments 92-113, wherein the isotopic element is N, C, S, or O.

116. The system of any one of embodiments 92-113, wherein the isotopic element is Mo, Cr, Te, C, or Hg.

117. The system of any one of embodiments 112-116, wherein the plurality of isotopic features comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic features.

118. The system of any one of embodiments 112-117, wherein the plurality of isotopic features are determined from at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic elements.

119. The system of any one of embodiments 112-118, wherein the plurality of isotopic features comprises fractional abundances and deltas.

120. The system of embodiment 119, wherein the fractional abundance and delta values are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

121. The system of any one of embodiments 112-120, wherein the plurality of isotopic features comprises fractional abundances, atomic percents, and/or isotope ratios.

122. The system of embodiment 121, wherein the fractional abundances, atomic percents, and/or isotope ratios are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

123. The system of any one of embodiments 118-122, wherein the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, and/or S.

124. The system of any one of embodiments 118-123, wherein the isotopic elements comprise N, C, S, and/or O.

125. The system of any one of embodiments 118-124, wherein the isotopic elements comprise Mo, Cr, Te, C, and/or Hg.

126. The system of any one of embodiments 92-125, wherein at least one isotopic feature comprises a concentration ratio.

127. The system of embodiment 126, wherein the concentration ratio is N/C, S/C, O/C, S/N, N/O, or S/O.

128. The system of any one of embodiments 112-127, wherein the plurality of isotopic features comprises at least one concentration ratio.

129. The system of embodiment 128, wherein the at least one concentration ratio comprises N/C, S/C, O/C, S/N, N/O, and/or S/O.

130. The system of any one of embodiments 92-129, wherein the at least one isotopic feature comprises at least 1, 2, 3, 4, 5, 6, 7, 8, 9, or 10 of the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (F³⁴S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO).

131. The system of any one of embodiments 92-130, wherein the at least one isotopic feature comprises at least 1, 2, 3, 4, 5, 6, 7, 8, 9, 10, 11, 12, 13, 14, 15, 16, 17, 18, 19, 20, or more of the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷I, ¹²⁸Te, ¹³⁶Ba, ¹³⁷Ba, ¹³C, ²³⁵U, ²³⁸U, ²³Na, ²⁷Al, ³⁴S, ³⁹K, ⁴⁴Ca, ⁴⁸Ti, ⁵⁴Cr, ⁵⁴Fe, ⁵⁷Fe, ⁶²Ni, ⁶³Cu, ⁶⁶Zn, ⁶⁷Zn, ⁶⁸Zn, ⁷⁰Ge, ⁷⁴Ge, ⁷⁴Se, ⁷⁶Se, ⁷⁷Se, ⁷⁹Br, ⁸⁰Se, ⁸²Se, ⁸⁹Y, ⁹⁴Mo, ⁹⁷Mo, and ⁹⁸Mo, each relative to stable isotopes of the isotopic element of the isotope.

132. The system of any one of embodiments 92-131, wherein the at least one isotopic feature comprises at least 1, 2, 3, 4, 5, 6, 7, 8, 9, 10, or more of the fractional abundance, atomic percent, or isotope ratio of ¹⁰⁷Ag, ¹⁰⁹Ag, ¹¹⁸Sn, ¹²⁷I, ¹³C, ²³⁵U, ²⁷Al, ³⁴S, 44Ca, ⁵⁷Fe, ⁶⁷Zn, ⁷⁴Se, ⁹⁴Mo, and ⁹⁷Mo, each relative to stable isotopes of the isotopic element of the isotope.

133. The system of any one of embodiments 92-132, wherein the at least one isotopic feature comprises the fractional abundance, atomic percent, or isotope ratio of ¹²⁷I. relate to stables isotopes of I and the fractional abundance, atomic percent, or isotope ratio of ⁴⁴Ca relative to stable isotopes of Ca.

134. The system of any one of embodiments 92-133, wherein the machine learning algorithm is a supervised machine learning algorithm.

135. The system of any one of embodiments 92, 93, and 95-134, wherein the prediction model is a classification model.

136. The system of any one of embodiments 92, 93, and 95-135, wherein the prediction model is a binary classification model.

137. The system of any one of embodiments 92, 93, and 95-135, wherein the prediction model is a multiclass classification model.

138. The system of any one of embodiments 92, 93, and 95-137, wherein the machine learning algorithm is Logistic Regression (e.g., Ridge, Lasso, or ElasticNet), Naive Bayes Classifier, GaussianNB, GaussianNBisotonic, GaussianNBsigmoid, Random Forest Classifier, GradientBoosting Classifier, XGBoost Classifer, HistGradientBoosting Classifier, Deep Neural Network Classifier, DecisionTree Classifier, Bagging Classifier, ExtraTrees Classifier, AdaBoost Classifier, CatBoost Classifier, LGBM Classifier, Support Vector Classifier, Perceptron Classifier (e.g., Multi-layer Perceptron Classifier), Deep Learning Classifier for tabular data (e.g., TabNet or TabML), PassiveAggressiveClassifier, or SGD Classifier, or an ensemble algorithm comprising a combination of any of the foregoing.

139. The system of any one of embodiments 92, 93, and 95-138, wherein the machine learning algorithm is a support vector classifier, a decision tree-based ensemble classifier, or a multilayer perceptron classifier.

140. The system of any one of embodiments 93-134, wherein the prediction model is a regression model.

141. The system of any one of embodiments 92-140, wherein the machine learning algorithm is an unsupervised machine learning algorithm.

142. The system of any one of embodiments 92-141, wherein the subject is a mammal.

143. The system of any one of embodiments 92-142, wherein the subject is a human.

144. The system of any one of embodiments 92-143, wherein the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

145. The system of any one of embodiments 92-144, wherein the cancer is prostate cancer, bladder cancer, or kidney cancer.

146. The system of any one of embodiments 92-145, wherein the prediction model is built by:
(i) determining the values for each of the at least one isotopic feature for the plurality of reference samples; and
(ii) using the values for each of the at least one isotopic feature for the plurality of reference samples to train the machine learning algorithm.

147. The system of any one of embodiments 92-146, wherein some of the plurality of reference subjects are known to have cancer, and some of the plurality of reference subjects are assumed to not have cancer.

148. The system of any one of embodiments 92-140 and 142-147, wherein the machine learning algorithm is trained using a plurality of labels, the plurality of labels comprising a label for each of the plurality of reference samples.

149. The system of embodiment 148, wherein the label indicates if the reference sample is from a reference subject known to have cancer or from a reference subject assumed to not have cancer.

150. The system of embodiment 148, wherein the label indicates the characteristic of the cancer in the reference subject from which the reference sample was obtained.

151. The system of embodiment 148 or embodiment 150, wherein the label indicates the level of tumor burden of the cancer in the reference subject from which the reference sample was obtained.

152. The system of any one of embodiments 92-151, wherein the values for each of the at least one isotopic feature are preprocessed prior to training the machine learning algorithm.

153. The system of embodiment 152, wherein the preprocessing comprises controlled imputation of missing values or replacing missing values with a zero.

154. The system of embodiment 152 or embodiment 153, wherein the preprocessing comprises data normalization.

155. The system of any one of embodiments 92 and 95-154, wherein cancer is detected in the subject if the prediction model indicates the presence of cancer.

156. A method of building a prediction model for detecting whether a subject has a cancer, the method comprising:
(a) determining, for a plurality of reference samples from a plurality of reference subjects, values for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples; and
(b) building a prediction model configured to predict the presence or absence of a cancer in a subject, wherein the prediction model comprises at least one machine learning algorithm, and the building comprises training the machine learning algorithm using the values for each of the at least one isotopic feature for the plurality of reference samples.

157. A method of building a prediction model for assessing a characteristic of a cancer in a subject, the method comprising:
(a) determining, for a plurality of reference samples from a plurality of reference subjects, the value for each of at least one isotopic feature, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples; and
(b) building a prediction model configured to predict a characteristic of a cancer in a subject, wherein the prediction model comprises at least one machine learning algorithm, and the building comprises training the machine learning algorithm using the values for each of the at least one isotopic feature for the plurality of reference samples.

158. The method of embodiment 157, wherein the characteristic of the cancer is the level of tumor burden of the cancer in the subject.

159. The method of any one of embodiments 156-158, wherein the determining the values for each of the at least one isotopic feature comprises mass spectrometry analysis of liquid samples prepared by digestion of one or more of the reference samples and/or solid samples of one or more of the reference samples that are analyzed via combustion.

160. The method of any one of embodiments 156-159, wherein the determining the values for each of the at least one isotopic feature comprises:
(i) preparing liquid samples of one or more of the reference samples for analysis by mass spectrometry, wherein the liquid samples are prepared by digestion of the one or more reference samples;
(ii) introducing the prepared liquid samples into a mass spectrometer to detect one or more ionization products of isotopic elements present in the prepared liquid samples; and (iii) analyzing the detected one or more ionization products of isotopic elements to generate values for one or more of the at least one isotopic feature for the prepared liquid samples.

161. The method of any one of embodiments 156-160, wherein the determining the values for each of the at least one isotopic feature comprises:
(i) preparing solid samples of one or more of the reference samples for analysis by mass spectrometry;
(ii) introducing the prepared solid samples into a mass spectrometer to detect one or more ionization products of isotopic elements present in the prepared solid samples, wherein the detecting comprises combustion of the prepared solid samples; and
(iii) analyzing the detected one or more ionization products of isotopic elements to generate values for one or more of the at least one isotopic feature for the prepared solid samples.

162. The method of any one of embodiments 156-161, wherein the determining and/or building is performed by a processor of a computing device.

163. A method of building a prediction model for detecting whether a subject has a cancer, the method comprising:
(a) receiving, at one or more processors, values for each of at least one isotopic feature for a plurality of reference samples from a plurality of reference subjects, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples; and
(b) building, using at least one processor, a prediction model configured to predict the presence or absence of a cancer in a subject, wherein the prediction model comprises at least one machine learning algorithm, and the building comprises training the machine learning algorithm using the values for each of the at least one isotopic feature for the plurality of reference samples.

164. A method of building a prediction model for assessing a characteristic of a cancer in a subject, the method comprising:
(a) receiving, at one or more processors, values for each of at least one isotopic feature for each of a plurality of reference samples from a plurality of reference subjects, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element measured in a reference sample of the plurality of reference samples; and
(b) building, using at least one processor, a prediction model configured to predict a characteristic of a cancer in a subject, wherein the prediction model comprises at least one machine learning algorithm, and the building comprises training the machine learning algorithm using the values for each of the at least one isotopic feature for the plurality of reference samples.

165. The method of embodiment 164, wherein the characteristic of the cancer is the level of tumor burden of the cancer in the subject.

166. The method of any one of embodiments 163-165, wherein the values for each of the at least one isotopic feature is obtained by mass spectrometry analysis of liquid samples prepared by digestion of one or more of the reference samples and/or solid samples of one or more of the reference samples that are analyzed via combustion.

167. The method of any one of embodiments 163-166, wherein prior to step (a), the method comprises determining the values for each of the at least one isotopic feature, wherein the determining comprises:
(i) preparing liquid samples of one or more of the reference samples for analysis by mass spectrometry, wherein the liquid samples are prepared by digestion of the one or more reference samples;
(ii) introducing the prepared liquid samples into a mass spectrometer to detect one or more ionization products of isotopic elements present in the prepared liquid samples; and (iii) analyzing the detected one or more ionization products of isotopic elements to generate values for one or more of the at least one isotopic feature for the prepared liquid samples.

168. The method of any one of embodiments 163-167, wherein prior to step (a), the method comprises determining the values for each of the at least one isotopic feature, wherein the determining comprises:
(i) preparing solid samples of one or more of the reference samples for analysis by mass spectrometry;
(ii) introducing the prepared solid samples into a mass spectrometer to detect one or more ionization products of isotopic elements present in the prepared solid samples, wherein the detecting comprises combustion of the prepared solid samples; and
(iii) analyzing the detected one or more ionization products of isotopic elements to generate values for one or more of the at least one isotopic feature for the prepared solid samples.

169. The method of any one of embodiments 160-162, 167, and 168, wherein the analyzing the detected one or more ionization products of isotopic elements is with reference to one or more ionization products of elements present in standards comprising a plurality of calibration standards and a blank standard.

170. The method of any one of embodiments 160-162 and 167-169, wherein the analyzing the detected one or more ionization products of isotopic elements is with reference to one or more ionization products of elements present in standards comprising an internal standard and/or a reference material.

171. The method of embodiment 169 or embodiment 170, wherein the mass spectrometer is further introduced with the standards, wherein the mass spectrometer detects one or more ionization products of elements present in the standards and the prepared liquid or solid samples.

172. A method of building a prediction model for detecting whether a subject has a cancer, the method comprising:
(a) providing a plurality of reference samples obtained from a plurality of reference subjects;
(b) preparing liquid and/or solid samples for analysis by mass spectrometry from the plurality of reference samples, wherein the liquid samples are prepared by digestion of one or more of the reference samples;
(c) providing standards comprising a plurality of calibration standards, a blank standard, an internal standard, and/or a reference material;
(d) introducing the standards and the prepared liquid or solid samples individually into a mass spectrometer to detect one or more ionization products of elements present in each of the standards and the prepared liquid or solid samples;
(e) analyzing the detected one or more ionization products of isotopic elements to generate values for each of at least one isotopic feature for the plurality of reference samples, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element;
(f) receiving, at one or more processors, the values for each of the at least one isotopic feature; and
(g) building, using at least one processor, a prediction model configured to predict the presence or absence of a cancer in a subject, wherein the prediction model comprises at least one machine learning algorithm, and the building comprises training the machine learning algorithm using the values for each of the at least one isotopic feature for the plurality of reference samples.

173. A method of building a prediction model for assessing a characteristic of a cancer in a subject, the method comprising:
(a) providing a plurality of reference samples obtained from a plurality of reference subjects;
(b) preparing liquid and/or solid samples for analysis by mass spectrometry from the plurality of reference samples, wherein the liquid samples are prepared by digestion of one or more of the reference samples;
(c) providing standards comprising a plurality of calibration standards, a blank standard, an internal standard, and/or a reference material;
(d) introducing the standards and the prepared liquid or solid samples individually into a mass spectrometer to detect one or more ionization products of elements present in each of the standards and the prepared liquid or solid samples;
(e) analyzing the detected one or more ionization products of isotopic elements to generate values for each of at least one isotopic feature for the plurality of reference samples, wherein the at least one isotopic feature comprises the overall concentration of an isotopic element, a chemical feature determined from the overall concentration of an isotopic element, the concentration of an isotope of an isotopic element, or a chemical feature determined from the concentration of an isotope of an isotopic element;
(f) receiving, at one or more processors, the values for each of the at least one isotopic feature; and
(g) building, using at least one processor, a prediction model configured to predict a characteristic of a cancer in a subject, wherein the prediction model comprises at least one machine learning algorithm, and the building comprises training the machine learning algorithm using the values for each of the at least one isotopic feature for the plurality of reference samples.

174. The method of embodiment 173, wherein the characteristic of the cancer is the level of tumor burden of the cancer in the subject.

175. The method of any one of embodiments 156-174, wherein the plurality of reference samples each comprise a keratinous tissue, optionally a hard keratinous tissue.

176. The method of any one of embodiments 156-175, wherein the plurality of reference samples are noninvasive samples, optionally hair samples and/or nail samples.

177. The method of any one of embodiments 159-162 and 166-176, wherein the digestion is by acid digestion and/or thermal digestion.

178. The method of any one of embodiments 159-162 and 166-177, wherein the digestion is by acid digestion and thermal digestion.

179. The method of embodiment 177 or embodiment 178, wherein the acid digestion is with nitric acid (HNO₃).

180. The method of any one of embodiments 177-179, wherein the acid digestion is with nitric acid (HNO₃) and hydrogen peroxide (H₂O₂).

181. The method of any one of embodiments 177-180, wherein the thermal digestion is by a microwave.

182. The method of any one of embodiments 159-162 and 166-181, wherein the mass spectrometry is Inductively Coupled Plasma Mass Spectrometry (ICP-MS).

183. The method of embodiment 182, wherein the ICP-MS is carried out with kinetic energy discrimination (KED).

184. The method of any one of embodiments 156-183, wherein the at least one isotopic feature comprises an atomic percent, isotope ratio, fractional abundance, delta, overall concentration, and/or concentration ratio.

185. The method of any one of embodiments 156-184, wherein the at least one isotopic feature comprises an atomic percent for at least one isotope of an isotopic element, an isotope ratio for at least one isotope of an isotopic element, a fractional abundance for at least one isotope of an isotopic element, and/or a delta for at least one isotope of an isotopic element.

186. The method of any one of embodiments 156-185, wherein the at least one isotopic feature is a plurality of isotopic features each independently selected from the group consisting of atomic percents, isotope ratios, fractional abundances, deltas, overall concentrations, and concentration ratios.

187. The method of embodiment 186, wherein the plurality of isotopic features comprises an atomic percent for at least one isotope of an isotopic element, an isotope ratio for at least one isotope of an isotopic element, a fractional abundance for at least one isotope of an isotopic element, and/or a delta for at least one isotope of an isotopic element.

188. The method of any one of embodiments 156-187, wherein the isotopic element is Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S.

189. The method of any one of embodiments 156-187, wherein the isotopic element is N, C, S, or O.

190. The method of any one of embodiments 156-187, wherein the isotopic element is Mo, Cr, Te, C, or Hg.

191. The method of any one of embodiments 186-190, wherein the plurality of isotopic features comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic features.

192. The method of any one of embodiments 186-191, wherein the plurality of isotopic features are determined from at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more isotopic elements.

193. The method of any one of embodiments 186-192, wherein the plurality of isotopic features comprises fractional abundances and deltas.

194. The method of embodiment 193, wherein the fractional abundance and delta values are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

195. The method of any one of embodiments 186-194, wherein the plurality of isotopic features comprises fractional abundances, atomic percents, and/or isotope ratios.

196. The method of embodiment 195, wherein the fractional abundances, atomic percents, and/or isotope ratios are obtained for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more isotopes of isotopic elements.

197. The method of any one of embodiments 192-196, wherein the isotopic elements comprise Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, and/or S.

198. The method of any one of embodiments 192-197, wherein the isotopic elements comprise N, C, S, and/or O.

199. The method of any one of embodiments 192-198, wherein the isotopic elements comprise Mo, Cr, Te, C, and/or Hg.

200. The method of any one of embodiments 156-199, wherein at least one isotopic feature comprises a concentration ratio.

201. The method of embodiment 200, wherein the concentration ratio is N/C, S/C, O/C, S/N, N/O, or S/O.

202. The method of any one of embodiments 186-201, wherein the plurality of isotopic features comprises at least one concentration ratio.

203. The method of embodiment 202, wherein the at least one concentration ratio comprises N/C, S/C, O/C, S/N, N/O, and/or S/O.

204. The method of any one of embodiments 156-203, wherein the at least one isotopic feature comprises at least 1, 2, 3, 4, 5, 6, 7, 8, 9, or 10 of the fractional abundance, atomic percent, or isotope ratio of ¹³C (F13C) relative to stable isotopes of C, the fractional abundance, atomic percent, or isotope ratio of ¹⁸O (F18O) relative to stable isotopes of O, the concentration ratio of sulfur over nitrogen (SdN), the concentration ratio of nitrogen over carbon (NdC), the fractional abundance, atomic percent, or isotope ratio of ³⁴S (F³⁴S) relative to stable isotopes of S, the fractional abundance, atomic percent, or isotope ratio of ¹⁵N (F15N) relative to stable isotopes of N, the concentration ratio of oxygen over carbon (OdC), the concentration ratio of nitrogen over oxygen (NdO), the concentration ratio of sulfur over carbon (SdC), and the concentration ratio of sulfur over oxygen (SdO).

205. The method of any one of embodiments 156-204, wherein the at least one isotopic feature comprises at least 1, 2, 3, 4, 5, 6, 7, 8, 9, 10, 11, 12, 13, 14, 15, 16, 17, 18, 19, 20, or more of the fractional abundance, atomic percent, or isotope ratio of ¹⁰¹Ru, ¹⁰⁷Ag, ¹⁰⁹Ag, ¹⁰B, ¹¹⁸Sn, ¹¹⁹Sn, ¹²⁴Sn, ¹²⁴Te, ¹²⁷I, ¹²⁸Te, ¹³⁶Ba, ¹³⁷Ba, ¹³C, ²³⁵U, ²³⁸U, ²³Na, ²⁷Al, ³⁴S, ³⁹K ⁴⁴Ca ⁴⁸Ti, ⁵⁴C, ⁵⁴Fe, ⁵⁷Fe, ⁶²Ni, ⁶³Cu, ⁶⁶Zn, ⁶⁷Zn, ⁶⁸Zn, ⁷⁰Ge, ⁷⁴Ge, ⁷⁴Se, ⁷⁶Se, ⁷⁷Se, ⁷⁹Br, ⁸⁰Se, ⁸²Se, ⁸⁹Y, ⁹⁴Mo, ⁹⁷Mo, and ⁹⁸Mo, each relative to stable isotopes of the isotopic element of the isotope.

206. The method of any one of embodiments 156-205, wherein the at least one isotopic feature comprises at least 1, 2, 3, 4, 5, 6, 7, 8, 9, 10, or more of the fractional abundance, atomic percent, or isotope ratio of ¹⁰⁷Ag, ¹⁰⁹Ag, ¹¹⁸Sn, ¹²⁷I, ¹³C, ²³⁵U, ²⁷Al, ³⁴S, ⁴⁴Ca, ⁵⁷Fe, ⁶⁷Zn, ⁷⁴Se, ⁹⁴Mo, and ⁹⁷Mo, each relative to stable isotopes of the isotopic element of the isotope.

207. The method of any one of embodiments 156-206, wherein the at least one isotopic feature comprises the fractional abundance, atomic percent, or isotope ratio of ¹²⁷I relate to stables isotopes of I and the fractional abundance, atomic percent, or isotope ratio of ⁴⁴Ca relative to stable isotopes of Ca.

208. The method of any one of embodiments 156-207, wherein the machine learning algorithm is a supervised machine learning algorithm.

209. The method of any one of embodiments 156, 157, 159-164, 166-173, and 175-208, wherein the prediction model is a classification model.

210. The method of any one of embodiments 156, 157, 159-164, 166-173, and 175-209, wherein the prediction model is a binary classification model.

211. The method of any one of embodiments 156, 157, 159-164, 166-173, and 175-209, wherein the prediction model is a multiclass classification model.

212. The method of any one of embodiments 156, 157, 159-164, 166-173, and 175-211, wherein the machine learning algorithm is Logistic Regression (e.g., Ridge, Lasso, or ElasticNet), Naive Bayes Classifier, GaussianNB, GaussianNBisotonic, GaussianNBsigmoid, Random Forest Classifier, GradientBoosting Classifier, XGBoost Classifer, HistGradientBoosting Classifier, Deep Neural Network Classifier, DecisionTree Classifier, Bagging Classifier, ExtraTrees Classifier, AdaBoost Classifier, CatBoost Classifier, LGBM Classifier, Support Vector Classifier, Perceptron Classifier (e.g., Multi-layer Perceptron Classifier), Deep Learning Classifier for tabular data (e.g., TabNet or TabML), PassiveAggressiveClassifier, or SGD Classifier, or an ensemble algorithm comprising a combination of any of the foregoing.

213. The method of any one of embodiments 156, 157, 159-164, 166-173, and 175-212, wherein the machine learning algorithm is a support vector classifier, a decision tree-based ensemble classifier, or a multilayer perceptron classifier.

214. The method of any one of embodiments 157-162, 164-172, and 174-208, wherein the prediction model is a regression model.

215. The method of any one of embodiments 156-207, wherein the machine learning algorithm is an unsupervised machine learning algorithm.

216. The method of any one of embodiments 156-215, wherein the plurality of reference subjects are mammals.

217. The method of any one of embodiments 156-216, wherein the plurality of reference subjects are humans.

218. The method of any one of embodiments 156-217, wherein the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

219. The method of any one of embodiments 156-218, wherein the cancer is prostate cancer, bladder cancer, or kidney cancer.

220. The method of any one of embodiments 156-219, wherein some of the plurality of reference subjects are known to have cancer, and some of the plurality of reference subjects are assumed to not have cancer.

221. The method of any one of embodiments 156-214 and 216-220, wherein the building comprises training the machine learning algorithm using a plurality of labels, the plurality of labels comprising a label for each of the plurality of reference samples.

222. The method of embodiment 221, wherein the label indicates if the reference sample is from a reference subject known to have cancer or from a reference subject assumed to not have cancer.

223. The method of embodiment 221, wherein the label indicates the characteristic of the cancer in the reference subject from which the reference sample was obtained.

224. The method of embodiment 221 or embodiment 223, wherein the label indicates the level of tumor burden of the cancer in the reference subject from which the reference sample was obtained.

225. The method of any one of embodiments 156-224, wherein the method further comprises preprocessing the values for each of the at least one isotopic feature prior to the training the machine learning algorithm.

226. The method of embodiment 225, wherein the preprocessing comprises controlled imputation of missing values or replacing missing values with a zero.

227. The method of embodiment 225 or embodiment 226, wherein the preprocessing comprises data normalization.

228. The method of any one of embodiments 156, 59-163, 166-172, 175-222, and 225-227, wherein cancer is detected in the subject if the prediction model indicates the presence of cancer.

229. The method of any one of embodiments 156-228, wherein the prediction model is built using a computer-program product tangibly embodied in a non-transitory machine-readable storage medium comprising instructions configured to cause one or more data processors to build the prediction model.

### VI. EXAMPLES

The following examples are included for illustrative purposes only and are not intended to limit the scope of the invention.

### Example 1: Machine Learning Methods Involving Isotopic Features of Hair and Nail Samples to Predict Cancer in Subjects

### Collection of Samples

Samples were collected at sites at Lund University (LU), Sweden and Brady Urological Institute, Johns Hopkins School of Medicine (JHU), USA; IRB NA_00087094. Patients and controls were asked to deliver hair and nail clippings in pre-labelled vials. The samples were sent to LU and thereafter analyzed (destructively) for major and minor elemental concentrations at Chalmers University of Technology (SE) and Iso-Analytical Ltd (UK). Cancer types covered in the collection were from patients with prostate (236), bladder, (18) and kidney (8) cancer (total 262). Control samples were collected from 171 individuals without known cancer.

### Chemistry

Major elements (C, N, S, O) were analyzed at Iso-Analytical in the United Kingdom. Certified internal standards IA-R068, IA-R038, IA-R069, IA-R046/IAEA-C7, IA-R061, and IAEA-SO-5 were used, where maximum uncertainty was 0.23‰ for δ¹⁵N, 0.26%, for δ¹³C, 0.42‰ for δ³⁴S, and 0.23‰ for δ¹⁸O.

Chemical elements were analyzed at Chalmers University of Technology at 20 Oct. 2020, 18 Dec. 2020, 28 Apr. 2021, 7 Sep. 2021, and 13 Dec. 2021. Samples were prepared by weighing out 0.2-0.5 mg into vessels for digestion. The samples were digested using 0.25 mL HNO3 65% (digest for 2 days), 2.0 mL internal standard Scandium and Indium (2 ppb) in HNO3 0.5M, and 2.75 mL HNO3 0.5M. The total volume Vₜₒₜₐₗ was 5 mL and the dilution factor 5. The clear solutions (no precipitate) were filtrated through 0.45 μm syringe filter (polypropylene) before Inductively Coupled Plasma Mass Spectrometry (ICP-MS) analysis.

Although not the method of digestion used in this example, in some embodiments, samples for isotopic elemental analysis can be prepared by weighing approximately 50 mg of the sample or the entire sample if lower than 50 mg. After an optional wash, samples can be transferred to a vessel, e.g., a PFA vessel, followed by addition of 0.5 mL of HNO₃ and 0.5 mL of H₂O₂. The vessels are closed, mounted in sleeves, and heated in a microwave oven at 325 W power for 30 min. It should be appreciated by the person skilled in the art that other digestion means can be used to digest the hair and/or nails for isotopic elemental analysis.

The analysis occurred with a Thermo iCAP Q-ICP-MS in KED mode (kinetic energy discrimination) to reduce polyatomic ion interferences derived from the plasma or vacuum interface in collision cell ICP-MS. The elements analyzed were: Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y and Zn. Four standard solutions were used (0, 4, 10, and 50 ppb) containing 20 metals from stock solutions of 10 000 ppb. Samples were run, together with certified and matrix-relevant (hair) reference materials (IAEA-085, ERM-DB001) and a matrix relevant reference material (hair). Uncertainty for total concentrations generally ranged within 1-5%. When uncertainty was >20-30% for some minors (e.g., Rb), this data was disregarded. All the isotopes of the same elements will have the same concentration, and therefore the isotopic concentration should be compensated manually for the natural abundance. In principle, the isotopic fractional abundance should be reflected in the signal intensity. For example, the intensity ratios between Ti isotopes should be close to the ratio of natural abundance percentage of Ti isotopes. The concentrations (ppb) were used instead of signal to calculate isotope ratios (based on natural abundance) since the signal does not necessarily follow natural abundance. With ICP-QMS, interferences on certain mass numbers can cause faulty concentrations. For example, carbon-12 is abundant everywhere, it has a very strong signal on mass 12 that may intrude a bit on mass number 11. Therefore, as a skilled artisan readily understands ¹¹B interferes with ¹²C, and ¹¹B is eliminated from the study for this reason. The instrument has a certain finite mass resolution. Strong signals may interfere with an adjacent mass number.

### Machine Learning

Data preprocessing: From the 36 elements, 91 isotopic fractional abundances and delta values were used from 171 controls (healthy subjects) and 262 cancer patients were used. Missing values were imputed using recently developed XGBoost powered R implementation mixgb [30].

Feature selection: All features were used for this example, however, if needed features can be selected using Model-X knockoffs implemented in DeepLINK [31] and using SequentialFeatureSelector (scikit-learn python implementation).

Models: Ten different machine learning algorithms (Ridge, Lasso, Elastic Net, Linear SVC, SVC Poly, Random Forest, Cat Boost, XGBoost, MLPC, and TabNet) were used to build binary prediction models. All 91 features (4 features from major elements and 87 features from minor elements) were used to build each model. When data from both nails and hair was used, matrix geometry information (hair and nail) was incorporated using one-hot encoding (scikit-learn python implementation). Data from 80% of the samples was used to build or train the model (including validation data) and 20% was used for testing the model performances, i.e., 346 samples were used to build the model and 87 samples were used for testing. Model parameters were optimized following the guidelines provided by the algorithm developer.

To identify which features are informative for the prediction, the python package SHAP was used [32].

### Results

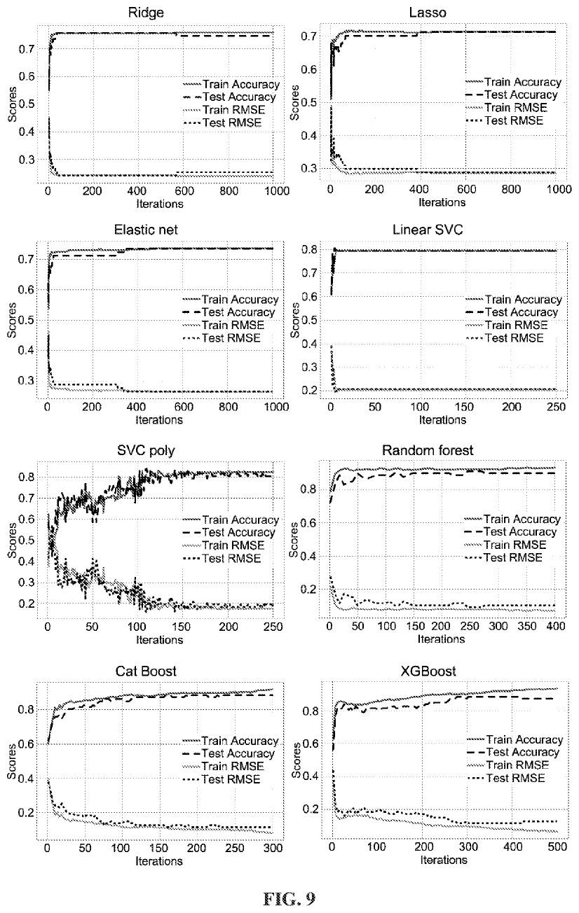

Eight models (Ridge, Lasso, Elastic Net, Linear SVC, SVC Poly, Random Forest, Cat Boost, XGBoost) were tested using accuracy and root-mean-square error (RMSE) scores to verify performance differences between train and test samples. All models displayed acceptable performances (FIG. 9).

Next, all eight models (Ridge, Lasso, Elastic Net, Linear SVC, SVC Poly, Random Forest, Cat Boost, XGBoost) were tested using different portions of samples (FIG. 8). In this case, test samples (20% from all samples) were sampled in 500 different ways (corresponding 80% samples were used to develop model). Therefore, 500 models were built for each algorithm and tested using 500 different test samples (more specifically sample combinations). Receiver Operating Characteristic (ROC) curves were used to assess the models' performance, and average area under the curve (AUC) was calculated. ROC matrices are plots of the true positive rates on the Y-axis and false positives on the X-axis. On average, 0.684 AUC for Ridge, 0.639 AUC for lasso, 0.663 AUC for elastic net, 0.732 AUC for linear SVC, 0.741 for SVC Poly, 0.821 AUC for random forest, 0.848 AUC for cat boost, and 0.814 AUC for XGBoost was achieved (FIG. 8).

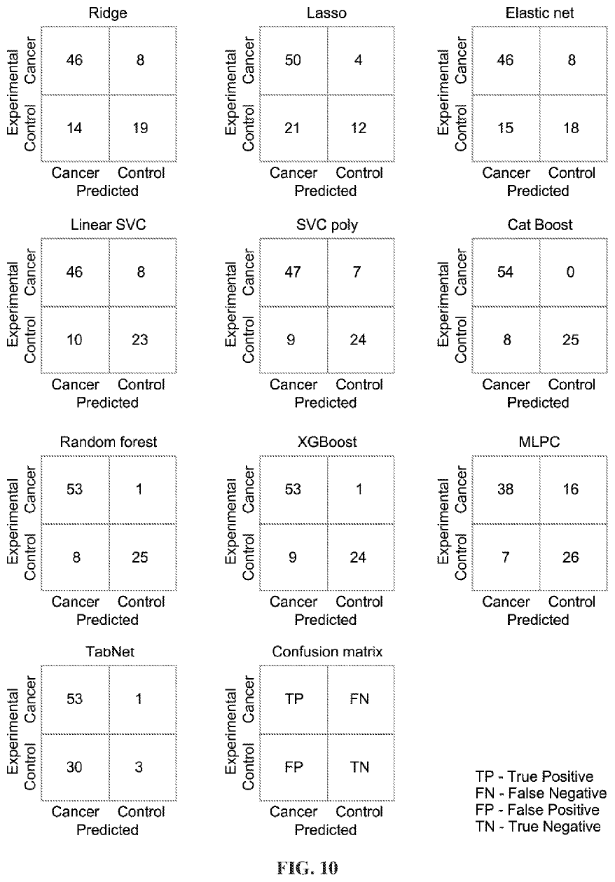

Models were further assessed using confusion matrices (FIG. 10) and accuracy, precision (positive predictive value, PPV), sensitivity (recall or true positive rate, TPR or detection rate, DR), specificity, negative predictive value (NPV), harmonic mean of precision and recall (F1 score), false discovery rate (FDR), and Matthew's correlation coefficient (MCC) scores (Table 1).

TABLE 1
Model Performance Assessment

| | | Precision | Sensitivity | | | | | |
|---|---|---|---|---|---|---|---|---|
| Classifier | Accuracy | (PPV) | (TPR/DR) | Specificity | NPV | F1 | FDR | MCC |
| Ridge | 74.71 | 76.67 | 85.19 | 57.58 | 70.37 | 80.70 | 23.33 | 43.09 |
| Lasso | 71.26 | 70.42 | 92.59 | 36.36 | 75.00 | 80.00 | 29.58 | 38.81 |
| Elastic | 73.56 | 75.41 | 85.19 | 54.55 | 69.23 | 80.00 | 24.59 | 41.12 |
| Net | | | | | | | | |
| Linear | 79.31 | 82.14 | 85.19 | 69.70 | 74.19 | 83.64 | 17.86 | 50.09 |
| SVC | | | | | | | | |
| SVC poly | 81.61 | 83.93 | 87.04 | 72.73 | 77.42 | 85.45 | 16.07 | 53.78 |
| Random | 89.66 | 86.89 | 98.15 | 75.76 | 96.15 | 92.17 | 13.11 | 69.28 |
| Forest | | | | | | | | |
| Cat Boost | 90.80 | 87.10 | 100.00 | 75.76 | 100.00 | 93.10 | 12.90 | 71.96 |
| XGBoost | 88.51 | 85.48 | 98.15 | 72.73 | 96.00 | 91.38 | 14.52 | 68.19 |
| Multi- | 73.56 | 84.44 | 70.37 | 78.79 | 61.90 | 76.77 | 15.56 | 40.03 |
| layer | | | | | | | | |
| Perceptron | | | | | | | | |
| TabNet | 64.37 | 63.86 | 98.15 | 9.09 | 75.00 | 77.37 | 36.14 | 20.42 |

DR: Detection Rate
F1: Harmonic mean of precision and sensitivity (recall)
FDR: False Discovery Rate
MCC: Matthew's Correlation Coefficient
NPV: Negative Predictive Value
PPV: Positive Predictive Value
SVC: Support Vector Classification
SVC poly: SVC polynomial kernel
TPR: True Positive Rate

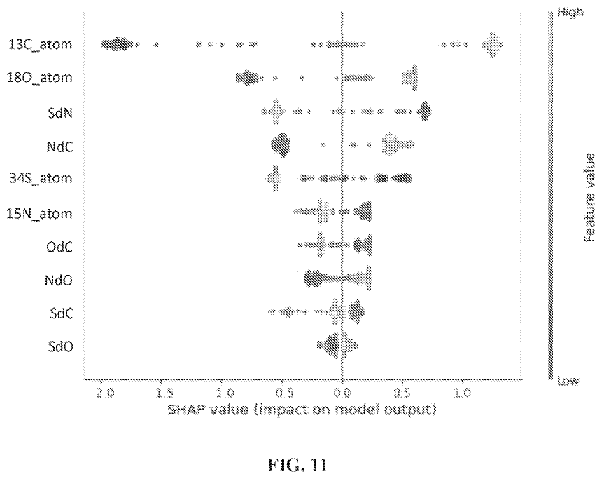

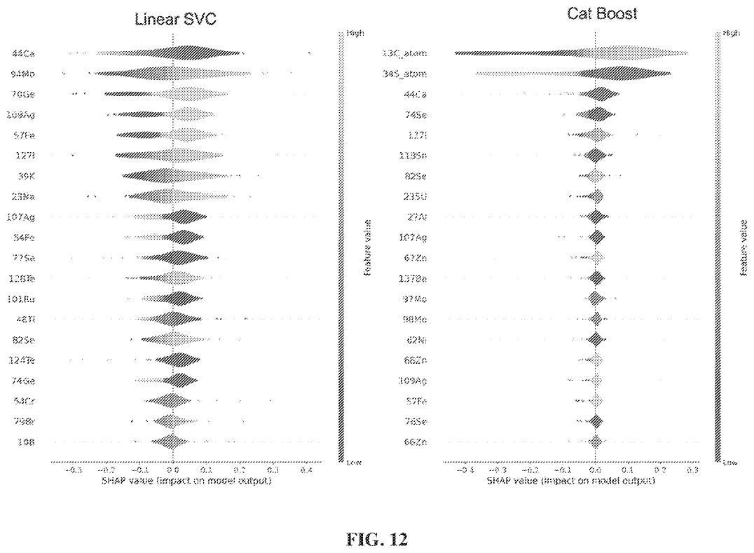

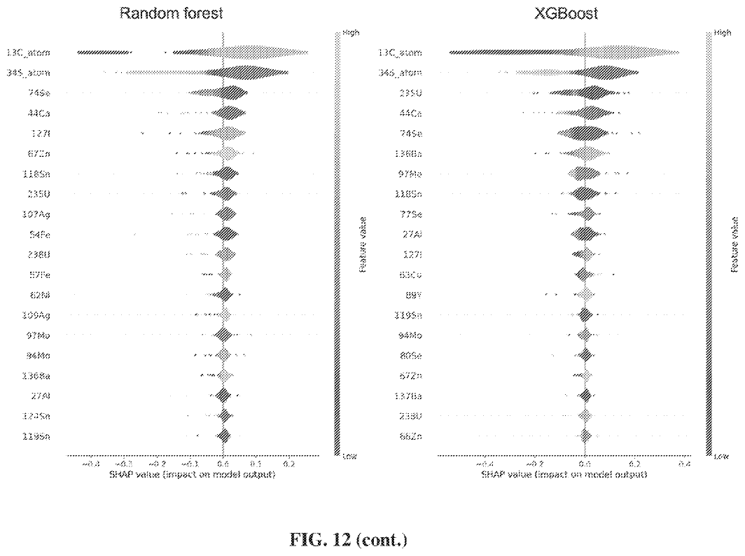

SHAP indicates what features give leverage to the prediction of cancer, wherein positive values on the x-axis contribute to the detection of cancer and negative values on the x-axis contribute to the detection of control. FIG. 11 illustrates SHAP values for the XGBoost Classifiers using test samples from prostate cancer only, analyzed for major elements only and their isotopes (fractional abundances, delta values, and overall concentration ratios). FIG. 12 illustrates SHAP values for four different models calculated using test samples from all cancer types in the study that were analyzed for majors and minor elements and their isotopes (fractional abundances, delta values, and overall concentration ratios).

### Discussion

This disclosure provides a proof-of-concept using 36 isotopic elements (91 isotopic features) from a collection of 433 samples (control/healthy: 171 and cancer: 262).

Isotope fingerprinting is a highly sensitive method and every small change in isotope abundance can hold a meaningful insight. However, analyzing higher dimensional data can be challenging. Preliminary data was initially analyzed using hierarchical cluster analysis and principal component analysis (PCA). With these limited dimensions, none of the methods were able to separate cancer samples from controls, suggesting a possibly non-linear relationship between features and outcomes.

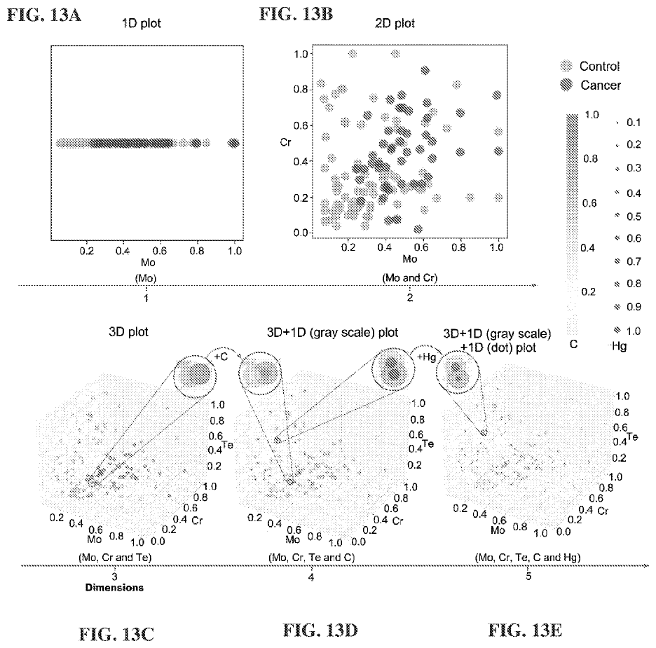

It was then observed that increasing the number of elements in the analysis increased the probability of separation of cancer and control groups. For example, while molybdenum (Mo) alone could only separate a few samples (FIG. 13A), the addition of chromium (Cr) slightly increased the probability (FIG. 13B). Predictive power was further improved by the addition of tellurium (Te) (FIG. 13C). Three dimensions are the limit of real-world dimensions that are easy to visualize and as such, carbon (C) was added as the color gradient of dots which further improved separation (FIG. 13D). Although a cancer sample can be plotted very close to a control sample, adding C values separated the two (see, e.g., that the control has a higher (white) value than cancer (brown) in FIG. 13D). By adding a fifth element, Hg, samples could be further separated as shown in FIG. 13E, where different sizes of circles correspond to the concentration of Hg. Collectively, this observation suggests that the addition of a number of dimensions of each sample increases the probability of separation of each group.

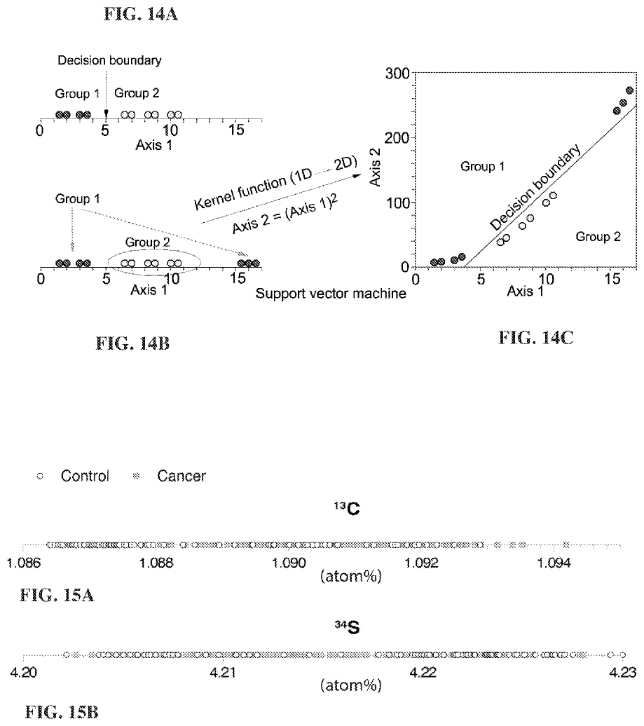

The human brain is capable of making a decision if there is a clear separation between groups of data points (e.g., FIG. 14A). Unfortunately, any complexity in the data makes the decision boundary almost impossible for the human brain (FIG. 14B). This requires a Kernel function to increase the dimension to identify the decision boundary (see hypothetical example in FIG. 14C). In the present example, there were 91 dimensions (i.e., 91 isotopic features), and the complexity of just two features, ¹³C and ³⁴S in FIG. 15A and FIG. 15B, respectively, necessitates the use of computer algorithms. Computer algorithms can work with thousands of dimensions and therefore can separate complex samples in a way that is beyond the human ability to correlate and comprehend data.

Considering the complexity of the data, it was hypothesized that an algorithm that evaluates multiple features at the same time would be useful to distinguish differences between cancer and control samples. Therefore, machine learning algorithms were used so that multiple features could be used to build the model for prediction.

Towards that end, the isotope abundance of 36 elements for 171 controls and 262 cancer samples were used to build a proof-of-concept model. Using algorithms from different classes of machine learning methods, we developed ten independent models. Models' performances were extensively tested using multiple parameters (FIG. 7, Table 1) and different sampling methods (FIG. 8). With a 80% training and 20% testing method, three regularized linear models (Ridge, Lasso, and Elastic net) displayed >70% accuracy, >70% precision, and >85% sensitivity, suggesting that the models can classify cancer samples well. However, all three models (Ridge, Lasso, and Elastic net) displayed specificity <60% with control samples. Two support vector classifiers (Linear SVC and SVC Poly), three tree-based ensemble classifiers (random forest, cat boost and XGBoost), and MLPC displayed higher predictive performance for both cancer and control samples. Across all 10 models, performance of TabNet was the worst. Overall, at least six models from three different classes of algorithms displayed considerable predictive performances (Table 1).

### Example 2: Further Validation of Machine Learning Models Involving Isotopic Features of Hair and Nail Samples to Predict Cancer in Subjects

This Example describes further validation of methods described in Example 1 to predict cancer in subjects by machine learning methods. In this Example, samples from additional subjects across different geographical regions and with different cancer types were used to prepare samples for analysis of isotopic features, and the data was used as input to train machine learning models. In the described methods, different strategies for normalizing the chemical data were used. In addition, the data was input to different machine learning models by either a general model with mixed data or specialized models in which data was specialized on different groups (e.g., geographical region, cancer type, gender, etc.) and accuracy for predicting cancer was determined on trained data and test data. The results demonstrate the methods can consistently predict cancer with an accuracy of greater than 70%, often with only a single isotopic feature, and that the accuracy of predictions can be further improved with additional isotopic features and/or different predictive models.

### Collection of Samples

Samples were collected at sites at Lund University (LU), Sweden and at the Brady Urological Institute at Johns Hopkins School of Medicine (JHU), USA. Cancer types covered in the collection were from patients with prostate, bladder, kidney, head-and-neck, leukemia, and breast cancer. Control samples were collected from individuals without a known cancer. Ethical permits for collections were: IRB00355957, NA_00087094 and IRB00372886 in the US and 2022-01350-01 in Sweden. Patients and controls were asked to deliver hair in pre-labelled vials. The samples were sent to LU and thereafter analyzed (destructively) for major and minor elemental concentrations using two different mass balance technologies. The mass balance technologies were inductively coupled plasma mass spectrometry (ICP-MS) and isotope-ratio mass spectrometry (IRMS).

### Reference Materials (Certified and Internal)

An internal reference material was developed from hair collected from control individuals in Sweden. The hair was homogenized at Chalmers University of Technology, using a ball mill (PVC) and shaker. The volume of powdered reference material ~2 liters in 2020. This internal reference material is called InH.

Certified and matrix-relevant (hair) reference materials were acquired and analyzed alongside samples and the internal reference material InH. The certified reference materials were IAEA-085 (certified for total concentrations of Hg, Fe, and Zn, see IAEA) and ERM-DB001 (certified for total concentrations of As, Cd, Cu, Hg, Pb, Se and Zn, see ERM). Measurements of InH were regularly compared to the certified reference standards.

### Preparation of a Multielement Calibration Standard and Blanks

A multielement standard was prepared using certified reference standards of high purity (Accustandard). Blank samples were prepared by acid in same glass vials were microwaved and kept in fridge until measurements with ICP-MS. Acid used for dilutions and calibration standards was of trace metal grade. Preparations of multielement standard and blank acid were done in the Hammarlund Laboratory, at Lund University, Sweden.

Chemical Analyses The hair samples and reference material were subsampled and analyzed in two separate processes. On one hand, samples were digested and then analyzed for the amount of minor element isotopes using ICP-MS. On the other hand, samples were analyzed for the amount of major element (C, N, O, S) isotopes using IRMS.

### A. Analyses of Minor Elements

The minor (trace) elements analyzed in hair samples and reference materials were: Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y and Zn.

### 1. Digestion of Hair Samples and Reference Material

Vials made of quartz glass (Milestone) were placed on the balance; and tared. A hair sample of 7.5 mg is weighed up in the glass vial. We used gloves to prevent fingerprints on the vials and anti-static bracelet at the balance. The vials were placed in a rack numbered 1-22, making one batch. After weighing up 20 hair samples and one InH sample, the rack was transferred to the fume hood. Into each vial, 4 ml 0.5 M nitric acid (69% Ultrapure from VWR) was added, using an acid proof dispenser. The vials were then moved to the 22-vial-rack of a UltraWave MA149 microwave (Milestone). Strands of hair not covered by the acid were pushed down using a glass stick. A last (22^{nd}) blank sample (4 ml 0.5 M nitric acid) was added to the batch. The vials were capped with teflon lids. In the Ultrawave, a baseload (150 ml of milliQ water and 5 ml 65% nitric acid) was mixed into the PTFE vessel. The outside of the PTFE vessel was carefully dried before placed in the microwave. The thermowell stick was placed into the recess of the rack, and the microwave and cooler were started. The 30 min program (200° C., 60 Bar) was started. After the run, the rack with samples was moved to its numbered dripping tray. The microwave was cleaned according to instructions each day. Samples were transferred to 15 ml tubes (Falcon). This solute was pooled and kept cool until analyses.

### 2. Materials

Water used during preparation was purified through ultra-filtration and ion exchange filter (Milli-Q quality, Millipore). Acid used for blank samples and for dilution of multielement calibration standards was prepared beforehand, using the same digestion procedure as for the hair samples (see Preparation of multielement standard and blanks). Calibrations was based on the multielement certified reference standards of high purity (Accustandard) prepared in the Hammarlund laboratory (see Preparation of multielement standard and blanks).

### 3. Analyses Methods

Samples were analyzed using a variety of ICP-MS analysis methods.

a. Method 1

ICP-MS analysis was performed on a Thermo iCAP TQe with a cyclonic quartz spray chamber, and a Micromist PFA nebulizer. Sample and skimmer cones used was of nickel and the insert was the High Matrix version. For sample introduction was an ESI SC-4DX FAST autosampler used in direct infusion mode (no loop-injection was used). Argon gas was used for the plasma and nebulizer while helium was used as collision gas.

For analysis, each sample was prepared at a 10× dilution. For the dilution; 500 μL of the sample solution obtained from the digest was transferred to a tube (13 mL polypropylene, screw cap) and then diluted with 4.5 mL of 2% nitric acid in water. To all tubes was added 50 μL internal standard solution, 200 ppb ppm of Terbium (Tb) and Iridium (Ir) in 2% nitric acid in water.

For calibration mixed standards CAL1, CAL2, CAL3, CAL4 was used (all from Accustandard). Calibrations was made at four concentrations, 10 parts per trillion (ppt), 100 ppt, 1 part per billion (ppb), and 10 ppb. To all tubes was added 50 μL internal standard solution, 200 ppb of Tb and Ir in 2% nitric acid in water. For the calibration curve, the multielement standard solution was diluted with the prepared blank to achieve four standard solutions (0, 4, 10, and 50 ppb). For every 22 samples, one sample of digested InH reference material was measured.

b. Method 2

Element analyses were done using a Bruker Aurora Elite quadrupole. Instrument was tuned using the Analytik Jena Tuning solution (with elements Be, Mg, Co, In, Ba, Ce, Tl, Pb, Th), aimed at obtaining high and stable signal counts on relevant isotopes and on low oxide production (below 2%) and low double charged signal (below 3%). Argon gas was used for the plasma and nebulizer while helium was used as collision gas. Due to high signals/baselines, the following isotopes were permanently attenuated to avoid detector over range signals: ²⁴Mg, ²⁷Al, ⁴⁴Ca, ⁴⁵Sc, ⁵²Cr, ⁵⁶Fe, ⁵⁸Ni, ⁶³Cu, ⁶⁴Zn, ⁶⁵Cu, ⁶⁶Zn, ⁶⁸Zn, ⁸⁸Sr, ¹³⁸Ba. Standard polyatomic interference correction was used, as included in the Quantum software.

Analytical sequences were setup to run automatically, starting with three blanks for warmup followed by four calibrations stands (diluted 1-500 times).

Internal standardization using Bismuth (Bi) and Praseodymium (Pr), at 3 ppb solution in 2% HNO₃, was applied via a Y-connection to the sample solution. For the calibration curve, the multielement standard solution was diluted with the prepared blank to achieve four standard solutions (0, 4, 10, and 50 ppb). After analyzing eight samples the sequence was recalibrated by analyzing one of the multielement calibration standard. A total of 24 samples were run per analytical sequence. For every 22 samples, one sample of digested internal reference material (hair) was measured.

c. Method 3

The analysis occurred with a Thermo iCAP Q-ICP-MS in KED mode (kinetic energy discrimination) to reduce polyatomic ion interferences derived from the plasma or vacuum interface in collision cell ICP-MS. Instrument was used in single-quadrupole mode but with collision gas (He) i.e., kinetic energy discrimination mode (no reaction gas was used). Radio-frequency (RF) generator power was 1550 W, optimal settings for gas flows, torch position and mass spectrometer settings were adjusted through the autotune procedure before start of analysis. Elements (mass-to-charge ratios, m/z) monitored was from m/z=6 (corresponding to lithium's lightest isotope) to m/z=238 (corresponding to uranium's heaviest isotope). Instrument was operated in peak-hopping mode, 50 sweeps/mass, with a dwell time of 30 ms.

The total volume Vtotal was 5 mL and the dilution factor 5.

2.0 mL internal standard Scandium (Sc) and Indium (In) at 2 ppb in HNO3 0.5M, and 2.75 mL HNO₃ 0.5M. For every 15 samples, one sample of digested internal reference material (hair) was measured.

### 4. Interferences

The ICP analyzes returns peak area of counts per second along a mass spectrum. However, isobaric elements can have the same weight, or complexes of elements or molecules can achieve the same weight as an element of interested. Therefore, the study excludes data on for example ⁸⁰Se since the complexation of two argon atoms (⁴⁰Ar+⁴⁰Ar) has the same weight. Also, the study excluded Germanium from the multielement standard since ⁷⁴Ge is of less interest than ⁷⁴Se. If measurements of masses are overlapping but with enough intensity, the software can assist in separating the overlap. For example, the intensity of ⁵¹Cr is high, such that ⁵¹Ti can be subtracted and ignored. However, the intensity of ⁵¹V is so low that no correction is robust, and its data should therefore be disregarded.

### 5. Accounting for Natural Abundances of Isotopes

The amount measured for each isotope is correlated to the calibration curve of concentrations. However, the calibration curve accounts for all isotopes of the element of interest. The fraction of individual isotopes within this element is known to follow its ‘natural abundance’. Therefore, the isotopic concentration should be corrected manually for the natural abundance of each isotope. To correct, the concentration achieved from the calibration curve was multiplied with the natural abundance of that isotope. For example, say ⁴⁷Ti amounts to 100 ppb when using the calibration curve for all Ti. Out of all Ti in nature, ⁴⁷Ti naturally accounts for 73.7% of all Ti isotopes. Therefore, this sample has 73.7 ppb of ⁴⁷Ti (100 ppb multiplied with 0.737). This correction was performed for all isotopes, using the concentration data.

For analyses under Method 3, ¹¹B was eliminated from the study since it interfered with ¹²C. The ICP-QMS instrument has a certain finite mass resolution, which means that strong signals may interfere with an adjacent mass number. For example, carbon-12 has a very strong signal on mass 12 that may intrude a on mass number 11.

### 6. Variation

Repeated analyses of the internal reference material (hair) allow monitoring of variance in the measurements. Variance is expressed as coefficient of variance (CV %) and calculated as the standard deviation divided with the mean. Usually, a CV % below 30 is acceptable [48]. For our measurements, CV % is generally >15 (e.g., Ca, Ti, Cr, Fe, Rb, Sr, Hg, and U isotopes) and 15-30 (e.g., Se isotopes). Isotopes that for sometimes fall above 30 are ⁹⁴Mo and Ni isotopes.

### B. Analyses of Major Elements (C, N, O, S)

The major elements analyzed in the hair samples and reference materials were C, N, S, and O.

### 1. Preparation and Analyses

Hair samples were cut in smaller bits into pre-weighted metal capsules 5×8 mm. Sample weights were noted. For C/N analyses, the metal capsules were packed up to small spheres. Powdered study-specific reference material (hair) was packed into capsules every 22 samples, weight noted. For S analyses, vanadium oxide V₅O was added before packing capsules to spheres. For O analyses, metal capsules are left open for −7 days to equilibrate. Standards were calibrated against certified reference materials. The samples were combusted in an elemental analyzer (EA) and then analyzed in an IRMS. The gases and standards involved depend on the element of interest (see below). Values are reported in the conventional δ-notation in per mille (also referred to herein as “permil,”, “per mill,” and “per mil,” which are used interchangeably) relative to agreed-upon standards. These standards are VPDB (Vienna Pee Dee Belemnite) for δ¹³C_{org}, relative to atmospheric N₂ for δ¹⁵N, relative to VCDT (Vienna Canon Diabolo Troilite) for δ³⁴S, and relative to V-SMOW for δ¹⁸O.

### 2. Analyses Methods

Samples were analyzed using a variety of IRMS analysis methods.

a. Method 1
i. Carbon and Nitrogen

Samples were weighed and wrapped in 5×9 mm pressed tin capsules and loaded into an autosampler, after which the samples and resulting gases were under continuous flow from an Ultrapure Helium gas stream at 180 ml/min. Samples were combusted with an aliquot of oxygen in a Flash IRMS EA Isolink (ThermoScientific). The combustion furnace was maintained at 1020° C. Water was removed with a magnesium perchlorate trap, CO₂ and N₂ gases were separated in a GC Column maintained at 70° C. Separated gases were interfaced via a ThermoScientific Conflo IV for introduction to a Delta V advantage mass spectrometer. Reproducibility of the analyses was checked by replicate analyses of laboratory standards (acetalinide and urea) calibrated to international standard IAEA 600 (caffeine) and was better than ±0.06 for δ¹³C_{org} and ±0.22 for δ¹⁵N. Values are reported in the conventional δ-notation in permil relative to VPDB (Vienna Pee Dee Belemnite) for δ¹³C_{org} and relative to atmospheric N₂ for δ¹⁵N.

b. Method 2

Samples were transferred to glass screw-capped vials and homogenized with surgical scissors. For δ¹³C and δ¹⁵N analysis 1.0±0.1 mg amounts of sample material was weighed into tin capsules (8×5 mm). For δ³⁴S analysis 1.0±0.1 mg amounts of sample material plus ca. 5 mg of vanadium pentoxide catalyst was weighed into tin capsules (8×5 mm). For δ¹⁸O analysis 2.0±0.1 mg amounts of sample material was weighed into silver capsules (8×5 mm).

i. Carbon and Nitrogen

Carbon and nitrogen isotope analysis of hair samples was undertaken by Elemental Analysis—Isotope Ratio Mass Spectrometry (EA-IRMS). Samples and references were weighed into tin capsules, sealed, and loaded into an auto-sampler on a Europa Scientific elemental analyzer. They were then dropped in sequence into a furnace held at 1000° C. and combusted in the presence of oxygen. The tin capsules were flash combusted, raising the temperature in the region of the sample to −1700° C. The combusted gases were swept in a helium stream over combustion catalyst (Cr₂O₃), copper oxide wires (to oxidize hydrocarbons), and silver wool to remove sulfur and halides. The resultant gases, N₂, NOₓ, H₂O, O₂, and CO₂, were swept through a reduction stage of pure copper wires held at 600° C. This removes any oxygen and converts NOₓ species to N₂. A magnesium perchlorate chemical trap was used to remove water. Nitrogen and carbon dioxide were separated using a packed column gas chromatograph held at a constant temperature of 65° C. The resultant nitrogen peak entered the ion source of the Europa Scientific 20-20 IRMS first, where it was ionized and accelerated. Nitrogen gas species of different mass were separated in a magnetic field then simultaneously measured using a Faraday cup collector array to measure the isotopomers of N₂ at m/z 28, 29, and 30. After a delay, the carbon dioxide peak entered the ion source and was ionized and accelerated. Carbon dioxide gas species of different mass were separated in a magnetic field then simultaneously measured using a Faraday cup collector array to measure the isotopomers of CO₂ at m/z 44, 45, and 46.

Both references and samples were converted to N₂ and CO₂ and analyzed using this method. The analysis proceeds in a batch process by which a reference was analyzed followed by a number of samples and then another reference. The reference material used for δ¹³C and δ¹⁵N analysis of hair samples was IA-R068 (soy protein, δ¹³C_{V-PDB}=−25.22‰, δ¹⁵N_{AIR}=0.99‰). IA-R068, IA-R038 (L-alanine, δ¹³C_{V-PDB}=−24.99‰, δ¹⁵N_{AIR}=−0.65‰), IA-R069 (tuna protein, δ¹³C_{V-PDB}=−18.88‰, δ¹⁵N_{AIR}=11.60‰) and a mixture of IAEA-C7 (oxalic acid, δ¹³C_{V-PDB}=−14.48‰) and IA-R046 (ammonium sulphate, δ¹⁵N_{AIR}=22.04‰) were run as quality control check samples during analysis of hair samples. IA-R068, IA-R038 and IA-R069 are calibrated against and traceable to IAEA-CH-6 (sucrose, δ¹³C_{V-PDB}=−10.449‰) and IAEA-N-1 (ammonium sulphate, δ¹⁵N_{AIR}=0.40‰). IA-R046 was calibrated against and traceable to IAEA-N-1. IAEA-C7, IAEA-CH-6, and IAEA-N-1, which are inter-laboratory comparison standards distributed by the International Atomic Energy Agency, Vienna.

ii. Sulfur

Sulfur isotope analysis of hair samples was undertaken by Elemental Analysis-Isotope Ratio Mass Spectrometry (EA-IRMS). Tin capsules containing reference or sample material plus vanadium pentoxide catalyst were loaded into an auto-sampler on a Europa Scientific elemental analyzer. From where they were dropped, in sequence, into a furnace held at 1080° C. and combusted in the presence of oxygen. Tin capsules flash combust, raising the temperature in the region of the sample to ~1700° C. The combusted gases were then swept in a helium stream over combustion catalysts (tungstic oxide/zirconium oxide) and through a reduction stage of high purity copper wires to produce SO₂, N₂, CO₂, and water. Water was removed using a Nafion™ membrane. Sulfur dioxide was resolved from N₂ and CO₂ on a packed GC column at a temperature of 32° C. The resultant SO₂ peak entered the ion source of the Europa Scientific 20-20 IRMS where upon it was ionized and accelerated. Gas species of different mass were separated in a magnetic field then simultaneously measured on a Faraday cup universal collector array. Analysis was based on monitoring of m/z 48, 49 and 50 of SO⁺ produced from SO₂ in the ion source.

Both references and samples were converted to SO₂ and analyzed using this method. The analysis proceeded in a batch process by which a reference was analyzed followed by a number of samples and then another reference. The reference material used for sulfur isotope analysis of hair samples was IA-R061 (barium sulphate, δ³⁴S_{V-CDT}=+20.33 ‰). IA-R061, IA-R025 (barium sulphate, δ³⁴S_{V-CDT}=+8.53‰) and IA-R026 (silver sulfide, δ³⁴S_{V-CDT}=+3.96‰) were used for calibration and correction of the ¹⁸O contribution to the SO⁺ ion beam. IA-R061, IA-R025 and IA-R026 are in-house standards calibrated against and traceable to NBS-127 (barium sulphate, δ³⁴S_{CDT}=+20.3‰) and IAEA-S-1 (silver sulfide, δ³⁴S_{V-CDT}=−0.30‰). For quality control purposes test samples of IA-R061, IAEA-SO-5 (barium sulphate, δ³⁴S_{V-CDT}=+0.50‰), IA-R068 (soy protein, δ³⁴S_{V-CDT}=+5.25‰) and IA-R069 (tuna protein, δ³⁴S_{V-CDT}=+18.91‰) were measured as quality control checks during batch analysis of hair samples. IA-R068 and IA-R069 are in-house standards calibrated against and traceable to NBS-127 and IAEA-SO-5. NBS-127, IAEA-S-1 and IAEA-SO-5 are inter-laboratory comparison standards distributed by the International Atomic Energy Agency (IAEA) with internationally accepted δ³⁴S values.

iii. Oxygen

Oxygen isotope analysis of hair samples was undertaken by Elemental Analyzer-Isotope Ratio Mass Spectrometry (EA-IRMS). Silver capsules containing sample or reference material were loaded into a zero blank autosampler, continually flushed with helium, on a Europa Scientific elemental analyzer. They were then dropped in sequence into a furnace at 1080° C. fitted with a quartz reactor tube lined with a glassy carbon film, which is filled to a height of 165 mm with glassy carbon chips and topped with a 15 mm layer of 50% nickelized carbon causing the samples to thermally decompose to H₂ and CO. Carbon monoxide and nitrogen were separated on a GC column packed with molecular sieve 5A at a temperature of 45° C. The IRMS used was a Europa Scientific 20-20 with triple Faraday cup collector array to monitor the masses 28, 29 and 30.

The reference material used for δ¹⁸O analysis of hair samples was IA-R006 (cane sugar, δ¹⁸O_{V-SMOW}=35.23‰). Samples of IA-R006, IAEA-CH-6 (sucrose, δ¹⁸O_{V-SMOW}=36.40‰), IAEA-CH-3 (cellulose, δ¹⁸O_{V-SMOW}=32.20% v) and IAEA-601 (benzoic acid, δ¹⁸O_{V-SMOW}=23.30‰) were measured along with hair samples as quality control check samples. IA-R006, IAEA-CH-6 and IAEA-CH-3 were oven dried at 60° C. for 7 days prior to analysis. Because IAEA-601 is liable to decompose on heating, it was not oven dried prior to analysis. IA-R006 has been calibrated against and is traceable to IAEA-CH-6. IAEA-CH-6, IAEA-CH-3 and IAEA-601 are inter-laboratory comparison standards distributed by the International Atomic Energy Agency, for which there are generally agreed δ¹⁸O values. Hair samples were comparatively equilibrated for 7 days prior to analysis. Four keratin standards: USGS42 (human hair, δ¹⁸O_{V-SMOW}=8.56‰); USGS43 (human hair, δ¹⁸O_{V-SMOW}=14.11‰); USGS CBS (caribou hoof, δ¹⁸O_{V-SMOW}=2.39‰) and USGS KHS (kudu horn, δ¹⁸O_{V-SMOW}=21.21‰) were comparatively equilibrated and analyzed alongside your samples as controls. The measured δ¹⁸O results for your samples were normalized using a 4-point linear calibration with USGS42, USGS43, USGS CBS and USGS KHS. USGS42, USGS43, USGS CBS and USGS KHS are inter-laboratory comparison standards distributed by the United States Geological Survey. c. Method 3

i. Carbon and Nitrogen

Stable isotope measurements were performed using a Thermo Scientific™ EA IsoLink™ IRMS System for CNSOH known as Flash EA. This is based on a flash dynamic combustion method, which produces complete combustion of the sample within a high temperature oxidation reactor, which allows the complete conversion of all samples to elemental gasses (N₂ and CO₂). The separation of dedicated gasses was carried out on the chromatography column, after gas separation, the gasses were introduced to the mass spectrometer by helium carrier gas. The FlashEA 1112 was connected with an IRMS system (Thermo Finnigan DeltaPLUS XP continuous-flow isotope ratio mass spectrometer) for the accurate determination of nitrogen and carbon isotope ratios.

Samples (0.25±0.04 mg) were weighed into ultra clean aluminium capsules for carbon analysis, then dropped into the autosampler of the elemental analyzer. Results were expressed in conventional delta notation as follows, δ (%)=(Rₛₐₘₚₗₑ/R_{reference}−1)*1000, where R is the ¹⁵N/¹⁴N, ¹³C/¹²C or ³⁴S/³²S ratio of the sample or reference standard and the δ¹⁵N, δ¹³C and δ³⁴S values are relative to AIR, VPDB and VCDT international reference points, respectively.

Combustion: The analytical method was based on the complete and instantaneous oxidation of the sample by flash combustion at 1020° C. which converts all sample substances into combustion products. Vanadium(V)oxide was weighed into the aluminium capsules next to the sample to increase the oxidation. The resulting combustion gases (in this case N₂ and CO₂) are swept into the chromatographic column by the carrier gas (helium). The gases were separated in the column and detected by the MS. Testing was performed by an ISO/IEC 17025 accredited external service provider.

d. Method 4
i. Carbon and Nitrogen

Samples for carbon and nitrogen isotopic analysis were converted to N₂ and CO₂ with an elemental analyzer; these two gases were separated with a GC, and the gases were analyzed with a continuous flow isotope ratio mass spectrometer [39]-[40]. Isotopic reference materials are interspersed with samples for calibration.

Reporting of Relative Carbon Isotope Ratios: Relative carbon isotopic results are reported in permil relative to VPDB (Vienna Peedee belemnite) and normalized (Coplen and others, 2006) on a scale such that the relative carbon isotope ratios of L-SVEC LiCO3 carbonate and NBS 19 CaCO3 are −46.6 and +1.95 permil, respectively. Although LSVEC is no longer used, stable carbon-isotope results are reported on the VPDB-LSVEC scale via normalization with other internationally distributed isotopic reference materials. The carbon isotopic compositions of carbon-bearing internationally distributed isotopic reference materials, had they been analyzed in any laboratory with any samples, are in accord with [41]. These relative carbon isotope ratios are shown in Table 2.

TABLE 2
Relative Carbon Isotope Ratios

| Reference | | Delta value (‰) for element |
|---|---|---|
| material name | Reference material | of interest (O, C, N) |
| NBS 19 | CaCO₃ | +1.95 |
| NBS 18 | CaCO₃ | −5.01 |
| IAEA-CO-1 | CaCO₃ | +2.49 |
| IAEA-CO-8 | CaCO₃ | −5.76 |
| IAEA-CO-9 | CaCO₃ | −47.32 |
| USGS24 | Graphite | −16.05 |
| NBS 22 | Oil | −30.02 |
| IAEA-CH-3 | Cellulose | −24.72 |
| IAEA-CH-6 | Sucrose | −10.45 |
| IAEA-CH-7 | Polyethylene | −32.15 |
| IAEA-600 | Caffeine | −27.73 |
| IAEA-601 | Benzoic acid | −28.81 |
| USGS40 | Glutamic acid | −26.39 |
| USGS41a | Glutamic acid | +36.55 |
| RM 8562 | CO₂ | −3.72 |
| RM 8563 | CO₂ | −41.59 |
| RM 8564 | CO₂ | −10.45 |

The 2-sigma uncertainty of carbon isotopic results was 0.5 permil unless otherwise indicated. This means that if the same sample were resubmitted for isotopic analysis, the newly measured value would lie within the uncertainty bounds 95 percent of the time.

Reporting of Nitrogen Isotope Ratios: Nitrogen isotope ratios are reported in parts per thousand (permil) relative to N2 in air. The nitrogen isotopic compositions of nitrogen-bearing internationally distributed isotopic reference materials, had they been analyzed in any laboratory with any samples, are in accord with [42]-[44]. These relative nitrogen isotope ratios are shown in Table 3.

TABLE 3
Relative Nitrogen Isotope Ratios

| Reference | | Delta value (‰) of element |
|---|---|---|
| material name | Reference material | of interest (S, O, N) |
| N2 in air | | 0 (exactly) |
| IAEA-N-1 | (NH₄)₂SO₄ | +0.43 |
| IAEA-N-2 | (NH₄)₂SO₄ | +20.41 |
| IAEA-NO-3 | KNO₃ | +4.72 |
| USGS25 | (NH₄)₂SO₄ | −30.41 |
| USGS26 | (NH₄)₂SO₄ | +53.70 |
| USGS32 | KNO₃ | +180 (exactly) |
| USGS34 | KNO₃ | −1.8 |
| USGS35 | NaNO₃ | +2.7 |
| USGS40 | Glutamic acid | −4.52 |
| USGS41a | Glutamic acid | +47.55 |

The 2-sigma uncertainty of nitrogen isotopic results was 0.5 permil, unless otherwise indicated. This means that if the same sample were resubmitted for isotopic analysis, the newly measured value would lie within the uncertainty bounds 95 percent of the time.

ii. Sulfur

Dissolved sulfide samples are collected and prepared for isotopic analysis [45]. Samples are analyzed by conversion to sulfur dioxide with an elemental analyzer and by analysis with a continuous flow isotope ratio mass spectrometer [46]. Samples were analyzed with Ag2S reference materials, and no correction for oxygen isotopic composition is made.

Reporting of Sulfur Isotope Ratios: Sulfur isotope ratios are reported in parts per thousand (permil) relative to VCDT, defined by assigning a value of −0.3 permil exactly (Coplen and Krouse, 1998) to IAEA-S-1 silver sulfide (previously known as NZ-1). The sulfur isotopic compositions of sulfur-bearing internationally distributed isotopic reference materials, had they been analyzed in any laboratory with any samples, are in accord with [47]. These relative sulfur isotope ratios are shown in Table 4.

TABLE 4
Relative Sulfur Isotope Ratios

| Reference | | Delta value (‰) of element |
|---|---|---|
| material name | Reference material | of interest (S) |
| IAEA-S-1 | Ag₂S | −0.3 (exactly) |
| IAEA-S-2 | Ag₂S | +22.67 |
| IAEA-S-3 | Ag₂S | −32.55 |
| IAEA-S-5 | BaSO₄ | +0.5 |
| IAEA-S-6 | BaSO₄ | −34.05 |
| NBS 127 | BaSO₄ | +21.1 |
| Soufre de Lacq | Elemental S | +16.90 |

The 2-sigma uncertainty of sulfur isotopic results was 0.4 permil, unless otherwise indicated. This means that if the same sample were resubmitted for isotopic analysis, the newly measured value would lie within the uncertainty bounds 95 percent of the time.

e. Method 5
i. Carbon and Nitrogen

Stable isotopes were measured on an Thermo Scientific™ EA IsoLink™ IRMS System for CNSOH known as Flash EA, which is an enhanced elemental analyzer for nitrogen and carbon isotope analyses as well. This is based on a flash dynamic combustion method, which produces complete combustion of the sample within a high temperature oxidation reactor, which allows the complete conversion of all samples, to elemental gases (N₂ and CO₂). The separation of dedicated gases was carried out on chromatography column, after gas separation, and the gases were introduced to the mass spectrometer by helium carrier gas. The FlashEA 1112 was connected with an IRMS system (ThermoScientific DeltaQ continuous-flow isotope ratio mass spectrometer) for the accurate determination of nitrogen and carbon isotope ratios.

Samples (0.25±0.30 mg) were weighed into ultra clean aluminum capsules for carbon analysis, then dropped into the autosampler of the elemental analyzer. Results are expressed in conventional delta notation as follows, δ (%)=(Rₛₐₘₚₗₑ/R_{reference}−1)*1000, where R is the ¹⁵N/¹⁴N or ¹³C/¹²C ratio of the sample or reference standard and the δ¹⁵N and δ¹³C values are relative to AIR and VPDB international reference points, respectively. Samples were measured using international reference material for δ¹³C standardization the use of IAEA-600 and IVA33802159. IAEA-600 and IVA33802155 were used for δ¹⁵N standardization. Sulfanilamide in-house laboratory reference material was used for δ¹⁵N and δ¹³C measurement quality control which calibrated using of IAEA-600 IVA 33802151 and IVA33802159. The external precision based on the reproducibility of quality control samples during analysis of samples was better than 0.15‰ for δ¹³C and 0.15%, for δ¹⁵N.

Combustion: The analytical method was based on the complete and instantaneous oxidation of the sample by flash combustion at 1020° C. which converts all sample substances into combustion products. Vanadium(V)oxide were weighted into the aluminum capsules next to the sample to increase the oxidation. The resulting combustion gases (in this case N₂ and CO₂) were swept into the chromatographic column by the carrier gas (helium). The gases were separated in the column and detected by MS.

ii. Sulfur

Stable isotope measurements are performed using a Thermo Scientific™ EA IsoLink™ IRMS System for CNSOH known as Flash EA, which is an enhanced elemental analyzer for sulfur isotope analyses. This is based on a flash dynamic combustion method, which produces complete combustion of the sample within a high temperature oxidation reactor, which allows the complete conversion of all samples to elemental gases (SO₂). The separation of dedicated gases was carried out on chromatography column, after gas separation, and the gases are introduced to the mass spectrometer by helium carrier gas. The FlashEA 1112 was connected to an IRMS system (Thermo Finnigan Delta^{PLUS} XP continuous-flow isotope ratio mass spectrometer) for the accurate determination of sulfur isotope ratios.

Samples (0.45-0.50 mg) were weighed into ultra clean aluminum capsules for sulfur analysis, then dropped into the autosampler of the elemental analyzer. Results are expressed in conventional delta notation as follows, δ (%)=(Rₛₐₘₚₗₑ/R_{reference-1})*1000, where R is the ³⁴S/³²S ratio of the sample or reference standard δ³⁴S values are relative to VCDT international reference points. The following international reference materials were used for normalization of the measured samples: IAEA-SO-5, IAEA-SO-6 and NBS-127. Sulfanilamide in-house laboratory reference material was used for δ³⁴S measurement as quality control sample. The external precision obtained for our test samples during analysis of samples was better than 0.4% c.

Combustion: The analytical method was based on the complete and instantaneous oxidation of the sample by flash combustion at 1020° C. which converts all sample substances into combustion products. Vanadium(V)oxide is weighed into the aluminum capsules next to the sample to increase the oxidation. The resulting combustion gases (N₂, CO₂ and SO₂) were swept into the chromatographic column by the carrier gas (helium). The gases (N₂, CO₂ and SO₂) were separated in the column and the measurement dedicated gas or gases are detected by the MS.

### 3. Variation

a. Carbon

For analyses done by Method 1, performance was better than ±0.06 for δ¹³C_{org}. For analyses done by Method 2, maximum uncertainty was 0.23‰ for δ15N, 0.26‰ for δ13C, 0.42‰ for δ34S, and 0.23‰ for δ¹⁸O. For analyses Method 4, the 2-sigma uncertainty of C isotopic results is 0.5% o. For analyses done by Method 5, the external precision based on the reproducibility of quality control samples during analysis of samples was better than 0.15‰ for δ¹³C.

b. Nitrogen

For analyses done by Method 1, performance was better than ±0.22 for δ¹⁵N. For analyses done by Method 2, maximum uncertainty was 0.23‰ for δ15N. For analyses at Method 4, the 2-sigma uncertainty of N isotopic results is 0.5% o. For analyses done by Method 5, the external precision based on the reproducibility of quality control samples during analysis of samples was better than 0.15‰ for δ¹⁵N.

c. Sulfur

For analyses done by Method 2, maximum uncertainty was 0.42‰ for δ³⁴S. For analyses at Method 4, The 2-sigma uncertainty of sulfur isotopic results is 0.4 permil. For analyses done by Method 5, the external precision obtained for our test samples during analysis of samples was better than 0.4% c.

d. Oxygen

For analyses done by Method 2, maximum uncertainty was 0.23‰ for δ18O.

### Machine Learning

### A. Data Preprocessing

The samples were subjected to isotopic analysis of 39 minor elements (Li, B, Na, Mg, Al, P, K, Sc, Ca, Ti, V, Cr, Mn, Fe, Co, Ni, Cu, Zn, Ge, As, Se, Br, Rb, Sr, Y, Zr, Nb, Mo, Ru, Ag, Cd, In, Sn, I, Te, Ba, Hg, Pb, U) and four major elements (C, N, S, O). However, due to the availability of significant amounts of data, isotopes from only 24 elements (Li, B, Mg, Ca, Ti, Cr, Fe, Ni, Cu, Zn, Se, Br, Rb, Sr, Mo, Ag, Sn, Ba, Hg, Pb, U, C, N, S) were included in the final analysis. The missing values in the dataset were addressed through a two-step imputation process. Initially, missing values in the minor isotopes' concentration data were imputed using the IterativeImputer from Sci-Kit Learn. The IterativeImputer was also employed to address missing values in the major isotopes' fractional abundance data.

### B. Alignment and Normalization of Chemical Data

The isotopic abundances and their relationship to each other can be expressed by normalization in a number of different formats. The formats include fractional abundances (normalized ratio of one isotope to other isotopes of the same element), atomic percent, or delta values (a fractional abundance of isotope in a sample is normalized to the same or a different fractional abundance in a reference material). The delta values can be expressed in a number of different formats. In the conventional format, a fractional abundance in the sample is normalized to the same fractional abundance in a reference material (certified or study-specific). The study-specific reference material used here (InH) is developed from homogenized hair and continuously analyzed along with samples. In this study, non-similar element-based delta values are calculated by fractional abundances in the sample being normalized to the isotopic balance of nitrogen isotopes in the sample. The isotopic balance of nitrogen isotopes in the samples is calculated in two formats. For a simple-nitrogen (non-similar) based delta value (SimpleNδ), the ratio in the denominator is ¹⁵N in the sample normalized to ¹⁴N in the sample. For a pseudo-nitrogen (non-similar) based delta value (PseudoNδ), the ratio in the denominator is ¹⁵N in the sample normalized to ¹⁴N+¹⁵N in the sample. A fractional abundance (ⁿX) in the sample can therefore be normalized to nitrogen mass balance in the sample in two ways (SimpleNδⁿX and PseudoNδⁿX). These conversions to delta values (using study-specific reference material or non-similar based delta values) allows us to amalgamate results for the isotopic composition of major element (C, N, S, O) that are reported in delta values (using agreed-upon standards). However, we can also amalgamate data in the form of fractional abundances. Then we have to convert the delta values reported for major elements, as explained below.

1. Fractional Abundance Values: As Calculated from Concentrations

Fractional abundances were calculated using measured concentrations and imputed values of isotopes of major elements (C, N, S) and of minor elements. Fractional abundance was calculated by dividing the amount (concentration) of the elemental isotope of interest (e.g., ¹³C) by the amount (concentration) of one or more stable isotopes of interest. For example, a fractional abundance can be calculated by dividing the amount (concentration) of the elemental isotope of interest (e.g., ¹³C) by the total amount (concentration) of one or more stable isotopes of interest of that element (e.g., ¹²C+¹³C). Here, the ratio between the concentration of a specific isotope and the sum of concentrations of all measured isotopes for that element was calculated. This results in the fractional abundances for C, N and S (3 features) and minor elements (72 features), totaling 75 different isotopic features. Fractional abundance can also be calculated by dividing the amount (concentration) of the elemental isotope of interest (e.g., ¹³C) by 1 minus the total amount (concentration) of the same isotope of interest (e.g., ¹³C).

### 2. Delta Values

For major elements C, N, and S, the acquired delta values are generated through a comparison of the fractional abundance in the sample and the same fractional abundance in an agreed upon standard or reference material (e.g., V-CDT, V-SMOW or V-PDB). The comparison was calculated for element X with mass n as in the formula below:

$${δ}^{n} {X}_{Standard} = \frac{{}^{n} {R}_{Sample}}{{n}^{} {R}_{Standard}} - 1.$$

To convert delta values back to fractional abundances, the agreed-upon ‘absolute isotopic ratio’ or R value for the certified standards for the isotopes of interest (ⁿR_{standard}) was used. For example, the R value for ³⁴S/³²S in the agreed-upon standard (a meteorite) is 0.04416259 [49]. Delta values can also be converted to atomic % of ³⁴S by a simpler canonical method with R(³⁴S/³²S)Std=0.0450045, which is an older accepted value reported by [50]. The same conversion method was used for all elements with δ³⁴S, δ¹³C, or δ¹⁵N values, regardless of the laboratory in which the analysis was performed. δ³⁴S δ¹³C δ¹⁵N values were converted using the conversion method of [37]. Then, the ‘absolute isotopic ratio’ R value for the sample was used to calculate the fractional abundance value (F):

$${}^{n} {R}_{Standard} = {}^{n} F / ( 1 - {}^{n} F ) .$$

That means that, for example, a δ¹³C value can be directly converted to a fractional abundance for ¹³C by the following formula:

$${δ}^{13} {C}_{Standard} = [ \frac{{{(}^{13} F / ( 1 - {}^{13} F ) )}_{Sample}}{{{(}^{13} F / ( 1 - {}^{13} F ) )}_{Standard}} - 1 ] .$$

3. Atomic % Values: As Converted from Fractional Abundance Values

The fractional abundance can be converted to atomic % values by diving the value with 100.

### 4. Constructing Additional Delta Values

In this study, two additional different normalization strategies were developed. This normalization builds on the same rationale as delta values developed within the geoscience community. The difference is that reference material used in the present Example is not yet agreed upon by the geoscience community or certified by an organization like the IEAE. In this study, the first strategy uses the study-specific reference material (InH) that has been homogenized and mixed in a larger quantity. The second strategy uses the fractional abundance of nitrogen preserved in each hair sample to normalize the fractional abundances of other isotopes. These two normalization strategies are called ‘Study-specific delta values’ and ‘Nitrogen-based delta values’.

a. Study-Specific Delta Values

The fractional abundance of an isotope can also be compared to the same fractional abundance, where the latter serves as a reference value. Therefore, the same fractional abundance values were calculated for the internal reference material (InH), as for the hair samples from individuals with and without cancer.

$${δ}^{n} {X}_{InH} = [ \frac{{{(}^{n} F / ( 1 - {}^{n} F ) )}_{Sample}}{{{(}^{n} F / ( 1 - {}^{n} F ) )}_{InH}} - 1 ] .$$

Just like with conventional delta values, this study-specific delta value can be expressed as is or as permil (‰), that implies a multiplication of the value by 1000. For example, a δ⁴⁸Ti_{InH} value can be expressed as 0.0001 or 0.1% o. The R values (¹⁰B: 0.2516, ¹¹B: 3.9742, ²⁴Mg: 3.7577, ²⁵Mg: 0.1109, ²⁶Mg: 0.1241, 43Ca: 0.0857, 44Ca: 11.6743, 46Ti: 0.0875, ⁴⁷Ti: 0.0727, ⁴⁸Ti: 4.0009, ⁴⁹Ti: 0.0545, ⁵⁰Cr: 0.0384, ⁵²Cr: 6.4160, ⁵³Cr: 0.1084, ⁵⁶Fe: 43.8579, ⁵⁷Fe: 0.0228, ⁵⁸Ni: 4.8796, ⁶⁰Ni: 0.1672, ⁶¹Ni: 0.0069, ⁶²Ni: 0.0204, ⁶³Cu: 2.2334, ⁶⁵Cu: 0.4477, ⁶⁴Zn: 1.0914, ⁶⁶Zn: 0.3579, ⁶⁷Zn: 0.0400, ⁶⁸Zn: 0.2138, ⁷⁷Se: 0.2274, ⁷⁸Se: 1.4846, ⁸²Se: 0.2774, ⁷⁹Br: 0.9912, ⁸¹Br: 1.0089, ⁸⁵Rb: 4.6229, ⁸⁷Rb: 0.2163, ⁸⁴Sr: 0.0063, ⁸⁶Sr: 0.1037, ⁸⁷Sr: 0.0761, ⁸⁸Sr: 4.8507, ¹⁰⁷Ag: 1.0915, ¹⁰⁹Ag: 0.9162, ¹¹⁶Sn: 0.2001, ¹¹⁷Sn: 0.0968, ¹¹⁸Sn: 0.3862, ¹¹⁹Sn: 0.1012, ¹²⁰Sn: 0.5986, ¹³⁵Ba: 0.0736, ¹³⁶Ba: 0.0885, ¹³⁷Ba: 0.1315, ¹³⁸Ba: 2.7577, ¹⁹⁹Hg: 0.2588, ²⁰⁰Hg: 0.3870, ²⁰¹Hg: 0.1841, ²⁰²Hg: 0.5624, ²⁰⁶Pb: 0.3249, ²⁰⁷Pb: 0.2855, ²⁰⁸Pb: 1.1400, ²³⁵U: 0.0284, ²³⁸U: 35.2487) for the reference material InH that we have used calculation for examples presented here can be changed.

b. Non-Similar Element-Based Delta Value (e.g., Nitrogen-Based Delta Values)

Isotope ratios of trace elements (X) can also be compared to the fractional abundance of nitrogen (F¹⁵N) in the hair sample (from one or several patients) that takes into account both ¹⁴N and ¹⁵N in the fractional abundance in the denominator. This nitrogen-based calculation was called PseudoN-delta values, and calculated using the formula:

$$PseudoN {δ}^{n} X = [ ( \frac{{F}^{n} X}{{F}^{15} N} - 1 ) / 1000 ]$$

In this study, all minor elements were converted to these F¹⁵N-based PseudoN-delta values, but δ¹³C and δ³⁴S were retained as standard delta values. A nitrogen-based delta value that only takes into account ¹⁴N in the fractional abundance in the denominator was also calculated. This is called simple-nitrogen-based delta value, calculated using the formula:

$$SimpleN {δ}^{n} X = [ \frac{( {F}^{n} X / ( 1 - {F}^{n} X ) )}{( {F}^{15} N / ( 1 - {F}^{15} N ) )} - 1 ] / 1000$$

### C. Feature Selection, in General

Fractional abundance values, atomic % values, or delta values (conventional and developed) for the selected major and minor elements were amalgamated in one dataset.

Features can be selected systematically using machine learning algorithms, for example, starting from one feature and then adding one after another, or using statistical modelling, for example, Model-X knockoffs and scikit-learn python implementations for feature selection. In the following tests, one to all features were tested for during training and performance tests of the algorithms. When needed, features can be selected using Model-X knockoffs implemented in DeepLINK [31] and using SequentialFeatureSelector (scikit-learn python implementation).

### D. Models, In General

Ten different machine learning algorithms (Ridge, Lasso, Elastic Net, Linear SVC, SVC Poly, Random Forest, Cat Boost, XGBoost, MLPC and TabNet) were used to build binary prediction models. Out of 87 features (delta of C and S, fractional abundances, and nitrogen-normalized delta-values), a different number of parameters were used to build each test. Data from 80% of the samples was used to build or train the model (including validation data), and data from 20% of the samples was used for testing the model's performance. Model parameters were optimized following the guidelines provided by the algorithm developer. To identify which features are informative for the prediction, the python package SHAP was used [32].

### Overview: Three Tests

The sample and chemical data was binned for three separate strategies of test development. The strategies area built on, in short:
1. Test 1: A first cohort with chemical data from hair obtained from US men with and without prostate cancer was used for training an algorithm. This cohort encompassed hair samples from 282 prostate cancer patients and 396 control subjects. Here, tests were separately developed, using 1, 2, 3, 4 and more parameters. The accuracy for this algorithm to detect other cancers, or prostate cancer from patients from different geographical regions, was explored.
2. Test 2: A second cohort with chemical data from hair obtained from US men and women with and without different cancers was used for training an algorithm. Specifically, this cohort encompassed hair samples from US men and women with and without basal cell skin cancer (n=2), bladder cancer (n=58), colon cancer (n=1), Hodgkin's lymphoma (n=1), kidney cancer (n=6), leukemia (n=1), liver cancer (n=2), liver and bone cancer (n=1), lung cancer (n=1), lymphoma (n=1), melanoma (n=1), prostate cancer (n=282), prostate cancer and chronic lymphocytic leukemia (n=1), rectal cancer and non-Hodgkin's lymphoma (n=1), squamous cell carcinoma (SCC) and basal cell carcinoma (BCC) (n=1), skin cancer (n=3), skin-melanoma and prostate cancer (n=1), skin cancer and maybe prostate cancer (n=1), testicular cancer (n=3), thyroid cancer (n=1), tonsil cancer (n=1), and cancer not disclosed (n=3). This cohort encompassed hair samples from 373 total patients, (12 females, 334 males, and 27 not disclosed) and 437 control subjects (395 men, 16 women, and 26 not disclosed). The accuracy for this algorithm to detect cancer from samples from patients from different geographical regions was explored.
3. Test 3: A third cohort with chemical data from hair obtained from US and Sweden men and women with and without different cancers was used for training an algorithm. Specifically, this cohort encompassed hair samples from patients having basal cell skin cancer (n=2), bladder cancer (n=61), breast cancer (n=15), colon cancer (n=1), Hodgkin's lymphoma (n=1), kidney cancer (n=6), leukemia (n=1), liver cancer (n=2), liver and bone cancer (n=1), lung cancer (n=1), lymphoma (n=1), melanoma (n=1), prostate cancer (n=347), prostate cancer and chronic lymphocytic leukemia (n=1), renal cancer (n=2), renal cell carcinoma (n=1), SCC and BCC (n=1), skin cancer (n=3), skin-melanoma and prostate cancer (n=1), skin and maybe prostate cancer (n=1), testicular cancer (n=3), thyroid cancer (n=1), cancer not disclosed (n=3), and tonsil cancer (n=1). This cohort encompassed hair samples from both US cancer patients (n=373) and US control subjects (n=437), and Sweden cancer patients (n=83) and Sweden control subjects (n=245). The cohort was comprised of 400 male cancer patients, 448 male control subjects, 27 female cancer patients, and 147 female control subjects. The accuracy for this algorithm to detect cancer from samples from patients from different geographical regions was explored.
   A. Test 1: Cohort of US Males with or without Prostate Cancer

### Results for Test 1A: Single Parameter Test

The approach initially involved conducting a single (n=1) parameter (e.g., isotopic feature) search within the dataset to determine if any specific isotope of an isotopic element could effectively identify cancer. The data were randomly divided into two groups: a training group (comprising 203 cancer cases and 271 control cases) and a test group (comprising 79 cancer cases and 125 control cases). Single isotopes from a single isotopic element, specifically F⁶⁴Zn and F⁶⁶Zn, yielded train accuracies exceeding 80% (F⁶⁶Zn: train accuracy 80.6%, test accuracy 81.9%; F⁶⁴Zn: train accuracy 80.2%, test accuracy 80.4%).

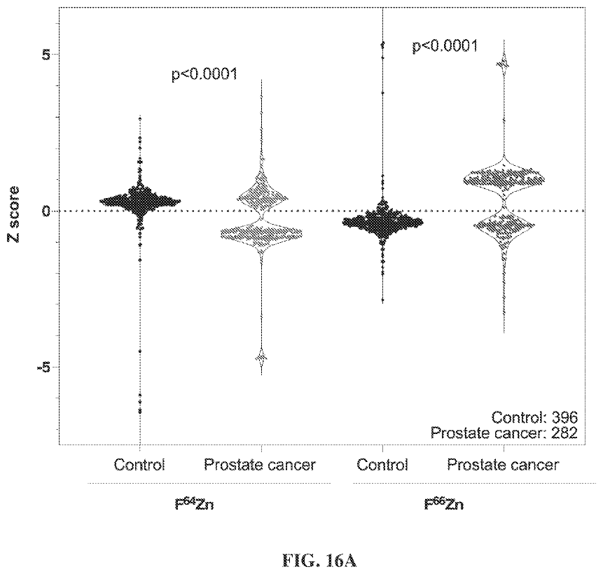

As illustrated in FIG. 16A, it was observed that control and cancer samples could be distinguished by using only e.g., F⁶Zn or F⁶⁶Zn with a high accuracy. For a single isotopic features fractional abundance of an isotope of isotopic element), accuracy for training and testing for the top 10 isotopic features are listed in Table 5.

TABLE 5
Accuracy for Top 10 Features for Single Parameter Test.

| Feature 1 | Accuracy Training | Accuracy Testing |
|---|---|---|
| F66Zn | 0.806 | 0.819 |
| F64Zn | 0.802 | 0.804 |
| F87Sr | 0.795 | 0.789 |
| F88Sr | 0.772 | 0.760 |
| F47Ti | 0.724 | 0.735 |
| F48Ti | 0.743 | 0.706 |
| F46Ti | 0.715 | 0.730 |
| F68Zn | 0.698 | 0.730 |
| F50Cr | 0.724 | 0.701 |
| F86Sr | 0.709 | 0.711 |

### Results for Test 1B: The 2-4 Parameter Test

Next, an XGBoost classifier was implemented to evaluate combinations of two isotopic features (n=2), encompassing 1,711 possible pairs of isotopic features. Adding an isotope of a second isotopic element to either f⁶⁴Zn or f⁶⁶Zn significantly enhanced the predictive performance. For instance, pairing F⁶⁴Zn with F¹¹⁶Sn resulted in a train accuracy of 86.5% and a test accuracy of 84.3%, while combining it with F³⁴S achieved a train accuracy of 82.3% and a test accuracy of 84.3%. Similarly, combining F⁶⁶Zn with F¹¹⁶Sn yielded a train accuracy of 87.1% and a test accuracy of 84.3%, and pairing it with F³⁴S led to a train accuracy of 85.0% and a test accuracy of 86.3%. For combinations of two isotopic features (fractional abundance of isotopes of isotopic elements), accuracy for training and testing for the top 10 isotopic features are listed in Table 6.

TABLE 6
Accuracies for Top 10 Features for Two Parameter Test.

| Feature 1 | Feature 2 | Accuracy Training | Accuracy Testing |
|---|---|---|---|
| F66Zn | F116Sn | 0.871 | 0.843 |
| F34S | F66Zn | 0.850 | 0.863 |
| F64Zn | F116Sn | 0.865 | 0.843 |
| F87Sr | F116Sn | 0.861 | 0.843 |
| F66Zn | F119Sn | 0.854 | 0.833 |
| F66Zn | F120Sn | 0.857 | 0.828 |
| F88Sr | F116Sn | 0.857 | 0.828 |
| F64Zn | F119Sn | 0.844 | 0.838 |
| F66Zn | F118Sn | 0.846 | 0.833 |
| F34S | F64Zn | 0.829 | 0.848 |

Notably, including three isotopic features (n=3), such as F⁶⁴Zn, F³⁴S, and F¹¹⁶Sn (train accuracy: 91.1%, test accuracy: 90.2%) or F⁶⁶Zn, F³⁴S, and F¹¹⁶Sn (train accuracy: 90.5%, test accuracy: 91.2%), significantly improved the predictive performance. For the combinations of three isotopic features (fractional abundance of isotopes of isotopic elements), accuracy for training and testing for the top 10 isotopic features are listed in Table 7.

TABLE 7
Accuracies for Top 10 Features for Three Parameter Test.

| | | | Accuracy | |
|---|---|---|---|---|
| Feature 1 | Feature 2 | Feature 3 | Training | Accuracy Testing |
| F34S | F66Zn | F116Sn | 0.905 | 0.912 |
| F34S | F64Zn | F116Sn | 0.911 | 0.902 |
| F34S | F66Zn | F201Hg | 0.905 | 0.902 |
| F34S | F66Zn | F235U | 0.888 | 0.917 |
| F34S | F66Zn | F238U | 0.888 | 0.917 |
| F66Zn | F116Sn | F135Ba | 0.895 | 0.902 |
| F10B | F66Zn | F116Sn | 0.888 | 0.907 |
| F11B | F66Zn | F116Sn | 0.888 | 0.907 |
| F66Zn | F79Br | F116Sn | 0.905 | 0.887 |
| F66Zn | F81Br | F116Sn | 0.905 | 0.887 |

In addition, combinations of any four isotopic features (n=4), amounting to 455,126 combinations, were explored. For the combinations of four isotopic features (fractional abundance of isotopes of isotopic elements), accuracy for training and testing for the top 10 isotopic features are listed in Table 8.

TABLE 8
Accuracies for Top 10 Features for Four Parameter Test.

| | | | | Accuracy | Accuracy |
|---|---|---|---|---|---|
| Feature 1 | Feature 2 | Feature 3 | Feature 4 | Training | Testing |
| F34S | F66Zn | F116Sn | F200Hg | 0.907 | 0.907 |
| F34S | F53Cr | F66Zn | F116Sn | 0.907 | 0.907 |
| F34S | F66Zn | F116Sn | F135Ba | 0.905 | 0.907 |
| F34S | F66Zn | F116Sn | F138Ba | 0.905 | 0.907 |
| F34S | F66Zn | F116Sn | F137Ba | 0.905 | 0.907 |
| F34S | F66Zn | F116Sn | F208Pb | 0.905 | 0.907 |
| F34S | F66Zn | F116Sn | F117Sn | 0.905 | 0.907 |
| F34S | F66Zn | F82Se | F116Sn | 0.905 | 0.907 |
| F34S | F62Ni | F66Zn | F116Sn | 0.905 | 0.907 |
| F34S | F66Zn | F116Sn | F206Pb | 0.905 | 0.907 |

### Results for Test IC: The >4 Parameter Test for Prostate Cancer

XGBoost classifier was implemented and a systematic increase in the number of isotopic features was used, resulting in a gradual improvement in prediction accuracy. For example, a combination of F⁶⁶Zn, F³⁴S, F¹¹⁶Sn, F⁵³Cr, and F⁵²Cr (n=5) achieved a training accuracy of 91.4% and a testing accuracy of 90.7%, while a combination of F⁶⁶Zn, F³⁴S, F¹¹⁶Sn, F⁵³Cr, and F⁴³Ca (n=5) reached training and testing accuracies of 90.9% and 90.2%, respectively. Furthermore, the addition of F¹⁰B or F¹¹B to the combination (including F⁶⁶Zn, F³⁴S, F¹¹⁶Sn, F⁵³Cr, F⁵²Cr, and F¹⁰B or F¹¹B; n=6) resulted in an increase to 92.4% training and 91.7% testing accuracies.

An eight-feature (n=8) signature, comprising F⁶⁶Zn, F³⁴S, F¹¹⁶Sn, F⁵³Cr, F⁵²Cr, F¹⁰Br, F⁶⁷Zn and F⁸⁷Sr, led to training and testing accuracies of 93.2% and 93.1%, respectively. The accuracy further improved to 94.1% for both training and testing with a ten-feature (n=10) signature (F⁶⁶Zn, F³⁴S, F¹¹⁶Sn, F⁵³Cr, F⁵²Cr, F¹⁰Br, F⁶⁷Zn, F⁸⁷Sr, F¹³C and F²⁰⁷Pb).

The accuracy further improved to 94.1% for both training and testing with a 12-feature (n=12) signature (F⁶⁶Zn, F³⁴S, F¹¹⁶Sn, F⁵³Cr, F⁵²Cr, F¹⁰Br, F⁶⁷Zn, F⁸⁷Sr, F¹³C, F²⁰⁷Pb, F²⁶Mg and F⁷⁹Br or F⁸¹Br).

Extending the feature set to n=14 (including F⁶⁶Zn, F³⁴S, F¹¹⁶Sn, F⁵³Cr, F⁵²Cr, F¹⁰Br, F⁶⁷Zn, F⁸⁷Sr, F¹³C, F²⁰⁷Pb, F²⁶Mg, F⁷⁹Br, F⁵⁰Cr, and F⁵⁶Fe or F⁵⁷Fe) achieved 95.6% training and 95.1% testing accuracies. With n=16 features (adding F⁴³Ca or F⁴⁴Ca and F⁸⁵Rb or F⁸⁷Rb to the previous combination), the accuracies were 96.4% for training and 94.1% for testing. Adding F⁸²Se and F⁶¹Ni or F⁶²Ni (n=18) achieved 95.1% testing accuracies and increasing feature number to 20 by adding F²⁵Mg or F⁸⁶Sr (n=20) achieved 95.6% testing accuracies. Further addition (n=22 and n=23) provided the same accuracy (FIG. 16B).

Using the same XGBoost training parameters and a 20-feature set, another random sampling yielded 94.6% accuracy, while using 59 features we achieved 93.6% accuracy (Table 9).

TABLE 9
Accuracy for Testing using 20 or 59 Isotopic Features.

| Metrics | 20 Features | 59 Features |
|---|---|---|
| Accuracy | 0.946 | 0.936 |
| AUC | 0.94 | 0.927 |
| Cohen's Kappa | 0.886 | 0.864 |
| F1 Score | 0.929 | 0.915 |
| Jaccard | 0.867 | 0.843 |
| MCC | 0.556 | 0.539 |
| NPV | 0.945 | 0.931 |
| Precision | 0.947 | 0.946 |
| Sensitivity | 0.911 | 0.886 |
| Specificity | 0.968 | 0.968 |
| Average Precision Score | 0.958 | 0.954 |

Together, the above results demonstrate that any of a variety of combinations of isotopic features can result in a high accuracy for predicting cancer.

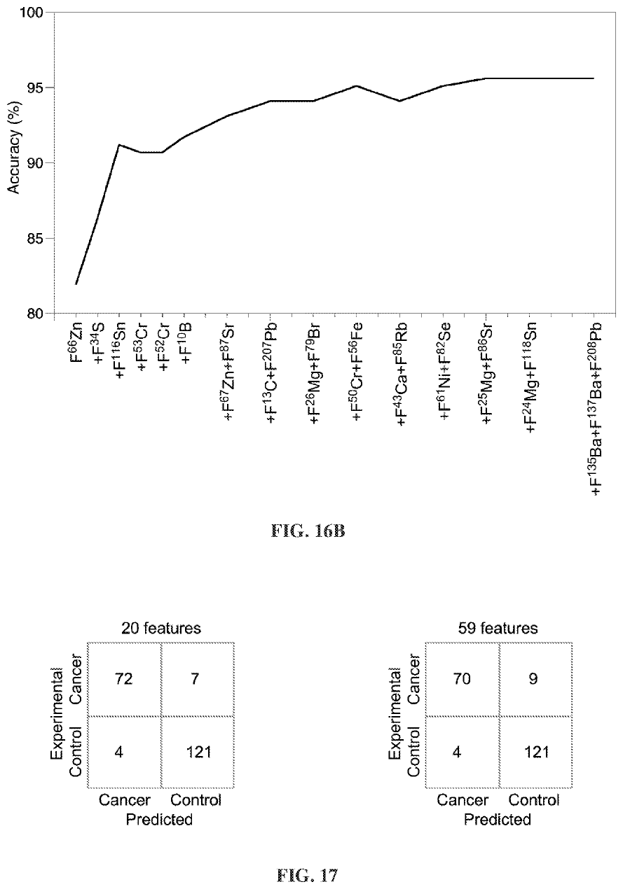

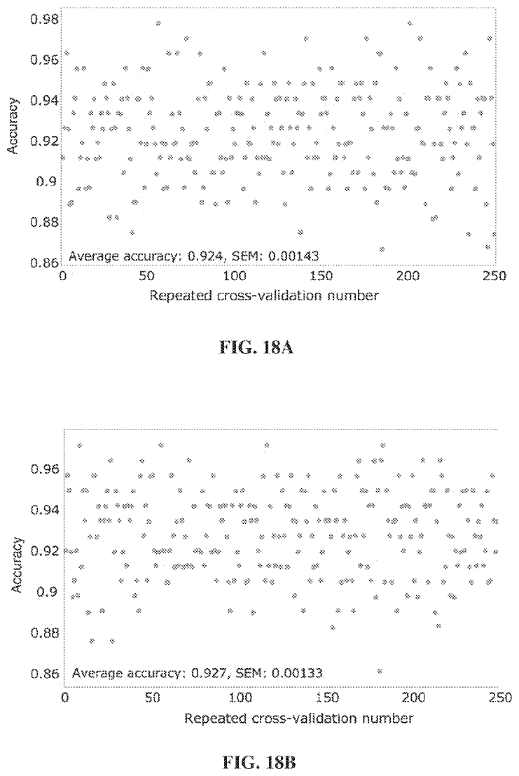

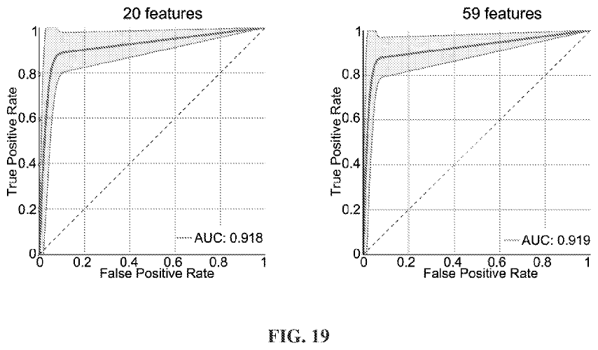

FIG. 17 shows performance of the algorithm tested with 79 cancer samples and 125 control samples, using 20 or 59 features. With n=20 features identified from Test 1, a repeated cross-validation experiment showed an average accuracy of 92.4% (FIG. 18A). These top 20 features were: F⁶⁶Zn, F³⁴S, F¹¹⁶Sn, F⁵³Cr, F⁵²Cr, F¹⁰B, F⁶⁷Zn, F⁸⁷Sr, F¹³C, F²⁰⁷Pb, F²⁶Mg, F⁷⁹Br, F⁵⁰Cr, F⁵⁶Fe, F⁴³Ca, F⁸⁵Rb, F⁸²Se, F⁶¹Ni, F²⁵Mg, and F⁸⁶Sr. With n=59 features, a repeated cross-validation experiment showed an average accuracy of 92.7% (FIG. 18B). These 59 features were: F¹³C, F³⁴S, F¹⁰B, F¹¹B, F²⁴Mg, F²⁵Mg, F²⁶Mg, F⁴³Ca, F44Ca, F⁴⁶Ti, F⁴⁷Ti, F⁴⁸Ti, F⁴⁹Ti, F⁵⁰Cr, F⁵²Cr, F⁵³Cr, F⁵⁶Fe, F⁵⁷Fe, F⁵⁸Ni, F⁶⁰Ni, F⁶¹Ni, F⁶²Ni, F⁶³Cu, F⁶⁵Cu, F⁶⁴Zn, F⁶⁶Zn, F⁶⁷Zn, F⁶⁸Zn, F⁷⁷Se, F⁷⁸Se, F⁸²Se, F⁷⁹Br, F⁸¹Br, F⁸⁵Rb, F⁸⁷Rb, F⁸⁴Sr, F⁸⁶Sr, F⁸⁷Sr, F⁸⁸Sr, F¹⁰⁷Ag, F¹⁰⁹Ag, F¹¹⁶Sn, F¹¹⁷Sn, F¹¹⁸Sn, F¹¹⁹Sn, F¹²⁰Sn, F¹³⁵Ba, F¹³⁶Ba, F¹³⁷Ba, F¹³⁸Ba, F¹⁹⁹Hg, F²⁰⁰Hg, F²⁰¹Hg, F²⁰²Hg, F²⁰⁶Pb, F²⁰⁷Pb, F²⁰⁸Pb, F²³⁵U, and F²³⁸U. FIG. 19 shows that the model's true positive rate (sensitivity) increases faster than its false positive rate (1-specificity) with n=20 or n=59 features, and that area under the curve (AUC) was much greater than random (0.5).

### Discussion

In a hair sample, one single isotopic feature (e.g., fractional abundance of an isotope of an isotopic element) can predict whether a man from the US has prostate cancer with an accuracy of more than 80%. These chemical features are F⁶⁴Zn and F⁶⁶Zn. However, increasing the number of isotopic features the algorithm may also increase its performance. For example, using 2 isotopic features (encompassing 1,711 possible pairs) offered accuracies of max 86.3%, 5 features ~90% accuracy, 10 features 95.4% accuracy, 14-16 features an accuracy of 95% accuracy. The increase in performance with the number of isotopic features is also clear from the number of combinations that yield accuracy over a threshold. For example, a combination of 3 isotopic features (n=3) yielded 32,509 combinations with test accuracy over 70%, and when combining 4 isotopic features this number was increased to 455,126 combinations, across different samples. XGBoost training parameters and a 20-feature set yielded 94.6% accuracy, and a repeated cross-validation experiment showed an average accuracy of 92.4%.

B. Test 2: Cohort of US Males and Females with or without Various Cancers

A pan-cancer model was developed using the data from patients and controls in the US. The patients were affected by basal cell skin cancer, bladder cancer, colon cancer, Hodgkin's lymphoma, kidney cancer, leukemia, liver cancer, liver and bone cancer, lung cancer, lymphoma, melanoma, prostate cancer, prostate cancer and chronic lymphocytic leukemia, rectal cancer and non-Hodgkin's lymphoma, SCC and BCC, skin cancer, skin-melanoma and prostate cancer, skin cancer and maybe prostate cancer, testicular cancer, thyroid cancer, tonsil cancer, and cancer not disclosed. The patients were both men and women, and the XGBoost Classifier was used.

### Results

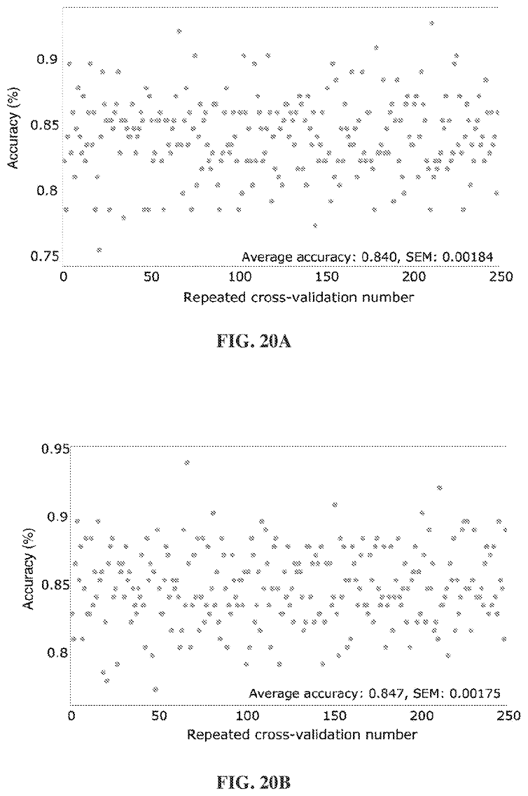

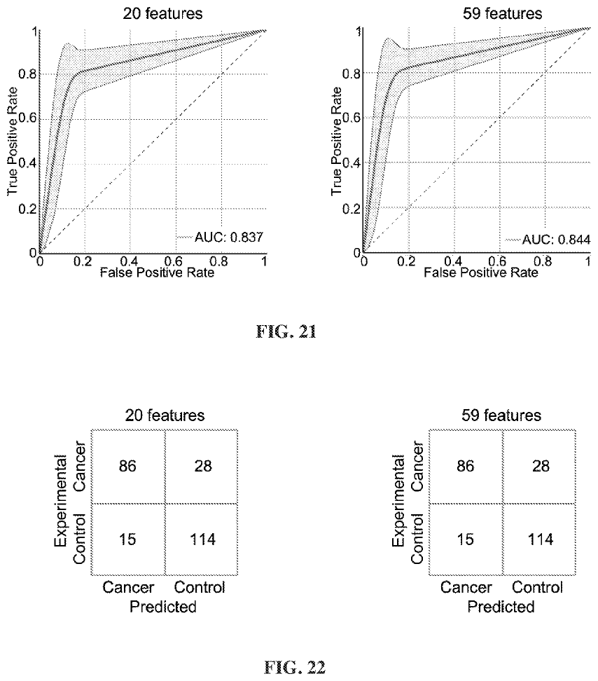

With n=20 features identified from Test 1, a repeated cross-validation experiment showed an average accuracy of 84% (FIG. 20A). These 20 features were: F⁶⁶Zn, F³⁴S, F¹¹⁶Sn, F⁵³Cr, F⁵²Cr, F¹⁰B, F⁶⁷Zn, F⁸⁷Sr, F¹³C, F²⁰⁷Pb, F²⁶Mg, F⁷⁹Br, F⁵⁰Cr, F⁵⁶Fe, F⁴³Ca, F⁸⁵Rb, F⁸²Se, F⁶¹Ni, F²⁵Mg, and F⁸⁶Sr. With n=59 features, a repeated cross-validation experiment showed an average accuracy of 84.7% (FIG. 20B). These 59 features were: F¹³C, F³⁴S, F¹⁰B, F¹¹B, F²⁴Mg, F²⁵Mg, F²⁶Mg, F⁴³Ca, F⁴⁴Ca, F⁴⁶Ti, F⁴⁷Ti, F⁴⁸Ti, F⁴⁹Ti, F⁵⁰Cr, F⁵²Cr, F⁵³Cr, F⁵⁶Fe, F⁵⁷Fe, F⁵⁸Ni, F⁶⁰Ni, F⁶¹Ni, F⁶²Ni, F⁶³Cu, F⁶⁵Cu, F⁶⁴Zn, F⁶⁶Zn, F⁶⁷Zn, F⁶⁸Zn, F⁷⁷Se, F⁷⁸Se, F⁸²Se, F⁷⁹Br, F⁸¹Br, F⁸⁵Rb, F⁸⁷Rb, F⁸⁴Sr, F⁸⁶Sr, F⁸⁷Sr, F⁸⁸Sr, F¹⁰⁷Ag, F¹⁰⁹Ag, F¹¹⁶Sn, F¹¹⁷Sn, F¹¹Sn, F¹¹⁹Sn, F¹²⁰Sn, F¹³⁵Ba, F¹³⁶Ba, F¹³⁷Ba, F¹³⁸Ba, F¹⁹⁹Hg, F²⁰⁰Hg, F²⁰¹Hg, F²⁰²Hg, F²⁰⁶Pb, F²⁰⁷Pb, F²⁰⁸Pb, F²³⁵U, and F²³⁸U. FIG. 21 shows that the model's true positive rate (sensitivity) increases faster than its false positive rate (1-specificity) with n=20 or n=59 features, and that is performance (0.837 and 0.844) was much greater than random (0.5).

FIG. 22 and Table 10 show that when testing with 114 cancer samples 129 control samples, we achieved the same predictive performance with n=20 or n=59 features, which can be a coincidence as one would expect a minimum difference. Using Test 2, the predictive performance for testing using 20 or 50 isotopic features resulted in many of the same performance metrics as shown in Table 10.

TABLE 10
Predictive Performance for Testing
using 20 or 59 Isotopic Features.

| Metrics | 20 Features | 59 Features |
|---|---|---|
| Accuracy | 0.823 | 0.823 |
| AUC | 0.819 | 0.819 |
| Cohen's Kappa | 0.642 | 0.642 |
| F1 Score | 0.8 | 0.8 |
| Jaccard | 0.667 | 0.667 |
| MCC | 0.459 | 0.459 |
| NPV | 0.803 | 0.803 |
| Precision | 0.851 | 0.851 |
| Sensitivity | 0.754 | 0.754 |
| Specificity | 0.884 | 0.884 |
| Average Precision Score | 0.881 | 0.894 |

### Discussion

This example demonstrates a predictive model built using various cancer types from both men and women the US. The predictive model attained more than 84% average accuracy. There was not a noticeable difference when predictive models were built using 20 and 59 features using Test 2.

C. Test 3: Cohort of US and Sweden Males and Females with or without Various Cancers

A pan-cancer model was developed using the data from patients and controls in the US and Sweden. The patients were affected by basal cell skin cancer, bladder cancer, breast cancer, colon cancer, Hodgkin's lymphoma, kidney cancer, leukemia, liver cancer, liver and bone cancer, lung cancer, lymphoma, melanoma, prostate cancer, prostate cancer and chronic lymphocytic leukemia, rectal and non-Hodgkin's, SCC and BCC, skin cancer, skin-melanoma and prostate cancer, skin and maybe prostate cancer, testicular cancer, thyroid cancer, cancer not disclosed, and tonsil cancer. The patients were both men and women, and the XGBoost Classifier was used.

### Results

Several mixed predictive models were built using cancer samples from different types of cancers from both US and Swedish patients.

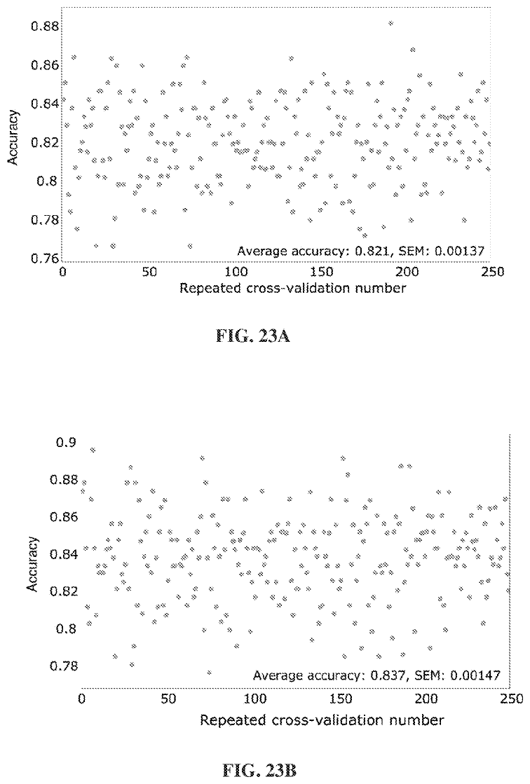

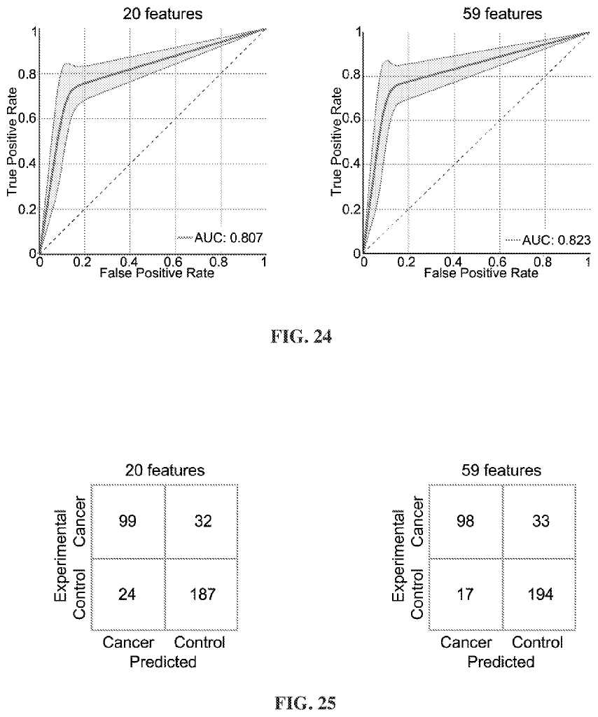

With n=20 features identified from Test 1, a repeated cross-validation experiment showed an average accuracy of 82.1% (FIG. 23A). These 20 features were: F⁶⁶Zn, F³⁴S, F¹¹⁶Sn, F⁵³Cr, F⁵²Cr, F¹⁰B, F⁶⁷Zn, F⁸⁷Sr, F¹³C, F²⁰⁷Pb, F²⁶Mg, F⁷⁹Br, F⁵⁰Cr, F⁵⁶Fe, F⁴³Ca, F⁸⁵Rb, F⁸²Se, F⁶¹Ni, F²⁵Mg, and F⁸⁶Sr. With n=59 features, a repeated cross-validation experiment showed an average accuracy of 84.7% (FIG. 23B). These 59 features were: F³C, F³⁴S, F¹⁰B, F¹¹B, F²⁴Mg, F²⁵Mg, F²⁶Mg, F⁴³Ca, F⁴⁴Ca, F⁴⁶Ti, F⁴⁷Ti, F⁴⁸Ti, F⁴⁹Ti, F⁵⁰Cr, F⁵²Cr, F⁵³Cr, F⁵⁶Fe, F⁵⁷Fe, F⁵⁸Ni, F⁶⁰Ni, F⁶¹Ni, F⁶²Ni, F⁶³Cu, F⁶⁵Cu, F⁶⁴Zn, F⁶⁶Zn, F⁶⁷Zn, F⁶⁸Zn, F⁷⁷Se, F⁷⁸Se, F⁸²Se, F⁷⁹Br, F⁸¹Br, F⁸⁵Rb, F⁸⁷Rb, F⁸⁴Sr, F⁸⁶Sr, F⁸⁷Sr, F⁸⁸Sr, F¹⁰⁷Ag, F¹⁰⁹Ag, F¹¹⁶Sn, F¹¹⁷Sn, F¹¹⁸Sn, F¹¹⁹Sn, F¹²⁰Sn, F¹³⁵Ba, F¹³⁶Ba, F¹³⁷Ba, F¹³⁸Ba, F¹⁹⁹Hg, F²⁰⁰Hg, F²⁰¹Hg, F²⁰²Hg, F²⁰⁶Pb, F²⁰⁷Pb, F²⁰⁸Pb, F²³⁵U, and F²³⁸U. FIG. 24 shows that the model's true positive rate (sensitivity) increases faster than the false positive rate (1-specificity), with n=20 or n=59 features, and that area under the curve (AUC) of (0.807 and 0.823) was much greater than random (0.5).

This predictive model achieved a level of prediction accuracy (average: 85.4%), when testing with 131 cancer samples and 211 control samples (Table 11 and FIG. 25), which comparable to that of predictive model of Test 1 which uses prostate cancer samples from US males.

TABLE 11
Predictive Performance for Testing
using 20 or 59 Isotopic Features.

| Metrics | 20 Features | 59 Features |
|---|---|---|
| Accuracy | 0.836 | 0.854 |
| AUC | 0.821 | 0.834 |
| Cohen's Kappa | 0.65 | 0.683 |
| F1 Score | 0.78 | 0.797 |
| Jaccard | 0.639 | 0.662 |
| MCC | 0.426 | 0.431 |
| NPV | 0.854 | 0.855 |
| Precision | 0.805 | 0.852 |
| Sensitivity | 0.756 | 0.748 |
| Specificity | 0.886 | 0.919 |
| Average Precision Score | 0.884 | 0.906 |

Other normalization strategies in addition to fractional abundance, including PseudoNδ, SimpleNδ and δ_InH, displayed similar predictive performances. Accuracy values achieved when using XGBoost Classifier for test samples with data composed of 59 isotopic features (fractional abundances, PseudoNδ, SimpleNδ and δ_InH values) of major and minor elements in hair from US and Sweden males and females with and without basal cell skin cancer, bladder cancer, breast cancer, colon cancer, Hodgkin's lymphoma, kidney cancer, leukemia, liver cancer, liver and bone cancer, lung cancer, lymphoma, melanoma, prostate cancer, prostate cancer and chronic lymphocytic leukemia, rectal and non-Hodgkin's, renal cancer, renal cell carcinoma, SCC and BCC, skin cancer, skin-melanoma and prostate cancer, skin and maybe prostate cancer, testicular cancer, thyroid cancer, cancer not disclosed, and tonsil cancer, are shown in Table 12.

TABLE 12
Predictive Performance for Testing using 59 Isotopic Features.

| | Fractional | | | |
|---|---|---|---|---|
| Metrics | Abundance | PseudoNδ | SimpleNδ | δ_InH |
| Accuracy | 0.854 | 0.810 | 0.822 | 0.854 |
| AUC | 0.834 | 0.788 | 0.811 | 0.835 |
| Cohen's Kappa | 0.683 | 0.589 | 0.622 | 0.684 |
| F1 Score | 0.797 | 0.737 | 0.766 | 0.798 |
| Jaccard | 0.662 | 0.583 | 0.621 | 0.664 |
| MCC | 0.431 | 0.383 | 0.423 | 0.435 |
| NPV | 0.855 | 0.823 | 0.854 | 0.858 |
| Precision | 0.852 | 0.784 | 0.769 | 0.846 |
| Sensitivity | 0.748 | 0.695 | 0.763 | 0.756 |
| Specificity | 0.919 | 0.882 | 0.858 | 0.915 |
| Average Precision Score | 0.906 | 0.849 | 0.883 | 0.910 |

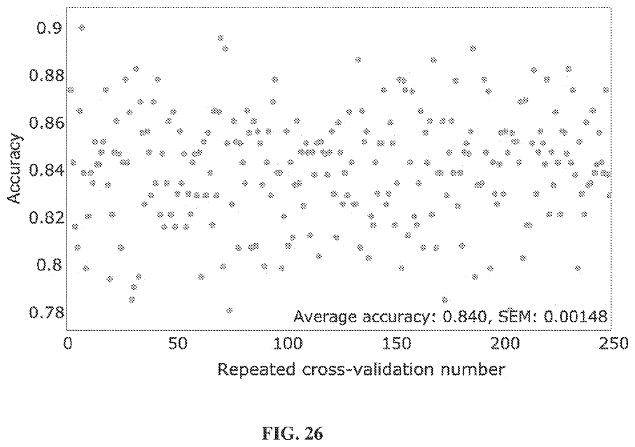

With n=59 features, a repeated cross-validation experiment showed an average accuracy of 84% (FIG. 26). These 59 features were: F¹³C, F³⁴S, F¹⁰B, F¹¹B, F²⁴Mg, F²⁵Mg, F²⁶Mg, F⁴³Ca, F⁴⁴Ca, F⁴⁶Ti, F⁴⁷Ti, F⁴⁸Ti, F⁴⁹Ti, F⁵⁰Cr, F⁵²Cr, F⁵³Cr, F⁵⁶Fe, F⁵⁷Fe, F⁵⁸Ni, F⁶⁰Ni, F⁶¹Ni, F⁶²Ni, F⁶³Cu, F⁶⁵Cu, F⁶⁴Zn, F⁶⁶Zn, F⁶⁷Zn, F⁶⁸Zn, F⁷⁷Se, F⁷⁸Se, F⁸²Se, F⁷⁹Br, F⁸¹Br, F⁸⁵Rb, F⁸⁷Rb, F⁸⁴Sr, F⁸⁶Sr, F⁸⁷Sr, F⁸⁸Sr, F¹⁰⁷Ag, F¹⁰⁹Ag, F¹¹⁶Sn, F¹¹⁷Sn, F¹¹⁸Sn, F¹¹⁹Sn, F¹²⁰Sn, F¹³⁵Ba, F¹³⁶Ba, F¹³⁷Ba, F¹³⁸Ba, F¹⁹⁹Hg, F²⁰⁰Hg, F²⁰¹Hg, F²⁰²Hg, F²⁰⁶Pb, F²⁰⁷Pb, F²⁰⁸Pb, F²³⁵U, and F²³⁸U.

### Discussion

Using a mixed US and Swedish cancer model for various cancers including basal cell skin cancer, bladder cancer, breast cancer, colon cancer, Hodgkin's lymphoma, kidney cancer, leukemia, liver cancer, liver and bone cancer, lung cancer, lymphoma, melanoma, prostate cancer, prostate cancer and chronic lymphocytic leukemia, rectal and non-Hodgkin's, SCC and BCC, skin cancer, skin-melanoma and prostate cancer, skin and maybe prostate cancer, testicular cancer, thyroid cancer, cancer not disclosed, and tonsil cancer, two selected sets of features (n=20 and n=59) achieved more than 81% prediction accuracy. Different data normalization methods were also tested. While a minimum variation in predictive performance was observed, all data normalization models achieved more than 80% accuracy when applying the δ_InH normalization method.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and claims hereafter set forth.

### REFERENCES

1. Cancer (who.int)
2. https://www.mayoclinic.org/diseases-conditions/cancer/diagnosis-treatment/drc-20370594
3. https://www.cancer.org/cancer/prostate-cancer/detection-diagnosis-staging/how-diagnosed.html
4. Dahl, T. W., et al. 2010. Devonian rise in atmospheric oxygen correlated to the radiations of terrestrial plants and large predatory fish. Proc Natl Acad Sci USA, 107:17911-17915.
5. Lane, A. N., et al. 2009. Prospects for clinical cancer metabolomics using stable isotope tracers. Exp Mol Pathol, 86:165-173.
6. Balter, V., et al. 2015. Natural variations of copper and sulfur stable isotopes in blood of hepatocellular carcinoma patients. Proc Natl Acad Sci USA, 112:982-985.
7. Larner, F., et al. 2015. Zinc isotopic compositions of breast cancer tissue. Metallomics, 7:112-117.
8. Walczyk, T., and von Blanckenburg, F. 2002. Natural iron isotope variations in human blood. Science, 295:2065-2066.
9. Maruyama, A., et al. 2018. Hairs in old books isotopically reconstruct the eating habits of early modem Japan. Sci Rep, 8:12152.
10. Ehleringer, J. R., et al. 2008. Hydrogen and oxygen isotope ratios in human hair are related togeography. Proc Natl Acad Sci USA, 105:2788-2793.
11. Rodiouchkina, K., Rodushkin, I., Vanhaecke, F., and Goderis, S. 2021. Longitudinal isotope ratio variations in human hair. Goldschmidt Abstract, doi.org/10.7185/gold2021.4748.
12. Rodiouchkina, K., Rodushkin, I., Goderis, S., and Vanhaecke, F. 2022. Longitudinal isotope ratio variations in human hair and nails. Sci Total Environ, 808:152059.
13. Podgorelec, V., Kokol, P., Stiglic, B., and Rozman, I. 2002. Decision Trees: An Overview and Their Use in Medicine. Journal of Medical Systems, 26:445-463.
14. Breiman, L. 2001. Random Forests. Machine Learning, 45:5-32.
15. Chen, R. C., Dewi, C., Huang, S. W., and Caraka, R. E. 2020. Selecting critical features for data classification based on machine learning methods. Journal of Big Data, 7:52.
16. LeCun, Y., Bengio, Y., and Hinton, G. 2015. Deep learning. Nature, 521:436-444.
17. Way, G. P., and Greene, C. S. 2018. Extracting a biologically relevant latent space from cancer transcriptomes with variational autoencoders. Pac Symp Biocomput, 23:80-91.
18. Li, M., et al. 2021. DeepDSC: A Deep Learning Method to Predict Drug Sensitivity of Cancer Cell Lines. IEEE/ACM Trans Comput Biol Bioinform. 18:575-582.
19. Candes, E., Fan, Y., Janson, L., and Lv, J. 2018. Panning for gold: ‘model-X’ knockoffs for high dimensional controlled variable selection. J R Statist Soc B, 80:551-577.
20. Zhu, Z., et al. 2021. DeepLINK: Deep learning inference using knockoffs with applications to genomics. Proc Natl Acad Sci USA, 118(36), e2104683118.
21. Chen, T., Guestrin, C. (2016). XGBoost: A Scalable Tree Boosting System. Proceedings of the 22nd ACM SIGKDD International Conference on Knowledge Discovery And Data Mining. https://doi.org/10.1145/2939672.2939785
22. scikit-learn. sklearn.linear_model.LogisticRegression. scikit. Retrieved Oct. 7, 2022
23. Ho, Tin Kam (1995). Random Decision Forests (PDF). Proceedings of the 3rd International Conference on Document Analysis and Recognition, Montreal, QC, 14-16 Aug. 1995. pp. 278-282.
24. Prokhorenkova, L., Gusev, G., Vorobev, A., Dorogush, A. V., Gulin, A. (2019). CatBoost: Unbiased boosting with categorical features. arXiv.org.
25. Guolin Ke, Qi Meng, “Thomas Finley, Taifeng Wang, Wei Chen, Weidong Ma, Qiwei Ye,” Tie-Yan Liu. “LightGBM: A Highly Efficient Gradient Boosting Decision Tree”. Advances in Neural Information Processing Systems 30 (NIPS 2017), pp. 3149-3157.
26. Yin Lou, Rich Caruana, Johannes Gehrke, and Giles Hooker. Accurate intelligible models with pairwise interactions. In Proceedings of the 19th ACM SIGKDD international conference on Knowledge discovery and data mining, 623-631. 2013.
27. scikit-learn. Support Vector Machines. scikit. Retrieved Oct. 7, 2022.
28. Arik, S. O., Pfister, T. (2020). TabNet: Attentive interpretable tabular learning. 29. Rafique, R., Riazul Islam, S. M., Kazi, J. U., Computational and Structural Biotechnology Journal 19 (2021) 4003-4017.
30. Deng, Y. and Lumley T. arXiv 2021 DOI: 10.48550/arXiv.2106.01574
31. Zhu, Z., Fan, Y., Kong, Y., Lv, J., and Sun, F. Proc Natl Acad Sci UAS (2021) 118: e2104683118
32. Lundberg, S. M. et al. Nat Mach Intell (2020) 2:56-67.
33. Ishak et al. Malays J Med Sci (2015) 22:11-20.
34. Holmström et al. BMJ (2009) 339:b3537.
35. Hayes, J. M. (2004). An Introduction to Isotopic Calculations. Woods Hole Oceanographic Institution.
36. Rodushkin I. and Axelsson M. D. Application Of Double Focusing Sector Field ICP-MS For Multielemental Characterization Of Human Hair And Nails. Sci Total Environ.

2000; 250(1):83-100. doi: org/10.1016/s0048-9697(00)00369-7.
37. Skrzypek G. and Dunn P. J. H. Recalculation of stable isotope expressions for HCNOS:EasylsoCalculator. Rapid Commun Mass Spectrom. 2020; 34:e8892.
38. Rasmussen C M Ø et al. J. Cascading trend of Early Paleozoic marine radiations paused by Late Ordovician extinctions. Proc Natl Acad Sci USA. 2019 Apr. 9; 116(15):7207-7213.
39. Révész, Kinga, and Qi, Haiping, 2006, Determination of the delta(15N/14N) and delta(13C/12C) of total N and C in solids: RSIL lab code 1832. C5 of Révész, Kinga, and Coplen, Tyler B., eds., Methods of the Reston Stable Isotope Laboratory: Reston, Virginia, U.S. Geological Survey, Techniques and Methods, book 10, sec. C, chap. 5, p. 30.
40. Brenna, J. T., et al., 1997, High-precision continuous-flow isotope ratio mass spectrometry: Mass Spectrometry Reviews, v. 16, p. 227-258.
41. Coplen, T. B., et al., 2006, New guidelines for delta 13C measurements: Analytical Chemistry, v. 78, p. 2439-2441.
42. Böhlke, J. K., and Coplen, T. B., 1995, Interlaboratory comparison of secondary reference materials for nitrogen-isotope-ratio measurements, in Reference and intercomparison materials for stable isotopes of light elements: Vienna, Austria, International Atomic Energy Agency, IAEA-TECDOC-825, p. 51-66.
43. Böhlke, J. K., Mroczkowski, S. J., and Coplen, T. B., 2003, Oxygen isotopes in nitrate: New reference materials for 180:170:160 measurements and observations on nitrate-water equilibration: Rapid Communications in Mass Spectrometry, v. 17, p. 1835-1846.
44. Qi, H., et al., 2003, Two new organic reference materials for delta 13C and delta 15N measurements and a new value for the delta 13C of NBS 22 oil: Rapid Communications in Mass Spectrometry, v. 17, p. 2483-2487.
45. Carmody, R. W., et al., 1997. Methods for collection of dissolved sulfate and sulfide and analysis of their sulfur isotopic composition, U.S. Geological Survey Open-File Report 97-234, p. 91.
46. Brenna, J. T., et al., 1997, High-precision continuous-flow isotope ratio mass spectrometry: Mass Spectrometry Reviews, v. 16, p. 227-258.
47. Coplen, T. B., et al., 2001, Compilation of minimum and maximum isotope ratios of selected elements in naturally occurring terrestrial materials and reagents, U.S. Geological Survey Water-Resources Investigations Report 01-4222, p. 98.
48. Campbell, M. J., Machin, D., & Walters, S. J. (2010). Medical statistics: a textbook for the health sciences. John Wiley & Sons.
49. Ding, T., et. al. “Calibrated sulfur isotope abundance ratios of three IAEA sulfur isotope reference materials and V-CDT with a reassessment of the atomic weight of sulfur”, Geochimica et Cosmochimica Acta, vol. 65, no. 15, pp. 2433-2437, 2001.
50. Thode, H. G., et. al., Sulphur isotope geochemistry, Geochimica et Cosmochimica Acta, Volume 25, Issue 3, 1961, Pages 159-174.

What is claimed is:

1. A method of detecting whether a subject has a cancer, the method comprising:
(a) determining the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject;
(b) inputting the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(c) determining, using the prediction model, whether the subject has the cancer.

2. A method of detecting a cancer in a subject, the method comprising:
(a) determining the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject;
(b) inputting the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(c) detecting the cancer in the subject using the prediction model.

3. A method of assessing a characteristic of a cancer in a subject, the method comprising:
(a) determining the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject;
(b) inputting the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and
(c) determining, using the prediction model, the characteristic of the cancer in the subject.

4. The method of claim 3, wherein the characteristic of the cancer is the level of tumor burden of the cancer in the subject.

5. The method of any one of claims 1-4, wherein the sample is a solid sample, and wherein the determining the value for each of the at least one isotopic feature comprises mass spectrometry analysis of the solid sample, optionally analyzed via combustion, or a liquid sample prepared by digestion of the solid sample.

6. The method of any one of claims 1-5, wherein the value is a normalized value, and wherein the determining the value for each of the at least one isotopic feature comprises calculating the normalized value from the mass spectrometry analysis.

7. The method of any one of claims 1-6, wherein the sample is a solid sample, and wherein the determining the value for each of the at least one isotopic feature comprises:
(i) preparing a liquid sample of the solid sample for analysis by mass spectrometry, wherein the liquid sample is prepared by digestion of the solid sample;
(ii) introducing the prepared liquid sample into a mass spectrometer to detect one or more ionization products of elements and isotopic elements present in the prepared liquid sample; and
(iii) analyzing the detected one or more ionization products of elements and isotopic elements to generate the value for each of the at least one isotopic feature.

8. The method of any one of claims 1-6, wherein the sample is a solid sample, and wherein the determining the value for each of the at least one isotopic feature comprises:
(i) preparing the solid sample for analysis by mass spectrometry;
(ii) introducing the prepared solid sample into a mass spectrometer to detect one or more ionization products of elements and isotopic elements present in the prepared solid sample, optionally wherein the detecting comprises combustion of the prepared solid sample; and
(iii) analyzing the detected one or more ionization products of elements and isotopic elements to generate the value for each of the at least one isotopic feature.

9. The method of any one of claims 1-8, wherein the determining in step (a), the inputting in step (b), and/or the determining in step (c) is performed by a processor of a computing device.

10. A method of detecting whether a subject has a cancer, the method comprising:
(a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject;
(b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(c) determining, using the prediction model, whether the subject has the cancer.

11. A method of detecting a cancer in a subject, the method comprising:
(a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject;
(b) inputting, using at least one processor, the value for each of the at least one isotopic feature from the subject into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(c) detecting the cancer in the subject using the prediction model.

12. A method of assessing a characteristic of a cancer in a subject, the method comprising:
(a) receiving, at one or more processors, the value for each of at least one isotopic feature for a sample from a subject, wherein the value for each of the at least one isotopic feature is: (1) a concentration of an element, (2) a concentration of an isotopic element, (3) a concentration of an isotope of the isotopic element, or (4) a normalized value of any of (1)-(3), measured in the sample of the subject;
(b) inputting, using at least one processor, the value for each of the at least one isotopic feature from the subject into a prediction model configured to predict a characteristic of a cancer in a subject, wherein the prediction model comprises at least one machine learning algorithm trained using values for each of the at least one isotopic feature from a plurality of reference samples from a plurality of reference subjects; and
(c) determining, using the prediction model, the characteristic of the cancer in the subject.

13. The method of claim 12, wherein the characteristic of the cancer is the level of tumor burden of the cancer in the subject.

14. The method of any one of claims 10-13, wherein the sample is a solid sample, and wherein the value for each of the at least one isotopic feature is obtained by mass spectrometry analysis of the solid sample, optionally analyzed via combustion, or a liquid sample prepared by digestion of the solid sample.

15. The method of any one of claims 10-14, wherein the value is a normalized value, and wherein the determining the value for each of the at least one isotopic feature comprises calculating the normalized value from the mass spectrometry analysis.

16. The method of any one of claims 10-15, wherein the sample is a solid sample, and wherein prior to step (a), the method comprises determining the value for each of the at least one isotopic feature, wherein the determining comprises:
(i) preparing a liquid sample of the solid sample for analysis by mass spectrometry, wherein the liquid sample is prepared by digestion of the solid sample;
(ii) introducing the prepared liquid sample into a mass spectrometer to detect one or more ionization products of elements isotopic elements present in the prepared liquid sample; and
(iii) analyzing the detected one or more ionization products of elements and isotopic elements to generate the value for each of the at least one isotopic feature.

17. The method of any one of claims 10-15, wherein the sample is a solid sample, and wherein prior to step (a), the method comprises determining the value for each of the at least one isotopic feature, wherein the determining comprises:
(i) preparing the solid sample for analysis by mass spectrometry;
(ii) introducing the prepared solid sample into a mass spectrometer to detect one or more ionization products of elements and isotopic elements present in the prepared solid sample, optionally wherein the detecting comprises combustion of the prepared solid sample; and
(iii) analyzing the detected one or more ionization products of elements and isotopic elements to generate the value for each of the at least one isotopic feature.

18. The method of any one of claims 7-9, 16, and 17, wherein the analyzing the detected one or more ionization products of elements and isotopic elements is with reference to one or more ionization products of elements and isotopic elements present in standards comprising a plurality of calibration standards and a blank standard.

19. The method of any one of claims 7-9 and 16-18, wherein the analyzing the detected one or more ionization products of elements and isotopic elements is with reference to one or more ionization products of elements and isotopic elements present in standards comprising an internal standard and/or a reference material.

20. The method of claim 18 or claim 19, wherein the mass spectrometer is further introduced with the standards, wherein the mass spectrometer detects one or more ionization products of elements and isotopic elements present in the standards and the prepared liquid or solid sample.

21. A method of detecting whether a subject has a cancer, the method comprising:
(a) providing a solid sample obtained from a subject;
(b) preparing a liquid sample or the solid sample for analysis by mass spectrometry, wherein the liquid sample is prepared by digestion of the solid sample;
(c) providing standards comprising a plurality of calibration standards, a blank standard, an internal standard, and/or a reference material;
(d) introducing the standards and the prepared liquid or solid sample individually into a mass spectrometer to detect one or more ionization products of elements and isotopic elements present in each of the standards and the prepared liquid or solid sample;
(e) analyzing the detected one or more ionization products of elements and isotopic elements to generate the value for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3);
(f) receiving, at one or more processors, the value for each of the at least one isotopic feature;
(g) inputting, using at least one processor, the value for each of the at least one isotopic feature into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(h) determining, using the prediction model, whether the subject has the cancer.

22. A method of detecting a cancer in a subject, the method comprising:
(a) providing a solid sample obtained from a subject;
(b) preparing a liquid sample or the solid sample for analysis by mass spectrometry, wherein the liquid sample is prepared by digestion of the solid sample;
(c) providing standards comprising a plurality of calibration standards, a blank standard, an internal standard, and/or a reference material;
(d) introducing the standards and the prepared liquid or solid sample individually into a mass spectrometer to detect one or more ionization products of elements and isotopic elements present in each of the standards and the prepared liquid or solid sample;
(e) analyzing the detected one or more ionization products of elements and isotopic elements to generate the value for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3);
(f) receiving, at one or more processors, the value for each of the at least one isotopic feature;
(g) inputting, using at least one processor, the value for each of the at least one isotopic feature into a prediction model configured to predict the presence or absence of a cancer in a subject, wherein the prediction model comprises at least one machine learning algorithm trained using values for each of the at least one isotopic feature from a plurality of reference samples from a plurality of reference subjects; and
(h) detecting the cancer in the subject using the prediction model.

23. A method of assessing a characteristic of a cancer in a subject, the method comprising:
(a) providing a solid sample obtained from a subject;
(b) preparing a liquid sample or the solid sample for analysis by mass spectrometry, wherein the liquid sample is prepared by digestion of the solid sample;
(c) providing standards comprising a plurality of calibration standards, a blank standard, an internal standard, and/or a reference material;
(d) introducing the standards and the prepared liquid or solid sample individually into a mass spectrometer to detect one or more ionization products of elements and isotopic elements present in each of the standards and the prepared liquid or solid sample;
(e) analyzing the detected one or more ionization products of elements and isotopic elements to generate the value for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3);
(f) receiving, at one or more processors, the value for each of the at least one isotopic feature;
(g) inputting, using at least one processor, the value for each of the at least one isotopic feature into a prediction model configured to predict a characteristic of a cancer in a subject, wherein the prediction model comprises at least one machine learning algorithm trained using values for each of the at least one isotopic feature from a plurality of reference samples from a plurality of reference subjects; and
(h) determining, using the prediction model, the characteristic of the cancer in the subject.

24. The method of claim 23, wherein the characteristic of the cancer is the level of tumor burden of the cancer in the subject.

25. The method of any one of claims 21-24, wherein the value is a normalized value, and wherein the determining the value for each of the at least one isotopic feature comprises calculating the normalized value from the mass spectrometry analysis.

26. The method of any one of claims 1-25, wherein the sample comprises a keratinous tissue, optionally a hard keratinous tissue.

27. The method of any one of claims 1-26, wherein the sample is a noninvasive sample.

28. The method of any one of claims 1-27, wherein the sample is a hair sample or a nail sample.

29. The method of any one of claims 5, 7, 8, 14, 16, and 17-28, wherein the digestion is by acid digestion and/or thermal digestion, optionally wherein the digestion is by acid digestion and thermal digestion.

30. The method of claim 29, wherein the acid digestion is with nitric acid (HNO₃) or is with nitric acid (HNO₃) and hydrogen peroxide (H₂O₂).

31. The method of any one of claims 29-30, wherein the thermal digestion is by a microwave.

32. The method of any one of claims 5-9 and 14-31, wherein the mass spectrometry is Inductively Coupled Plasma Mass Spectrometry (ICP-MS), optionally wherein the ICP-MS is carried out with kinetic energy discrimination (KED).

33. The method of any one of claims 1-32, wherein the value of each of the at least one isotopic feature is a normalized value of any of (1)-(3).

34. The method of any one of claims 1-33, wherein the value of each of the at least one isotopic feature is a normalized value of a concentration of an isotopic element.

35. The method of any one of claims 1-33, wherein the value of each of the at least one isotopic feature is a normalized value of a concentration of an isotope of the isotopic element.

36. The method of any one of claims 1-35, wherein the normalized value of each of the at least one isotopic feature is an atomic percent (atom %), fractional abundance (F), isotope ratio (R), concentration ratio (CR), or a delta value (delta), or a combination of any of the foregoing.

37. The method of any one of claims 1-36, wherein the normalized value of each of the least one isotopic feature is a fractional abundance (F).

38. The method of any one of claims 1-36, wherein the normalized value of each of the at least one isotopic feature is a delta value.

39. The method of claim 36 or claim 38, wherein the delta value is a conventional delta value, a study-specific delta value (InH), a non-similar simple-nitrogen based delta value (SimpleNδ), or a non-similar pseudo-nitrogen based delta value (PseudoNδ).

40. The method of any one of claims 1-39, wherein the at least one isotopic feature is one isotopic feature.

41. The method of any one of claims 1-40, wherein the isotope of the at least one isotopic feature comprises ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr.

42. The method of any one of claims 1-39, wherein the at least one isotopic feature is a plurality of isotopic features.

43. The method of claim 42, wherein the plurality of isotopic features is 2-100 isotopic features, optionally wherein the plurality of isotopic features comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or more isotopic features.

44. The method of claim 42 or claim 43, wherein the at least one isotopic feature is a plurality of isotopic features determined from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or more elements or isotopic elements.

45. The method of any one of claims 1-44, wherein the element or the isotopic element of the at least one isotopic feature comprises Li, B, Na, Mg, Al, P, K, Sc, Ca, Ti, V, Cr, Mn, Fe, Co, Ni, Cu, Zn, Ge, As, Se, Br, Rb, Sr, Y, Zr, Nb, Mo, Ru, Ag, Cd, In, Sn, I, Te, Ba, Hg, Pb, U, C, N, O, or S, or a combination of any of the foregoing.

46. The method of any one of claims 1-45, wherein the element or the isotopic element of the at least one isotopic feature comprises Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S, or a combination of any of the foregoing.

47. The method of any one of claims 1-46, wherein the element or the isotopic element of the at least one isotopic feature comprises Li, B, Mg, Ca, Ti, Cr, Fe, Ni, Cu, Zn, Se, Br, Rb, Sr, Mo, Ag, Sn, Ba, Hg, Pb, U, C, N, S, or a combination of any of the foregoing.

48. The method of any one of claims 1-47, wherein the isotope of the at least one isotopic feature comprises ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing.

49. The method of any one of claims 1-48, wherein the isotopic element of the at least one isotopic feature comprises Zn.

50. The method of any one of claims 1-49, wherein the isotope of the at least one isotopic feature comprises ⁶Zn and/or ⁶⁶Zn.

51. The method of any one of claims 1-48, wherein the isotopic element of the at least one isotopic feature comprises N, C, S, or O.

52. The method of any one of claims 1-48 and 51, wherein the isotopic element of the at least one isotopic feature comprises ³⁴S.

53. The method of any one of claims 1-52, wherein the machine learning algorithm is a supervised machine learning algorithm.

54. The method of any one of claims 1-3, 5-11, 13-21, and 23-53, wherein the prediction model is a classification model.

55. The method of any one of claims 1-3, 5-11, 13-21, and 23-54, wherein the prediction model is a binary classification model.

56. The method of any one of claims 1-3, 5-11, 13-21, and 23-54, wherein the prediction model is a multiclass classification model.

57. The method of any one of claims 1-3, 5-11, 13-21, and 23-56, wherein the machine learning algorithm is Logistic Regression (e.g., Ridge, Lasso, or ElasticNet), Naive Bayes Classifier, GaussianNB, GaussianNBisotonic, GaussianNBsigmoid, Random Forest Classifier, GradientBoosting Classifier, XGBoost Classifer, HistGradientBoosting Classifier, Deep Neural Network Classifier, DecisionTree Classifier, Bagging Classifier, ExtraTrees Classifier, AdaBoost Classifier, CatBoost Classifier, LGBM Classifier, Support Vector Classifier, Perceptron Classifier (e.g., Multi-layer Perceptron Classifier), Deep Learning Classifier for tabular data (e.g., TabNet or TabML), PassiveAggressiveClassifier, or SGD Classifier, or an ensemble algorithm comprising a combination of any of the foregoing.

58. The method of any one of claims 1-3, 5-11, 13-21, and 23-57, wherein the machine learning algorithm is a support vector classifier, a decision tree-based ensemble classifier, or a multilayer perceptron classifier.

59. The method of any one of claims 3-8, 11-18, and 21-53, wherein the prediction model is a regression model.

60. The method of any one of claims 1-52, wherein the machine learning algorithm is an unsupervised machine learning algorithm.

61. The method of any one of claims 1-60, wherein the prediction model is a general model.

62. The method of claim 61, wherein at least one subject of the plurality of reference subjects lives in a different geographical region, is a different sex, is a different age, is from a different cultural or ethnic group, has a different diet, and/or has a different cancer type, compared to another subject of the plurality of reference subjects.

63. The method of claim 62, wherein the plurality of reference subjects are from at least 2, 3, 4, 5, 6, or more, different countries.

64. The method of any one of claims 61-63, wherein at least one subject of the plurality of reference subjects is a different sex compared to another subject of the plurality of reference subjects.

65. The method of any one of claims 1-60, wherein the prediction model is a specialized model.

66. The method of claim 65, wherein each subject of the plurality of reference subjects live in the same geographical region, are the same sex, are the same age, and/or have the same cancer type.

67. The method of claim 65 or claim 66, wherein each subject of the plurality of reference subjects are the same sex.

68. The method of any one of claims 65-67, wherein each subject of the plurality of reference subjects are male.

69. The method of any one of claims 65-67, wherein each subject of the plurality of reference subjects are female.

70. The method of any one of claims 65-69, wherein each subject of the plurality of reference subjects have the same cancer type.

71. The method of any one of claims 1-70, wherein the subject is a mammal.

72. The method of any one of claims 1-71, wherein the subject is a human.

73. The method of any one of claims 1-72, wherein the cancer is a blood cancer or is a solid tumor.

74. The method of claim 73, wherein the blood cancer is a leukemia, a lymphoma or a multiple myeloma.

75. The method of any one of claims 1-74, wherein the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

76. The method of any one of claims 1-75, wherein the cancer is prostate cancer, bladder cancer, kidney cancer, head-and-neck cancer, breast cancer or leukemia.

77. The method of any one of claims 1-76, wherein the method is noninvasive.

78. The method of any one of claims 1-77, wherein the prediction model is built by:
(i) determining the values for each of the at least one isotopic feature for the plurality of reference samples; and
(ii) using the values for each of the at least one isotopic feature for the plurality of reference samples to train the machine learning algorithm.

79. The method of any one of claims 1-78, wherein some of the plurality of reference subjects are known to have cancer, and some of the plurality of reference subjects are assumed to not have cancer.

80. The method of any one of claims 1-59 and 61-79, wherein the machine learning algorithm is trained using a plurality of labels, the plurality of labels comprising a label for each of the plurality of reference samples.

81. The method of claim 80, wherein the label indicates if the reference sample is from a reference subject known to have cancer or from a reference subject assumed to not have cancer.

82. The method of claim 80, wherein the label indicates the characteristic of the cancer in the reference subject from which the reference sample was obtained.

83. The method of claim 80 or claim 82, wherein the label indicates the level of tumor burden of the cancer in the reference subject from which the reference sample was obtained.

84. The method of any one of claims 1-83, wherein the method further comprises, prior to step (a), training the machine learning algorithm using the values for each of the at least one isotopic feature for the plurality of reference samples.

85. The method of any one of claims 1-59 and 61-84, the method further comprises, prior to step (a), training the machine learning algorithm using the plurality of labels and the values for each of the at least one isotopic feature for the plurality of reference samples.

86. The method of claim 84 or claim 85, wherein the method further comprises preprocessing the values for each of the at least one isotopic feature prior to the training the machine learning algorithm.

87. The method of claim 86, wherein the preprocessing comprises controlled imputation of missing values or replacing missing values with a zero.

88. The method of claim 86 or claim 87, wherein the preprocessing comprises data normalization.

89. The method of any one of claims 1, 2, 5-10, 13-20, 23-81, and 84-88, wherein cancer is detected in the subject if the prediction model indicates the presence of cancer.

90. The method of any one of claims 1-89, wherein the accuracy of predicting cancer by the method is greater than 50%, greater than 60%, greater than 70%, greater than 80% or greater than 90%, or greater than 95%.

91. The method of any one of claims 1-90, wherein the accuracy of predicting cancer by the method is greater than about 70%.

92. The method of any one of claims 1-91, wherein the accuracy of predicting cancer is greater than 90%, such as greater than 91%, greater than 92%, greater than 93%, greater than 94% or greater than 95%.

93. The method of any one of claims 89-92, wherein the method further comprises verification of the detected cancer by a method selected from the group consisting of a blood test, a urine test, a biopsy, an endoscopic exam, a lumbar puncture, a pap test, surgery, genetic testing, and imaging.

94. The method of any one of claims 1, 2, 5-10, 13-20, 23-81, and 84-93, wherein the method is for diagnosing cancer in the subject, wherein the subject is diagnosed with cancer if the prediction model indicates the presence of cancer.

95. The method of any one of claims 1-94, wherein the subject has one or more symptoms that indicate a cancer may be present in the subject.

96. The method of any one of claims 1, 2, 5-10, 13-20, 23-81, and 84-94, wherein the method is a preventive screening method for cancer in the subject.

97. The method of any one of claims 1, 2, 5-10, 13-20, 23-81, and 84-96, wherein if the subject is diagnosed with cancer, the subject undergoes treatment for the cancer.

98. The method of any one of claims 1, 2, 5-10, 13-20, 23-81, and 84-97, wherein if the subject is diagnosed with cancer, the method further comprises treating the subject for the cancer.

99. A method of treating a cancer in a subject, comprising:
(a) selecting a subject diagnosed with a cancer according to the method of any one of claims 1, 2, 5-10, 13-20, 23-81, and 84-98, wherein the prediction model indicates the presence of the cancer; and
(b) treating the subject for the cancer with a treatment for the cancer.

100. The method of any one of claims 1-93 and 95, wherein the method is for monitoring cancer treatment in the subject.

101. The method of any one of claims 1-93, 95, and 100, wherein the subject has been previously diagnosed with cancer and is undergoing treatment, or has been previously diagnosed with cancer and is believed to be in remission.

102. The method of claim 100 or claim 101, wherein if cancer is detected in the subject, cancer treatment for the subject is continued or re-started.

103. The method of any one of claims 100-102, wherein if cancer is detected in the subject, the method further comprises continuing or re-starting treatment of the subject for the cancer.

104. A method of treating a cancer in a subject, comprising:
(a) selecting a subject in which a cancer is detected according to the method of any one of claims 1, 2, 5-10, 13-20, 23-81, 84-93, 95, and 100-103, wherein the prediction model indicates the presence of the cancer, and the subject has been previously diagnosed with the cancer and is undergoing treatment for the cancer, or has been previously diagnosed with the cancer and is believed to be in remission; and
(b) treating the subject for the cancer with a treatment for the cancer.

105. The method of any one of claims 97-104, wherein the treatment comprises chemotherapy, radiotherapy, cryotherapy, photodynamic therapy, laser therapy, immunotherapy, targeted therapy, hormone therapy, surgical procedures to remove the cancer, stem cell transplant, bone marrow transplant, or a combination of any of the foregoing.

106. The method of any one of claims 1-105, wherein the prediction model is built and applied using a computer-program product tangibly embodied in a non-transitory machine-readable storage medium comprising instructions configured to cause one or more data processors to build and apply the prediction model.

107. A method of training a prediction model for detecting whether a subject has a cancer, the method comprising:
(a) determining, for a plurality of reference samples from a plurality of reference subjects, values for each of at least one isotopic feature, wherein the value for each of the at least one isotopic feature is: (1) a concentration of an element, (2) a concentration of an isotopic element, (3) a concentration of an isotope of the isotopic element, or (4) a normalized value of any of (1)-(3), measured in a reference sample of the plurality of reference samples; and
(b) applying the values for each of the at least one isotopic feature for the plurality of reference samples as input to train a prediction model configured to predict the presence or absence of a cancer in a subject, wherein the prediction model comprises at least one machine learning algorithm.

108. A method of training a prediction model for assessing a characteristic of a cancer in a subject, the method comprising:
(a) determining, for a plurality of reference samples from a plurality of reference subjects, values for each of at least one isotopic feature, (1) a concentration of an element, (2) a concentration of an isotopic element, (3) a concentration of an isotope of the isotopic element, or (4) a normalized value of any of (1)-(3), measured in a reference sample of the plurality of reference samples; and
(b) applying the values for each of the at least one isotopic feature for the plurality of reference samples as input for training a prediction model configured to predict a characteristic of a cancer in a subject, wherein the prediction model comprises at least one machine learning algorithm.

109. The method of claim 108, wherein the characteristic of the cancer is the level of tumor burden of the cancer in the subject.

110. The method of any one of claims 107-109, wherein the reference samples are solid samples, and wherein the determining the values for each of the at least one isotopic feature comprises mass spectrometry analysis of the solid reference sample, optionally analyzed via combustion, and/or liquid samples prepared by digestion of one or more of the solid reference samples.

111. The method of any one of claims 107-110, wherein the reference samples are solid samples, and wherein the determining the values for each of the at least one isotopic feature comprises:
(i) preparing liquid samples of one or more of the solid reference samples for analysis by mass spectrometry, wherein the liquid samples are prepared by digestion of the one or more solid reference samples;
(ii) introducing the prepared liquid samples into a mass spectrometer to detect one or more ionization products of elements and isotopic elements present in the prepared liquid samples; and
(iii) analyzing the detected one or more ionization products of elements and isotopic elements to generate values for one or more of the at least one isotopic feature for the prepared liquid samples.

112. The method of any one of claims 107-111, wherein the reference samples are solid samples, and wherein determining the values for each of the at least one isotopic feature comprises:
(i) preparing solid samples of one or more of the solid reference samples for analysis by mass spectrometry;
(ii) introducing the prepared solid samples into a mass spectrometer to detect one or more ionization products of elements and isotopic elements present in the prepared solid samples, optionally wherein the detecting comprises combustion of the prepared solid samples; and
(iii) analyzing the detected one or more ionization products of elements and isotopic elements to generate values for one or more of the at least one isotopic feature for the prepared solid samples.

113. The method of any one of claims 107-112, wherein the determining and/or training is performed by a processor of a computing device.

114. A method of training a prediction model for detecting whether a subject has a cancer, the method comprising:
(a) receiving, at one or more processors, values for each of at least one isotopic feature for a plurality of reference samples from a plurality of reference subjects, wherein the value for each of the at least one isotopic feature is: (1) a concentration of an element, (2) a concentration of an isotopic element, (3) a concentration of an isotope of the isotopic element, or (4) a normalized value of any of (1)-(3) measured in a reference sample of the plurality of reference samples; and
(b) applying the values for each of the at least one isotopic feature for the plurality of reference samples as input for training, using at least one processor, a prediction model configured to predict the presence or absence of a cancer in a subject, wherein the prediction model comprises at least one machine learning algorithm.

115. A method of training a prediction model for assessing a characteristic of a cancer in a subject, the method comprising:
(a) receiving, at one or more processors, values for each of at least one isotopic feature for a plurality of reference samples from a plurality of reference subjects, wherein the value for each of the at least one isotopic feature is: (1) a concentration of an element, (2) a concentration of an isotopic element, (3) a concentration of an isotope of the isotopic element, or (4) a normalized value of any of (1)-(3), measured in a reference sample of the plurality of reference samples; and
(b) applying the values for each of the at least one isotopic feature for the plurality of reference samples as input for training, using at least one processor, a prediction model configured to predict a characteristic of a cancer in a subject, wherein the prediction model comprises at least one machine learning algorithm.

116. The method of claim 115, wherein the characteristic of the cancer is the level of tumor burden of the cancer in the subject.

117. The method of any one of claims 114-116, wherein the values for each of the at least one isotopic feature is obtained by mass spectrometry analysis of liquid samples prepared by digestion of one or more of the reference samples and/or solid samples of one or more of the reference samples that are optionally analyzed via combustion.

118. The method of any one of claims 114-117, wherein the reference samples are solid samples, and wherein prior to step (a), the method comprises determining the values for each of the at least one isotopic feature, wherein the determining comprises:
(i) preparing liquid samples of one or more of the solid reference samples for analysis by mass spectrometry, wherein the liquid samples are prepared by digestion of the one or more solid reference samples;
(ii) introducing the prepared liquid samples into a mass spectrometer to detect one or more ionization products of elements and isotopic elements present in the prepared liquid samples; and
(iii) analyzing the detected one or more ionization products of elements and isotopic elements to generate values for one or more of the at least one isotopic feature for the prepared liquid samples.

119. The method of any one of claims 114-118, wherein the reference samples are solid samples, and wherein prior to step (a), the method comprises determining the values for each of the at least one isotopic feature, wherein the determining comprises:
(i) preparing solid samples of one or more of the solid reference samples for analysis by mass spectrometry;
(ii) introducing the prepared solid samples into a mass spectrometer to detect one or more ionization products of elements and isotopic elements present in the prepared solid samples, optionally wherein the detecting comprises combustion of the prepared solid samples; and
(iii) analyzing the detected one or more ionization products of elements and isotopic elements to generate values for one or more of the at least one isotopic feature for the prepared solid samples.

120. The method of any one of claims 111-113, 118, and 119, wherein the analyzing the detected one or more ionization products of isotopic elements is with reference to one or more ionization products of elements present in standards comprising a plurality of calibration standards and a blank standard.

121. The method of any one of claims 111-113 and 118-120, wherein the analyzing the detected one or more ionization products of elements and isotopic elements is with reference to one or more ionization products of elements and isotopic elements present in standards comprising an internal standard and/or a reference material.

122. The method of claim 120 or claim 121, wherein the mass spectrometer is further introduced with the standards, wherein the mass spectrometer detects one or more ionization products of elements and isotopic elements present in the standards and the prepared liquid or solid samples.

123. A method of training a prediction model for detecting whether a subject has a cancer, the method comprising:
(a) providing a plurality of solid reference samples obtained from a plurality of reference subjects;
(b) preparing liquid samples and/or solid samples for analysis by mass spectrometry from the plurality of solid reference samples, wherein the liquid samples are prepared by digestion of one or more of the solid reference samples;
(c) providing standards comprising a plurality of calibration standards, a blank standard, an internal standard, and/or a reference material;
(d) introducing the standards and the prepared liquid or solid samples individually into a mass spectrometer to detect one or more ionization products of elements and isotopic elements present in each of the standards and the prepared liquid or solid samples;
(e) analyzing the detected one or more ionization products of isotopic elements and elements to generate values for each of at least one isotopic feature for the plurality of reference samples, wherein the value for each of the at least one isotopic feature is: (1) a concentration of an element, (2) a concentration of an isotopic element, (3) a concentration of an isotope of the isotopic element, or (4) a normalized value of any of (1)-(3);
(f) receiving, at one or more processors, the values for each of the at least one isotopic feature; and
(g) applying the values for each of the at least one isotopic feature for the plurality of reference samples as input for training, using at least one processor, a prediction model configured to predict the presence or absence of a cancer in a subject, wherein the prediction model comprises at least one machine learning algorithm.

124. A method of training a prediction model for assessing a characteristic of a cancer in a subject, the method comprising:
(a) providing a plurality of solid reference samples obtained from a plurality of reference subjects;
(b) preparing liquid samples and/or solid samples for analysis by mass spectrometry from the plurality of solid reference samples, wherein the liquid samples are prepared by digestion of one or more of the solid reference samples;
(c) providing standards comprising a plurality of calibration standards, a blank standard, an internal standard, and/or a reference material;
(d) introducing the standards and the prepared liquid or solid samples individually into a mass spectrometer to detect one or more ionization products of elements and isotopic elements present in each of the standards and the prepared liquid or solid samples;
(e) analyzing the detected one or more ionization products of elements and isotopic elements to generate values for each of at least one isotopic feature for the plurality of reference samples, wherein the value for each of the at least one isotopic feature is: (1) a concentration of an element, (2) a concentration of an isotopic element, (3) a concentration of an isotope of the isotopic element, or (4) a normalized value of any of (1)-(3);
(f) receiving, at one or more processors, the values for each of the at least one isotopic feature; and
(g) applying the values for each of the at least one isotopic feature for the plurality of reference samples as input for training, using at least one processor, a prediction model configured to predict a characteristic of a cancer in a subject, wherein the prediction model comprises at least one machine learning algorithm.

125. The method of claim 124, wherein the characteristic of the cancer is the level of tumor burden of the cancer in the subject.

126. The method of any one of claims 107-125, wherein the plurality of reference samples each comprise a keratinous tissue, optionally a hard keratinous tissue.

127. The method of any one of claims 107-126, wherein the plurality of reference samples are noninvasive samples, optionally hair samples and/or nail samples.

128. The method of any one of claims 110-113 and 117-127, wherein the digestion is by acid digestion and/or thermal digestion.

129. The method of any one of claims 110-113 and 117-128, wherein the digestion is by acid digestion and thermal digestion.

130. The method of claim 128 or claim 129, wherein the acid digestion is with nitric acid (HNO₃).

131. The method of any one of claims 128-130, wherein the acid digestion is with nitric acid (HNO₃) and hydrogen peroxide (H₂O₂).

132. The method of any one of claims 128-131, wherein the thermal digestion is by a microwave.

133. The method of any one of claims 110-113 and 117-132, wherein the mass spectrometry is Inductively Coupled Plasma Mass Spectrometry (ICP-MS).

134. The method of claim 133, wherein the ICP-MS is carried out with kinetic energy discrimination (KED).

135. The method of any one of claims 107-134, wherein the value of each of the at least one isotopic feature is a normalized value of any of (1)-(3).

136. The method of any one of claims 107-135, wherein the value of each of the at least one isotopic feature is a normalized value of a concentration of an isotopic element.

137. The method of any one of claims 107-135, wherein the value of each of the at least one isotopic feature is a normalized value of a concentration of an isotope of the isotopic element.

138. The method of any one of claims 107-137, wherein the normalized value of each of the at least one isotopic feature is an atomic percent (atom %), fractional abundance (F), isotope ratio (R), concentration ratio (CR), or a delta value (delta), or a combination of any of the foregoing.

139. The method of any one of claims 107-138, wherein the normalized value of each of the least one isotopic feature is a fractional abundance (F).

140. The method of any one of claims 107-138, wherein the normalized value of each of the at least one isotopic feature is a delta value.

141. The method of claim 138 or claim 140, wherein the delta value is a conventional delta value, a study-specific delta value (InH), a non-similar simple-nitrogen based delta value (SimpleNδ), or a non-similar pseudo-nitrogen based delta value (PseudoNδ).

142. The method of any one of claims 107-141, wherein the at least one isotopic feature is one isotopic feature.

143. The method of any one of claims 107-142, wherein the isotope of the at least one isotopic feature comprises ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr.

144. The method of any one of claims 107-141, wherein the at least one isotopic feature is a plurality of isotopic features.

145. The method of claim 144, wherein the plurality of isotopic features is 2-100 isotopic features.

146. The method of claim 144 or claim 145, wherein the plurality of isotopic features comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or more isotopic features.

147. The method of claim 144 or claim 145, wherein the at least one isotopic feature is a plurality of isotopic features determined from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or more elements or isotopic elements.

148. The method of any one of claims 107-147, wherein the element or the isotopic element of the at least one isotopic feature comprises Li, B, Na, Mg, Al, P, K, Sc, Ca, Ti, V, Cr, Mn, Fe, Co, Ni, Cu, Zn, Ge, As, Se, Br, Rb, Sr, Y, Zr, Nb, Mo, Ru, Ag, Cd, In, Sn, I, Te, Ba, Hg, Pb, U, C, N, O, or S, or a combination of any of the foregoing.

149. The method of any one of claims 107-148, wherein the element or the isotopic element of the at least one isotopic feature comprises Ag, Al, B, Ba, Br, Ca, Co, Cr, Cu, Fe, Ge, Hg, I, K, Li, Mg, Mn, Mo, Na, Ni, Pb, Rb, Ru, Se, Sn, Sr, Te, Ti, U, V, Y, Zn, C, N, O, or S, or a combination of any of the foregoing.

150. The method of any one of claims 107-149, wherein the element or the isotopic element of the at least one isotopic feature comprises Li, B, Mg, Ca, Ti, Cr, Fe, Ni, Cu, Zn, Se, Br, Rb, Sr, Mo, Ag, Sn, Ba, Hg, Pb, U, C, N, S, or a combination of any of the foregoing.

151. The method of any one of claims 107-150, wherein the isotope of the at least one isotopic feature comprises ¹³C, ⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing.

152. The method of any one of claims 107-151, wherein the isotopic element of the at least one isotopic feature comprises Zn.

153. The method of any one of claims 107-152, wherein the isotope of the at least one isotopic feature comprises ⁶⁴Zn and/or ⁶⁶Zn.

154. The method of any one of claims 107-149, wherein the isotopic element of the at least one isotopic feature comprises N, C, S, or O.

155. The method of any one of claims 107-151 and 154, wherein the isotopic element of the at least one isotopic feature comprises ³⁴S.

156. The method of any one of claims 107-155, wherein the machine learning algorithm is a supervised machine learning algorithm.

157. The method of any one of claims 107, 108, 110-115, 117-124, and 126-156, wherein the prediction model is a classification model.

158. The method of any one of claims 107, 108, 110-115, 117-124, and 126-157, wherein the prediction model is a binary classification model.

159. The method of any one of claims 107, 108, 110-115, 117-124, and 126-157, wherein the prediction model is a multiclass classification model.

160. The method of any one of claims 107, 108, 110-115, 117-124, and 126-159, wherein the machine learning algorithm is Logistic Regression (e.g., Ridge, Lasso, or ElasticNet), Naive Bayes Classifier, GaussianNB, GaussianNBisotonic, GaussianNBsigmoid, Random Forest Classifier, GradientBoosting Classifier, XGBoost Classifer, HistGradientBoosting Classifier, Deep Neural Network Classifier, DecisionTree Classifier, Bagging Classifier, ExtraTrees Classifier, AdaBoost Classifier, CatBoost Classifier, LGBM Classifier, Support Vector Classifier, Perceptron Classifier (e.g., Multi-layer Perceptron Classifier), Deep Learning Classifier for tabular data (e.g., TabNet or TabML), PassiveAggressiveClassifier, or SGD Classifier, or an ensemble algorithm comprising a combination of any of the foregoing.

161. The method of any one of claims 107, 108, 110-115, 117-124, and 126-160, wherein the machine learning algorithm is a support vector classifier, a decision tree-based ensemble classifier, or a multilayer perceptron classifier.

162. The method of any one of claims 108-113, 115, and 125-156, wherein the prediction model is a regression model.

163. The method of any one of claims 107-155, wherein the machine learning algorithm is an unsupervised machine learning algorithm.

164. The method of any one of claims 107-163, wherein the prediction model is a general model.

165. The method of claim 164, wherein at least one subject of the plurality of reference subjects lives in a different geographical region, is a different sex, is a different age, is from a different cultural or ethnic group, has a different diet, and/or has a different cancer type, compared to another subject of the plurality of reference subjects.

166. The method of claim 165, wherein the plurality of reference subjects are from at least 2, 3, 4, 5, 6, or more, different countries.

167. The method of any one of claims 164-166, wherein at least one subject of the plurality of reference subjects is a different sex compared to another subject of the plurality of reference subjects.

168. The method of any one of claims 107-163, wherein the prediction model is a specialized model.

169. The method of claim 168, wherein each subject of the plurality of reference subjects live in the same geographical region, are the same sex, are the same age, and/or have the same cancer type.

170. The method of claim 168 or claim 169, wherein each subject of the plurality of reference subjects are the same sex.

171. The method of any one of claims 168-170, wherein each subject of the plurality of reference subjects are male.

172. The method of any one of claims 168-170, wherein each subject of the plurality of reference subjects are female.

173. The method of any one of claims 168-172, wherein each subject of the plurality of reference subjects have the same cancer type.

174. The method of any one of claims 107-173, wherein the plurality of reference subjects are mammals.

175. The method of any one of claims 107-174, wherein the plurality of reference subjects are humans.

176. The method of any one of claims 107-175, wherein the cancer is a blood cancer or is a solid tumor.

177. The method of claim 176, wherein the blood cancer is a leukemia, a lymphoma or a multiple myeloma.

178. The method of any one of claims 107-177, wherein the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

179. The method of any one of claims 107-178, wherein the cancer is prostate cancer, bladder cancer, kidney cancer, head-and-neck cancer, breast cancer or leukemia.

180. The method of any one of claims 107-179, wherein some of the plurality of reference subjects are known to have cancer, and some of the plurality of reference subjects are assumed to not have cancer.

181. The method of any one of claims 107-162 and 164-180, wherein the training the machine learning algorithm uses a plurality of labels, the plurality of labels comprising a label for each of the plurality of reference samples.

182. The method of claim 181, wherein the label indicates if the reference sample is from a reference subject known to have cancer or from a reference subject assumed to not have cancer.

183. The method of claim 181, wherein the label indicates the characteristic of the cancer in the reference subject from which the reference sample was obtained.

184. The method of claim 181 or claim 183, wherein the label indicates the level of tumor burden of the cancer in the reference subject from which the reference sample was obtained.

185. The method of any one of claims 107-184, wherein the method further comprises preprocessing the values for each of the at least one isotopic feature prior to the training the machine learning algorithm.

186. The method of claim 185, wherein the preprocessing comprises controlled imputation of missing values or replacing missing values with a zero.

187. The method of claim 185 or claim 186, wherein the preprocessing comprises data normalization.

188. The method of any one of claims 107, 110-114, 117-123, 126-182, and 185-187, wherein cancer is detected in the subject if the prediction model indicates the presence of cancer.

189. The method of any one of claims 107-188, wherein the prediction model is built using a computer-program product tangibly embodied in a non-transitory machine-readable storage medium comprising instructions configured to cause one or more data processors to build the prediction model.

190. A method for determining a value of at least one isotopic feature of an isotope in a solid sample from a subject, the method comprising:
(a) detecting, by mass spectrometry, one or more ionization products of isotopic elements present in a solid sample from a subject, optionally wherein the solid sample is analyzed via combustion, or in a liquid sample prepared by digestion of the solid sample, wherein the one or more ionization products are at least one isotope selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or a combination of any of the foregoing; and
(b) determining a value of at least one isotopic feature, wherein the at least one isotopic feature is a concentration of the at least one isotope or a normalized value thereof.

191. The method of claim 190, wherein the value of each of the at least one isotopic feature is a normalized value of the concentration of the at least one isotope.

192. The method of claim 190 or claim 191, further comprising normalizing the value of each of the at least one isotopic feature.

193. The method of any one of claims 190-192, wherein the normalized value of each of the at least one isotopic feature is an atomic percent (atom %), fractional abundance (F), isotope ratio (R), or a delta value (delta), or a combination of any of the foregoing.

194. A method for determining a value of at least one isotopic feature of an isotope in a solid sample from a subject, the method comprising:
(a) detecting, by mass spectrometry, one or more ionization products of isotopic elements present in a solid sample from a subject, optionally wherein the solid sample is analyzed via combustion, or in a liquid sample prepared by digestion of the solid sample, wherein the one or more ionization products are at least one isotope selected from ³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or a combination of any of the foregoing;
(b) determining a value of at least one isotopic feature, wherein the at least one isotopic feature is a concentration of the at least one isotope;
(c) normalizing the value the concentration of the at least one isotope, wherein the normalized value of each of the at least one isotopic feature is an atomic percent (atom %), fractional abundance (F), isotope ratio (R), or a delta value (delta), or a combination of any of the foregoing.

195. The method of any one of claims 190-194, wherein the normalized value of each of the least one isotopic feature is a fractional abundance (F).

196. The method of any one of claims 190-195, wherein the normalized value of each of the at least one isotopic feature is a delta value.

197. The method of any one of claims 193, 194, and 196, wherein the delta value is a conventional delta value, a study-specific delta value (InH), a non-similar simple-nitrogen based delta value (SimpleNδ), or a non-similar pseudo-nitrogen based delta value (PseudoNδ).

198. The method of any one of claims 190-197, wherein the determining the value of each of the at least one isotopic feature is with reference to one or more isotopic feature present in standards comprising a plurality of calibration standards and a blank standard.

199. The method of any one of claims 190-198, wherein the determining the value of each of the at least one isotopic feature is with reference to one or more isotopic feature present in standards comprising an internal standard and/or a reference material.

200. The method of claim 198 or claim 199, wherein the method further comprises introducing the standards into the mass spectrometer, wherein the mass spectrometer detects one or more ionization products of isotopic elements present in the standards and the prepared liquid or solid sample.

201. The method of any one of claims 190-200, wherein the sample comprises a keratinous tissue, optionally a hard keratinous tissue.

202. The method of any one of claims 190-201, wherein the sample is a noninvasive sample, optionally a hair sample or a nail sample.

203. The method of any one of claims 190-202, wherein the digestion is by acid digestion and/or thermal digestion.

204. The method of any one of claims 190-203, wherein the digestion is by acid digestion and thermal digestion.

205. The method of claim 203 or claim 204, wherein the acid digestion is with nitric acid (HNO₃).

206. The method of any one of claims 203-205, wherein the acid digestion is with nitric acid (HNO₃) and hydrogen peroxide (H₂O₂).

207. The method of any one of claims 203-206, wherein the thermal digestion is by a microwave.

208. The method of any one of claims 190-207, wherein the mass spectrometry is Inductively Coupled Plasma Mass Spectrometry (ICP-MS).

209. The method of claim 208, wherein the ICP-MS is carried out with kinetic energy discrimination (KED).

210. The method of any one of claims 190-209, wherein the at least one isotope is one isotope.

211. The method of any one of claims 190-210, wherein the at least one isotope is ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr.

212. The method of any one of claims 190-210, wherein the isotope is ⁶⁴Zn, ⁶⁶Zn, or ³⁴S.

213. The method of any one of claims 190-212, wherein the at least one isotope is a plurality of isotopes.

214. The method of claim 213, wherein the plurality of isotopes is 2-59 isotopes.

215. The method of claim 213 or claim 214, wherein the at least one isotope is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 isotopes.

216. The method of any one of claims 190-215, wherein the subject is suspected of having cancer.

217. The method of any one of claims 190-216, further comprising selecting a subject that is suspected of having a cancer.

218. The method of any one of claims 190-217, wherein the subject has one or more symptoms that indicate a cancer may be present in the subject.

219. The method of any one of claims 190-218, wherein the cancer is a blood cancer or is a solid tumor.

220. The method of claim 219, wherein the blood cancer is a leukemia, a lymphoma or a multiple myeloma.

221. The method of any one of claims 216-220, wherein the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

222. The method of any one of claims 216-221, wherein the cancer is prostate cancer, bladder cancer, kidney cancer, head-and-neck cancer, breast cancer or leukemia.

223. The method of any one of claims 190-222, wherein the subject is a mammal.

224. The method of any one of claims 190-223, wherein the subject is a human.

225. The method of any one of claims 190-224, wherein the method is noninvasive.

226. A kit for determining a value of at least one isotopic feature in a sample from a subject, wherein the at least one isotopic feature is a concentration of at least one isotope or a normalized value thereof, wherein the at least one isotope is selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, ²³⁸U, or any combination of the foregoing, and wherein the kit comprises:
(i) a reference material comprising a verified standard for the at least one isotope;
(ii) packaging material; and
(iii) instructions for using the kit, wherein the instructions are for determining a value of the at least one isotopic feature.

227. The kit of claim 226, wherein the value of each of the at least one isotopic feature is a normalized value of the concentration of the at least one isotope.

228. The kit of claim 226 or claim 227, wherein the normalized value of each of the at least one isotopic feature is an atomic percent (atom %), fractional abundance (F), isotope ratio (R), or a delta value (delta), or a combination of any of the foregoing.

229. The kit of any one of claims 226-228, wherein the normalized value of each of the least one isotopic feature is a fractional abundance (F).

230. The kit of any one of claims 226-228, wherein the normalized value of each of the at least one isotopic feature is a delta value.

231. The kit of claim 228 or claim 230, wherein the delta value is a conventional delta value, a study-specific delta value (InH), a non-similar simple-nitrogen based delta value (SimpleNδ), or a non-similar pseudo-nitrogen based delta value (PseudoNδ).

232. The kit of any one of claims 226-231, wherein the value of each of the at least one isotopic feature is with reference to one or more isotopic feature present in the reference material.

233. The kit of any one of claims 226-232, wherein the kit further comprises a calibration standard, a blank standard, and/or an internal standard.

234. The kit of any one of claims 226-233, wherein the sample comprises a keratinous tissue, optionally a hard keratinous tissue.

235. The kit of any one of claims 226-234, wherein the sample is a noninvasive sample, optionally a hair sample or a nail sample.

236. The kit of any one of claims 226-235, wherein the sample is a digested sample.

237. The kit of any one of claims 226-236, wherein the kit comprises instructions for digesting the sample.

238. The kit of claim 236 or claim 237, wherein the digesting is by acid digestion and/or thermal digestion.

239. The kit of any one of claims 236-238, wherein digesting by acid digestion and thermal digestion.

240. The kit of claim 238 or claim 239, wherein the acid digestion is with nitric acid (HNO₃).

241. The kit of any one of claims 238-240, wherein the acid digestion is with nitric acid (HNO₃) and hydrogen peroxide (H₂O₂).

242. The kit of any one of claims 238-241, wherein the thermal digestion is by a microwave.

243. The kit of any one of claims 226-242, wherein the at least one isotope is one isotope.

244. The kit of any one of claims 226-242, wherein the at least one isotope is ⁶⁶Zn, ⁶⁴Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr

245. The kit of any one of claims 226-243, wherein the at least one isotope is ⁶⁴Zn, ⁶⁶Zn, or ³⁴S.

246. The kit of any one of claims 226-245, wherein the at least one isotope is a plurality of isotopes.

247. The kit of claim 246, wherein the plurality of isotopes is 2-59 isotopic features.

248. The kit of claim 246 or claim 247, wherein the plurality of isotopes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 isotopic features.

249. The kit of any one of claims 246-248, wherein the plurality of isotopes is a concentration of ⁶⁴Zn, ⁶⁶Zn, and/or ³⁴S.

250. The kit of any one of claims 226-249, wherein the subject is suspected of having cancer.

251. The kit of any one of claims 226-250, wherein the subject has one or more symptoms that indicate a cancer may be present in the subject.

252. The kit of claim 250 or claim 251, wherein the cancer is a blood cancer or is a solid tumor.

253. The kit of claim 252, wherein the blood cancer is a leukemia, a lymphoma or a multiple myeloma.

254. The kit of any one of claims 250-253, wherein the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

255. The kit of any one of claims 250-254, wherein the cancer is prostate cancer, bladder cancer, kidney cancer, head-and-neck cancer, breast cancer or leukemia.

256. The kit of any one of claims 226-255, wherein the subject is a mammal.

257. The kit of any one of claims 226-256, wherein the subject is a human.

258. A method for determining the value of the at least one isotopic feature from the sample from the subject, comprising using the kit of any one of claims 226-257 according to the instructions.

259. An isotope profile identified by mass spectrometry, wherein the isotope profile is from a solid sample from a subject, wherein the isotope profile comprises a value of at least one isotopic feature of at least one isotope selected from ¹³C, ³⁴S, ¹⁰B, ¹¹B, ²⁴Mg ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se ⁷⁹Br, ⁸¹Br, ⁸⁵Rb, ⁸⁷Rb, ⁸⁴Sr ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷P, ²⁰⁸Pb, ²³⁵U, or ²³⁸U, or any combination of the foregoing, and wherein the at least one isotopic feature is a concentration of the at least one isotope.

260. An isotope profile identified by mass spectrometry, wherein the isotope profile is from a solid sample from a subject, wherein the isotope profile comprises a value of at least one isotopic feature of at least one isotope selected from ¹³C, ³⁴S, ¹⁰B, ¹¹B, ²⁴Mg, ²⁵Mg, ²⁶Mg, ⁴³Ca, ⁴⁴Ca, ⁴⁶Ti, ⁴⁷Ti, ⁴⁸Ti, ⁴⁹Ti, ⁵⁰Cr, ⁵²Cr, ⁵³Cr, ⁵⁶Fe, ⁵⁷Fe, ⁵⁸Ni, ⁶⁰Ni, ⁶¹Ni, ⁶²Ni, ⁶³Cu, ⁶⁵Cu, ⁶⁴Zn, ⁶⁶Zn, ⁶⁷Zn, ⁶⁸Zn, ⁷⁷Se, ⁷⁸Se, ⁸²Se, ⁷⁹Br, ⁸¹Br, ⁸⁵Rb, ⁸⁷Rb, ⁸⁴Sr, ⁸⁶Sr, ⁸⁷Sr, ⁸⁸Sr, ¹⁰⁷Ag, ¹⁰⁹Ag, ¹¹⁶Sn, ¹¹⁷Sn, ¹¹⁸Sn, ¹¹⁹Sn, ¹²⁰Sn, ¹³⁵Ba, ¹³⁶Ba, ¹³⁷Ba, ¹³⁸Ba, ¹⁹⁹Hg, ²⁰⁰Hg, ²⁰¹Hg, ²⁰²Hg, ²⁰⁶Pb, ²⁰⁷Pb, ²⁰⁸Pb, ²³⁵U, or ²³⁸U, or any combination of the foregoing, and wherein the at least one isotopic feature is a concentration of the at least one isotope or a normalized value of the concentration of the at least one isotope.

261. The isotope profile of claim 260, wherein the value of the at least one isotopic feature is a normalized value of the concentration of the at least one isotope.

262. The isotope profile of claim 260 or claim 261, wherein the normalized value is an atomic percent (atom %), fractional abundance (F), isotope ratio (R), or a delta value (delta), or a combination of any of the foregoing.

263. The isotope profile of any one of claims 260-262, wherein the normalized value is a fractional abundance (F).

264. The isotope profile of any one of claims 260-262, wherein the normalized value is a delta value.

265. The isotope profile of claim 262 or claim 264, wherein the delta value is a conventional delta value, a study-specific delta value (InH), a non-similar simple-nitrogen based delta value (SimpleNδ), or a non-similar pseudo-nitrogen based delta value (PseudoNδ).

266. The isotope profile of any one of claims 259-265, wherein the solid sample comprises a keratinous tissue, optionally a hard keratinous tissue.

267. The isotope profile of any one of claims 259-266, wherein the solid sample is a noninvasive sample, optionally a hair sample or a nail sample.

268. The isotope profile of any one of claims 259-267, wherein the solid sample is digested into a prepared liquid sample for mass spectrometry.

269. The isotope profile of claim 268, wherein the digestion is by acid digestion and/or thermal digestion.

270. The isotope profile of claim 268 or claim 269, wherein the digestion is by acid digestion and thermal digestion.

271. The isotope profile of claim 269 or claim 270, wherein the acid digestion is with nitric acid (HNO₃).

272. The isotope profile of any one of claims 269-271, wherein the acid digestion is with nitric acid (HNO₃) and hydrogen peroxide (H₂O₂).

273. The isotope profile of any one of claims 269-272, wherein the thermal digestion is by a microwave.

274. The isotope profile of any one of claims 259-273, wherein the mass spectrometry is Inductively Coupled Plasma Mass Spectrometry (ICP-MS).

275. The isotope profile of claim 274, wherein the ICP-MS is carried out with kinetic energy discrimination (KED).

276. The isotope profile of any one of claims 259-275, wherein the at least one isotope is one isotope.

277. The isotope profile of any one of claims 259-276, wherein the at least one isotope is ⁶⁶Zn, ⁶⁷Zn, ⁶⁸Zn, ⁸⁷Sr, ⁸⁸Sr, ⁸⁶Sr, ⁴⁷Ti, ⁴⁸Ti, ⁴⁶Ti, and/or ⁵⁰Cr.

278. The isotope profile of any one of claims 259-276, wherein the at least one isotope is ⁶⁴Zn, ⁶⁶Zn, or ³⁴S.

279. The isotope profile of any one of claims 259-275, wherein the at least one isotope is a plurality of isotopes.

280. The isotope profile of claim 279, wherein the plurality of isotopes is 2-59 isotopes.

281. The isotope profile of claim 279 or claim 280, wherein the plurality of isotopes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 isotopes.

282. The isotope profile of any one of claims 279-281, wherein the plurality of isotopes comprises a concentration of ⁴Zn, ⁶⁶Zn, and/or ³⁴S.

283. The isotope profile of any one of claims 259-282, wherein the subject is suspected of having cancer.

284. The isotope profile of any one of claims 259-283, wherein the subject has one or more symptoms that indicate a cancer may be present in the subject.

285. The isotope profile of claim 283 or claim 284, wherein the cancer is a blood cancer or is a solid tumor.

286. The isotope profile of claim 285, wherein the blood cancer is a leukemia, a lymphoma or a multiple myeloma.

287. The isotope profile of any one of claims 283-286, wherein the cancer is selected from the group consisting of lung cancer, thyroid cancer, ovarian cancer, colorectal cancer, prostate cancer, cancer of the pancreas, cancer of the esophagus, liver cancer, breast cancer, skin cancer, mesothelioma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, multiple myeloma, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, gall bladder cancer, lung carcinoma, small cell lung carcinoma, bladder carcinoma, epithelial carcinoma, glioma, astrocytoma, glioblastoma multiforme, medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, retinoblastoma, kidney cancer, hepatocellular carcinoma (HCC), hepatoblastoma, colorectal carcinoma, colon cancer, rectal cancer, anal cancer, non-small cell lung cancer (NSCLC), small cell lung cancer (SCLC), ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, Ewing sarcoma, anaplastic thyroid cancer, tonsil cancer, adrenocortical carcinoma, pancreatic ductal carcinoma, pancreatic adenocarcinoma, gastrointestinal/stomach (GIST) cancer, lymphoma, squamous cell carcinoma of the head and neck (SCCHN), salivary gland cancer, brain cancer, and neurofibromatosis-1 associated malignant peripheral nerve sheath tumors (MPNST).

288. The isotope profile of any one of claims 283-287, wherein the cancer is prostate cancer, bladder cancer, kidney cancer, head-and-neck cancer, breast cancer or leukemia.

289. The isotope profile of any one of claims 259-288, wherein the subject is a mammal.

290. The isotope profile of any one of claims 259-289, wherein the subject is a human.

291. A method for determining whether a subject has a cancer, the method comprising the step of comparing one or more isotopic features of an isotope profile of any one of claims 259-290 to one or more corresponding isotopic features of (i) individuals characterized as having cancer, (ii) individuals characterized as being cancer free, or (iii) both individuals characterized as having cancer and individuals characterized as being cancer free.

292. The method of claim 291, wherein the step of comparing is carried out by inputting the value for one or more isotopic features from the isotope profile into a prediction model configured to predict the presence or absence of a cancer in the subject, wherein the prediction model comprises at least one machine learning algorithm trained using values for one or more corresponding isotopic features from a plurality of reference samples from a plurality of reference subjects.

293. The method of claim 292, wherein the prediction model determines whether the subject has cancer.

294. The method of claim 292 or claim 293, wherein the plurality of reference subjects comprises (i) individuals characterized as having cancer, (ii) individuals characterized as being cancer free, or (iii) both individuals characterized as having cancer and individuals characterized as being cancer free.

295. The method of any one of claims 291-294, wherein the isotope profile is received as a data file that is communicated electronically from a physician, laboratory, or other service provider.

296. The method of any one of claims 291-295, wherein the determination of whether the subject has cancer is communicated electronically to a physician, laboratory, or other service provider, optionally wherein the electronic communication comprises (i) a comparison of isotopic features of the subject's isotope profile with corresponding isotopic features from a plurality of reference samples from a plurality of reference subjects, (ii) a determination of the probability that the subject has cancer, or (iii) both (i) and (ii).

297. The method of any one of claims 291-296, wherein the isotope profile comprises at least one normalized value for the concentration of at least one isotope of the isotope profile.

298. The method of claim 297, wherein the normalized value is an atomic percent (atom %), fractional abundance (F), isotope ratio (R), or a delta value (delta), or a combination of any of the foregoing.

299. The method of claim 297 or claim 298, wherein the normalized value is a fractional abundance (F).

300. The method of claim 297 or claim 298, wherein the normalized value is a delta value.

301. The method of claim 300, wherein the delta value is a conventional delta value, a study-specific delta value (InH), a non-similar simple-nitrogen based delta value (SimpleNδ), or a non-similar pseudo-nitrogen based delta value (PseudoNδ).

302. A method for determining whether a subject has a cancer, the method comprising:
(a) determining a normalized value for the concentration of the at least one isotope of the isotope profile of any one of claims 259-290,
(b) determining, based on the normalized value, whether the subject has a cancer.

303. The method of claim 302, wherein the normalized value is an atomic percent (atom %), fractional abundance (F), isotope ratio (R), or a delta value (delta), or a combination of any of the foregoing.

304. The method of claim 302 or claim 303, wherein the normalized value is a fractional abundance (F).

305. The method of claim 302 or claim 303, wherein the normalized value is a delta value.

306. The method of claim 303 or claim 305, wherein the delta value is a conventional delta value, a study-specific delta value (InH), a non-similar simple-nitrogen based delta value (SimpleNδ), or a non-similar pseudo-nitrogen based delta value (PseudoNδ).

307. A method of treating a cancer in a subject, comprising:
(a) selecting a subject detected as having a cancer according to the method of any one of claims 291-306; and
(b) treating the subject for the cancer with a treatment for the cancer.

308. The method of claim 307, wherein the treatment comprises chemotherapy, radiotherapy, cryotherapy, photodynamic therapy, laser therapy, immunotherapy, targeted therapy, hormone therapy, surgical procedures to remove the cancer, stem cell transplant, bone marrow transplant, or a combination of any of the foregoing.
